(19) United States
(12) Patent Application Publication
(10) Pub. No.: US 2026/0048116 A1
(43) Pub. Date: Feb. 19, 2026

(54) PROTEIN ANTIGENS AND USES THEREOF

(71) Applicant: BioNTech US Inc., Cambridge, MA (US)

(72) Inventor: Michael Steven Rooney, Boston, MA (US)

(21) Appl. No.: 19/178,348

(22) Filed: Apr. 14, 2025

Related U.S. Application Data
(63) Continuation of application No. 16/500,707, now Pat. No. 12,303,561.
(60) Provisional application No. 62/480,593, filed on Apr. 3, 2017.
(60) Provisional application No. 62/480,596, filed on Apr. 3, 2017.
(60) Provisional application No. 62/480,597, filed on Apr. 3, 2017.

(51) Int. Cl. A61K 39/245; A61K 39/00; A61K 40/19; A61K 40/24; A61K 40/42; A61K 45/06; A61P 35/00

(52) U.S. Cl. CPC A61K 39/245; A61K 39/0011; A61K 40/19; A61K 40/24; A61K 40/42; A61K 45/06; A61P 35/00

(57) ABSTRACT

The filed of the present invention relates to immunotherapeutic peptides, peptide binding agents, and their use, for example, in the immunotherapy of cancer.

### CROSS REFERENCE

This application is a continuation of U.S. application Ser. No. 16/500,707, filed Oct. 3, 2019, which is a National Stage Entry of International Application No. PCT/US2018/025933, filed Apr. 3, 2018, which claims priority to U.S. Provisional Application No. 62/480,593, filed Apr. 3, 2017, U.S. Provisional Application No. 62/480,596, filed Apr. 3, 2017, and U.S. Provisional Application No. 62/480,597, filed Apr. 3, 2017, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jun. 1, 2023, is named 50401-705.7611_SL.xml and is 836,830 bytes in size.

### FIELD

The field of the present invention relates to immunotherapeutic peptides, nucleic acids encoding the peptides, peptide binding agents, and their use, for example, in the immunotherapy of cancer. In one aspect, the invention provides non-mutated protein epitopes expressed in cancer cells, useful alone or in combination with other tumor-associated peptides, anti-cancer, or immunomodulatory agents to treat cancer.

### BACKGROUND

Tumor vaccines are typically composed of tumor antigens and immunostimulatory molecules (e.g., adjuvants, cytokines or TLR ligands) that work together to induce antigen-specific cytotoxic T cells (CTLs) that recognize and lyse tumor cells. Such vaccines contain either shared tissue restricted tumor antigens or a mixture of shared and patient-specific antigens in the form of whole tumor cell preparations. The shared tissue restricted tumor antigens are ideally immunogenic proteins with selective expression in tumors across many individuals and are commonly delivered to patients as synthetic peptides or recombinant proteins. In contrast, whole tumor cell preparations are delivered to patients as autologous irradiated cells, cell lysates, cell fusions, heat-shock protein preparations or total mRNA. Since whole tumor cells are isolated from the autologous patient, the cells may include patient-specific tumor antigens as well as shared tumor antigens. Finally, there is a third class of tumor antigens, neoantigens, which consists of proteins with tumor-specific mutations (which can be patient-specific or shared) that result in altered amino acid sequences. Accordingly, there is still a need for developing additional cancer therapeutics.

### SUMMARY

Provided herein an isolated antigenic peptide comprising an epitope from a sequence in Table 1 or 2. The present disclosure is also directed to an isolated antigenic peptide 100 amino acids or less in length which comprises an epitope from a sequence in Table 1 or 2. The present disclosure is also directed to an isolated antigenic peptide comprising an epitope from a sequence in Table 3 or 4. The present disclosure is also directed to an isolated antigenic peptide 100 amino acids or less in length which comprises an epitope from a sequence in Table 3 or 4. The present disclosure is also directed to an isolated antigenic peptide comprising an epitope from a sequence in Table 5 or 6. The present disclosure is also directed to an isolated antigenic peptide 100 amino acids or less in length which comprises an epitope from a sequence in Table 5 or 6.

In one embodiment, the isolated antigenic peptide is a retroviral antigen. In another embodiment, the isolated antigenic peptide is a non-mutated overexpressed antigen. In another embodiment, the isolated antigenic peptide is a viral antigen.

In one embodiment, the isolated antigenic peptide is between about 5 to about 50 amino acids in length. In another embodiment, the isolated antigenic peptide is between about 15 to about 35 amino acids in length. In another embodiment, the isolated antigenic peptide is about 15 amino acids or less in length. In another embodiment, the isolated antigenic peptide is between about 8 and about 11 amino acids in length. In another embodiment, the isolated antigenic peptide is 9 or 10 amino acids in length. In one embodiment, the isolated antigenic peptide binds major histocompatibility complex (MHC) class I. In another embodiment, the isolated antigenic peptide binds MHC class I with a binding affinity of less than about 500 nM.

In one embodiment, the isolated antigenic peptide is about 30 amino acids or less in length. In another embodiment, the isolated antigenic peptide is between about 6 and about 25 amino acids in length. In another embodiment, the isolated antigenic peptide is between about 15 and about 24 amino acids in length. In another embodiment, the isolated antigenic peptide is between about 9 and about 15 amino acids in length. In one embodiment, the isolated antigenic peptide binds MHC class II. In another embodiment, the isolated antigenic peptide binds MHC class II with a binding affinity of less than about 1000 nM.

In one embodiment, the isolated antigenic peptide further comprises flanking amino acids. In another embodiment, the flanking amino acids are not native flanking amino acids. In one embodiment, the isolated antigenic peptide is linked to at least a second antigenic peptide. In another embodiment, the peptides are linked using a poly-glycine or poly-serine linker. In another embodiment, the second antigenic peptide binds MHC class I or class II with a binding affinity of less than about 1000 nM. In another embodiment, the second antigenic peptide binds MHC class I or class II with a binding affinity of less than about 500 nM. In another embodiment, both of the epitopes bind to human leukocyte antigen (HLA)-A, -B, -C, -DP, -DQ, or -DR. In another embodiment, the isolated antigenic peptide binds a class I HLA and the second antigenic peptide binds a class II HLA. In another embodiment, the isolated antigenic peptide binds a class II HLA and the second antigenic peptide binds a class I HLA.

In one embodiment, the isolated antigenic peptide further comprises modifications which increase in vivo half-life, cellular targeting, antigen uptake, antigen processing, MHC affinity, MHC stability, or antigen presentation. In another embodiment, the modification is conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. In one embodiment, the cells that are targeted are antigen presenting cells. In another embodiment, the antigen presenting cells are dendritic cells. In another embodiment, the dendritic cells are targeted using DEC205, XCR1, CD197, CD80, CD86, CD123, CD209, CD273, CD283, CD289, CD184, CD85h, CD85j, CD85k, CD85d, CD85g, CD85a, CD141, CD11c, CD83, TSLP receptor, or CD1a marker. In another embodiment, the dendritic cells are targeted using the CD141, DEC205, or XCR1 marker.

In one embodiment, provided herein is an in vivo delivery system comprising an isolated antigenic peptide described herein. In another embodiment, the delivery system includes cell-penetrating peptides, nanoparticulate encapsulation, virus like particles, or liposomes. In another embodiment, the cell-penetrating peptide is TAT peptide, herpes simplex virus VP22, transportan, or Antp.

In one embodiment, provided herein is a cell comprising an isolated antigenic peptide described herein. In another embodiment, the cell is an antigen presenting cell. In another embodiment, the cell is a dendritic cell.

In one embodiment, provided herein is a composition comprising an isolated antigenic peptide described herein. In another embodiment, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the isolated antigenic peptides comprising a tumor-specific epitope defined in Table 1 or 2. In another embodiment, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the isolated antigenic peptides comprising a tumor-specific epitope defined in Table 3 or 4. In another embodiment, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the isolated antigenic peptides comprising a tumor-specific epitope defined in Table 5 or 6. In another embodiment, the composition comprises between 2 and 20 antigenic peptides. In another embodiment, the composition further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, at least 26, at least 27, at least 28, at least 29, or at least 30 additional antigenic peptides. In another embodiment, the composition comprises between about 4 and about 20 additional antigenic peptides. In another embodiment, the additional antigenic peptide is specific for an individual patient's tumor. In another embodiment, an antigenic peptide is selected by identifying differences in expression between the transcriptome or proteome of the patient's tumor sample and the transcriptome or proteome of a non-tumor sample. In another embodiment, the samples are fresh or formalin-fixed paraffin embedded tumor tissues, freshly isolated cells, or circulating tumor cells. In some embodiments, the sequences of the antigenic peptides are determined by Next Generation Sequencing.

In one embodiment, provided herein is an isolated polynucleotide encoding the isolated antigenic peptide described herein. In another embodiment, the isolated polynucleotide is RNA, optionally a self-amplifying RNA. In another embodiment, the RNA is modified to increase stability, increase cellular targeting, increase translation efficiency, adjuvanticity, cytosol accessibility, and/or decrease cytotoxicity. In another embodiment, the modification is conjugation to a carrier protein, conjugation to a ligand, conjugation to an antibody, codon optimization, increased GC-content, incorporation of modified nucleosides, incorporation of 5′-cap or cap analog, and/or incorporation of an unmasked poly-A sequence.

In one embodiment, provided herein is a cell comprising a polynucleotide described herein.

In one embodiment, provided herein is a vector comprising a polynucleotide described herein. In another embodiment, the polynucleotide is operably linked to a promoter. In another embodiment, the vector is a self-amplifying RNA replicon, plasmid, phage, transposon, cosmid, virus, or virion. In another embodiment, the vector is an adeno-associated virus, herpesvirus, lentivirus, or pseudotypes thereof.

In one embodiment, provided herein is an in vivo delivery system comprising an isolated polynucleotide described herein. In another embodiment, the delivery system includes spherical nucleic acids, viruses, virus-like particles, plasmids, bacterial plasmids, or nanoparticles.

In one embodiment, provided herein is a cell comprising a vector or delivery system described herein. In another embodiment, the cell is an antigen presenting cell. In another embodiment, the cell is a dendritic cell. In another embodiment, the cell is an immature dendritic cell.

In one embodiment, provided herein is a composition comprising at least one polynucleotide described herein. In another embodiment, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the isolated polynucleotides. In another embodiment, the composition comprises between about 2 and about 20 polynucleotides. In another embodiment, the composition further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 additional antigenic polynucleotides encoding for additional antigenic peptides. In another embodiment, the composition comprises between about 4 and about 20 additional antigenic polynucleotides. In another embodiment, the isolated polynucleotides and the additional antigenic polynucleotides are linked. In another embodiment, the polynucleotides are linked using nucleic acids that encode a poly-glycine or poly-serine linker. In another embodiment, at least one of the additional antigenic peptide is specific for an individual patient's tumor. In another embodiment, an antigenic peptide is selected by identifying differences in expression between the transcriptome or proteome of the patient's tumor sample and the transcriptome or proteome of a non-tumor sample. In another embodiment, the samples are fresh or formalin-fixed paraffin embedded tumor tissues, freshly isolated cells, or circulating tumor cells. In some embodiments, the sequences of the antigenic peptides are determined by Next Generation Sequencing.

In one embodiment, provided herein is a T cell receptor (TCR) capable of binding at least one antigenic peptide described herein. In another embodiment, the TCR is capable of binding the isolated antigenic peptide in the context of MHC class I or class II.

In one embodiment, provided herein is a chimeric antigen receptor comprising: (i) a T cell activation molecule; (ii) a transmembrane region; and (iii) an antigen recognition moiety capable of binding an isolated antigenic peptide described herein. In another embodiment, CD3-zeta is the T cell activation molecule. In another embodiment, the chimeric antigen receptor further comprises at least one costimulatory signaling domain. In another embodiment, the signaling domain is CD28, 4-1BB, ICOS, OX40, ITAM, or Fc epsilon RI-gamma. In another embodiment, the antigen recognition moiety is capable of binding the isolated antigenic peptide in the context of MHC class I or class II. In another embodiment, the chimeric antigen receptor comprises the CD3-zeta, CD28, CTLA-4, ICOS, BTLA, KIR, LAG3, CD137, OX40, CD27, CD40L, Tim-3, A2aR, or PD-1 transmembrane region. In another embodiment, the tumor-specific epitope is located in the extracellular domain of a tumor associated polypeptide.

In one embodiment, provided herein is a T cell comprising the T cell receptor or chimeric antigen receptor described herein. In one embodiment, the T cell is a helper or cytotoxic T cell.

In one embodiment, provided herein is a nucleic acid comprising a promoter operably linked to a polynucleotide encoding a T cell receptor described herein. In another embodiment, the TCR is capable of binding the at least one antigenic peptide in the context of major histocompatibility complex (MHC) class I or class II. In one embodiment, the nucleic acid comprises a promoter operably linked to a polynucleotide encoding a chimeric antigen receptor described herein. In another embodiment, the antigen recognition moiety is capable of binding the at least one antigenic peptide in the context of major histocompatibility complex (MHC) class I or class II. In another embodiment, the tumor-specific epitope is located in the extracellular domain of a tumor associated polypeptide. In another embodiment, the nucleic acid comprises the CD3-zeta, CD28, CTLA-4, ICOS, BTLA, KIR, LAG3, CD137, OX40, CD27, CD40L, Tim-3, A2aR, or PD-1 transmembrane region.

In one embodiment, provided herein is an antibody capable of binding at least one antigenic peptide listed in Table 1 or 2. In another embodiment, provided herein is an antibody capable of binding at least one antigenic peptide listed in Table 3 or 4. In another embodiment, provided herein is an antibody capable of binding at least one antigenic peptide listed in Table 5 or 6. In another embodiment, the at least one antigenic peptide listed in Table 1 or 2 is a retroviral antigenic peptide. In another embodiment, the at least one antigenic peptide listed in Table 3 or 4 is a non-mutated overexpressed antigenic peptide. In another embodiment, the at least one antigenic peptide listed in Table 5 or 6 is a viral antigenic peptide.

In one embodiment, provided herein is a modified cell transfected or transduced with a nucleic acid described herein. In one embodiment, the modified cell is a T cell, tumor infiltrating lymphocyte, NK-T cell, TCR-expressing cell, CD4+ T cell, CD8+ T cell, or NK cell.

In one embodiment, provided herein is a composition comprising a T cell receptor or chimeric antigen receptor described herein. In another embodiment, a composition comprises autologous patient T cells containing a T cell receptor or chimeric antigen receptor described herein. In another embodiment, the composition further comprises an immune checkpoint inhibitor. In another embodiment, the composition further comprises at least two immune checkpoint inhibitors. In another embodiment, each of the immune checkpoint inhibitors inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof. In another embodiment, each of the immune checkpoint inhibitors interacts with a ligand of a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof.

In one embodiment, the composition further comprises an immune modulator or adjuvant. In another embodiment, the immune modulator is a co-stimulatory ligand, a TNF ligand, an Ig superfamily ligand, CD28, CD80, CD86, ICOS, CD40L, OX40, CD27, GITR, CD30, DR3, CD69, or 4-1BB. In another embodiment, the immune modulator is at least one cancer cell or cancer cell extract. In another embodiment, the cancer cell is autologous to the subject in need of the composition. In another embodiment, the cancer cell has undergone lysis or been exposed to UV radiation. In another embodiment, the composition further comprises an adjuvant. In another embodiment, the adjuvant is selected from the group consisting of: Poly(I:C), Poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312 VG, Montanide ISA 206 VG, Montanide ISA 50 V2, Montanide ISA 51 VG, OK-432, OM-174, OM-197-MP-EC, ISA-TLR2 agonist, ONTAK, PepTel®. vector system, PLG microparticles, resiquimod, SRL172, virosomes and other virus-like particles, YF-17D, VEGF trap, R848, beta-glucan, Pam3Cys, Pam3CSK4, acrylic or methacrylic polymers, copolymers of maleic anhydride, and QS21 stimulon. In another embodiment, the adjuvant induces a humoral when administered to a subject. In another embodiment, the adjuvant induces a T helper cell type 1 when administered to a subject.

In one embodiment, provided herein is a method of inhibiting growth of a tumor cell expressing a tumor-specific epitope defined in Table 1 or 2, comprising contacting a tumor cell with a peptide, polynucleotide, delivery system, vector, composition, antibody, or cells of the invention. In another embodiment, provided herein is a method of inhibiting growth of a tumor cell expressing a tumor-specific epitope defined in Table 3 or 4, comprising contacting the tumor cell with the peptide, polynucleotide, delivery system, vector, composition, antibody, or cells of the invention. In another embodiment, provided herein is a method of inhibiting growth of a tumor cell expressing a tumor-specific epitope defined in Table 5 or 6, comprising contacting the tumor cell with the peptide, polynucleotide, delivery system, vector, composition, antibody, or cells of the invention.

In one embodiment, provided herein is a method of treating cancer or initiating, enhancing, or prolonging an anti-tumor response in a subject in need thereof comprising administering to the subject the peptide, polynucleotide, vector, composition, antibody, or cells described herein. In one embodiment, the cancer is selected from the group consisting of CRC, head and neck, stomach, lung squamous, lung adeno, Prostate, Bladder. stomach, renal cell carcinoma, and uterine. In one embodiment, the cancer is selected from the group consisting of melanoma, lung squamous, DLBCL, uterine, head and neck, uterine, liver, and CRC. In one embodiment, the cancer is selected from the group consisting of cervical, head and neck, anal, stomach, Burkitt's lymphoma, and nasopharyngeal carcinoma.

In one embodiment, the subject is a human. In another embodiment, the subject has cancer. In another embodiment, the cancer is selected from the group consisting of urogenital, gynecological, lung, gastrointestinal, head and neck cancer, malignant glioblastoma, malignant mesothelioma, non-metastatic or metastatic breast cancer, malignant melanoma, triple-negative breast cancer (TNBC), smoldering myeloma (SMM), Merkel Cell Carcinoma or bone and soft tissue sarcomas, hematologic neoplasias, multiple myeloma, acute myelogenous leukemia, chronic myelogenous leukemia, myelodysplastic syndrome and acute lymphoblastic leukemia, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, esophageal cancers, hepatocellular cancers, cholangiocellular cancers, head and neck squamous cell cancer soft tissue sarcoma, and small cell lung cancer. In another embodiment, the subject has undergone surgical removal of the tumor. In another embodiment, the peptide, polynucleotide, vector, composition, or cells is administered via intravenous, intraperitoneal, intratumoral, intradermal, or subcutaneous administration. In another embodiment, the peptide, polynucleotide, vector, composition, or cells is administered into an anatomic site that drains into a lymph node basin. In another embodiment, the administration is into multiple lymph node basins. In another embodiment, the administration is by a subcutaneous or intradermal route.

In one embodiment of the method, a peptide is administered. In another embodiment, the administration is intratumorally. In another embodiment of the method, a polynucleotide, optionally RNA, is administered. In another embodiment, the polynucleotide is administered intravenously. In one embodiment of the method, a cell is administered. In another embodiment, the cell is a T cell or dendritic cell. In another embodiment, the peptide or polynucleotide comprises an antigen presenting cell targeting moiety.

One embodiment of the method further comprises administering at least one immune checkpoint inhibitor to a subject. In another embodiment, the checkpoint inhibitor is a biologic therapeutic or a small molecule. In another embodiment, the checkpoint inhibitor is selected from the group consisting of a monoclonal antibody, a humanized antibody, a fully human antibody and a fusion protein or a combination thereof. In another embodiment, the checkpoint inhibitor inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof. In another embodiment, the checkpoint inhibitor interacts with a ligand of a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof. In another embodiment, two or more checkpoint inhibitors are administered. In another embodiment, the checkpoint inhibitors are: (i) ipilimumab or tremelimumab, and (ii) nivolumab. In another embodiment, the checkpoint inhibitor and the composition are administered simultaneously or sequentially in any order. In another embodiment, the peptide, polynucleotide, vector, composition, or cells is administered prior to the checkpoint inhibitor. In another embodiment, the peptide, polynucleotide, vector, composition, or cells is administered after the checkpoint inhibitor. In another embodiment, administration of the checkpoint inhibitor is continued throughout antigen peptide, polynucleotide, vector, composition, or cell therapy. In another embodiment, the antigen peptide, polynucleotide, vector, composition, or cell therapy is administered to subjects that only partially respond or do not respond to checkpoint inhibitor therapy. In another embodiment, the composition is administered intravenously or subcutaneously. In another embodiment, the checkpoint inhibitor is administered intravenously or subcutaneously. In another embodiment, the checkpoint inhibitor is administered subcutaneously within about 2 cm of the site of administration of the composition. In another embodiment, the composition is administered into the same draining lymph node as the checkpoint inhibitor.

In one embodiment of the method, an additional agent is administered. In another embodiment, the agent is a chemotherapeutic agent, an immunomodulatory drug, an immune metabolism modifying drug, a targeted therapy, radiation an anti-angiogenesis agent, or an agent that reduces immune-suppression. In another embodiment, the chemotherapeutic agent is an alkylating agent, a topoisomerase inhibitor, an anti-metabolite, or an anti-mitotic agent. In another embodiment, the additional agent is an anti-glucocorticoid induced tumor necrosis factor family receptor (GITR) agonistic antibody or antibody fragment, ibrutinib, docetaxeol, cisplatin, or cyclophosphamide. In another embodiment, the administration elicits a CD4+ T cell immune response. In another embodiment, the administration elicits a CD4+ T cell immune response and a CD8+ T cell immune response.

In one embodiment, provided herein is a method for stimulating an immune response in a subject, comprising administering an effective amount of modified cells or composition described herein. In another embodiment, the immune response is cytotoxic and/or humoral immune response. In another embodiment, the method stimulates a T cell-mediated immune response in a subject. In another embodiment, the T cell-mediated immune response is directed against a target cell. In another embodiment, the target cell is a tumor cell. In another embodiment, the modified cells are transfected or transduced in vivo. In another embodiment, the modified cells are transfected or transduced ex vivo. In another embodiment, the modified cells are autologous patient T cells. In another embodiment, the autologous patient T cells are obtained from a patient that has received an antigen peptide or nucleic acid vaccine. In another embodiment, the antigen peptide or nucleic acid vaccine comprises at least one personalized antigen. In another embodiment, the antigen peptide or nucleic acid vaccine comprises at least one additional antigenic peptide listed in Table 1 or 2. In another embodiment, the antigen peptide or nucleic acid vaccine comprises at least one additional antigenic peptide listed in Table 3 or 4. In another embodiment, the antigen peptide or nucleic acid vaccine comprises at least one additional antigenic peptide listed in Table 5 or 6. In another embodiment, the at least one additional antigenic peptide listed in Table 1 or 2 is a retroviral antigenic peptide. In another embodiment, the at least one additional antigenic peptide listed in Table 3 or 4 is a non-mutated overexpressed antigenic peptide. In another embodiment, the at least one additional antigenic peptide listed in Table 5 or 6 is a viral antigenic peptide. In another embodiment, the patient received a chemotherapeutic agent, an immunomodulatory drug, an immune metabolism modifying drug, targeted therapy or radiation prior to and/or during receipt of the antigen peptide or nucleic acid vaccine. In another embodiment, the patient receives treatment with at least one checkpoint inhibitor. In another embodiment, the autologous T cells are obtained from a patient that has already received at least one round of T cell therapy containing an antigen. In another embodiment, the method further comprises adoptive T cell therapy. In another embodiment, the adoptive T cell therapy comprises autologous T-cells. In another embodiment, the autologous T-cells are targeted against tumor antigens. In another embodiment, the adoptive T cell therapy further comprises allogenic T-cells. In another embodiment, the allogenic T-cells are targeted against tumor antigens. In another embodiment, the adoptive T cell therapy is administered before the checkpoint inhibitor.

In one embodiment, provided herein is a method for evaluating the efficacy of treatment comprising: (i) measuring the number or concentration of target cells in a first sample obtained from the subject before administering the modified cell, (ii) measuring the number concentration of target cells in a second sample obtained from the subject after administration of the modified cell, and (iii) determining an increase or decrease of the number or concentration of target cells in the second sample compared to the number or concentration of target cells in the first sample. In another embodiment, the treatment efficacy is determined by monitoring a clinical outcome; an increase, enhancement or prolongation of anti-tumor activity by T cells; an increase in the number of anti-tumor T cells or activated T cells as compared with the number prior to treatment; B cell activity; CD4 T cell activity; or a combination thereof. In another embodiment, the treatment efficacy is determined by monitoring a biomarker. In another embodiment, the biomarker is selected from the group consisting of CEA, Her-2/neu, bladder tumor antigen, thyroglobulin, alpha-fetoprotein, PSA, CA 125, CA19.9, CA 15.3, leptin, prolactin, osteopontin, IGF-II, CD98, fascin, sPIgR, 14-3-3 eta, troponin I, and b-type natriuretic peptide. In another embodiment, the clinical outcome is selected from the group consisting of tumor regression; tumor shrinkage; tumor necrosis; anti-tumor response by the immune system; tumor expansion, recurrence or spread; or a combination thereof. In another embodiment, the treatment effect is predicted by presence of T cells or by presence of a gene signature indicating T cell inflammation or a combination thereof.

In one embodiment, provided herein is a method of treating cancer or initiating, enhancing, or prolonging an anti-tumor response in a subject in need thereof comprising administering to the subject: (a) the peptide, polynucleotide, vector, composition, antibody, or cells described herein; and (b) at least one checkpoint inhibitor. In another embodiment, the method further comprises administration of an immunomodulator or adjuvant. In another embodiment, the immunomodulator or adjuvant is selected from the group consisting of Poly(I:C), Poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312 VG, Montanide ISA 206 VG, Montanide ISA 50 V2, Montanide ISA 51 VG, OK-432, OM-174, OM-197-MP-EC, ISA-TLR2 agonist, ONTAK, PepTel® vector system, PLG microparticles, resiquimod, SRL172, virosomes and other virus-like particles, YF-17D, VEGF trap, R848, beta-glucan, Pam3Cys, Pam3CSK4, acrylic or methacrylic polymers, copolymers of maleic anhydride, and QS21 stimulon. a co-stimulatory ligand, a TNF ligand, an Ig superfamily ligand, CD28, CD80, CD86, ICOS, CD40L, OX40, CD27, GITR, CD30, DR3, CD69, or 4-1BB. In another embodiment, the immunomodulator or adjuvant is Poly-ICLC. In another embodiment, the checkpoint inhibitor is an anti-PD1 antibody or antibody fragment. In another embodiment, the inhibitor of the PD-1 pathway is nivolumab. In another embodiment, the checkpoint inhibitor is an anti-CTLA4 antibody or antibody fragment. In another embodiment, the anti-CTLA4 antibody is ipilimumab or tremelimumab. In another embodiment, the method comprises administering both an anti-PD1 antibody and an anti-CTLA4 antibody. In another embodiment, the administration of the checkpoint inhibitor is initiated before initiation of administration of the peptide, polynucleotide, vector, composition, antibody, or cell. In another embodiment, the administration of the checkpoint inhibitor is initiated after initiation of administration of the peptide, polynucleotide, vector, composition, antibody, or cell. In another embodiment, the administration of the checkpoint inhibitor is initiated simultaneously with the initiation of administration of the peptide, polynucleotide, vector, composition, antibody, or cell. In another embodiment, the peptide, polynucleotide, vector, composition, antibody, or cell is administered intravenously or subcutaneously. In another embodiment, the checkpoint inhibitor is administered intravenously or subcutaneously. In another embodiment, the checkpoint inhibitor is administered subcutaneously within about 2 cm of the site of administration of the peptide, polynucleotide, vector, composition, antibody, or cell. In another embodiment, the peptide, polynucleotide, vector, composition, antibody, or cell is administered into the same draining lymph node as the checkpoint inhibitor.

In one embodiment of the therapeutic methods, the additional therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, an angiogenesis inhibitor, such as hydroxy angiostatin K 1-3, DL-a-Difluoromethyl-omithine, endostatin, fumagillin, genistein, minocycline, staurosporine, and thalidomide; a DNA intercalator/cross-linker, such as Bleomycin, Carboplatin, Carmustine, Chlorambucil, Cyclophosphamide, cis-Diammineplatinum (II) dichloride (Cisplatin), Melphalan, Mitoxantrone, and Oxaliplatin; a DNA synthesis inhibitor, such as (+)-Amethopterin (Methotrexate), 3-Amino-1,2,4-benzotriazine 1,4-dioxide, Aminopterin, Cytosine j-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(-+-)-Camptothecin, Curcumin, (−)-Deguelin, 5,6-Dichlorobenzimidazole 1-β-D-ribofuranoside, Etoposide, Formestane, Fostriecin, Hispidin, 2-Immo-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatin15, Nocodazole, Paclitaxel, Podophyllotoxin, Rhizoxin, Vinblastine, Vincristine, Vindesine, and Vinorelbine (Navelbine); and an unclassified therapeutic agent, such as 17-(Allylamino)-17-demethoxygeldanamycin, 4-Amino-1,8-naphthalimide, Apigenin, Brefeldin A, Cimetidine, Dichioromethylene-diphosphonic acid, Leuprolide (Leuprorelin), Luteinizing Hormone-Releasing Hormone, Pifithrin-a, Rapamycin, Sex hormone-binding globulin, Thapsigargin, and Urinary trypsin inhibitor fragment (Bikunin). The therapeutic agent may be altretamine, amifostine, asparaginase, capecitabine, cladribine, cisapride, cyiarahirse, dacarbazine (DT1C), dactinomycin, dronabinol, epoetin alpha, “filgrastim, fludarabine, gemcitabine, granisetron, ifosfamide, irinotecan, lansoprazole, levamisole, leucovorin, megestrol, mesna, metoclopramide, mitotane, omeprazole, ondansetron, pilocarpine, prochloroperazine, or topotecan hydrochloride. The therapeutic agent may be a monoclonal antibody such as rituximab (Rituxan®), alemtuzumab (Campath®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 1311-tosit.umomab, ado-trastuzumab emtansine, lapatinib (Tykerb®), pertuzumab (Perjeta™), ado-trastuzumab emtansine (Adcyla™), regorafenib (Stivarga®), sunitinib (Sutent®), Denosumab (Xgeva®), sorafenib (Nexavar®), pazopanib (Votrient®), axitinib (Inita®), dasatinib (Sprycel®), nilotinib (Tasigna®), bosutinib (Bosulif®), ofatumumab (Arzerra®), obinutuzumab (Gazyva™), ibrutinib (Imbruvica™), idelalisib (Zydelig®), crizotinib (Xalkori®), erlotinib (Tarceva®), afatimb dimaleate (Giiotrif®), ceritinib (LDK378/Zykadia), Tositumomab and 1311-tositumomab (Bexxar®), ibritumomab tiuxetan (Zevalin®), brentuximab vedotin (Adcetris®), bortezomib (Velcade®), siltuximab (Sylvant™), trametinib (Ekinist®), dabrafenib (Tafmlar®), pembrolizimiab (Keytruda®), carfilzomib (Kyprolis®), Ramucirumab (Cyramza™), Cabozantinib (Cometriq™), vandetanib (Caprelsa®), Optionally, the therapeutic agent is a neoantigen. The therapeutic agent may be a cytokine such as interferons (INFs), interlcukins (ILs), or hematopoietic growth factors. The therapeutic agent may be INF-α, IL-2, Aldesleukin, IL-2, Erythropoietin, Granulocyte-macrophage colony-stimulating factor (GM-CSF) or granulocyte colony-stimulating factor. The therapeutic agent may be a targeted therapy such as toremifene (Fareston®), fulvestrant (Faslodex®), anastrozole (Arimidex®), exemestane (Aromasin®), letrozole (Femara®), ziv-aflibercept (Zaltrap®), Aiitretinoin (Panretin®), temsirolimus (Torisel®), Tretinoin (Vesanoid®), denileukin diftitox (Ontak®), vorinostat (Zoiinza®), romidepsin (Istodax®), bexarotene (Targretin®), pralatrexate (Foiotyn®), lenaliomide (Revlimid®), belinostat (Beleodaq™) lenaliomide (Revlimid®), pomalidomide (Pomalyst®), Cabazitaxel (Jevtana®), enzaluiamide (Xtandi®), abiraterone acetate (Zytiga®), radium 223 chloride (Xofigo®), or everolimus (Afiniior®). Additionally, the therapeutic agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Vorinostat (Zoiinza), Romidepsin (Istodax), or Ruxolitinib (Jakafi). For prostate cancer treatment, a preferred chemotherapeutic agent with which anti-CTLA-4 can be combined is paclitaxel (TAXOL).

In one embodiment, provided herein is a kit comprising any antigen therapeutic described herein.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### DETAILED DESCRIPTION

Described herein are novel immunotherapeutic agents and uses thereof based on the discovery of non-mutated protein epitopes expressed in cancer cells. Accordingly, the invention described herein provides peptides, polynucleotides encoding the peptides, and peptide binding agents, that can be used, for example, to stimulate an immune response to a tumor associated antigen, to create an immunogenic composition or cancer vaccine for use in treating disease.

### I. Definitions

To facilitate an understanding of the present invention, a number of terms and phrases are defined below.

“Non-mutated protein antigens” refer to antigens expressed in cancers either specifically or at a level higher than in non-cancer tissue. They include, but are not limited to, antigens of exogenous viruses, antigens of endogenous retroviruses and overexpressed antigens that do not comprise somatic mutations.

“Viral antigens” refer to antigens encoded by a exogenous virus.

“Retroviral antigens” refer to antigens encoded by an endogenous retroviral sequence.

“Non-mutated overexpressed antigens” refer to non-mutated antigens encoded by a genome of a cancer cell that are expressed at a level higher than in non-cancer tissue.

A “tumor-specific epitope” refers to an epitope that is either not expressed in non-cancer or germline cells but is found expressed in cancer cells, or that is expressed at a higher level in cancer cells than in non-cancer cells.

A “reference” can be used to correlate and compare the results obtained in the methods of the invention from a tumor specimen. Typically the “reference” may be obtained on the basis of one or more normal specimens, in particular specimens which are not affected by a cancer disease, either obtained from a patient or one or more different individuals, for example, healthy individuals, in particular individuals of the same species. A “reference” can be determined empirically by testing a sufficiently large number of normal specimens.

The term “mutation” refers to a change of or difference in the nucleic acid sequence (nucleotide substitution, addition or deletion) compared to a reference. A “somatic mutation” can occur in any of the cells of the body except the germ cells (sperm and egg) and therefore are not passed on to children. These alterations can (but do not always) cause cancer or other diseases. In some embodiments, a mutation is a non-synonymous mutation. The term “non-synonymous mutation” refers to a mutation, for example, a nucleotide substitution, which does result in an amino acid change such as an amino acid substitution in the translation product.

Throughout this disclosure, “binding data” results can be expressed in terms of “IC₅₀.” IC₅₀ is the concentration of the tested peptide in a binding assay at which 50% inhibition of binding of a labeled reference peptide is observed. Given the conditions in which the assays are run (i.e., limiting HLA protein and labeled reference peptide concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC₅₀, relative to the IC₅₀ of a reference standard peptide.

Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)).

“Cross-reactive binding” indicates that a peptide is bound by more than one HLA molecule; a synonym is degenerate binding.

The term “derived” when used to discuss an epitope is a synonym for “prepared.” A derived epitope can be isolated from a natural source, or it can be synthesized according to standard protocols in the art. Synthetic epitopes can comprise artificial amino acid residues “amino acid mimetics,” such as D isomers of natural occurring L amino acid residues or non-natural amino acid residues such as cyclohexylalanine. A derived or prepared epitope can be an analog of a native epitope.

A “diluent” includes sterile liquids, such as water and oils, including those of petroleum, animal, vegetable or synthetic origin, such as peanut oil, soybean oil, mineral oil, sesame oil and the like. Water is also a diluent for pharmaceutical compositions. Saline solutions and aqueous dextrose and glycerol solutions can also be employed as diluents, for example, in injectable solutions.

An “epitope” is the collective features of a molecule, such as primary, secondary and tertiary peptide structure, and charge, that together form a site recognized by, for example, an immunoglobulin, T cell receptor, HLA molecule, or chimeric antigen receptor. Alternatively, an epitope can be defined as a set of amino acid residues which is involved in recognition by a particular immunoglobulin, or in the context of T cells, those residues necessary for recognition by T cell receptor proteins, chimeric antigen receptors, and/or Major Histocompatibility Complex (MHC) receptors. Epitopes can be prepared by isolation from a natural source, or they can be synthesized according to standard protocols in the art. Synthetic epitopes can comprise artificial amino acid residues, “amino acid mimetics,” such as D isomers of naturally-occurring L amino acid residues or non-naturally-occurring amino acid residues such as cyclohexylalanine. Throughout this disclosure, epitopes may be referred to in some cases as peptides or peptide epitopes.

It is to be appreciated that proteins or peptides that comprise an epitope or an analog described herein as well as additional amino acid(s) are still within the bounds of the invention. In certain embodiments, the peptide comprises a fragment of an antigen.

In certain embodiments, there is a limitation on the length of a peptide of the invention. The embodiment that is length-limited occurs when the protein or peptide comprising an epitope described herein comprises a region (i.e., a contiguous series of amino acid residues) having 100% identity with a native sequence. In order to avoid the definition of epitope from reading, e.g., on whole natural molecules, there is a limitation on the length of any region that has 100% identity with a native peptide sequence. Thus, for a peptide comprising an epitope described herein and a region with 100% identity with a native peptide sequence, the region with 100% identity to a native sequence generally has a length of less than or equal to 600 amino acid residues, less than or equal to 500 amino acid residues, less than or equal to 400 amino acid residues, less than or equal to 250 amino acid residues, less than or equal to 100 amino acid residues, less than or equal to 85 amino acid residues, less than or equal to 75 amino acid residues, less than or equal to 65 amino acid residues, and less than or equal to 50 amino acid residues. In certain embodiments, an “epitope” described herein is comprised by a peptide having a region with less than 51 amino acid residues that has 100% identity to a native peptide sequence, in any increment down to 5 amino acid residues; for example 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues.

“Human Leukocyte Antigen” or “HLA” is a human class I or class II Major Histocompatibility Complex (MHC) protein (see, e.g., Stites, et al., IMMUNOLOGY, 8TH ED., Lange Publishing, Los Altos, Calif. (1994).

An “HLA supertype or HLA family”, as used herein, describes sets of HLA molecules grouped on the basis of shared peptide-binding specificities. HLA class I molecules that share somewhat similar binding affinity for peptides bearing certain amino acid motifs are grouped into such HLA supertypes. The terms HLA superfamily, HLA supertype family, HLA family, and HLA xx-like molecules (where “xx” denotes a particular HLA type), are synonyms.

The terms “identical” or percent “identity,” in the context of two or more peptide sequences or antigen fragments, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

An “immunogenic” peptide or an “immunogenic” epitope or “peptide epitope” is a peptide that comprises an allele-specific motif such that the peptide will bind an HLA molecule and induce a cell-mediated or humoral response, for example, cytotoxic T lymphocyte (CTL), helper T lymphocyte (HTL) and/or B lymphocyte response. Thus, immunogenic peptides described herein are capable of binding to an appropriate HLA molecule and thereafter inducing a CTL (cytotoxic) response, or a HTL (and humoral) response, to the peptide.

As used herein, a “chimeric antigen receptor” or “CAR” refers to an antigen binding protein in that includes an immunoglobulin antigen binding domain (e.g., an immunoglobulin variable domain) and a T cell receptor (TCR) constant domain. As used herein, a “constant domain” of a TCR polypeptide includes a membrane-proximal TCR constant domain, and may also include a TCR transmembrane domain and/or a TCR cytoplasmic tail. For example, in some embodiments, the CAR is a dimer that includes a first polypeptide comprising a immunoglobulin heavy chain variable domain linked to a TCR-beta constant domain and a second polypeptide comprising an immunoglobulin light chain variable domain (e.g., a κ or λ variable domain) linked to a TCRα constant domain. In some embodiments, the CAR is a dimer that includes a first polypeptide comprising a immunoglobulin heavy chain variable domain linked to a TCRα constant domain and a second polypeptide comprising an immunoglobulin light chain variable domain (e.g., a κ or λ variable domain) linked to a TCRβ constant domain.

The phrases “isolated” or “biologically pure” refer to material which is substantially or essentially free from components which normally accompany the material as it is found in its native state. Thus, isolated peptides described herein do not contain some or all of the materials normally associated with the peptides in their in situ environment. An “isolated” epitope refers to an epitope that does not include the whole sequence of the antigen from which the epitope was derived. Typically the “isolated” epitope does not have attached thereto additional amino acid residues that result in a sequence that has 100% identity over the entire length of a native sequence. The native sequence can be a sequence such as a tumor-associated antigen from which the epitope is derived. Thus, the term “isolated” means that the material is removed from its original environment (e.g., the natural environment if it is naturally occurring). For example, a naturally-occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. Isolated RNA molecules include in vivo or in vitro RNA transcripts of the DNA molecules described herein, and further include such molecules produced synthetically.

“Major Histocompatibility Complex” or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, FUNDAMENTAL IMMUNOLOGY, 3.sup.RD ED., Raven Press, New York (1993).

A “native” or a “wild type” sequence refers to a sequence found in nature. Such a sequence can comprise a longer sequence in nature.

A “T-cell epitope” is to be understood as meaning a peptide sequence which can be bound by the MHC molecules of class I or II in the form of a peptide-presenting MHC molecule or MHC complex and then, in this form, be recognized and bound by cytotoxic T-lymphocytes or T-helper cells, respectively.

A “receptor” is to be understood as meaning a biological molecule or a molecule grouping capable of binding a ligand. A receptor may serve, to transmit information in a cell, a cell formation or an organism. The receptor comprises at least one receptor unit, for example, where each receptor unit may consist of a protein molecule. The receptor has a structure which complements that of a ligand and may complex the ligand as a binding partner. The information is transmitted in particular by conformational changes of the receptor following complexation of the ligand on the surface of a cell. In some embodiments, a receptor is to be understood as meaning in particular proteins of MHC classes I and II capable of forming a receptor/ligand complex with a ligand, in particular a peptide or peptide fragment of suitable length.

A “ligand” is to be understood as meaning a molecule which has a structure complementary to that of a receptor and is capable of forming a complex with this receptor. In some embodiments, a ligand is to be understood as meaning a peptide or peptide fragment which has a suitable length and suitable binding motifs in its amino acid sequence, so that the peptide or peptide fragment is capable of forming a complex with proteins of MHC class I or MHC class II.

In some embodiments, a “receptor/ligand complex” is also to be understood as meaning a “receptor/peptide complex” or “receptor/peptide fragment complex”, including a peptide- or peptide fragment-presenting MHC molecule of class I or of class II.

“Proteins or molecules of the major histocompatibility complex (MHC)”, “MHC molecules”, “MHC proteins” or “HLA proteins” are to be understood as meaning proteins capable of binding peptides resulting from the proteolytic cleavage of protein antigens and representing potential lymphocyte epitopes, (e.g., T cell epitope and B cell epitope) transporting them to the cell surface and presenting them there to specific cells, in particular cytotoxic T-lymphocytes, T-helper cells, or B cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The cellular biology and the expression patterns of the two MHC classes are adapted to these different roles.

The terms “peptide” and “peptide epitope” are used interchangeably with “oligopeptide” in the present specification to designate a series of residues connected one to the other, typically by peptide bonds between the α-amino and carboxyl groups of adjacent amino acid residues.

“Synthetic peptide” refers to a peptide that is obtained from a non-natural source, e.g., is man-made. Such peptides can be produced using such methods as chemical synthesis or recombinant DNA technology. “Synthetic peptides” include “fusion proteins.”

A “PanDR binding” peptide, a “PanDR binding epitope” is a member of a family of molecules that binds more than one HLA class II DR molecule.

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

The term “motif” refers to a pattern of residues in an amino acid sequence of defined length, for example, a peptide of less than about 15 amino acid residues in length, or less than about 13 amino acid residues in length, for example, from about 8 to about 13 amino acid residues (e.g., 8, 9, 10, 11, 12, or 13) for a class I HLA motif and from about 6 to about 25 amino acid residues (e.g., 6, 7, 8, 9, 10, 11, 12, 13, 14, 15, 16, 17, 18, 19, 20, 21, 22, 23, 24, or 25) for a class II HLA motif, which is recognized by a particular HLA molecule. Motifs are typically different for each HLA protein encoded by a given human HLA allele. These motifs differ in their pattern of the primary and secondary anchor residues. In some embodiments, an MHC class I motif identifies a peptide of 9, 10, or 11 amino acid residues in length.

A “supermotif” is a peptide binding specificity shared by HLA molecules encoded by two or more HLA alleles. In some embodiments, a supermotif-bearing peptide described herein is recognized with high or intermediate affinity (as defined herein) by two or more HLA antigens.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

According to the invention, the term “vaccine” relates to a pharmaceutical preparation (pharmaceutical composition) or product that upon administration induces an immune response, for example, a cellular or humoral immune response, which recognizes and attacks a pathogen or a diseased cell such as a cancer cell. A vaccine may be used for the prevention or treatment of a disease. The term “individualized cancer vaccine” or “personalized cancer vaccine” concerns a particular cancer patient and means that a cancer vaccine is adapted to the needs or special circumstances of an individual cancer patient.

A “protective immune response” or “therapeutic immune response” refers to a CTL and/or an HTL response to an antigen derived from an pathogenic antigen (e.g., a tumor antigen), which in some way prevents or at least partially arrests disease symptoms, side effects or progression. The immune response can also include an antibody response which has been facilitated by the stimulation of helper T cells.

“Antigen processing” or “processing” refers to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, for example, antigen presenting cells, to specific T cells.

“Antigen presenting cells” (APC) are cells which present peptide fragments of protein antigens in association with MHC molecules on their cell surface. Some APCs may activate antigen specific T cells. Professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen presenting cell. An additional co-stimulatory signal is then produced by the antigen presenting cell, leading to activation of the T cell. The expression of co-stimulatory molecules is a defining feature of professional antigen-presenting cells.

The main types of professional antigen-presenting cells are dendritic cells, which have the broadest range of antigen presentation, and are probably the most important antigen presenting cells, macrophages, B-cells, and certain activated epithelial cells.

Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity.

Dendritic cells are conveniently categorized as “immature” and “mature” cells, which can be used as a simple way to discriminate between two well characterized phenotypes. However, this nomenclature should not be construed to exclude all possible intermediate stages of differentiation.

Immature dendritic cells are characterized as antigen presenting cells with a high capacity for antigen uptake and processing, which correlates with the high expression of Fey receptor and mannose receptor. The mature phenotype is typically characterized by a lower expression of these markers, but a high expression of cell surface molecules responsible for T cell activation such as class I and class II MHC, adhesion molecules (e.g. CD54 and CD11) and costimulatory molecules (e.g., CD40, CD80, CD86 and 4-1 BB).

The term “residue” refers to an amino acid residue or amino acid mimetic residue incorporated into a peptide or protein by an amide bond or amide bond mimetic, or nucleic acid (DNA or RNA) that encodes the amino acid or amino acid mimetic.

The nomenclature used to describe peptides or proteins follows the conventional practice wherein the amino group is presented to the left (the amino- or N-terminus) and the carboxyl group to the right (the carboxy- or C-terminus) of each amino acid residue. When amino acid residue positions are referred to in a peptide epitope they are numbered in an amino to carboxyl direction with position one being the residue located at the amino terminal end of the epitope, or the peptide or protein of which it can be a part.

In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acid residues having D-forms is represented by a lower case single letter or a lower case three letter symbol. However, when three letter symbols or full names are used without capitals, they can refer to L amino acid residues. Glycine has no asymmetric carbon atom and is simply referred to as “Gly” or “G”. The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine.)

The terms “polynucleotide” and “nucleic acid” are used interchangeably herein and refer to polymers of nucleotides of any length, and include DNA and RNA, for example, mRNA. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered is mRNA.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic acids or polypeptides described herein are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value 2 between. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate peptide function are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure. In one embodiment, an “isolated polynucleotide” encompasses a PCR or quantitative PCR reaction comprising the polynucleotide amplified in the PCR or quantitative PCR reaction.

The term “substantially pure” as used herein refers to material which is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already with the disorder; those prone to have the disorder; and those in whom the disorder is to be prevented.

As used in the present disclosure and claims, the singular forms “a”, “an” and “the” include plural forms unless the context clearly dictates otherwise.

It is understood that terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. Nothing herein is intended as a promise.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Non-Mutated Protein Antigens Expressed in Cancer Cells

Applicants have discovered antigens expressed by cancer cells encoded by the following genes: ERVH-2 matrix protein, ERVH-2 gag, ERVH48-1 coat protein, ERVH48-1 syncytin, ERVE-4 reverse transcriptase, ERVK-5 gag, env, pol protein, and ERVI-1 envelope protein.

Applicants have discovered antigens expressed by cancer encoded by the following genes: TYR, MAGEC1, MAGEA10, MAGEB17, MAGEA4, MABEB16, MAGEA1, MAGEA8, MAGEB4, CT45A5, ALPPL2, MMP13, CTAG1B, DCT, CLDN6, MLANA, AFP, DKK4, ASCL2, GAGE1, GAGE10, SLC45A2, PAGE5, PAGE2, and PMEL.

Applicants have discovered antigens expressed by cancer encoded by the following genes: HPV-16, E6, HPV-16 E7, EBV LF2, EBV BALF5, EBV RPMS1, EBV A73, EBV BALF4, EBV BALF3, and EBV BARF0.

### Non-Mutated Protein Epitope Polypeptides

In aspects, the invention provides isolated peptides that comprise a non-mutated protein epitope expressed in a cancer cell. In some embodiments, the non-mutated protein epitope is a retroviral antigen. In some embodiments, the non-mutated protein epitope is a non-mutated overexpressed antigen. In some embodiments, the non-mutated protein epitope is a viral antigen.

In aspects, the invention provides an isolated peptide that comprises a peptide from Tables 1-6. The term “peptide” is used in the present specification to designate a series of residues, typically L-amino acids, connected one to the other, typically by peptide bonds between the α-amino and carboxyl groups of adjacent amino acids. Similarly, the term “polypeptide” is used in the present specification to designate a series of residues, e.g., L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

In some embodiments, sequencing methods are used to identify tumor specific epitopes. Any suitable sequencing method can be used according to the invention, for example, Next Generation Sequencing (NGS) technologies. Third Generation Sequencing methods might substitute for the NGS technology in the future to speed up the sequencing step of the method. For clarification purposes: the terms “Next Generation Sequencing” or “NGS” in the context of the present invention mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, for example, within 1-7 days or within less than 24 hours and allow, in principle, single cell sequencing approaches. Multiple NGS platforms which are commercially available or which are mentioned in the literature can be used in the context of the invention e.g. those described in detail in WO 2012/159643.

In certain embodiments a non-mutated protein epitope peptide described herein molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino acid residues, and any range derivable therein. In specific embodiments, a non-mutated protein epitope peptide molecule is equal to or less than 100 amino acids.

In some embodiments, non-mutated protein epitope peptides and polypeptides described herein for MHC Class I are 13 residues or less in length and usually consist of between about 8 and about 11 residues, particularly 9 or 10 residues. In some embodiments, non-mutated protein epitope peptides and polypeptides described herein for MHC Class II are 9-24 residues in length.

A longer non-mutated protein epitope peptide can be designed in several ways. In some embodiments, when HLA-binding peptides are predicted or known, a longer non-mutated protein epitope peptide could consist of (1) individual binding peptides with extensions of 2-5 amino acids toward the N- and C-terminus of each corresponding peptide; or (2) a concatenation of some or all of the binding peptides with extended sequences for each. In some embodiments, use of a longer peptide is presumed to allow for endogenous processing by patient cells and can lead to more effective antigen presentation and induction of T cell responses. In some embodiments, two or more peptides can be used, where the peptides overlap and are tiled over the long non-mutated protein epitope peptide.

In some embodiments, the non-mutated protein epitope peptides and polypeptides bind an HLA protein (e.g., HLA class I or HLA class II). In specific embodiments the non-mutated protein epitope peptide or polypeptide has an IC₅₀ of at least less than 5000 nM, at least less than 500 nM, at least less than 100 nM, at least less than 50 nM or less.

In some embodiments, a non-mutated protein epitope peptide described herein can comprise carriers such as those well known in the art, e.g., thyroglobulin, albumins such as human serum albumin, tetanus toxoid, polyamino acid residues such as poly L-lysine, poly L-glutamic acid, influenza virus proteins, hepatitis B virus core protein, and the like.

In some embodiments, a non-mutated protein epitope peptide described herein can be modified by terminal-NH₂ acylation, e.g., by alkanoyl (C₁-C₂₀) or thioglycolyl acetylation, terminal-carboxyl amidation, e.g., ammonia, methylamine, etc. In some embodiments these modifications can provide sites for linking to a support or other molecule.

In some embodiments, a non-mutated protein epitope peptide described herein can contain modifications such as but not limited to glycosylation, side chain oxidation, biotinylation, phosphorylation, addition of a surface active material, e.g. a lipid, or can be chemically modified, e.g., acetylation, etc. Moreover, bonds in the peptide can be other than peptide bonds, e.g., covalent bonds, ester or ether bonds, disulfide bonds, hydrogen bonds, ionic bonds, etc.

In some embodiments, a non-mutated protein epitope peptide described herein can contain substitutions to modify a physical property (e.g., stability or solubility) of the resulting peptide. For example, non-mutated protein epitope peptides can be modified by the substitution of a cysteine (C) with u-amino butyric acid (“B”). Due to its chemical nature, cysteine has the propensity to form disulfide bridges and sufficiently alter the peptide structurally so as to reduce binding capacity. Substituting α-amino butyric acid for C not only alleviates this problem, but actually improves binding and crossbinding capability in certain instances. Substitution of cysteine with u-amino butyric acid can occur at any residue of a non-mutated protein epitope peptide, e.g., at either anchor or non-anchor positions of an epitope or analog within a peptide, or at other positions of a peptide.

In some embodiments, a non-mutated protein epitope peptide described herein can comprise amino acid mimetics or unnatural amino acid residues, e.g. D- or L-naphylalanine; D- or L-phenylglycine; D- or L-2-thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoro-methyl)-phenylalanine; D-.rho.-fluorophenylalanine; D- or L-.rho.-biphenyl-phenylalanine; D- or L-p-methoxybiphenylphenylalanine; D- or L-2-indole(allyl)alanines; and, D- or L-alkylalanines, where the alkyl group can be a substituted or unsubstituted methyl, ethyl, propyl, hexyl, butyl, pentyl, isopropyl, iso-butyl, sec-isotyl, iso-pentyl, or a non-acidic amino acid residues. Aromatic rings of a non-natural amino acid include, e.g., thiazolyl, thiophenyl, pyrazolyl, benzimidazolyl, naphthyl, furanyl, pyrrolyl, and pyridyl aromatic rings. Modified peptides that have various amino acid mimetics or unnatural amino acid residues are particularly useful, as they tend to manifest increased stability in vivo. Such peptides can also possess improved shelf-life or manufacturing properties.

Peptide stability can be assayed in a number of ways. For instance, peptidases and various biological media, such as human plasma and serum, have been used to test stability. See, e.g., Verhoef, et al., Eur. J. Drug Metab. Pharmacokinetics 11:291 (1986). Half-life of the peptides described herein is conveniently determined using a 25% human serum (v/v) assay. The protocol is as follows: pooled human serum (Type AB, non-heat inactivated) is dilapidated by centrifugation before use. The serum is then diluted to 25% with RPMI-1640 or another suitable tissue culture medium. At predetermined time intervals, a small amount of reaction solution is removed and added to either 6% aqueous trichloroacetic acid (TCA) or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

In some embodiments, a non-mutated protein epitope peptide described herein can be in solution, lyophilized, or can be in crystal form.

In some embodiments, a non-mutated protein epitope peptide described herein can be prepared synthetically, by recombinant DNA technology or chemical synthesis, or can be isolated from natural sources such as native tumors or pathogenic organisms. Epitopes can be synthesized individually or joined directly or indirectly in a peptide. Although a non-mutated protein epitope peptide described herein will be substantially free of other naturally occurring host cell proteins and fragments thereof, in some embodiments the peptide can be synthetically conjugated to be joined to native fragments or particles.

In some embodiments, a non-mutated protein epitope peptide described herein can be prepared in a wide variety of ways. In some embodiments, the peptides can be synthesized in solution or on a solid support according to conventional techniques. Various automatic synthesizers are commercially available and can be used according to known protocols. (See, for example, Stewart & Young, SOLID PHASE PEPTIDE SYNTHESIS, 2D. ED., Pierce Chemical Co., 1984). Further, individual peptides can be joined using chemical ligation to produce larger peptides that are still within the bounds of the invention.

Alternatively, recombinant DNA technology can be employed wherein a nucleotide sequence which encodes a peptide inserted into an expression vector, transformed or transfected into an appropriate host cell and cultivated under conditions suitable for expression. These procedures are generally known in the art, as described generally in Sambrook et al., MOLECULAR CLONING, A LABORATORY MANUAL, Cold Spring Harbor Press, Cold Spring Harbor, N.Y. (1989). Thus, recombinant peptides, which comprise or consist of one or more epitopes described herein, can be used to present the appropriate T cell epitope.

In one aspect, the invention described herein also provides compositions comprising one, at least two, or more than two non-mutated protein epitope peptides. In some embodiments a composition described herein contains at least two distinct peptides. In some embodiments, the at least two distinct peptides are derived from the same polypeptide. By distinct polypeptides is meant that the peptide vary by length, amino acid sequence or both. The peptides are derived from any polypeptide known to or have been found to contain a tumor specific epitope.

### Non-Mutated Protein Epitope Polynucleotides

Polynucleotides encoding each of the peptides described herein are also part of the invention. As appreciated by one of ordinary skill in the art, various nucleic acids will encode the same peptide due to the redundancy of the genetic code. Each of these nucleic acids falls within the scope of the present invention. This embodiment of the invention comprises DNA and RNA, for example, mRNA, and in certain embodiments a combination of DNA and RNA. In one embodiment, the mRNA is a self-amplifying mRNA. (Brito et al., Adv. Genet. 2015; 89:179-233). It is to be appreciated that any polynucleotide that encodes a peptide described herein falls within the scope of this invention.

The term “RNA” includes and in some embodiments relates to “mRNA”. The term “mRNA” means “messenger-RNA” and relates to a “transcript” which is generated by using a DNA template and encodes a peptide or polypeptide. Typically, an mRNA comprises a 5′-UTR, a protein coding region, and a 3′-UTR. mRNA only possesses limited half-life in cells and in vitro. In one embodiment, the mRNA is self-amplifying mRNA. In the context of the present invention, mRNA may be generated by in vitro transcription from a DNA template. The in vitro transcription methodology is known to the skilled person. For example, there is a variety of in vitro transcription kits commercially available.

The stability and translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448 incorporated herein by reference. In order to increase expression of the RNA used according to the present invention, it may be modified within the coding region, i.e. the sequence encoding the expressed peptide or protein, without altering the sequence of the expressed peptide or protein, so as to increase the GC-content to increase mRNA stability and to perform a codon optimization and, thus, enhance translation in cells.

The term “modification” in the context of the RNA used in the present invention includes any modification of an RNA which is not naturally present in said RNA. In one embodiment of the invention, the RNA used according to the invention does not have uncapped 5′-triphosphates. Removal of such uncapped 5′-triphosphates can be achieved by treating RNA with a phosphatase. The RNA according to the invention may have modified ribonucleotides in order to increase its stability and/or decrease cytotoxicity. For example, in one embodiment, in the RNA used according to the invention 5-methylcytidine is substituted partially or completely, for example, completely, for cytidine. Alternatively or additionally, in one embodiment, in the RNA used according to the invention pseudouridine is substituted partially or completely, for example, completely, for uridine.

In one embodiment the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, to the 7-methylguanosine cap (m G). In the context of the present invention, the term “5′-cap” includes a 5′-cap analog that resembles the RNA cap structure and is modified to possess the ability to stabilize RNA and/or enhance translation of RNA if attached thereto, in vivo and/or in a cell.

In certain embodiments, an mRNA encoding a non-mutated protein epitope is administered to a subject in need thereof. In one embodiment, the invention provides RNA, oligoribonucleotide, and polyribonucleotide molecules comprising a modified nucleoside, gene therapy vectors comprising same, gene therapy methods and gene transcription silencing methods comprising same. In one embodiment, the mRNA to be administered comprises at least one modified nucleoside.

The polynucleotides encoding peptides described herein can be synthesized by chemical techniques, for example, the phosphotriester method of Matteucci, et al., J. Am. Chem. Soc. 103:3185 (1981). Polynucleotides encoding peptides comprising or consisting of an analog can be made simply by substituting the appropriate and desired nucleic acid base(s) for those that encode the native epitope.

A large number of vectors and host systems suitable for producing and administering a non-mutated protein epitope peptide described herein are known to those of skill in the art, and are commercially available. The following vectors are provided by way of example. Bacterial: pQE70, pQE60, pQE-9 (Qiagen), pBS, pD10, phagescript, psiX174, pBluescript SK, pbsks, pNH8A, pNH16a, pNH18A, pNH46A (Stratagene); ptrc99a, pKK223-3, pKK233-3, pDR540, pRIT5 (Pharmacia); pCR (Invitrogen). Eukaryotic: pWLNEO, pSV2CAT, pOG44, pXT1, pSG (Stratagene) pSVK3, pBPV, pMSG, pSVL (Pharmacia); p75.6 (Valentis); pCEP (Invitrogen); pCEI (Epimmune). However, any other plasmid or vector can be used as long as it is replicable and viable in the host.

As representative examples of appropriate hosts, there can be mentioned: bacterial cells, such as E. coli, Bacillus subtilis, Salmonella typhimurium and various species within the genera Pseudomonas, Streptomyces, and Staphylococcus; fungal cells, such as yeast; insect cells such as Drosophila and Sf9; animal cells such as COS-7 lines of monkey kidney fibroblasts, described by Gluzman, Cell 23:175 (1981), and other cell lines capable of expressing a compatible vector, for example, the C127, 3T3, CHO, HeLa and BHK cell lines or Bowes melanoma; plant cells, etc. The selection of an appropriate host is deemed to be within the scope of those skilled in the art from the teachings herein.

Thus, the present disclosure is also directed to vectors, and expression vectors useful for the production and administration of the non-mutated protein epitope peptides described herein, and to host cells comprising such vectors.

Host cells are genetically engineered (transduced or transformed or transfected) with the vectors which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants or amplifying the polynucleotides. The culture conditions, such as temperature, pH and the like, are those previously used with the host cell selected for expression, and will be apparent to the ordinarily skilled artisan.

For expression of the non-mutated protein epitope peptides described herein, the coding sequence will be provided operably linked start and stop codons, promoter and terminator regions, and in some embodiments, and a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), acid phosphatase, or heat shock proteins, among others. The heterologous structural sequence is assembled in appropriate phase with translation initiation and termination sequences, and in some embodiments, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Yeast, insect or mammalian cell hosts can also be used, employing suitable vectors and control sequences. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. Such promoters can also be derived from viral sources, such as, e.g., human cytomegalovirus (CMV-IE promoter) or herpes simplex virus type-1 (HSV TK promoter). Nucleic acid sequences derived from the SV40 splice, and polyadenylation sites can be used to provide the nontranscribed genetic elements.

Polynucleotides encoding non-mutated protein epitope peptides described herein can also comprise a ubiquitination signal sequence, and/or a targeting sequence such as an endoplasmic reticulum (ER) signal sequence to facilitate movement of the resulting peptide into the endoplasmic reticulum.

Polynucleotides described herein can be administered and expressed in human cells (e.g., immune cells, including dendritic cells). A human codon usage table can be used to guide the codon choice for each amino acid. Such polynucleotides comprise spacer amino acid residues between epitopes and/or analogs, such as those described above, or can comprise naturally-occurring flanking sequences adjacent to the epitopes and/or analogs (and/or CTL, HTL, and B cell epitopes).

In some embodiments, a non-mutated protein epitope peptide described herein can also be administered/expressed by viral or bacterial vectors. Examples of expression vectors include attenuated viral hosts, such as vaccinia or fowlpox. As an example of this approach, vaccinia virus is used as a vector to express nucleotide sequences that encode the non-mutated protein epitope peptides described herein. Vaccinia vectors and methods useful in immunization protocols are described in, e.g., U.S. Pat. No. 4,722,848. Another vector is BCG (Bacille Calmette Guerin). BCG vectors are described by Stover et al., Nature 351:456-460 (1991). A wide variety of other vectors useful for therapeutic administration or immunization of the non-mutated protein epitope polypeptides described herein, e.g. adeno and adeno-associated virus vectors, retroviral vectors, Salmonella typhi vectors, detoxified anthrax toxin vectors, Sendai virus vectors, poxvirus vectors, canarypox vectors, and fowlpox vectors, and the like, will be apparent to those skilled in the art from the description herein. In some embodiments, the vector is Modified Vaccinia Ankara (VA) (e.g. Bavarian Noridic (MVA-BN)).

Standard regulatory sequences well known to those of skill in the art can be included in the vector to ensure expression in the human target cells. Several vector elements are desirable: a promoter with a downstream cloning site for polynucleotide, e.g., minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences. In some embodiments, the promoter is the CMV-IE promoter.

Polynucleotides described herein can comprise one or more synthetic or naturally-occurring introns in the transcribed region. The inclusion of mRNA stabilization sequences and sequences for replication in mammalian cells can also be considered for increasing polynucleotide expression.

In addition, a polynucleotide described herein can comprise immunostimulatory sequences (ISSs or CpGs). These sequences can be included in the vector, outside the polynucleotide coding sequence to enhance immunogenicity.

### Non-Mutated Protein Epitope Binding Peptides

In certain embodiments, the present invention provides a binding protein (e.g., an antibody or antigen-binding fragment thereof), or a T cell receptor (TCR), or a chimeric antigen receptor (CAR) capable of binding with a high affinity to a non-mutated protein epitope peptide:human leukocyte antigen (HLA) complex. In some embodiments, the present invention provides a CAR that is capable of binding with a high affinity to a non-mutated protein epitope peptide derived from the extracellular domain of a protein. In certain embodiments, an antigen-specific binding protein or TCR or CAR as described herein includes variant polypeptide species that have one or more amino acid substitutions, insertions, or deletions in the native amino acid sequence, provided that the binding protein retains or substantially retains its specific binding function. Conservative substitutions of amino acids are well known and may occur naturally or may be introduced when the binding protein or TCR is recombinantly produced. Amino acid substitutions, deletions, and additions may be introduced into a protein using mutagenesis methods known in the art (see, e.g., Sambrook et al., Molecular Cloning: A Laboratory Manual, 3d ed., Cold Spring Harbor Laboratory Press, N Y, 2001). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al., supra).

A variety of criteria known to persons skilled in the art indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine) In certain circumstances, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS, Align, the BLAST algorithm, or other algorithms described herein and practiced in the art).

In certain embodiments, anon-mutated protein epitope specific binding protein, TCR or CAR is capable of (a) specifically binding to an antigen:HLA complex on a cell surface independent or in the absence of CD8. In certain embodiments, a non-mutated protein epitope specific binding protein is a T cell receptor (TCR), a chimeric antigen receptor or an antigen-binding fragment of a TCR, any of which can be chimeric, humanized or human. In further embodiments, an antigen-binding fragment of the TCR comprises a single chain TCR (scTCR).

In certain embodiments, there is provided a composition comprising anon-mutated protein epitope-specific binding protein or high affinity recombinant TCR according to any one of the above embodiments and a pharmaceutically acceptable carrier, diluent, or excipient.

Methods useful for isolating and purifying recombinantly produced soluble TCR, by way of example, can include obtaining supernatants from suitable host cell/vector systems that secrete the recombinant soluble TCR into culture media and then concentrating the media using a commercially available filter. Following concentration, the concentrate can be applied to a single suitable purification matrix or to a series of suitable matrices, such as an affinity matrix or an ion exchange resin. One or more reverse phase HPLC steps may be employed to further purify a recombinant polypeptide. These purification methods can also be employed when isolating an immunogen from its natural environment. Methods for large scale production of one or more of the isolated/recombinant soluble TCR described herein include batch cell culture, which is monitored and controlled to maintain appropriate culture conditions. Purification of the soluble TCR may be performed according to methods described herein and known in the art.

### III. Immunogenic and Vaccine Compositions

In one embodiment, provided herein is an immunogenic composition, e.g., a vaccine composition capable of raising a non-mutated protein epitope-specific response (e.g., a humoral or cell-mediated immune response). In some embodiments, the immunogenic composition comprises non-mutated protein epitope therapeutics (e.g., peptides, polynucleotides, TCR, CAR, cells containing TCR or CAR, dendritic cell containing polypeptide, dendritic cell containing polynucleotide, antibody, etc.) described herein corresponding to tumor specific non-mutated protein epitope identified herein.

A person skilled in the art will be able to select non-mutated protein epitope therapeutics by testing, for example, the generation of T-cells in vitro as well as their efficiency and overall presence, the proliferation, affinity and expansion of certain T-cells for certain peptides, and the functionality of the T-cells, e.g. by analyzing the IFN-γ production or tumor killing by T-cells. The most efficient peptides can then combined as an immunogenic composition.

In one embodiment of the present invention the different non-mutated protein epitope peptides and/or polypeptides are selected so that one immunogenic composition comprises non-mutated protein epitope peptides and/or polypeptides capable of associating with different MHC molecules, such as different MHC class I molecule. In some embodiments, an immunogenic composition comprises non-mutated protein epitope peptides and/or polypeptides capable of associating with the most frequently occurring MHC class I molecules. Hence immunogenic compositions described herein comprise different peptides capable of associating with at least 2, at least 3, or at least 4 MHC class I or class II molecules.

In one embodiment, an immunogenic composition described herein is capable of raising a specific cytotoxic T-cells response, specific helper T-cell response, or a B cell response.

In some embodiments, an immunogenic composition described herein can further comprise an adjuvant and/or a carrier. Examples of useful adjuvants and carriers are given herein below. Polypeptides and/or polynucleotides in the composition can be associated with a carrier such as e.g. a protein or an antigen-presenting cell such as e.g. a dendritic cell (DC) capable of presenting the peptide to a T-cell or a B cell. In further embodiments, DC-binding peptides are used as carriers to target the non-mutated protein epitope peptides and polynucleotides encoding the non-mutated protein epitope peptides to dendritic cells (Sioud et al. FASEB J 27: 3272-3283 (2013)).

In embodiments, the non-mutated protein epitope polypeptides or polynucleotides can be provided as antigen presenting cells (e.g., dendritic cells) containing such polypeptides or polynucleotides. In other embodiments, such antigen presenting cells are used to stimulate T cells for use in patients.

In some embodiments, the antigen presenting cells are dendritic cells. In related embodiments, the dendritic cells are autologous dendritic cells that are pulsed with the non-mutated protein epitope peptide or nucleic acid. The non-mutated protein epitope peptide can be any suitable peptide that gives rise to an appropriate T-cell response. T-cell therapy using autologous dendritic cells pulsed with peptides from a tumor associated antigen is disclosed in Murphy et al. (1996) The Prostate 29, 371-380 and Tjua et al. (1997) The Prostate 32, 272-278. In some embodiments, the T cell is a CTL. In some embodiments, the T cell is a HTL.

Thus, one embodiment of the present invention an immunogenic composition containing at least one antigen presenting cell (e.g., a dendritic cell) that is pulsed or loaded with one or more non-mutated protein epitope polypeptides or polynucleotides described herein. In embodiments, such APCs are autologous (e.g., autologous dendritic cells). Alternatively, peripheral blood mononuclear cells (PBMCs) isolated from a patient can be loaded with non-mutated protein epitope peptides or polynucleotides ex vivo. In related embodiments, such APCs or PBMCs are injected back into the patient.

The polynucleotide can be any suitable polynucleotide that is capable of transducing the dendritic cell, thus resulting in the presentation of a non-mutated protein epitope peptide and induction of immunity. In one embodiment, the polynucleotide can be naked DNA that is taken up by the cells by passive loading. In another embodiment, the polynucleotide is part of a delivery vehicle, for example, a liposome, virus like particle, plasmid, or expression vector. In another embodiment, the polynucleotide is delivered by a vector-free delivery system, for example, high performance electroporation and high-speed cell deformation). In embodiments, such antigen presenting cells (APCs) (e.g., dendritic cells) or peripheral blood mononuclear cells (PBMCs) are used to stimulate a T cell (e.g., an autologous T cell). In related embodiments, the T cell is a CTL. In other related embodiments, the T cell is an HTL. Such T cells are then injected into the patient. In some embodiments, CTL is injected into the patient. In some embodiments, HTL is injected into the patient. In some embodiments, both CTL and HTL are injected into the patient. Administration of either therapeutic can be performed simultaneously or sequentially and in any order.

The pharmaceutical compositions (e.g., immunogenic compositions) described herein for therapeutic treatment are intended for parenteral, topical, nasal, oral or local administration. In some embodiments, the pharmaceutical compositions described herein are administered parenterally, e.g., intravenously, subcutaneously, intradermally, or intramuscularly. In embodiments, the composition can be administered intratumorally. The compositions can be administered at the site of surgical excision to induce a local immune response to the tumor. In some embodiments, described herein are compositions for parenteral administration which comprise a solution of the non-mutated protein epitope peptides and immunogenic compositions are dissolved or suspended in an acceptable carrier, for example, an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of non-mutated protein epitope peptides and polynucleotides described herein in the pharmaceutical formulations can vary widely, i.e., from less than about 0.1%, usually at or at least about 2% to as much as 20% to 50% or more by weight, and will be selected by fluid volumes, viscosities, etc., according to the particular mode of administration selected.

The non-mutated protein epitope peptides and polynucleotides described herein can also be administered via liposomes, which target the peptides to a particular cells tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the DEC205 antigen, or with other therapeutic or immunogenic compositions. Thus, liposomes filled with a desired peptide or polynucleotide described herein can be directed to the site of lymphoid cells, where the liposomes then deliver the selected therapeutic/immunogenic polypeptide/polynucleotide compositions. Liposomes can be formed from standard vesicle-forming lipids, which generally include neutral and negatively charged phospholipids and a sterol, for example, cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a non-mutated protein epitope polypeptides or polynucleotides to be incorporated into the liposome for cell surface determinants of the desired immune system cells. A liposome suspension containing a peptide can be administered intravenously, locally, topically, etc. in a dose which varies according to, inter alia, the manner of administration, the polypeptide or polynucleotide being delivered, and the stage of the disease being treated.

In some embodiments, non-mutated protein epitope polypeptides and polynucleotides are targeted to dendritic cells. In one embodiment, the non-mutated protein epitope polypeptides and polynucleotides are target to dendritic cells using the markers DEC205, XCR1, CD197, CD80, CD86, CD123, CD209, CD273, CD283, CD289, CD184, CD85h, CD85j, CD85k, CD85d, CD85g, CD85a, TSLP receptor, or CD1a.

For solid compositions, conventional or nanoparticle nontoxic solid carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more non-mutated protein epitope polypeptides or polynucleotides described herein at a concentration of 25%-75%.

For aerosol administration, the non-mutated protein epitope polypeptides or polynucleotides can be supplied in finely divided form along with a surfactant and propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides can be employed. The surfactant can constitute 0.1%-20% by weight of the composition, or 0.25-5%. The balance of the composition can be propellant. A carrier can also be included as desired, as with, e.g., lecithin for intranasal delivery.

Additional methods for delivering the non-mutated protein epitope polynucleotides described herein are also known in the art. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

For therapeutic or immunization purposes, mRNA encoding the non-mutated protein epitope peptides, or peptide binding agents can also be administered to the patient. In one embodiment, the mRNA is self-amplifying RNA. In a further embodiment, the self-amplifying RNA is a part of a synthetic lipid nanoparticle formulation (Geall et al., Proc Natl Acad Sci USA. 109: 14604-14609 (2012)).

The nucleic acids can also be delivered complexed to cationic compounds, such as cationic lipids. Lipid-mediated gene delivery methods are described, for instance, in WO 96/18372, WO 93/24640; Mannino & Gould-Fogerite, BioTechniques 6(7): 682-691 (1988); U.S. Pat. No. 5,279,833; WO 91/06309; and Felgner et al., Proc. Natl. Acad. Sci. USA 84: 7413-7414 (1987).

The non-mutated protein epitope peptides and polypeptides described herein can also be expressed by attenuated viruses, such as vaccinia or fowlpox. This approach involves the use of vaccinia virus as a vector to express nucleotide sequences that encode the peptide described herein. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for therapeutic administration or immunization of the peptides described herein will be apparent to those skilled in the art from the description herein.

Adjuvants are any substance whose admixture into the immunogenic composition increases or otherwise modifies the immune response to the therapeutic agent. Carriers are scaffold structures, for example a polypeptide or a polysaccharide, to which a non-mutated protein epitope polypeptide or polynucleotide, is capable of being associated. Optionally, adjuvants are conjugated covalently or non-covalently to the polypeptides or polynucleotides described herein.

The ability of an adjuvant to increase the immune response to an antigen is typically manifested by a significant increase in immune-mediated reaction, or reduction in disease symptoms. For example, an increase in humoral immunity can be manifested by a significant increase in the titer of antibodies raised to the antigen, and an increase in T-cell activity can be manifested in increased cell proliferation, or cellular cytotoxicity, or cytokine secretion. An adjuvant can also alter an immune response, for example, by changing a primarily humoral or T helper 2 response into a primarily cellular, or T helper 1 response.

Suitable adjuvants are known in the art (see, WO 2015/095811) and include, but are not limited to poly(I:C), poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®. vector system, PLG microparticles, resiquimod, SRL172, virosomes and other virus-like particles, YF-17D, VEGF trap, R848, beta-glucan, Pam3Cys, Pam3CSK4, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants also include incomplete Freund's or GM-CSF. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11) (Mosca et al. Frontiers in Bioscience, 2007; 12:4050-4060) (Gamvrellis et al. Immunol & Cell Biol. 2004; 82: 506-516). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, PGE1, PGE2, IL-1, IL-1b, IL-4, IL-6 and CD40L) (U.S. Pat. No. 5,849,589 incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. Importantly, it enhances dendritic cell maturation and differentiation, resulting in enhanced activation of TH1 cells and strong cytotoxic T-lymphocyte (CTL) generation, even in the absence of CD4 T-cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enabled the antigen doses to be reduced with comparable antibody responses to the full-dose vaccine without CpG in some experiments (Arthur M. Krieg, Nature Reviews, Drug Discovery, 5, June 2006, 471-484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a component of the pharmaceutical composition described herein. Other TLR binding molecules such as RNA binding TLR 7, TLR 8 and/or TLR 9 can also be used.

Other examples of useful adjuvants include, but are not limited to, chemically modified CpGs (e.g. CpR, Idera), Poly(I:C)(e.g. polyi:CI2U), non-CpG bacterial DNA or RNA, ssRNA40 for TLR8, as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which can act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

In some embodiments, an immunogenic composition according to the present invention can comprise more than one different adjuvants. Furthermore, the invention encompasses a therapeutic composition comprising any adjuvant substance including any of the above or combinations thereof. It is also contemplated that the non-mutated protein epitope therapeutic (e.g., a humoral or cell-mediated immune response). In some embodiments, the immunogenic composition comprises non-mutated protein epitope therapeutics (e.g., peptides, polynucleotides, TCR, CAR, cells containing TCR or CAR, dendritic cell containing polypeptide, dendritic cell containing polynucleotide, antibody, etc.) and the adjuvant can be administered separately in any appropriate sequence.

A carrier can be present independently of an adjuvant. The function of a carrier can for example be to increase the molecular weight of in particular mutant in order to increase their activity or immunogenicity, to confer stability, to increase the biological activity, or to increase serum half-life. Furthermore, a carrier can aid presenting peptides to T-cells. The carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. In one embodiment, the carrier comprises a human fibronectin type III domain (Koide et al. Methods Enzymol. 2012; 503:135-56). For immunization of humans, the carrier must be a physiologically acceptable carrier acceptable to humans and safe. However, tetanus toxoid and/or diptheria toxoid are suitable carriers in one embodiment of the invention. Alternatively, the carrier can be dextrans for example sepharose.

In some embodiments, the polypeptides can be synthesized as multiply linked peptides as an alternative to coupling a polypeptide to a carrier to increase immunogenicity. Such molecules are also known as multiple antigenic peptides (MAPS).

### IV. Combinations of CTL Peptides and HTL Peptides

Immunogenic or vaccine compositions comprising the non-mutated protein epitope polypeptides and polynucleotides described herein, or analogs thereof, which have immunostimulatory activity can be modified to provide desired attributes, such as improved serum half-life, or to enhance immunogenicity.

For instance, the ability of the non-mutated protein epitope peptides to induce CTL activity can be enhanced by linking the peptide to a sequence which contains at least one epitope that is capable of inducing a T helper cell response. In one embodiment, CTL epitope/HTL epitope conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the CTL peptide can be linked to the T helper peptide without a spacer.

Although the CTL peptide epitope can be linked directly to the T helper peptide epitope, CTL epitope/HTL epitope conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. The CTL peptide epitope can be linked to the T helper peptide epitope either directly or via a spacer either at the amino or carboxy terminus of the CTL peptide. The amino terminus of either the immunogenic peptide or the T helper peptide can be acylated.

HTL peptide epitopes can also be modified to alter their biological properties. For example, peptides comprising HTL epitopes can contain D-amino acids to increase their resistance to proteases and thus extend their serum half-life. Also, the epitope peptides can be conjugated to other molecules such as lipids, proteins or sugars, or any other synthetic compounds, to increase their biological activity. For example, the T helper peptide can be conjugated to one or more palmitic acid chains at either the amino or carboxyl termini.

In certain embodiments, the T helper peptide is one that is recognized by T helper cells present in the majority of the population. This can be accomplished by selecting amino acid sequences that bind to many, most, or all of the HLA class II molecules. These are known as “loosely HLA-restricted” or “promiscuous” T helper sequences. Examples of amino acid sequences that are promiscuous include sequences from antigens such as tetanus toxoid at positions 830-843 (QYIKANSKFIGITE (SEQ ID NO: 1)), Plasmodium falciparum: CS protein at positions 378-398 (DIEKKIAKMEKASSVFNVVNS (SEQ ID NO: 2)), and Streptococcus 18 kD protein at positions 116 (GAVDSILGGVATYGAA (SEQ ID NO: 3)). Other examples include peptides bearing a DR 1-4-7 supermotif, or either of the DR3 motifs.

Alternatively, it is possible to prepare synthetic peptides capable of stimulating T helper lymphocytes, in a loosely HLA-restricted fashion, using amino acid sequences not found in nature (see, e.g., PCT publication WO 95/07707). These synthetic compounds called Pan-DR-binding epitopes (e.g., PADRE, Epimmune, Inc., San Diego, CA) are designed to bind most HLA-DR (human HLA class II) molecules. For instance, a pan-DR-binding epitope peptide having the formula: aKXVWANTLKAAa (SEQ ID NO: 4), where “X” is either cyclohexylalanine, phenylalanine, or tyrosine, and a is either D-alanine or L-alanine, has been found to bind to most HLA-DR alleles, and to stimulate the response of T helper lymphocytes from most individuals, regardless of their HLA type. An alternative of a pan-DR binding epitope comprises all “L” natural amino acids and can be provided in the form of nucleic acids that encode the epitope.

In some embodiments it can be desirable to include in anon-mutated protein epitope therapeutic (e.g., peptides, polynucleotides, TCR, CAR, cells containing TCR or CAR, dendritic cell containing polypeptide, dendritic cell containing polynucleotide, antibody, etc.) in pharmaceutical compositions (e.g., immunogenic compositions) at least one component of which primes cytotoxic T lymphocytes. Lipids have been identified as agents capable of priming CTL in vivo against viral antigens. For example, palmitic acid residues can be attached to the ε- and α-amino groups of a lysine residue and then linked, e.g., via one or more linking residues such as Gly, Gly-Gly-, Ser, Ser-Ser, or the like, to an immunogenic non-mutated protein epitope peptide. The lipidated peptide can then be administered either directly in a micelle or particle, incorporated into a liposome, or emulsified in an adjuvant. In one embodiment, a particularly effective immunogenic construct comprises palmitic acid attached to ε- and α-amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P3CSS) can be used to prime virus specific CTL when covalently attached to an appropriate peptide. (See, e.g., Deres, et al., Nature 342:561, 1989). Non-mutated protein epitope peptides described herein can be coupled to P3CSS, for example, and the lipopeptide administered to an individual to specifically prime a CTL response to the target antigen. Moreover, because the induction of neutralizing antibodies can also be primed with P3CSS-conjugated epitopes, two such compositions can be combined to more effectively elicit both humoral and cell-mediated responses to infection.

As noted herein, additional amino acids can be added to the termini of anon-mutated protein epitope peptide to provide for ease of linking peptides one to another, for coupling to a carrier support or larger peptide, for modifying the physical or chemical properties of the peptide or oligopeptide, or the like. Amino acids such as tyrosine, cysteine, lysine, glutamic or aspartic acid, or the like, can be introduced at the C- or N-terminus of the peptide or oligopeptide. However, it is to be noted that modification at the carboxyl terminus of a T cell epitope can, in some cases, alter binding characteristics of the peptide. In addition, the peptide or oligopeptide sequences can differ from the natural sequence by being modified by terminal-NH2 acylation, e.g., by alkanoyl (C1-C20) or thioglycolyl acetylation, terminal-carboxyl amidation, e.g., ammonia, methylamine, etc. In some instances these modifications can provide sites for linking to a support or other molecule.

An embodiment of an immunogenic composition described herein comprises ex vivo administration of a cocktail of epitope-bearing non-mutated protein epitope polypeptide or polynucleotides to PBMC, or isolated DC therefrom, from the patient's blood. A pharmaceutical to facilitate harvesting of dendritic cells (DCs) can be used, including GM-CSF, IL-4, IL-6, IL-10, and TNFα. After pulsing the DCs with peptides or polynucleotides encoding the peptides, and prior to reinfusion into patients, the DC are washed to remove unbound peptides. In this embodiment, a vaccine or immunogenic composition comprises peptide-pulsed DCs which present the pulsed peptide epitopes complexed with HLA molecules on their surfaces. The composition is then administered to the patient. In other embodiments, such pulsed DCs are used to stimulate T cells suitable for use in T cell therapy.

### V. Multi-Epitope Immunogenic Compositions

A number of different approaches are available which allow simultaneous delivery of multiple epitopes. Nucleic acids encoding the non-mutated protein epitope peptides described herein are a particularly useful embodiment of the invention. In one embodiment, the nucleic acid is RNA. In some embodiments, minigene constructs encoding a non-mutated protein epitope peptide comprising one or multiple epitopes described herein are used to administer nucleic acids encoding the non-mutated protein epitope peptides described herein uses.

The use of multi-epitope minigenes is described An, L. and Whitton, J. L., J. Virol. 71:2292, 1997; Thomson, S. A. et al., J. Immunol. 157:822, 1996; Whitton, J. L. et al., J. Virol. 67:348, 1993; Hanke, R. et al., Vaccine 16:426, 1998. For example, a multi-epitope DNA plasmid encoding supermotif- and/or motif-bearing antigen peptides, a universal helper T cell epitope (or multiple tumor associated antigen HTL epitopes), and an endoplasmic reticulum-translocating signal sequence can be engineered.

The immunogenicity of a multi-epitopic minigene can be tested in transgenic mice to evaluate the magnitude of immune response induced against the epitopes tested. Further, the immunogenicity of DNA-encoded epitopes in vivo can be correlated with the in vitro responses of specific CTL lines against target cells transfected with the DNA plasmid. Thus, these experiments can show that the minigene serves to both: 1.) generate a cell mediated and/or humoral response and 2.) that the induced immune cells recognized cells expressing the encoded epitopes.

For example, to create a DNA sequence encoding the selected non-mutated protein epitope (minigene) for expression in human cells, the amino acid sequences of the epitopes can be reverse translated. A human codon usage table can be used to guide the codon choice for each amino acid. These non-mutated protein epitope-encoding DNA sequences can be directly adjoined, so that when translated, a continuous polypeptide sequence is created. To optimize expression and/or immunogenicity, additional elements can be incorporated into the minigene design. Examples of amino acid sequences that can be reverse translated and included in the minigene sequence include: HLA class I epitopes, HLA class II epitopes, a ubiquitination signal sequence, and/or an endoplasmic reticulum targeting signal. In addition, HLA presentation of CTL and HTL epitopes can be improved by including synthetic (e.g. poly-alanine) or naturally-occurring flanking sequences adjacent to the CTL or HTL epitopes; these larger peptides comprising the epitope(s) are within the scope of the invention.

The minigene sequence can be converted to DNA by assembling oligonucleotides that encode the plus and minus strands of the minigene. Overlapping oligonucleotides (30-100 bases long) can be synthesized, phosphorylated, purified and annealed under appropriate conditions using well known techniques. The ends of the oligonucleotides can be joined, for example, using T4 DNA ligase. This synthetic minigene, encoding the epitope polypeptide, can then be cloned into a desired expression vector.

Standard regulatory sequences well known to those of skill in the art can be included in the vector to ensure expression in the target cells. For example, a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences.

Additional vector modifications can be used to optimize minigene expression and immunogenicity. In some cases, introns are utilized for efficient gene expression, and one or more synthetic or naturally-occurring introns could be incorporated into the transcribed region of the minigene. The inclusion of mRNA stabilization sequences and sequences for replication in mammalian cells can also be considered for increasing minigene expression.

Once an expression vector is selected, the minigene can be cloned into the polylinker region downstream of the promoter. This plasmid is transformed into an appropriate E. coli strain, and DNA is prepared using standard techniques. The orientation and DNA sequence of the minigene, as well as all other elements included in the vector, can be confirmed using restriction mapping and DNA sequence analysis. Bacterial cells harboring the correct plasmid can be stored as a master cell bank and a working cell bank.

In addition, immunomodulatory sequences appear to play a role in the immunogenicity of DNA vaccines. These sequences can be included in the vector, outside the minigene coding sequence, if desired to enhance immunogenicity. In one embodiment, the sequences are immunostimulatory. In another embodiment, the sequences are ISSs or CpGs.

In some embodiments, a bi-cistronic expression vector which allows production of both the minigene-encoded epitopes and a second protein (included to enhance or decrease immunogenicity) can be used. Examples of proteins or polypeptides that could beneficially enhance the immune response if co-expressed include cytokines (e.g., IL-2, IL-12, GM-CSF), cytokine-inducing molecules (e.g., LeIF), costimulatory molecules, or for HTL responses, pan-DR binding proteins. Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-β) can be beneficial in certain diseases.

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well-known techniques. Plasmid DNA can be purified using standard bioseparation technologies such as solid phase anion-exchange resins supplied by QIAGEN, Inc. (Valencia, California). If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, known as “naked DNA,” is currently being used for intramuscular (IM) administration in clinical trials. To maximize the immunotherapeutic effects of minigene DNA vaccines, an alternative method for formulating purified plasmid DNA can be used. A variety of methods have been described, and new techniques can become available. Cationic lipids can also be used in the formulation (see, e.g., as described by WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In addition, glycolipids, fusogenic liposomes, peptides and compounds referred to collectively as protective, interactive, non-condensing compounds (PINC) could also be complexed to purified plasmid DNA to influence variables such as stability, intramuscular dispersion, or trafficking to specific organs or cell types.

In another embodiment, the nucleic acid is introduced into cells by use of high-speed cell deformation. During high-speed deformation, cells are squeezed such that temporary disruptions occur in the cell membrane, thus allowing the nucleic acid to enter the cell. Alternatively, protein can be produced from expression vectors—in a bacterial expression vector, for example, and the proteins can then be delivered to the cell.

Target cell sensitization can be used as a functional assay for expression and HLA class I presentation of minigene-encoded CTL epitopes. For example, the plasmid DNA is introduced into a mammalian cell line that is suitable as a target for standard CTL chromium release assays. The transfection method used will be dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 (⁵¹Cr) labeled and used as target cells for epitope-specific CTL lines; cytolysis, detected by ⁵¹Cr release, indicates both production of, and HLA presentation of, minigene-encoded CTL epitopes. Expression of HTL epitopes can be evaluated in an analogous manner using assays to assess HTL activity.

In vivo immunogenicity is a second approach for functional testing of minigene DNA formulations. Transgenic mice expressing appropriate human HLA proteins are immunized with the DNA product. The dose and route of administration are formulation dependent (e.g., IM for DNA in PBS, intraperitoneal (IP) for lipid-complexed DNA). An exemplary protocol is twenty-one days after immunization, splenocytes are harvested and restimulated for 1 week in the presence of peptides encoding each epitope being tested. Thereafter, for CTL effector cells, assays are conducted for cytolysis of peptide-loaded, ⁵¹Cr-labeled target cells using standard techniques. Lysis of target cells that were sensitized by HLA loaded with peptide epitopes, corresponding to minigene-encoded epitopes, demonstrates DNA vaccine function for in vivo induction of CTLs. Immunogenicity of HTL epitopes is evaluated in transgenic mice in an analogous manner.

Alternatively, the nucleic acids can be administered using ballistic delivery as described, for instance, in U.S. Pat. No. 5,204,253. Using this technique, particles comprised solely of DNA are administered. In a further alternative embodiment, DNA can be adhered to particles, such as gold particles.

### VI. Cells

In one aspect, the present invention also provides cells expressing a non-mutated protein epitope-recognizing receptor that activates an immunoresponsive cell (e.g., T cell receptor (TCR) or chimeric antigen receptor (CAR)), and methods of using such cells for the treatment of a disease that requires an enhanced immune response.

Such cells include genetically modified immunoresponsive cells (e.g., T cells, Natural Killer (NK) cells, cytotoxic T lymphocytes (CTL) cells, helper T lymphocyte (HTL) cells) expressing an antigen-recognizing receptor (e.g., TCR or CAR) that binds one of the non-mutated protein epitope peptides described herein, and methods of use therefore for the treatment of neoplasia and other pathologies where an increase in an antigen-specific immune response is desired. T cell activation is mediated by a TCR or a CAR targeted to an antigen.

The present invention provides cells expressing a combination of an antigen-recognizing receptor that activates an immunoresponsive cell (e.g., TCR, CAR) and a chimeric co-stimulating receptor (CCR), and methods of using such cells for the treatment of a disease that requires an enhanced immune response. In one embodiment, tumor antigen-specific T cells, NK cells, CTL cells or other immunoresponsive cells are used as shuttles for the selective enrichment of one or more co-stimulatory ligands for the treatment or prevention of neoplasia. Such cells are administered to a human subject in need thereof for the treatment or prevention of a particular cancer.

In one embodiment, the tumor antigen-specific human lymphocytes that can be used in the methods of the invention include, without limitation, peripheral donor lymphocytes genetically modified to express chimeric antigen receptors (CARs) (Sadelain, M., et al. 2003 Nat Rev Cancer 3:35-45), peripheral donor lymphocytes genetically modified to express a full-length tumor antigen-recognizing T cell receptor complex comprising the a and p heterodimer (Morgan, R. A., et al. 2006 Science 314:126-129), lymphocyte cultures derived from tumor infiltrating lymphocytes (TILs) in tumor biopsies (Panelli, M. C., et al. 2000 J Immunol 164:495-504; Panelli, M. C., et al. 2000 J Immunol 164:4382-4392), and selectively in vitro-expanded antigen-specific peripheral blood leukocytes employing artificial antigen-presenting cells (AAPCs) or pulsed dendritic cells (Dupont, J., et al. 2005 Cancer Res 65:5417-5427; Papanicolaou, G. A., et al. 2003 Blood 102:2498-2505). The T cells may be autologous, allogeneic, or derived in vitro from engineered progenitor or stem cells.

### Co-Stimulatory Ligands

In one embodiment, the cells of the invention are provided with at least one co-stimulatory ligand which is a non-antigen specific signal important for full activation of an immune cell. Co-stimulatory ligands include, without limitation, tumor necrosis factor (TNF) ligands, cytokines (such as IL-2, IL-12, IL-15 or IL21), and immunoglobulin (Ig) superfamily ligands.

Tumor necrosis factor (TNF) is a cytokine involved in systemic inflammation and stimulates the acute phase reaction. Its primary role is in the regulation of immune cells. Tumor necrosis factor (TNF) ligands share a number of common features. The majority of the ligands are synthesized as type II transmembrane proteins containing a short cytoplasmic segment and a relatively long extracellular region. TNF ligands include, without limitation, nerve growth factor (NGF), CD40L (CD40L)/CD154, CD137L/4-1BBL, tumor necrosis factor alpha (TNFα), CD134L/OX40L/CD252, CD27L/CD70, Fas ligand (FasL), CD30L/CD153, tumor necrosis factor β (TNF(3)/lymphotoxin-alpha (LTa), lymphotoxin-beta (ur(3), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, without limitation, CD80 and CD86, both ligands for CD28.

Compositions comprising genetically modified immunoresponsive cells of the invention can be provided systemically or directly to a subject for the treatment of a neoplasia. In one embodiment, cells of the invention are directly injected into an organ of interest (e.g., an organ affected by a tumor). Alternatively, compositions comprising genetically modified immunoresponsive cells are provided indirectly to the organ of interest, for example, by administration into the circulatory system (e.g., the tumor vasculature). Expansion and differentiation agents can be provided prior to, during or after administration of the cells to increase production of T cells, NK cells, or CTL cells in vitro or in vivo.

The modified cells can be administered in any physiologically acceptable vehicle, normally intravascularly, although they may also be introduced into bone or other convenient site where the cells may find an appropriate site for regeneration and differentiation (e.g., thymus). Genetically modified immunoresponsive cells of the invention can comprise a purified population of cells. Those skilled in the art can readily determine the percentage of genetically modified immunoresponsive cells in a population using various well-known methods, such as fluorescence activated cell sorting (FACS). Dosages can be readily adjusted by those skilled in the art (e.g., a decrease in purity may require an increase in dosage). The cells can be introduced by injection, catheter, or the like. If desired, factors can also be included, including, but not limited to, interleukins, e.g. IL-2, IL-3, IL-6, and IL-11, as well as the other interleukins, the colony stimulating factors, such as G-, M- and GM-CSF, interferons, e.g. γ-interferon and erythropoietin.

Compositions of the invention include pharmaceutical compositions comprising genetically modified immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. Peripheral blood derived immunoresponsive cells of the invention or their progeny (e.g., in vivo, ex vivo or in vitro derived) can be administered via localized injection, including catheter administration, systemic injection, localized injection, intravenous injection, or parenteral administration. When administering a therapeutic composition of the present invention (e.g., a pharmaceutical composition containing a genetically modified immunoresponsive cell), it will generally be formulated in a unit dosage injectable form (solution, suspension, emulsion).

### VII. Methods of Use and Pharmaceutical Compositions

The non-mutated protein epitope therapeutics (e.g., peptides, polynucleotides, TCR, CAR, cells containing TCR or CAR, dendritic cell containing polypeptide, dendritic cell containing polynucleotide, antibody, etc.) described herein are useful in a variety of applications including, but not limited to, therapeutic treatment methods, such as the treatment of cancer. In some embodiments, the therapeutic treatment methods comprise immunotherapy. In certain embodiments, a non-mutated protein epitope peptide is useful for activating, promoting, increasing, and/or enhancing an immune response, redirecting an existing immune response to a new target, increasing the immunogenicity of a tumor, inhibiting tumor growth, reducing tumor volume, increasing tumor cell apoptosis, and/or reducing the tumorigenicity of a tumor. The methods of use can be in vitro, ex vivo, or in vivo methods.

In some aspects, the present invention provides methods for activating an immune response in a subject using a non-mutated protein epitope therapeutic described herein. In some embodiments, the invention provides methods for promoting an immune response in a subject using a non-mutated protein epitope therapeutic described herein. In some embodiments, the invention provides methods for increasing an immune response in a subject using a non-mutated protein epitope peptide described herein. In some embodiments, the invention provides methods for enhancing an immune response using a non-mutated protein epitope peptide. In some embodiments, the activating, promoting, increasing, and/or enhancing of an immune response comprises increasing cell-mediated immunity. In some embodiments, the activating, promoting, increasing, and/or enhancing of an immune response comprises increasing T-cell activity or humoral immunity. In some embodiments, the activating, promoting, increasing, and/or enhancing of an immune response comprises increasing CTL or HTL activity. In some embodiments, the activating, promoting, increasing, and/or enhancing of an immune response comprises increasing NK cell activity. In some embodiments, the activating, promoting, increasing, and/or enhancing of an immune response comprises increasing T-cell activity and increasing NK cell activity. In some embodiments, the activating, promoting, increasing, and/or enhancing of an immune response comprises increasing CTL activity and increasing NK cell activity. In some embodiments, the activating, promoting, increasing, and/or enhancing of an immune response comprises inhibiting or decreasing the suppressive activity of Tregs. In some embodiments, the immune response is a result of antigenic stimulation. In some embodiments, the antigenic stimulation is a tumor cell. In some embodiments, the antigenic stimulation is cancer.

In some embodiments, the invention provides methods of activating, promoting, increasing, and/or enhancing of an immune response using a non-mutated protein epitope therapeutic described herein. In some embodiments, a method comprises administering to a subject in need thereof a therapeutically effective amount of a non-mutated protein epitope therapeutic that delivers a non-mutated protein epitope polypeptide or polynucleotide to a tumor cell. In some embodiments, a method comprises administering to a subject in need thereof a therapeutically effective amount of a non-mutated protein epitope therapeutic that binds the tumor associated antigen and is internalized by the tumor cell. In some embodiments, a method comprises administering to a subject in need thereof a therapeutically effective amount of a non-mutated protein epitope polypeptide that is internalized by a tumor cell, and the non-mutated protein epitope peptide is processed by the cell. In some embodiments, a method comprises administering to a subject in need thereof a therapeutically effective amount of a non-mutated protein epitope polypeptide that is internalized by a tumor cell, and an antigenic peptide is presented on the surface of the tumor cell. In some embodiments, a method comprises administering to a subject in need thereof a therapeutically effective amount of a non-mutated protein epitope polypeptide that is internalized by the tumor cell, is processed by the cell, and an antigenic peptide is presented on the surface of the tumor cell.

In some embodiments, a method comprises administering to a subject in need thereof a therapeutically effective amount of a non-mutated protein epitope polypeptide or polynucleotide described herein that delivers an exogenous polypeptide comprising at least one antigenic peptide to a tumor cell, wherein the antigenic peptide is presented on the surface of the tumor cell. In some embodiments, the antigenic peptide is presented on the surface of the tumor cell in complex with a MHC class I molecule. In some embodiments, the antigenic peptide is presented on the surface of the tumor cell in complex with a MHC class II molecule.

In some embodiments, a method comprises contacting a tumor cell with a non-mutated protein epitope polypeptide or polynucleotide described herein that delivers an exogenous polypeptide comprising at least one antigenic peptide to the tumor cell, wherein the antigenic peptide is presented on the surface of the tumor cell. In some embodiments, the antigenic peptide is presented on the surface of the tumor cell in complex with a MHC class I molecule. In some embodiments, the antigenic peptide is presented on the surface of the tumor cell in complex with a MHC class II molecule.

In some embodiments, a method comprises administering to a subject in need thereof a therapeutically effective amount of a non-mutated protein epitope polypeptide or polynucleotide described herein that delivers an exogenous polypeptide comprising at least one antigenic peptide to a tumor cell, wherein the antigenic peptide is presented on the surface of the tumor cell, and an immune response against the tumor cell is induced. In some embodiments, the immune response against the tumor cell is increased. In some embodiments, the non-mutated protein epitope polypeptide or polynucleotide delivers an exogenous polypeptide comprising at least one antigenic peptide to a tumor cell, wherein the antigenic peptide is presented on the surface of the tumor cell, and tumor growth is inhibited.

In some embodiments, a method comprises administering to a subject in need thereof a therapeutically effective amount of a non-mutated protein epitope polypeptide or polynucleotide described herein that delivers an exogenous polypeptide comprising at least one antigenic peptide to a tumor cell, wherein the antigenic peptide is presented on the surface of the tumor cell, and T-cell killing directed against the tumor cell is induced. In some embodiments, T-cell killing directed against the tumor cell is enhanced. In some embodiments, T-cell killing directed against the tumor cell is increased.

In some embodiments, a method of increasing an immune response in a subject comprises administering to the subject a therapeutically effective amount of a non-mutated protein epitope therapeutic described herein, wherein the agent is an antibody that specifically binds the non-mutated protein epitope described herein. In some embodiments, a method of increasing an immune response in a subject comprises administering to the subject a therapeutically effective amount of the antibody.

The present invention provides methods of redirecting an existing immune response to a tumor. In some embodiments, a method of redirecting an existing immune response to a tumor comprises administering to a subject a therapeutically effective amount of a non-mutated protein epitope therapeutic described herein. In some embodiments, the existing immune response is against a virus. In some embodiments, the virus is selected from the group consisting of measles virus, varicella-zoster virus (VZV; chickenpox virus), influenza virus, mumps virus, poliovirus, rubella virus, rotavirus, hepatitis A virus (HAV), hepatitis B virus (HBV), Epstein Barr virus (EBV), and cytomegalovirus (CMV). In some embodiments, the virus is varicella-zoster virus. In some embodiments, the virus is cytomegalovirus. In some embodiments, the virus is measles virus. In some embodiments, the existing immune response has been acquired after a natural viral infection. In some embodiments, the existing immune response has been acquired after vaccination against a virus. In some embodiments, the existing immune response is a cell-mediated response. In some embodiments, the existing immune response comprises cytotoxic T-cells (CTLs) or HTLs.

In some embodiments, a method of redirecting an existing immune response to a tumor in a subject comprises administering a fusion protein comprising (i) an antibody that specifically binds a non-mutated protein epitope and (ii) at least one non-mutated protein epitope peptide described herein, wherein (a) the fusion protein is internalized by a tumor cell after binding to the tumor-associated antigen; (b) the non-mutated protein epitope peptide is processed and presented on the surface of the tumor cell associated with a MHC class I molecule; and (c) the non-mutated protein epitope peptide/MHC Class I complex is recognized by cytotoxic T-cells. In some embodiments, the cytotoxic T-cells are memory T-cells. In some embodiments, the memory T-cells are the result of a vaccination with the non-mutated protein epitope peptide.

The present invention provides methods of increasing the immunogenicity of a tumor. In some embodiments, a method of increasing the immunogenicity of a tumor comprises contacting the tumor or tumor cells with an effective amount of a non-mutated protein epitope therapeutic described herein. In some embodiments, a method of increasing the immunogenicity of a tumor comprises administering to a subject a therapeutically effective amount of a non-mutated protein epitope therapeutic described herein.

The present invention also provides methods for inhibiting growth of a tumor using a non-mutated protein epitope therapeutic described herein. In certain embodiments, a method of inhibiting growth of a tumor comprises contacting a cell mixture with a non-mutated protein epitope therapeutic in vitro. For example, an immortalized cell line or a cancer cell line mixed with immune cells (e.g., T-cells) is cultured in medium to which a non-mutated protein epitope peptide is added. In some embodiments, tumor cells are isolated from a patient sample such as, for example, a tissue biopsy, pleural effusion, or blood sample, mixed with immune cells (e.g., T-cells), and cultured in medium to which an antigen therapeutic is added. In some embodiments, a non-mutated protein epitope therapeutic increases, promotes, and/or enhances the activity of the immune cells. In some embodiments, a non-mutated protein epitope therapeutic inhibits tumor cell growth. In some embodiments, a non-mutated protein epitope therapeutic activates killing of the tumor cells.

In certain embodiments, the subject is a human. In certain embodiments, the subject has a tumor or the subject had a tumor which was at least partially removed.

In some embodiments, a method of inhibiting growth of a tumor comprises redirecting an existing immune response to a new target, comprising administering to a subject a therapeutically effective amount of a non-mutated protein epitope therapeutic, wherein the existing immune response is against an antigenic peptide delivered to the tumor cell by the non-mutated protein epitope peptide.

In certain embodiments, the tumor comprises cancer stem cells. In certain embodiments, the frequency of cancer stem cells in the tumor is reduced by administration of the non-mutated protein epitope therapeutic. In some embodiments, a method of reducing the frequency of cancer stem cells in a tumor in a subject, comprising administering to the subject a therapeutically effective amount of a non-mutated protein epitope therapeutic is provided.

In addition, in some aspects the invention provides a method of reducing the tumorigenicity of a tumor in a subject, comprising administering to the subject a therapeutically effective amount of a non-mutated protein epitope therapeutic described herein. In certain embodiments, the tumor comprises cancer stem cells. In some embodiments, the tumorigenicity of a tumor is reduced by reducing the frequency of cancer stem cells in the tumor. In some embodiments, the methods comprise using the non-mutated protein epitope therapeutic described herein. In certain embodiments, the frequency of cancer stem cells in the tumor is reduced by administration of a non-mutated protein epitope therapeutic described herein.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a breast tumor. In some embodiments, the tumor is a lung tumor. In certain embodiments, the tumor is a pancreatic tumor. In certain embodiments, the tumor is a melanoma tumor. In some embodiments, the tumor is a solid tumor.

The present invention further provides methods for treating cancer in a subject comprising administering to the subject a therapeutically effective amount of a non-mutated protein epitope therapeutic described herein.

In some embodiments, a method of treating cancer comprises redirecting an existing immune response to a new target, the method comprising administering to a subject a therapeutically effective amount of non-mutated protein epitope therapeutic, wherein the existing immune response is against an antigenic peptide delivered to the cancer cell by the non-mutated protein epitope peptide.

The present invention provides for methods of treating cancer comprising administering to a subject a therapeutically effective amount of a non-mutated protein epitope therapeutic described herein (e.g., a subject in need of treatment). In certain embodiments, the subject is a human. In certain embodiments, the subject has a cancerous tumor. In certain embodiments, the subject has had a tumor at least partially removed.

In certain embodiments, the cancer is a cancer selected from the group consisting of colorectal cancer, renal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, glioblastoma, triple-negative breast cancer (TNBC), smoldering myeloma (SMM), and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma. In some embodiments, the cancer is a solid cancer. In some embodiments, the cancer comprises a solid tumor.

In some embodiments, the cancer is a hematologic cancer. In some embodiment, the cancer is selected from the group consisting of: acute myelogenous leukemia (AML), Hodgkin lymphoma, multiple myeloma, T-cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T-cell lymphoma (CTCL).

In some embodiments, the non-mutated protein epitope therapeutic is administered as a combination therapy. Combination therapy with two or more therapeutic agents uses agents that work by different mechanisms of action, although this is not required. Combination therapy using agents with different mechanisms of action can result in additive or synergetic effects. Combination therapy can allow for a lower dose of each agent than is used in monotherapy, thereby reducing toxic side effects and/or increasing the therapeutic index of the agent(s). Combination therapy can decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a non-mutated protein epitope therapeutic described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that can be administered in combination with the non-mutated protein epitope therapeutic described herein include chemotherapeutic agents. Thus, in some embodiments, the method or treatment involves the administration of an agent described herein in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, PA.

Useful classes of chemotherapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylomithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4 hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite utilized for normal biochemical reactions, yet different enough to interfere with one or more normal functions of cells, such as cell division. Anti-metabolites include, but are not limited to, gemcitabine, fluorouracil, capecitabine, methotrexate sodium, raltitrexed, pemetrexed, tegafur, cytosine arabinoside, thioguanine, 5-azacytidine, 6 mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In some embodiments, the additional therapeutic agent is albumin-bound paclitaxel.

In some embodiments, an additional therapeutic agent comprises an agent such as a small molecule. For example, treatment can involve the combined administration of an agent of the present invention with a small molecule that acts as an inhibitor against tumor-associated antigens including, but not limited to, EGFR, HER2 (ErbB2), and/or VEGF. In some embodiments, an agent is administered in combination with a protein kinase inhibitor selected from the group consisting of gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor. In another embodiment, the additional therapeutic agent is chemotherapy or other inhibitors that reduce the number of Treg cells. In certain embodiments, the therapeutic agent is cyclophosphamide or an anti-CTLA4 antibody. In another embodiment, the additional therapeutic reduces the presence of myeloid-derived suppressor cells. In a further embodiment, the additional therapeutic is carbotaxol. In another embodiment, the additional therapeutic agent shifts cells to a T helper 1 response. In a further embodiment, the additional therapeutic agent is ibrutinib.

In some embodiments, an additional therapeutic agent comprises a biological molecule, such as an antibody. For example, treatment can involve the combined administration of an agent of the present invention with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In certain embodiments, an additional therapeutic agent comprises a second immunotherapeutic agent. In some embodiments, the additional immunotherapeutic agent includes, but is not limited to, a colony stimulating factor, an interleukin, an antibody that blocks immunosuppressive functions (e.g., an anti-CTLA-4 antibody, anti-CD28 antibody, anti-CD3 antibody, anti-PD-1 antibody, anti-PD-L1 antibody, anti-TIGIT antibody), an antibody that enhances immune cell functions (e.g., an anti-GITR antibody, an anti-OX-40 antibody, an anti-CD40 antibody, or an anti-4-1BB antibody), a toll-like receptor (e.g., TLR4, TLR7, TLR9), a soluble ligand (e.g., GITRL, GITRL-Fc, OX-40L, OX-40L-Fc, CD40L, CD40L-Fc, 4-1BB ligand, or 4-1BB ligand-Fc), or a member of the B7 family (e.g., CD80, CD86). In some embodiments, the additional immunotherapeutic agent targets CTLA-4, CD28, CD3, PD-1, PD-L1, TIGIT, GITR, OX-40, CD-40, or 4-1BB.

In some embodiments, the additional therapeutic agent is an immune checkpoint inhibitor. In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAG3 antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-PD-1 antibody selected from the group consisting of: nivolumab (OPDIVO), pembrolizumab (KEYTRUDA), pidilzumab, MEDI0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MED14736), and avelumab (MSB0010718C). In some embodiments, the additional therapeutic agent is an anti-CTLA-4 antibody selected from the group consisting of: ipilimumab (YERVOY) and tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566.

In some embodiments, the non-mutated protein epitope therapeutic can be administered in combination with a biologic molecule selected from the group consisting of: adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF-β, TNF-α, VEGF, P1GF, gamma-IFN, IL-1, IL-2, IL-3, IL-4, IL-5, IL-6, IL-7, IL-12, IL-15, and IL-18.

In some embodiments, treatment with a non-mutated protein epitope therapeutic described herein can be accompanied by surgical removal of tumors, removal of cancer cells, or any other surgical therapy deemed necessary by a treating physician.

In certain embodiments, treatment involves the administration of a non-mutated protein epitope therapeutic described herein in combination with radiation therapy. Treatment with an agent can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously.

It will be appreciated that the combination of a non-mutated protein epitope therapeutic described herein and at least one additional therapeutic agent can be administered in any order or concurrently. In some embodiments, the agent will be administered to patients that have previously undergone treatment with a second therapeutic agent. In certain other embodiments, the non-mutated protein epitope therapeutic and a second therapeutic agent will be administered substantially simultaneously or concurrently. For example, a subject can be given an agent while undergoing a course of treatment with a second therapeutic agent (e.g., chemotherapy). In certain embodiments, a non-mutated protein epitope therapeutic will be administered within 1 year of the treatment with a second therapeutic agent. It will further be appreciated that the two (or more) agents or treatments can be administered to the subject within a matter of hours or minutes (i.e., substantially simultaneously).

For the treatment of a disease, the appropriate dosage of a non-mutated protein epitope therapeutic described herein depends on the type of disease to be treated, the severity and course of the disease, the responsiveness of the disease, whether the agent is administered for therapeutic or preventative purposes, previous therapy, the patient's clinical history, and so on, all at the discretion of the treating physician. The non-mutated protein epitope therapeutic can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates.

In some embodiments, a non-mutated protein epitope therapeutic can be administered at an initial higher “loading” dose, followed by one or more lower doses. In some embodiments, the frequency of administration can also change. In some embodiments, a dosing regimen can comprise administering an initial dose, followed by additional doses (or “maintenance” doses) once a week, once every two weeks, once every three weeks, or once every month. For example, a dosing regimen can comprise administering an initial loading dose, followed by a weekly maintenance dose of, for example, one-half of the initial dose. Or a dosing regimen can comprise administering an initial loading dose, followed by maintenance doses of, for example one-half of the initial dose every other week. Or a dosing regimen can comprise administering three initial doses for 3 weeks, followed by maintenance doses of, for example, the same amount every other week.

As is known to those of skill in the art, administration of any therapeutic agent can lead to side effects and/or toxicities. In some cases, the side effects and/or toxicities are so severe as to preclude administration of the particular agent at a therapeutically effective dose. In some cases, therapy must be discontinued, and other agents can be tried. However, many agents in the same therapeutic class display similar side effects and/or toxicities, meaning that the patient either has to stop therapy, or if possible, suffer from the unpleasant side effects associated with the therapeutic agent.

In some embodiments, the dosing schedule can be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

The present invention provides methods of administering to a subject a non-mutated protein epitope therapeutic described herein comprising using an intermittent dosing strategy for administering one or more agents, which can reduce side effects and/or toxicities associated with administration of an agent, chemotherapeutic agent, etc. In some embodiments, a method for treating cancer in a human subject comprises administering to the subject a therapeutically effective dose of a non-mutated protein epitope therapeutic in combination with a therapeutically effective dose of a chemotherapeutic agent, wherein one or both of the agents are administered according to an intermittent dosing strategy. In some embodiments, a method for treating cancer in a human subject comprises administering to the subject a therapeutically effective dose of a non-mutated protein epitope therapeutic in combination with a therapeutically effective dose of a second immunotherapeutic agent, wherein one or both of the agents are administered according to an intermittent dosing strategy. In some embodiments, the intermittent dosing strategy comprises administering an initial dose of a non-mutated protein epitope therapeutic to the subject, and administering subsequent doses of the agent about once every 2 weeks. In some embodiments, the intermittent dosing strategy comprises administering an initial dose of a non-mutated protein epitope therapeutic to the subject, and administering subsequent doses of the agent about once every 3 weeks. In some embodiments, the intermittent dosing strategy comprises administering an initial dose of a non-mutated protein epitope therapeutic to the subject, and administering subsequent doses of the agent about once every 4 weeks. In some embodiments, the agent is administered using an intermittent dosing strategy and the additional therapeutic agent is administered weekly.

The present invention provides compositions comprising the non-mutated protein epitope therapeutic described herein. The present invention also provides pharmaceutical compositions comprising a non-mutated protein epitope therapeutic described herein and a pharmaceutically acceptable vehicle. In some embodiments, the pharmaceutical compositions find use in immunotherapy. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining an antigen therapeutic of the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition. Exemplary formulations are listed in WO 2015/095811.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22st Edition, 2012, Pharmaceutical Press, London.). In one embodiment, the vehicle is 5% dextrose in water.

The pharmaceutical compositions described herein can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories.

The non-mutated protein epitope peptides described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22st Edition, 2012, Pharmaceutical Press, London.

In certain embodiments, pharmaceutical formulations include anon-mutated protein epitope therapeutic described herein complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the non-mutated protein epitope peptides described herein can be produced. Suitable examples of sustained-release preparations include semi-permeable matrices of solid hydrophobic polymers containing an agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

### VIII. Kits

The non-mutated protein epitope therapeutic described herein can be provided in kit form together with instructions for administration. Typically the kit would include the desired antigen therapeutic in a container, in unit dosage form and instructions for administration. Additional therapeutics, for example, cytokines, lymphokines, checkpoint inhibitors, antibodies, can also be included in the kit. Other kit components that can also be desirable include, for example, a sterile syringe, booster dosages, and other desired excipients.

The invention will be described in greater detail by way of specific examples. The following examples are offered for illustrative purposes, and are not intended to limit the invention in any manner. Those of skill in the art will readily recognize a variety of non-critical parameters that can be changed or modified to yield alternative embodiments according to the invention. All patents, patent applications, and printed publications listed herein are incorporated herein by reference in their entirety.

### EXAMPLES

### Example 1: Identification of Mutant Sequences with Immunogenic Potential

Applicants have discovered that the following epitopes are recurrent in cancer patients.

TABLE 1

| ERV element | | |
|---|---|---|
| (nomenclature see: | | |
| ncbi.nlm.nih.gov/ | | |
| pmc/articles/ | | |
| PMC3113919/) | Translated Sequence | SEQ ID NO: |
| ERVH-2: | MGNLPPSIPPSPLACVLKNLKPLQLTPDLKPKCLIFFCNTAWPQYKLDN | 5 |
| retroviral matrix | GSKWPENGTFDFSILQDLNNSCRKMGKWSEVPDVQAFFYTSVPS | |
| ERVH48-1: coat | MPNRAIRLQAVLEIITNQTASALEMLAQQQNQMRAAIYQNRLALDYLL | 6 |
| protein | AEEGAGCGKFNISNCCLNIGNNGEEVLEIASNIRKVARVPVQTWEGWD | |
| | PANLLGGWFSNLGGFKMLVGTVIFITGVLLFLPCGIPLKLLLKLQLTS | |
| ERVH48-1: | MACIYPTTFYTSLPTKSLNMGISLTTILILSVAVLLSTAAPPSCRECYQSL | 7 |
| syncytin | HYRGEMQQYFTYHTHIERSCYGNLIEECVESGKSYYKVKNLGVCGSRN | |
| | GAICPRGKQWLCFTKIGQWGVNTQVLEDIKREQIIAKAKASKPTTPPEN | |
| | RPRHFHSFIQKL | |
| ERVH-2: gag | MARSAATLRRFTALDPKRSKGRLILNIHFITQSAPDIK | 8 |
| ERVE-4: reverse | RLFLTKPGKEIGPALAQWWPKVCAEDNPPGLAVNQAPVLREVKPEAQ | 9 |
| transcriptase | PVRQNQYPVPREALEGIQVHLKHLRTFGIIVPCQSPWNTPLLPVPKPGT | |
| | KDYRPVQDLRLVNQATVTFHPTVPNPYTLLGLLPAKDSWFTCLDLKD | |
| | AFFSIRLAPESQKLFAFQWEDPGSGVTTHYTWTRLPQGFKNFPHHLWG | |
| | GTGSRPPKVSCQRPRLRVVPVHRQPPAGTPHGSRVRQRNRRPASAPGG | |
| | LWV | |
| ERVE-4: reverse | MAVGCVKGTDALLQHLEDYGYKVSKKKAQICRQQVRYLGFTIRQREC | 10 |
| transcriptase | SLGSERKQVICNLLEPKTRRQLRELLGAVGFCRLWIPNFAVLAKPLVPS | |
| | YKGG | |
| ERVE-4: protease | NADLLAAAIRGVPLKGQGNGGSRKNTQSDRPRLQRNQCAYCKETGH | 11 |
| | WKDKCPQLKEKQGGSEQKTPDKDEGALFNLAEGLLDRRGPGSRAPKE | |
| | PMVRMTVGGKDIKFLVNTGAEHSVVTTPVAPLSKKAIDIIGATGVLTK | |
| | QAFCLPRTCSVGGHEVIHQFLYIPDCPLPLLGRDLLSKLRAIFLYQARLF | |
| | TTEVAWNRSYHGPDSSPRGRVATLPNQTRQRDRASSGPVVAKSMRRR | |
| | QPSWIGSQSSSCTQGS | |
| ERVE-4: unknown | SLFLHKTSVREVLSATIPATFLGSLTWKRGD | 12 |
| ERVK-5: gag, env, | MQNEAIEQVRAICLRAWGKIQDPGTAFPINSIRQGSKEPYPDFVARLQD | 13 |
| pol | AAQKSITDDNARKVIVELMAYENANPECQSAIKPLKGKVPAGVDVITE | |
| | YVKACDGIGGAMHKAMLMAQAMRGLTLGGQVRTFGKKCYNCGQIG | |
| | HLKRSCPVLNKQNIINQAITAKNKKPSGLCPKCGKGKHWANQCHSKFD | |
| | KDGQPLSGNRKRGQPQAPQQTGAFPVQLFVPQGFQGQQPLQKIPPLQG | |
| | VSQLQQSNSCPAPQQAAPQ | |
| ERVI-1: envelope | MEWIKYSICTLNKSNCYACAHGRPEAQIVPFPLRWSSSRPSMGCMVAL | 14 |
| | FQDSTAWGNISCQALSLLYPEVQHPAGQPPRAIQLPSPNVSFISCLS | |

For each epitope, the full-length amino acid sequence of the non-mutated protein epitope was derived. Any constituent 9mer or 1 Omer not found in the germline protein sequence was flagged and scored for binding potential on six common HLA alleles (HLA-A01:01, HLA-A02:01. HLA-A03:01, HLA-A24:02, HLA-B07:02, and HLA-B08:01) using available algorithms. Any peptide scoring better than 1000 nM was nominated.

TABLE 2

| ERV element | | |
|---|---|---|
| (nomenclature see: | | |
| http://www.ncbi.nlm. | | |
| nih.gov/pmc/articles/ | | |
| PMC3113919/) | Translated Sequence | SEQ ID NO: |
| ERVH-2: retroviral | MGNLPPSIPPSPLACVLKNLKPLQLTPDLKPKCLIFFCNTAWPQYKLD | 5 |
| matrix | NGSKWPENGTFDFSILQDLNNSCRKMGKWSEVPDVQAFFYTSVPS | |
| ERVH48-1: coat | MPNRAIRLQAVLEIITNQTASALEMLAQQQNQMRAAIYQNRLALDY | 6 |
| protein | LLAEEGAGCGKFNISNCCLNIGNNGEEVLEIASNIRKVARVPVQTWE | |
| | GWDPANLLGGWFSNLGGFKMLVGTVIFITGVLLFLPCGIPLKLLLKL | |
| | QLTS | |
| ERVH48-1: syncytin | MACIYPTTFYTSLPTKSLNMGISLTTILILSVAVLLSTAAPPSCRECYQS | 7 |
| | LHYRGEMQQYFTYHTHIERSCYGNLIEECVESGKSYYKVKNLGVCG | |
| | SRNGAICPRGKQWLCFTKIGQWGVNTQVLEDIKREQIIAKAKASKPT | |
| | TPPENRPRHFHSFIQKL | |
| ERVH-2: gag | MARSAATLRRFTALDPKRSKGRLILNIHFITQSAPDIK | 8 |
| ERVE-4: reverse | RLFLTKPGKEIGPALAQWWPKVCAEDNPPGLAVNQAPVLREVKPEA | 9 |
| transcriptase | QPVRQNQYPVPREALEGIQVHLKHLRTFGIIVPCQSPWNTPLLPVPKP | |
| | GTKDYRPVQDLRLVNQATVTFHPTVPNPYTLLGLLPAKDSWFTCLD | |
| | LKDAFFSIRLAPESQKLFAFQWEDPGSGVTTHYTWTRLPQGFKNFPH | |
| | HLWGGTGSRPPKVSCQRPRLRVVPVHRQPPAGTPHGSRVRQRNRRP | |
| | ASAPGGLWV | |
| ERVE-4: reverse | MAVGCVKGTDALLQHLEDYGYKVSKKKAQICRQQVRYLGFTIRQR | 10 |
| transcriptase | ECSLGSERKQVICNLLEPKTRRQLRELLGAVGFCRLWIPNFAVLAKPL | |
| | VPSYKGG | |
| ERVE-4: protease | NADLLAAAIRGVPLKGQGNGGSRKNTQSDRPRLQRNQCAYCKETGH | 11 |
| | WKDKCPQLKEKQGGSEQKTPDKDEGALFNLAEGLLDRRGPGSRAPK | |
| | EPMVRMTVGGKDIKFLVNTGAEHSVVTTPVAPLSKKAIDIIGATGVL | |
| | TKQAFCLPRTCSVGGHEVIHQFLYIPDCPLPLLGRDLLSKLRAIFLYQ | |
| | ARLFTTEVAWNRSYHGPDSSPRGRVATLPNQTRQRDRASSGPVVAK | |
| | SMRRRQPSWIGSQSSSCTQGS | |
| ERVE-4: unknown | SLFLHKTSVREVLSATIPATFLGSLTWKRGD | 12 |
| ERVK-5: gag, env, | MQNEAIEQVRAICLRAWGKIQDPGTAFPINSIRQGSKEPYPDFVARLQ | 13 |
| pol | DAAQKSITDDNARKVIVELMAYENANPECQSAIKPLKGKVPAGVDVI | |
| | TEYVKACDGIGGAMHKAMLMAQAMRGLTLGGQVRTFGKKCYNCG | |
| | QIGHLKRSCPVLNKQNIINQAITAKNKKPSGLCPKCGKGKHWANQC | |
| | HSKFDKDGQPLSGNRKRGQPQAPQQTGAFPVQLFVPQGFQGQQPLQ | |
| | KIPPLQGVSQLQQSNSCPAPQQAAPQ | |
| | | 14 |
| ERVI-1: envelope | MEWIKYSICTLNKSNCYACAHGRPEAQIVPFPLRWSSSRPSMGCMVA | |
| | LFQDSTAWGNISCQALSLLYPEVQHPAGQPPRAIQLPSPNVSFISCLS | |

TABLE 3

| Over- | | | |
|---|---|---|---|
| expressed | UCSC | | |
| gene | ID | Full Sequence | SEQ ID NO: |
| TYR | uc001pcs.3 | MLLAVLYCLLWSFQTSAGHFPRACVSSKNLMEKECCPPWSGDRSP | 15 |
| | | CGQLSGRGSCQNILLSNAPLGPQFPFTGVDDRESWPSVFYNRTCQC | |
| | | SGNFMGFNCGNCKFGFWGPNCTERRLLVRRNIFDLSAPEKDKFFAY | |
| | | LTLAKHTISSDYVIPIGTYGQMKNGSTPMFNDINIYDLFVWMHYYV | |
| | | SMDALLGGSEIWRDIDFAHEAPAFLPWHRLFLLRWEQEIQKLTGDE | |
| | | NFTIPYWDWRDAEKCDICTDEYMGGQHPTNPNLLSPASFFSSWQIV | |
| | | CSRLEEYNSHQSLCNGTPEGPLRRNPGNHDKSRTPRLPSSADVEFCL | |
| | | SLTQYESGSMDKAANFSFRNTLEGFASPLTGIADASQSSMHNALHI | |
| | | YMNGTMSQVQGSANDPIFLLHHAFVDSIFEQWLRRHRPLQEVYPE | |
| | | ANAPIGHNRESYMVPFIPLYRNGDFFISSKDLGYDYSYLQDSDPDSF | |
| | | QDYIKSYLEQASRIWSWLLGAAMVGAVLTALLAGLVSLLCRHKRK | |
| | | QLPEEKQPLLMEKEDYHSLYQSHL | |
| MAGEC1 | uc004fbt.3 | MGDKDMPTAGMPSLLQSSSESPQSCPEGEDSQSPLQIPQSSPESDDT | 16 |
| | | LYPLQSPQSRSEGEDSSDPLQRPPEGKDSQSPLQIPQSSPEGDDTQSP | |
| | | LQNSQSSPEGKDSLSPLEISQSPPEGEDVQSPLQNPASSFFSSALLSIF | |
| | | QSSPESTQSPFEGFPQSVLQIPVSAASSSTLVSIFQSSPESTQSPFEGFP | |
| | | QSPLQIPVSRSFSSTLLSIFQSSPERTQSTFEGFAQSPLQIPVSPSSSSTL | |
| | | LSLFQSFSERTQSTFEGFAQSSLQIPVSPSFSSTLVSLFQSSPERTQSTF | |
| | | EGFPQSPLQIPVSSSSSSTLLSLFQSSPERTHSTFEGFPQSLLQIPMTSS | |
| | | FSSTLLSIFQSSPESAQSTFEGFPQSPLQIPGSPSFSSTLLSLFQSSPERT | |
| | | HSTFEGFPQSPLQIPMTSSFSSTLLSILQSSPESAQSAFEGFPQSPLQIP | |
| | | VSSSFSYTLLSLFQSSPERTHSTFEGFPQSPLQIPVSSSSSSSTLLSLFQ | |
| | | SSPECTQSTFEGFPQSPLQIPQSPPEGENTHSPLQIVPSLPEWEDSLSP | |
| | | HYFPQSPPQGEDSLSPHYFPQSPPQGEDSLSPHYFPQSPQGEDSLSPH | |
| | | YFPQSPPQGEDSMSPLYFPQSPLQGEEFQSSLQSPVSICSSSTPSSLPQ | |
| | | SFPESSQSPPEGPVQSPLHSPQSPPEGMHSQSPLQSPESAPEGEDSLSP | |
| | | LQIPQSPLEGEDSLSSLHFPQSPPEWEDSLSPLHFPQFPPQGEDFQSSL | |
| | | QSPVSICSSSTSLSLPQSFPESPQSPPEGPAQSPLQRPVSSFFSYTLASL | |
| | | LQSSHESPQSPPEGPAQSPLQSPVSSFPSSTSSSLSQSSPVSSFPSSTSS | |
| | | SLSKSSPESPLQSPVISFSSSTSLSPFSEESSSPVDEYTSSSDTLLESDSL | |
| | | TDSESLIESEPLFTYTLDEKVDELARFLLLKYQVKQPITKAEMLTNVI | |
| | | SRYTGYFPVIFRKAREFIEILFGISLREVDPDDSYVFVNTLDLTSEGCL | |
| | | SDEQGMSQNRLLILILSIIFIKGTYASEEVIWDVLSGIGVRAGREHFA | |
| | | FGEPRELLTKVWVQEHYLEYREVPNSSPPRYEFLWGPRAHSEVIKR | |
| | | KVVEFLAMLKNTVPITFPSSYKDALKDVEERAQAIIDTTDDSTATES | |
| | | ASSSVMSPSFSSE | |
| MAGEA10 | uc022cgz.1 | MPRAPKRQRCMPEEDLQSQSETQGLEGAQAPLAVEEDASSSTSTSS | 17 |
| | | SFPSSFPSSSSSSSSSCYPLIPSTPEEVSADDETPNPPQSAQIACSSPSV | |
| | | VASLPLDQSDEGSSSQKEESPSTLQVLPDSESLPRSEIDEKVTDLVQF | |
| | | LLFKYQMKEPITKAEILESVIRNYEDHFPLLFSEASECMLLVFGIDVK | |
| | | EVDPTGHSFVLVTSLGLTYDGMLSDVQSMPKTGILILILSIVFIEGYC | |
| | | TPEEVIWEALNMMGLYDGMEHLIYGEPRKLLTQDWVQENYLEYR | |
| | | QVPGSDPARYEFLWGPRAHAEIRKMSLLKFLAKVNGSDPRSFPLW | |
| | | YEEALKDEEERAQDRIATTDDTTAMASASSSATGSFSYPE | |
| MAGEB17 | uc031tgu.1 | MPRGQASKRRAREKRRQARGEDQCLGGAQATAAEKEKLPSSSSPA | 18 |
| | | CQSPPQSFPNAGIPQESQRASYPSSPASAVSLTSSDEGAKGQKGESP | |
| | | NSFHGPSSSESTGRDLLNTKTGELVQFLLNKYIRKEPITREAMLKVI | |
| | | NRKYKQHFPEILRRSTENVEVVFGLYLKEMDPSRQSYVLVGKLDFP | |
| | | NQGSLSDGGGFPLSGLLMVLLSTIFMHGNRATEEEMWECLNALGM | |
| | | YKGRKHFIYGEPQELVTKDLVREGYLEYQQVPSSDPPRYEFLWGPR | |
| | | ARAETSKMKVLEFVAKLNDTVASTYKSRYEEALREEEEQARARAV | |
| | | ARDSARARASRSFQP | |
| MAGEA4 | uc022cgu.1 | MLPLSVGLWVPIAQLLPALLPAALTRVIMSSEQKSQHCKPEEGVEA | 19 |
| | | QEEALGLVGAQAPTTEEQEAAVSSSSPLVPGTLEEVPAAESAGPPQS | |
| | | PQGASALPTTISFTCWRQPNEGSSSQEEEGPSTSPDAESLFREALSNK | |
| | | VDELAHFLLRKYRAKELVTKAEMLERVIKNYKRCFPVIFGKASESL | |
| | | KMIFGIDVKEVDPASNTYTLVTCLGLSYDGLLGNNQIFPKTGLLIIVL | |
| | | GTIAMEGDSASEEEIWEELGVMGVYDGREHTVYGEPRKLLTQDWV | |
| | | QENYLEYRQVPGSNPARYEFLWGPRALAETSYVKVLEHVVRVNAR | |
| | | VRIAYPSLREAALLEEEEGV | |
| MABEB16 | uc022bus.1 | MSQDQESPRCTHDQHLQTFSETQSLEVAQVSKALEKTLLSSSHPLV | 20 |
| | | PGKLKEAPAAKAESPLEVPQSFCSSSIAVTTTSSSESDEASSNQEEED | |
| | | SPSSSEDTSDPRNVPADALDQKVAFLVNFMLHKCQMKKPITKADM | |
| | | LKIIIKDDESHFSEILLRASEHLEMIFGLDVVEVDPTTHCYGLFIKLGL | |
| | | TYDGMLSGEKGVPKTGLLIIVLGVIFMKGNRATEEEVWEVLNLTGV | |
| | | YSGKKHFIFGEPRMLITKDFVKEKYLEYQQVANSDPARYEFLWGPR | |
| | | AKAETSKMKVLEFVAKVHGSYPHSFPSQYAEALKEEEERARARI | |
| MAGEA1 | uc022chs.1 | MSLEQRSLHCKPEEALEAQQEALGLVCVQAATSSSSPLVLGTLEEV | 21 |
| | | PTAGSTDPPQSPQGASAFPTTINFTRQRQPSEGSSSREEEGPSTSCILE | |
| | | SLFRAVITKKVADLVGFLLLKYRAREPVTKAEMLESVIKNYKHCFP | |
| | | EIFGKASESLQLVFGIDVKEADPTGHSYVLVTCLGLSYDGLLGDNQI | |
| | | MPKTGFLIIVLVMIAMEGGHAPEEEIWEELSVMEVYDGREHSAYGE | |
| | | PRKLLTQDLVQEKYLEYRQVPDSDPARYEFLWGPRALAETSYVKV | |
| | | LEYVIKVSARVRFFFPSLREAALREEEEGV | |
| MAGEA8 | uc022cgo.1 | MLLGQKSQRYKAEEGLQAQGEAPGLMDVQIPTAEEQKAASSSSTLI | 22 |
| | | MGTLEEVTDSGSPSPPQSPEGASSSLTVTDSTLWSQSDEGSSSNEEE | |
| | | GPSTSPDPAHLESLFREALDEKVAELVRFLLRKYQIKEPVTKAEMLE | |
| | | SVIKNYKNHFPDIFSKASECMQVIFGIDVKEVDPAGHSYILVTCLGL | |
| | | SYDGLLGDDQSTPKTGLLIIVLGMILMEGSRAPEEAIWEALSVMGL | |
| | | YDGREHSVYWKLRKLLTQEWVQENYLEYRQAPGSDPVRYEFLWG | |
| | | PRALAETSYVKVLEHVVRVNARVRISYPSLHEEALGEEKGV | |
| MAGEB4 | uc004dcb.3 | MPRGQKSKLRAREKRQRTRGQTQDLKVGQPTAAEKEESPSSSSSVL | 23 |
| | | RDTASSSLAFGIPQEPQREPPTTSAAAAMSCTGSDKGDESQDEENAS | |
| | | SSQASTSTERSLKDSLTRKTKMLVQFLLYKYKMKEPTTKAEMLKIIS | |
| | | KKYKEHFPEIFRKVSQRTELVFGLALKEVNPTTHSYILVSMLGPNDG | |
| | | NQSSAWTLPRNGLLMPLLSVIFLNGNCAREEEIWEFLNMLGIYDGK | |
| | | RHLIFGEPRKLITQDLVQEKYLEYQQVPNSDPPRYQFLWGPRAHAE | |
| | | TSKMKVLEFLAKVNDTTPNNFPLLYEEALRDEEERAGARPRVAAR | |
| | | RGTTAMTSAYSRATSSSSSQPM | |
| CT45A5 | uc011mvu.2 | MTDKTEKVAVDPETVFKRPRECDSPSYQKRQRMALLARKQGAGD | 24 |
| | | SLIAGSAMSKEKKLMTGHAIPPSQLDSQIDDFTGFSKDGMMQKPGS | |
| | | NAPVGGNVTSNFSGDDLECRGIASSPKSQQEINADIKCQVVKEIRCL | |
| | | GRKYEKIFEMLEGVQGPTAVRKRFFESIIKEAARCMRRDFVKHLKK | |
| | | KLKRMI | |
| ALPPL2 | uc002vss.4 | MQGPWVLLLLGLRLQLSLGIIPVEEENPDFWNRQAAEALGAAKKL | 25 |
| | | QPAQTAAKNLIIFLGDGMGVSTVTAARILKGQKKDKLGPETFLAMD | |
| | | RFPYVALSKTYSVDKHVPDSGATATAYLCGVKGNFQTIGLSAAARF | |
| | | NQCNTTRGNEVISVMNRAKKAGKSVGVVTTTRVQHASPAGAYAH | |
| | | TVNRNWYSDADVPASARQEGCQDIATQLISNMDIDVILGGGRKYM | |
| | | FPMGTPDPEYPDDYSQGGTRLDGKNLVQEWLAKHQGARYVWNRT | |
| | | ELLQASLDPSVTHLMGLFEPGDMKYEIHRDSTLDPSLMEMTEAALL | |
| | | LLSRNPRGFFLFVEGGRIDHGHHESRAYRALTETIMFDDAIERAGQL | |
| | | TSEEDTLSLVTADHSHVFSFGGYPLRGSSIFGLAPGKARDRKAYTVL | |
| | | LYGNGPGYVLKDGARPDVTESESGSPEYRQQSAVPLDGETHAGED | |
| | | VAVFARGPQAHLVHGVQEQTFIAHVMAFAACLEPYTACDLAPRAG | |
| | | TTDAAHPGPSVVPALLPLLAGTLLLLGTATAP | |
| MMP13 | uc001ph1.3 | MHPGVLAAFLFLSWTHCRALPLPSGGDEDDLSEEDLQFAERYLRSY | 26 |
| | | YHPTNLAGILKENAASSMTERLREMQSFFGLEVTGKLDDNTLDVM | |
| | | KKPRCGVPDVGEYNVFPRTLKWSKMNLTYRIVNYTPDMTHSEVEK | |
| | | AFKKAFKVWSDVTPLNFTRLHDGIADIMISFGIKEHGDFYPFDGPSG | |
| | | LLAHAFPPGPNYGGDAHFDDDETWTSSSKGYNLFLVAAHEFGHSL | |
| | | GLDHSKDPGALMFPIYTYTGKSHFMLPDDDVQGIQSLYGPGDEDPN | |
| | | PKHPKTPDKCDPSLSLDAITSLRGETMIFKDRFFWRLHPQQVDAELF | |
| | | LTKSFWPELPNRIDAAYEHPSHDLIFIFRGRKFWALNGYDILEGYPK | |
| | | KISELGLPKEVKKISAAVHFEDTGKTLLFSGNQVWRYDDTNHIMDK | |
| | | DYPRLIEEDFPGIGDKVDAVYEKNGYIYFFNGPIQFEYSIWSNRIVRV | |
| | | MPANSILWC | |
| CTAG1B | uc004fmf.1 | MQAEGRGTGGSTGDADGPGGPGIPDGPGGNAGGPGEAGATGGRG | 27 |
| | | PRGAGAARASGPGGGAPRGPHGGAASGLNGCCRCGARGPESRLLE | |
| | | FYLAMPFATPMEAELARRSLAQDAPPLPVPGVLLKEFTVSGNILTIR | |
| | | LTAADHRQLQLSISSCLQQLSLLMWITQCFLPVFLAQPPSGQRR | |
| DCT | uc010afh.3 | MSPLWWGFLLSCLGCKILPGAQGQFPRVCMTVDSLVNKECCPRLG | 28 |
| | | AESANVCGSQQGRGQCTEVRADTRPWSGPYILRNQDDRELWPRKF | |
| | | FHRTCKCTGNFAGYNCGDCKFGWTGPNCERKKPPVIRQNIHSLSPQ | |
| | | EREQFLGALDLAKKRVHPDYVITTQHWLGLLGPNGTQPQFANCSV | |
| | | YDFFVWLHYYSVRDTLLGPGRPYRAIDFSHQGPAFVTWHRYHLLC | |
| | | LERDLQRLIGNESFALPYWNFATGRNECDVCTDQLFGAARPDDPTL | |
| | | ISRNSRFSSWETVCDSLDDYNHLVTLCNGTYEGLLRRNQMGRNSM | |
| | | KLPTLKDIRDCLSLQKFDNPPFFQNSTFSFRNALEGFDKADGTLDSQ | |
| | | VMSLHNLVHSFLNGTNALPHSAANDPIFVVISNRLLYNATTNILEHV | |
| | | RKEKATKELPSLHVLVLHSFTDAIFDEWMKRFNPPADAWPQELAPI | |
| | | GHNRMYNMVPFFPPVTNEELFLTSDQLGYSYAIDLPVSVEETPGWP | |
| | | TTLLVVMGTLVALVGLFVLLAFLQYRRLRKGYTPLMETHLSSKRY | |
| | | TEEA | |
| CLDN6 | uc021tbb.1 | MASAGMQILGVVLTLLGWVNGLVSCALPMWKVTAFIGNSIVVAQ | 29 |
| | | VVWEGLWMSCVVQSTGQMQCKVYDSLLALPQDLQAARALCVIAL | |
| | | LVALFGLLVYLAGAKCTTCVEEKDSKARLVLTSGIVFVISGVLTLIP | |
| | | VCWTAHAIIRDFYNPLVAEAQKRELGASLYLGWAASGLLLLGGGL | |
| | | LCCTCPSGGSQGPSHYMARYSTSAPAISRGPSEYPTKNYV | |
| MLANA | uc003zjo.1 | MPREDAHFIYGYPKKGHGHSYTTAEEAAGIGILTVILGVLLLIGCWY | 30 |
| | | CRRRNGYRALMDKSLHVGTQCALTRRCPQEGFDHRDSKVSLQEK | |
| | | NCEPVVPNAPPAYEKLSAEQSPPPYSP | |
| AFP | uc003hgz.1 | MKWVESIFLIFLLNFTESRTLHRNEYGIASILDSYQCTAEISLADLATI | 31 |
| | | FFAQFVQEATYKEVSKMVKDALTAIEKPTGDEQSSGCLENQLPAFL | |
| | | EELCHEKEILEKYGHSDCCSQSEEGRHNCFLAHKKPTPASIPLFQVP | |
| | | EPVTSCEAYEEDRETFMNKFIYEIARRHPFLYAPTILLWAARYDKIIP | |
| | | SCCKAENAVECFQTKAATVTKELRESSLLNQHACAVMKNFGTRTF | |
| | | QAITVTKLSQKFTKVNFTEIQKLVLDVAHVHEHCCRGDVLDCLQD | |
| | | GEKIMSYICSQQDTLSNKITECCKLTTLERGQCIIHAENDEKPEGLSP | |
| | | NLNRFLGDRDFNQFSSGEKNIFLASFVHEYSRRHPQLAVSVILRVAK | |
| | | GYQELLEKCFQTENPLECQDKGEEELQKYIQESQALAKRSCGLFQK | |
| | | LGEYYLQNAFLVAYTKKAPQLTSSELMAITRKMAATAATCCQLSE | |
| | | DKLLACGEGAADIIIGHLCIRHEMTPVNPGVGQCCTSSYANRRPCFS | |
| | | SLVVDETYVPPAFSDDKFIFHKDLCQAQGVALQTMKQEFLINLVKQ | |
| | | KPQITEEQLEAVIADFSGLLEKCCQGQEQEVCFAEEGQKLISKTRAA | |
| | | LGV | |
| DKK4 | uc003xpb.3 | MVAAVLLGLSWLCSPLGALVLDFNNIRSSADLHGARKGSQCLSDT | 32 |
| | | DCNTRKFCLQPRDEKPFCATCRGLRRRCQRDAMCCPGTLCVNDVC | |
| | | TTMEDATPILERQLDEQDGTHAEGTTGHPVQENQPKRKPSIKKSQG | |
| | | RKGQEGESCLRTFDCGPGLCCARHFWTKICKPVLLEGQVCSRRGH | |
| | | KDTAQAPEIFQRCDCGPGLLCRSQLTSNRQHARLRVCQKIEKL | |
| ASCL2 | uc021qcf.1 | MDGGTLPRSAPPAPPVPVGCAARRRPASPELLRCSRRRRPATAETG | 33 |
| | | GGAAAVARRNERERNRVKLVNLGFQALRQHVPHGGASKKLSKVE | |
| | | TLRSAVEYIRALQRLLAEHDAVRNALAGGLRPQAVRPSAPRGPPGT | |
| | | TPVAASPSRASSSPGRGGSSEPGSPRSAYSSDDSGCEGALSPAERELL | |
| | | DFSSWLGGY | |
| GAGE1 | uc004dok.2 | MSWRGRSTYYWPRPRRYVQPPEMIGPMRPEQFSDEVEPATPEEGEP | 34 |
| | | ATQRQDPAAAQEGEDEGASAGQGPKPEADSQEQGHPQTGCECEDG | |
| | | PDGQEMDPPNPEEVKTPEEEMRSHYVAQTGILWLLMNNCFLNLSP | |
| | | RKP | |
| GAGE10 | uc010nir.1 | MSWRGRSTYRSRPRLYVEPPEMIGPMLPEQFSDEVEPATPEEGEPA | 35 |
| | | TQRQDPAAAQEGEDEGASAGQGPKPEADSQEQVHPKTGCECGDGP | |
| | | DGQEMGLPNPEEVKRPEEGEKQSQC | |
| SLC45A2 | uc003jid.3 | MGSNSGQAGRHIYKSLADDGPFDSVEPPKRPTSRLIMHSMAMFGRE | 36 |
| | | FCYAVEAAYVTPVLLSVGLPSSLYSIVWFLSPILGFLLQPVVGSASD | |
| | | HCRSRWGRRRPYILTLGVMMLVGMALYLNGATVVAALIANPRRK | |
| | | LVWAISVTMIGVVLFDFAADFIDGPIKAYLFDVCSHQDKEKGLHYH | |
| | | ALFTGFGGALGYLLGAIDWAHLELGRLLGTEFQVMFFFSALVLTLC | |
| | | FTVHLCSISEAPLTEVAKGIPPQQTPQDPPLSSDGMYEYGSIEKVKN | |
| | | GYVNPELAMQGAKNKNHAEQTRRAMTLKSLLRALVNMPPHYRYL | |
| | | CISHLIGWTAFLSNMLFFTDFMGQIVYRGDPYSAHNSTEFLIYERGV | |
| | | EVGCWGFCINSVFSSLYSYFQKVLVSYIGLKGLYFTGYLLFGLGTGF | |
| | | IGLFPNVYSTLVLCSLFGVMSSTLYTVPFNLITEYHREEEKERQQAP | |
| | | GGDPDNSVRGKGMDCATLTCMVQLAQILVGGGLGFLVNTAGTVV | |
| | | VVVITASAVALIGCCFVALFVRYVD | |
| PAGE5 | uc004duj.3 | MQAPWAGNRGWAGTREEVRDMSEHVTRSQSSERGNDQESSQPVG | 37 |
| | | PVIVQQPTEEKRQEEEPPTDNQGIAPSGEIKNEGAPAVQGTDVEAFQ | |
| | | QELALLKIEDAPGDGPDVREGTLPTFDPTKVLEAGEGQL | |
| PAGE2 | uc004duf.1 | MSELLRARSQSSERGNDQESSQPVGSVIVQEPTEEKRQEEEPPTDNQ | 38 |
| | | GIAPSGEIENQAVPAFQGPDMEAFQQELALLKIEDEPGDGPDVREGI | |
| | | MPTFDLTKVLEAGDAQP | |
| PMEL | uc001siq.3 | MDLVLKRCLLHLAVIGALLAVGATKVPRNQDWLGVSRQLRTKAW | 39 |
| | | NRQLYPEWTEAQRLDCWRGGQVSLKVSNDGPTLIGANASFSIALNF | |
| | | PGSQKVLPDGQVIWVNNTIINGSQVWGGQPVYPQETDDACIFPDGG | |
| | | PCPSGSWSQKRSFVYVWKTWGQYWQVLGGPVSGLSIGTGRAMLG | |
| | | THTMEVTVYHRRGSRSYVPLAHSSSAFTITDQVPFSVSVSQLRALD | |
| | | GGNKHFLRNQPLTFALQLHDPSGYLAEADLSYTWDFGDSSGTLISR | |
| | | ALVVTHTYLEPGPVTAQVVLQAAIPLTSCGSSPVPGTTDGHRPTAE | |
| | | APNTTAGQVPTTEVVGTTPGQAPTAEPSGTTSVQVPTTEVISTAPVQ | |
| | | MPTAESTGMTPEKVPVSEVMGTTLAEMSTPEATGMTPAEVSIVVLS | |
| | | GTTAAQVTTTEWVETTARELPIPEPEGPDASSIMSTESITGSLGPLLD | |
| | | GTATLRLVKRQVPLDCVLYRYGSFSVTLDIVQGIESAEILQAVPSGE | |
| | | GDAFELTVSCQGGLPKEACMEISSPGCQPPAQRLCQPVLPSPACQL | |
| | | VLHQILKGGSGTYCLNVSLADTNSLAVVSTQLIMPVPGILLTGQEA | |
| | | GLGQVPLIVGILLVLMAVVLASLIYRRRLMKQDFSVPQLPHSSSHW | |
| | | LRLPRIFCSCPIGENSPLLSGQQV | |

For each epitope, the full-length amino acid sequence of the non-mutated protein epitope was derived. Any constituent 9mer or 1 Omer not found in the germline protein sequence was flagged and scored for binding potential on six common HLA alleles (HLA-A01:01, HLA-A02:01. HLA-A03:01, HLA-A24:02, HLA-B07:02, and HLA-B08:01) using available algorithms. Any peptide scoring better than 1000 nM was nominated.

TABLE 4

| Over- | | | |
|---|---|---|---|
| expressed | | | |
| gene | UCSC ID | Full Sequence | SEQ ID NO: |
| TYR | uc001pcs.3 | MLLAVLYCLLWSFQTSAGHFPRACVSSKNLMEKECCPPWSGDRS | 15 |
| | | PCGQLSGRGSCQNILLSNAPLGPQFPFTGVDDRESWPSVFYNRTC | |
| | | QCSGNFMGFNCGNCKFGFWGPNCTERRLLVRRNIFDLSAPEKDK | |
| | | FFAYLTLAKHTISSDYVIPIGTYGQMKNGSTPMFNDINIYDLFVW | |
| | | MHYYVSMDALLGGSEIWRDIDFAHEAPAFLPWHRLFLLRWEQEI | |
| | | QKLTGDENFTIPYWDWRDAEKCDICTDEYMGGQHPTNPNLLSPA | |
| | | SFFSSWQIVCSRLEEYNSHQSLCNGTPEGPLRRNPGNHDKSRTPRL | |
| | | PSSADVEFCLSLTQYESGSMDKAANFSFRNTLEGFASPLTGIADAS | |
| | | QSSMHNALHIYMNGTMSQVQGSANDPIFLLHHAFVDSIFEQWLR | |
| | | RHRPLQEVYPEANAPIGHNRESYMVPFIPLYRNGDFFISSKDLGYD | |
| | | YSYLQDSDPDSFQDYIKSYLEQASRIWSWLLGAAMVGAVLTALL | |
| | | AGLVSLLCRHKRKQLPEEKQPLLMEKEDYHSLYQSHL | |
| MAGEC1 | uc004fbt.3 | MGDKDMPTAGMPSLLQSSSESPQSCPEGEDSQSPLQIPQSSPESDD | 16 |
| | | TLYPLQSPQSRSEGEDSSDPLQRPPEGKDSQSPLQIPQSSPEGDDTQ | |
| | | SPLQNSQSSPEGKDSLSPLEISQSPPEGEDVQSPLQNPASSFFSSALL | |
| | | SIFQSSPESTQSPFEGFPQSVLQIPVSAASSSTLVSIFQSSPESTQSPFE | |
| | | GFPQSPLQIPVSRSFSSTLLSIFQSSPERTQSTFEGFAQSPLQIPVSPS | |
| | | SSSTLLSLFQSFSERTQSTFEGFAQSSLQIPVSPSFSSTLVSLFQSSPE | |
| | | RTQSTFEGFPQSPLQIPVSSSSSSTLLSLFQSSPERTHSTFEGFPQSLL | |
| | | QIPMTSSFSSTLLSIFQSSPESAQSTFEGFPQSPLQIPGSPSFSSTLLSL | |
| | | FQSSPERTHSTFEGFPQSPLQIPMTSSFSSTLLSILQSSPESAQSAFEG | |
| | | FPQSPLQIPVSSSFSYTLLSLFQSSPERTHSTFEGFPQSPLQIPVSSSS | |
| | | SSSTLLSLFQSSPECTQSTFEGFPQSPLQIPQSPPEGENTHSPLQIVPS | |
| | | LPEWEDSLSPHYFPQSPPQGEDSLSPHYFPQSPPQGEDSLSPHYFPQ | |
| | | SPQGEDSLSPHYFPQSPPQGEDSMSPLYFPQSPLQGEEFQSSLQSPV | |
| | | SICSSSTPSSLPQSFPESSQSPPEGPVQSPLHSPQSPPEGMHSQSPLQ | |
| | | SPESAPEGEDSLSPLQIPQSPLEGEDSLSSLHFPQSPPEWEDSLSPLH | |
| | | FPQFPPQGEDFQSSLQSPVSICSSSTSLSLPQSFPESPQSPPEGPAQSP | |
| | | LQRPVSSFFSYTLASLLQSSHESPQSPPEGPAQSPLQSPVSSFPSSTS | |
| | | SSLSQSSPVSSFPSSTSSSLSKSSPESPLQSPVISFSSSTSLSPFSEESSS | |
| | | PVDEYTSSSDTLLESDSLTDSESLIESEPLFTYTLDEKVDELARFLL | |
| | | LKYQVKQPITKAEMLTNVISRYTGYFPVIFRKAREFIEILFGISLRE | |
| | | VDPDDSYVFVNTLDLTSEGCLSDEQGMSQNRLLILILSIIFIKGTYA | |
| | | SEEVIWDVLSGIGVRAGREHFAFGEPRELLTKVWVQEHYLEYRE | |
| | | VPNSSPPRYEFLWGPRAHSEVIKRKVVEFLAMLKNTVPITFPSSYK | |
| | | DALKDVEERAQAIIDTTDDSTATESASSSVMSPSFSSE | |
| MAGEA10 | uc022cgz.1 | MPRAPKRQRCMPEEDLQSQSETQGLEGAQAPLAVEEDASSSTSTS | 17 |
| | | SSFPSSFPSSSSSSSSSCYPLIPSTPEEVSADDETPNPPQSAQIACSSPS | |
| | | VVASLPLDQSDEGSSSQKEESPSTLQVLPDSESLPRSEIDEKVTDLV | |
| | | QFLLFKYQMKEPITKAEILESVIRNYEDHFPLLFSEASECMLLVFGI | |
| | | DVKEVDPTGHSFVLVTSLGLTYDGMLSDVQSMPKTGILILILSIVFI | |
| | | EGYCTPEEVIWEALNMMGLYDGMEHLIYGEPRKLLTQDWVQEN | |
| | | YLEYRQVPGSDPARYEFLWGPRAHAEIRKMSLLKFLAKVNGSDP | |
| | | RSFPLWYEEALKDEEERAQDRIATTDDTTAMASASSSATGSFSYP | |
| | | E | |
| MAGEB17 | uc031tgu.1 | MPRGQASKRRAREKRRQARGEDQCLGGAQATAAEKEKLPSSSSP | 18 |
| | | ACQSPPQSFPNAGIPQESQRASYPSSPASAVSLTSSDEGAKGQKGE | |
| | | SPNSFHGPSSSESTGRDLLNTKTGELVQFLLNKYIRKEPITREAML | |
| | | KVINRKYKQHFPEILRRSTENVEVVFGLYLKEMDPSRQSYVLVGK | |
| | | LDFPNQGSLSDGGGFPLSGLLMVLLSTIFMHGNRATEEEMWECL | |
| | | NALGMYKGRKHFIYGEPQELVTKDLVREGYLEYQQVPSSDPPRY | |
| | | EFLWGPRARAETSKMKVLEFVAKLNDTVASTYKSRYEEALREEE | |
| | | EQARARAVARDSARARASRSFQP | |
| MAGEA4 | uc022cgu.1 | MLPLSVGLWVPIAQLLPALLPAALTRVIMSSEQKSQHCKPEEGVE | 19 |
| | | AQEEALGLVGAQAPTTEEQEAAVSSSSPLVPGTLEEVPAAESAGP | |
| | | PQSPQGASALPTTISFTCWRQPNEGSSSQEEEGPSTSPDAESLFREA | |
| | | LSNKVDELAHFLLRKYRAKELVTKAEMLERVIKNYKRCFPVIFGK | |
| | | ASESLKMIFGIDVKEVDPASNTYTLVTCLGLSYDGLLGNNQIFPKT | |
| | | GLLIIVLGTIAMEGDSASEEEIWEELGVMGVYDGREHTVYGEPRK | |
| | | LLTQDWVQENYLEYRQVPGSNPARYEFLWGPRALAETSYVKVLE | |
| | | HVVRVNARVRIAYPSLREAALLEEEEGV | |
| MABEB16 | uc022bus.1 | MSQDQESPRCTHDQHLQTFSETQSLEVAQVSKALEKTLLSSSHPL | 20 |
| | | VPGKLKEAPAAKAESPLEVPQSFCSSSIAVTTTSSSESDEASSNQEE | |
| | | EDSPSSSEDTSDPRNVPADALDQKVAFLVNFMLHKCQMKKPITK | |
| | | ADMLKIIIKDDESHFSEILLRASEHLEMIFGLDVVEVDPTTHCYGLF | |
| | | IKLGLTYDGMLSGEKGVPKTGLLIIVLGVIFMKGNRATEEEVWEV | |
| | | LNLTGVYSGKKHFIFGEPRMLITKDFVKEKYLEYQQVANSDPARY | |
| | | EFLWGPRAKAETSKMKVLEFVAKVHGSYPHSFPSQYAEALKEEE | |
| | | ERARARI | |
| MAGEA1 | uc022chs.1 | MSLEQRSLHCKPEEALEAQQEALGLVCVQAATSSSSPLVLGTLEE | 21 |
| | | VPTAGSTDPPQSPQGASAFPTTINFTRQRQPSEGSSSREEEGPSTSCI | |
| | | LESLFRAVITKKVADLVGFLLLKYRAREPVTKAEMLESVIKNYKH | |
| | | CFPEIFGKASESLQLVFGIDVKEADPTGHSYVLVTCLGLSYDGLLG | |
| | | DNQIMPKTGFLIIVLVMIAMEGGHAPEEEIWEELSVMEVYDGREH | |
| | | SAYGEPRKLLTQDLVQEKYLEYRQVPDSDPARYEFLWGPRALAE | |
| | | TSYVKVLEYVIKVSARVRFFFPSLREAALREEEEGV | |
| MAGEA8 | uc022cgo.1 | MLLGQKSQRYKAEEGLQAQGEAPGLMDVQIPTAEEQKAASSSST | 22 |
| | | LIMGTLEEVTDSGSPSPPQSPEGASSSLTVTDSTLWSQSDEGSSSNE | |
| | | EEGPSTSPDPAHLESLFREALDEKVAELVRFLLRKYQIKEPVTKAE | |
| | | MLESVIKNYKNHFPDIFSKASECMQVIFGIDVKEVDPAGHSYILVT | |
| | | CLGLSYDGLLGDDQSTPKTGLLIIVLGMILMEGSRAPEEAIWEALS | |
| | | VMGLYDGREHSVYWKLRKLLTQEWVQENYLEYRQAPGSDPVR | |
| | | YEFLWGPRALAETSYVKVLEHVVRVNARVRISYPSLHEEALGEE | |
| | | KGV | |
| MAGEB4 | uc004dcb.3 | MPRGQKSKLRAREKRQRTRGQTQDLKVGQPTAAEKEESPSSSSS | 23 |
| | | VLRDTASSSLAFGIPQEPQREPPTTSAAAAMSCTGSDKGDESQDEE | |
| | | NASSSQASTSTERSLKDSLTRKTKMLVQFLLYKYKMKEPTTKAE | |
| | | MLKIISKKYKEHFPEIFRKVSQRTELVFGLALKEVNPTTHSYILVS | |
| | | MLGPNDGNQSSAWTLPRNGLLMPLLSVIFLNGNCAREEEIWEFLN | |
| | | MLGIYDGKRHLIFGEPRKLITQDLVQEKYLEYQQVPNSDPPRYQF | |
| | | LWGPRAHAETSKMKVLEFLAKVNDTTPNNFPLLYEEALRDEEER | |
| | | AGARPRVAARRGTTAMTSAYSRATSSSSSQPM | |
| CT45A5 | uc011mvu.2 | MTDKTEKVAVDPETVFKRPRECDSPSYQKRQRMALLARKQGAG | 24 |
| | | DSLIAGSAMSKEKKLMTGHAIPPSQLDSQIDDFTGFSKDGMMQKP | |
| | | GSNAPVGGNVTSNFSGDDLECRGIASSPKSQQEINADIKCQVVKEI | |
| | | RCLGRKYEKIFEMLEGVQGPTAVRKRFFESIIKEAARCMRRDFVK | |
| | | HLKKKLKRMI | |
| ALPPL2 | uc002vss.4 | MQGPWVLLLLGLRLQLSLGIIPVEEENPDFWNRQAAEALGAAKK | 25 |
| | | LQPAQTAAKNLIIFLGDGMGVSTVTAARILKGQKKDKLGPETFLA | |
| | | MDRFPYVALSKTYSVDKHVPDSGATATAYLCGVKGNFQTIGLSA | |
| | | AARFNQCNTTRGNEVISVMNRAKKAGKSVGVVTTTRVQHASPA | |
| | | GAYAHTVNRNWYSDADVPASARQEGCQDIATQLISNMDIDVILG | |
| | | GGRKYMFPMGTPDPEYPDDYSQGGTRLDGKNLVQEWLAKHQG | |
| | | ARYVWNRTELLQASLDPSVTHLMGLFEPGDMKYEIHRDSTLDPS | |
| | | LMEMTEAALLLLSRNPRGFFLFVEGGRIDHGHHESRAYRALTETI | |
| | | MFDDAIERAGQLTSEEDTLSLVTADHSHVFSFGGYPLRGSSIFGLA | |
| | | PGKARDRKAYTVLLYGNGPGYVLKDGARPDVTESESGSPEYRQQ | |
| | | SAVPLDGETHAGEDVAVFARGPQAHLVHGVQEQTFIAHVMAFA | |
| | | ACLEPYTACDLAPRAGTTDAAHPGPSVVPALLPLLAGTLLLLGTA | |
| | | TAP | |
| MMP13 | uc001ph1.3 | MHPGVLAAFLFLSWTHCRALPLPSGGDEDDLSEEDLQFAERYLRS | 26 |
| | | YYHPTNLAGILKENAASSMTERLREMQSFFGLEVTGKLDDNTLD | |
| | | VMKKPRCGVPDVGEYNVFPRTLKWSKMNLTYRIVNYTPDMTHS | |
| | | EVEKAFKKAFKVWSDVTPLNFTRLHDGIADIMISFGIKEHGDFYPF | |
| | | DGPSGLLAHAFPPGPNYGGDAHFDDDETWTSSSKGYNLFLVAAH | |
| | | EFGHSLGLDHSKDPGALMFPIYTYTGKSHFMLPDDDVQGIQSLYG | |
| | | PGDEDPNPKHPKTPDKCDPSLSLDAITSLRGETMIFKDRFFWRLHP | |
| | | QQVDAELFLTKSFWPELPNRIDAAYEHPSHDLIFIFRGRKFWALN | |
| | | GYDILEGYPKKISELGLPKEVKKISAAVHFEDTGKTLLFSGNQVW | |
| | | RYDDTNHIMDKDYPRLIEEDFPGIGDKVDAVYEKNGYIYFFNGPI | |
| | | QFEYSIWSNRIVRVMPANSILWC | |
| CTAG1B | uc004fmf.1 | MQAEGRGTGGSTGDADGPGGPGIPDGPGGNAGGPGEAGATGGR | 27 |
| | | GPRGAGAARASGPGGGAPRGPHGGAASGLNGCCRCGARGPESRL | |
| | | LEFYLAMPFATPMEAELARRSLAQDAPPLPVPGVLLKEFTVSGNI | |
| | | LTIRLTAADHRQLQLSISSCLQQLSLLMWITQCFLPVFLAQPPSGQ | |
| | | RR | |
| DCT | uc010afh.3 | MSPLWWGFLLSCLGCKILPGAQGQFPRVCMTVDSLVNKECCPRL | 28 |
| | | GAESANVCGSQQGRGQCTEVRADTRPWSGPYILRNQDDRELWPR | |
| | | KFFHRTCKCTGNFAGYNCGDCKFGWTGPNCERKKPPVIRQNIHSL | |
| | | SPQEREQFLGALDLAKKRVHPDYVITTQHWLGLLGPNGTQPQFA | |
| | | NCSVYDFFVWLHYYSVRDTLLGPGRPYRAIDFSHQGPAFVTWHR | |
| | | YHLLCLERDLQRLIGNESFALPYWNFATGRNECDVCTDQLFGAA | |
| | | RPDDPTLISRNSRFSSWETVCDSLDDYNHLVTLCNGTYEGLLRRN | |
| | | QMGRNSMKLPTLKDIRDCLSLQKFDNPPFFQNSTFSFRNALEGFD | |
| | | KADGTLDSQVMSLHNLVHSFLNGTNALPHSAANDPIFVVISNRLL | |
| | | YNATTNILEHVRKEKATKELPSLHVLVLHSFTDAIFDEWMKRFNP | |
| | | PADAWPQELAPIGHNRMYNMVPFFPPVTNEELFLTSDQLGYSYAI | |
| | | DLPVSVEETPGWPTTLLVVMGTLVALVGLFVLLAFLQYRRLRKG | |
| | | YTPLMETHLSSKRYTEEA | |
| CLDN6 | uc021tbb.1 | MASAGMQILGVVLTLLGWVNGLVSCALPMWKVTAFIGNSIVVA | 29 |
| | | QVVWEGLWMSCVVQSTGQMQCKVYDSLLALPQDLQAARALCV | |
| | | IALLVALFGLLVYLAGAKCTTCVEEKDSKARLVLTSGIVFVISGVL | |
| | | TLIPVCWTAHAIIRDFYNPLVAEAQKRELGASLYLGWAASGLLLL | |
| | | GGGLLCCTCPSGGSQGPSHYMARYSTSAPAISRGPSEYPTKNYV | |
| MLANA | uc003zjo.1 | MPREDAHFIYGYPKKGHGHSYTTAEEAAGIGILTVILGVLLLIGC | 30 |
| | | WYCRRRNGYRALMDKSLHVGTQCALTRRCPQEGFDHRDSKVSL | |
| | | QEKNCEPVVPNAPPAYEKLSAEQSPPPYSP | |
| AFP | uc003hgz.1 | MKWVESIFLIFLLNFTESRTLHRNEYGIASILDSYQCTAEISLADLA | 31 |
| | | TIFFAQFVQEATYKEVSKMVKDALTAIEKPTGDEQSSGCLENQLP | |
| | | AFLEELCHEKEILEKYGHSDCCSQSEEGRHNCFLAHKKPTPASIPL | |
| | | FQVPEPVTSCEAYEEDRETFMNKFIYEIARRHPFLYAPTILLWAAR | |
| | | YDKIIPSCCKAENAVECFQTKAATVTKELRESSLLNQHACAVMK | |
| | | NFGTRTFQAITVTKLSQKFTKVNFTEIQKLVLDVAHVHEHCCRGD | |
| | | VLDCLQDGEKIMSYICSQQDTLSNKITECCKLTTLERGQCIIHAEN | |
| | | DEKPEGLSPNLNRFLGDRDFNQFSSGEKNIFLASFVHEYSRRHPQL | |
| | | AVSVILRVAKGYQELLEKCFQTENPLECQDKGEEELQKYIQESQA | |
| | | LAKRSCGLFQKLGEYYLQNAFLVAYTKKAPQLTSSELMAITRKM | |
| | | AATAATCCQLSEDKLLACGEGAADIIIGHLCIRHEMTPVNPGVGQ | |
| | | CCTSSYANRRPCFSSLVVDETYVPPAFSDDKFIFHKDLCQAQGVA | |
| | | LQTMKQEFLINLVKQKPQITEEQLEAVIADFSGLLEKCCQGQEQE | |
| | | VCFAEEGQKLISKTRAALGV | |
| DKK4 | uc003xpb.3 | MVAAVLLGLSWLCSPLGALVLDFNNIRSSADLHGARKGSQCLSD | 32 |
| | | TDCNTRKFCLQPRDEKPFCATCRGLRRRCQRDAMCCPGTLCVND | |
| | | VCTTMEDATPILERQLDEQDGTHAEGTTGHPVQENQPKRKPSIKK | |
| | | SQGRKGQEGESCLRTFDCGPGLCCARHFWTKICKPVLLEGQVCSR | |
| | | RGHKDTAQAPEIFQRCDCGPGLLCRSQLTSNRQHARLRVCQKIEK | |
| | | L | |
| ASCL2 | uc021qcf.1 | MDGGTLPRSAPPAPPVPVGCAARRRPASPELLRCSRRRRPATAET | 33 |
| | | GGGAAAVARRNERERNRVKLVNLGFQALRQHVPHGGASKKLSK | |
| | | VETLRSAVEYIRALQRLLAEHDAVRNALAGGLRPQAVRPSAPRGP | |
| | | PGTTPVAASPSRASSSPGRGGSSEPGSPRSAYSSDDSGCEGALSPA | |
| | | ERELLDFSSWLGGY | |
| GAGE1 | uc004dok.2 | MSWRGRSTYYWPRPRRYVQPPEMIGPMRPEQFSDEVEPATPEEG | 34 |
| | | EPATQRQDPAAAQEGEDEGASAGQGPKPEADSQEQGHPQTGCEC | |
| | | EDGPDGQEMDPPNPEEVKTPEEEMRSHYVAQTGILWLLMNNCFL | |
| | | NLSPRKP | |
| GAGE10 | uc010nir.1 | MSWRGRSTYRSRPRLYVEPPEMIGPMLPEQFSDEVEPATPEEGEP | 35 |
| | | ATQRQDPAAAQEGEDEGASAGQGPKPEADSQEQVHPKTGCECG | |
| | | DGPDGQEMGLPNPEEVKRPEEGEKQSQC | |
| SLC45A2 | uc003jid.3 | MGSNSGQAGRHIYKSLADDGPFDSVEPPKRPTSRLIMHSMAMFG | 36 |
| | | REFCYAVEAAYVTPVLLSVGLPSSLYSIVWFLSPILGFLLQPVVGS | |
| | | ASDHCRSRWGRRRPYILTLGVMMLVGMALYLNGATVVAALIAN | |
| | | PRRKLVWAISVTMIGVVLFDFAADFIDGPIKAYLFDVCSHQDKEK | |
| | | GLHYHALFTGFGGALGYLLGAIDWAHLELGRLLGTEFQVMFFFS | |
| | | ALVLTLCFTVHLCSISEAPLTEVAKGIPPQQTPQDPPLSSDGMYEY | |
| | | GSIEKVKNGYVNPELAMQGAKNKNHAEQTRRAMTLKSLLRALV | |
| | | NMPPHYRYLCISHLIGWTAFLSNMLFFTDFMGQIVYRGDPYSAHN | |
| | | STEFLIYERGVEVGCWGFCINSVFSSLYSYFQKVLVSYIGLKGLYF | |
| | | TGYLLFGLGTGFIGLFPNVYSTLVLCSLFGVMSSTLYTVPFNLITE | |
| | | YHREEEKERQQAPGGDPDNSVRGKGMDCATLTCMVQLAQILVG | |
| | | GGLGFLVNTAGTVVVVVITASAVALIGCCFVALFVRYVD | |
| PAGE5 | uc004duj.3 | MQAPWAGNRGWAGTREEVRDMSEHVTRSQSSERGNDQESSQPV | 37 |
| | | GPVIVQQPTEEKRQEEEPPTDNQGIAPSGEIKNEGAPAVQGTDVEA | |
| | | FQQELALLKIEDAPGDGPDVREGTLPTFDPTKVLEAGEGQL | |
| PAGE2 | uc004duf.1 | MSELLRARSQSSERGNDQESSQPVGSVIVQEPTEEKRQEEEPPTDN | 38 |
| | | QGIAPSGEIENQAVPAFQGPDMEAFQQELALLKIEDEPGDGPDVR | |
| | | EGIMPTFDLTKVLEAGDAQP | |
| PMEL | uc001siq.3 | MDLVLKRCLLHLAVIGALLAVGATKVPRNQDWLGVSRQLRTKA | 39 |
| | | WNRQLYPEWTEAQRLDCWRGGQVSLKVSNDGPTLIGANASFSIA | |
| | | LNFPGSQKVLPDGQVIWVNNTIINGSQVWGGQPVYPQETDDACIF | |
| | | PDGGPCPSGSWSQKRSFVYVWKTWGQYWQVLGGPVSGLSIGTG | |
| | | RAMLGTHTMEVTVYHRRGSRSYVPLAHSSSAFTITDQVPFSVSVS | |
| | | QLRALDGGNKHFLRNQPLTFALQLHDPSGYLAEADLSYTWDFGD | |
| | | SSGTLISRALVVTHTYLEPGPVTAQVVLQAAIPLTSCGSSPVPGTT | |
| | | DGHRPTAEAPNTTAGQVPTTEVVGTTPGQAPTAEPSGTTSVQVPT | |
| | | TEVISTAPVQMPTAESTGMTPEKVPVSEVMGTTLAEMSTPEATG | |
| | | MTPAEVSIVVLSGTTAAQVTTTEWVETTARELPIPEPEGPDASSIM | |
| | | STESITGSLGPLLDGTATLRLVKRQVPLDCVLYRYGSFSVTLDIVQ | |
| | | GIESAEILQAVPSGEGDAFELTVSCQGGLPKEACMEISSPGCQPPA | |
| | | QRLCQPVLPSPACQLVLHQILKGGSGTYCLNVSLADTNSLAVVST | |
| | | QLIMPVPGILLTGQEAGLGQVPLIVGILLVLMAVVLASLIYRRRLM | |
| | | KQDFSVPQLPHSSSHWLRLPRIFCSCPIGENSPLLSGQQV | |

TABLE 5

| Virus, | Genbank | | |
|---|---|---|---|
| Gene | Accession | Sequence | SEQ ID NO: |
| HPV-16, | NC_001526 | MHQKRTAMFQDPQERPRKLPQLCTELQTTIHDIILECVYCKQQLL | 40 |
| E6 | | RREVYDFAFRDLCIVYRDGNPYAVCDKCLKFYSKISEYRHYCYS | |
| | | LYGTTLEQQYNKPLCDLLIRCINCQKPLCPEEKQRHLDKKQRFH | |
| | | NIRGRWTGRCMSCCRSSRTRRETQL | |
| HPV-16, | NC_001526 | MHGDTPTLHEYMLDLQPETTDLYCYEQLNDSSEEEDEIDGPAGQ | 41 |
| E7 | | AEPDRAHYNIVTFCCKCDSTLRLCVQSTHVDIRTLEDLLMGTLGI | |
| | | VCPICSQKP | |
| EBV, LF2 | KC207813 | MAEAYPGGAHAALASRRSSFRNSLRRLRPTEKPDTSFMRGVWK | 42 |
| | | YEIFPSYVRVTNKQVLQLDAQCQELPPCPSVGQILSFKLPSFSFNT | |
| | | TTYGSRYFTVAFLFFGAEDNEVFLKPFFVMHSDQDIVLSVLNPRS | |
| | | LFIEKGKFTWYIVPIRLVKNPYLYLQILPGQSDIQLTRSCTQSGDK | |
| | | LNTSEPQIFLSGSPVTSQDECLPYLLAQHTPPFLKSYARIHTFPGK | |
| | | VCPVNAIRRGKGYVRVSVDTPDLKREGPLNVKVGMTLLDDVIIA | |
| | | FRYNPYPKSHWRWDGESTDIRYFGSPVIIPPNFITELEYNNTYEAP | |
| | | LSSKITAIVVSHSSNPVFYVYPQEWKPGQTLKLTVRNISNNPITIV | |
| | | TGQSMAQAFFIYAGDPSISTIMRRYIQRQGCALTLPGNIVVESSSL | |
| | | PTFERINKTFNGNIVASEGTL | |
| EBV, | KC207813 | MSGGLFYNPFLRPNKGLLKKPDKEYLRLIPKCFQTPGAAGVVDV | 43 |
| BALF5 | | RGPQPPLCFYQDSLTVVGGDEDGKGMWWRQRAQEGTARPEAD | |
| | | THGSPLDFHVYDILETVYTHEKCAVIPSDKQGYVVPCGIVIKLLG | |
| | | RRKADGASVCVNVFGQQAYFYASAPQGLDVEFAVLSALKASTF | |
| | | DRRTPCRVSVEKVTRRSIMGYGNHAGDYHKITLSHPNSVCHVAT | |
| | | WLQDKHGCRIFEANVDATRRFVLDNDFVTFGWYSCRRAIPRLQ | |
| | | HRDSYAELEYDCEVGDLSVRREDSSWPSYQALAFDIECLGEEGF | |
| | | PTATNEADLILQISCVLWSTGEEAGRYRRILLTLGTCEDIEGVEVY | |
| | | EFPSELDMLYAFFQLIRDLSVEIVTGYNVANFDWPYILDRARHIY | |
| | | SINPASLGKIRAGGVCEVRRPHDAGKGFLRANTKVRITGLIPIDM | |
| | | YAVCRDKLSLSDYKLDTVARHLLGAKKEDVHYKEIPRLFAAGPE | |
| | | GRRRLGMYCVQDSALVMDLLNHFVIHVEVAEIAKIAHIPCRRVL | |
| | | DDGQQIRVFSCLLAAAQKENFILPMPSASDRDGYQGATVIQPLSG | |
| | | FYNSPVLVVDFASLYPSIIQAHNLCYSTMITPGEEHRLAGLRPGED | |
| | | YESFRLTGGVYHFVKKHVHESFLASLLTSWLAKRKAIKKLLAAC | |
| | | EDPRQRTILDKQQLAIKCTCNAVYGFTGVANGLFPCLSIAETVTL | |
| | | QGRTMLERAKAFVEALSPANLQALAPSPDAWAPLNPEGQLRVIY | |
| | | GDTDSLFIECRGFSESETLRFAEALAAHTTRSLFVAPISLEAEKTFS | |
| | | CLMLITKKRYVGVLTDGKTLMKGVELVRKTACKFVQTRCRRVL | |
| | | DLVLADARVKEAASLLSHRPFQESFTQGLPVGFLPVIDILNQAYT | |
| | | DLREGRVPMGELCFSTELSRKLSAYKSTQMPHLAVYQKFVERNE | |
| | | ELPQIHDRIQYVFVEPKGGVKGARKTEMAEDPAYAERHGVPVA | |
| | | VDHYFDKLLQGAANILQCLFDNNSGAALSVLQNFTARPPF | |
| EBV, | KC207813 | MAGARRRARCPASAGCAYSARPPPLSTRGRRISAGSGQPRWWP | 44 |
| RPMS1 | | WGSPPPLDTRYRRPGPGRRARSCLHAGPRGRPPHSRTRARRTSPG | |
| | | AGGGGWRGGSCTSQR | |
| EBV, A73 | KC207813 | MSMPPKGFLKKEMKPETRLLNKPPTVLTRPAMFCAWKLYSRKM | 45 |
| | | PSRSKTLEARCSSRPPCDSPACQTRDTGCPRRSGTGRRGWRARRL | |
| | | GKESWFADAWRMARYWGCAVKAAAQSAFSASTASPEEL | |
| EBV, | KC207813 | MTRRRVLSVVVLLAALACRLGAQTPEQPAPPATTVQPTATRQQT | 46 |
| BALF4 | | SFPFRVCELSSHGDLFRFSSDIQCPSFGTRENHTEGLLMVFKDNIIP | |
| | | YSFKVRSYTKIVTNILIYNGWYADSVTNRHEEKFSVDSYETDQM | |
| | | DTIYQCYNAVKMTKDGLTRVYVDRDGVNITVNLKPTGGLANGV | |
| | | RRYASQTELYDAPGWLIWTYRTRTTVNCLITDMMAKSNSPFDFF | |
| | | VTTTGQTVEMSPFYDGKNKETFHERADSFHVRTNYKIVDYDNR | |
| | | GTNPQGERRAFLDKGTYTLSWKLENRTAYCPLQHWQTFDSTIAT | |
| | | ETGKSIHFVTDEGTSSFVTNTTVGIELPDAFKCIEEQVNKTMHEK | |
| | | YEAVQDRYTKGQEAITYFITSGGLLLAWLPLTPRSLATVKNLTEL | |
| | | TTPTSSPPSSPSPPAPPAARGSTSAAVLRRRRRDAGNATTPVPPAA | |
| | | PGKSLGTLNNPATVQIQFAYDSLRRQINRMLGDLARAWCLEQKR | |
| | | QNMVLRELTKINPTTVMSSIYGKAVAAKRLGDVISVSQCVPVNQ | |
| | | ATVTLRKSMRVPGSETMCYSRPLVSFSFINDTKTYEGQLGTDNEI | |
| | | FLTKKMTEVCQATSQYYFQSGNEIHVYNDYHHFKTIELDGIATL | |
| | | QTFISLNTSLIENIDFASLELYSRDEQRASNVFDLEGIFREYNFQAQ | |
| | | NIAGLRKDLDNAVSNGRNQFVDGLGELMDSLGSVGQSITNLVST | |
| | | VGGLFSSLVSGFISFFKNPFGGMLILVLVAGVVILVISLTRRTRQM | |
| | | SQQPVQMLYPGIDELAQQHASGEGPGINPISKTELQAIMLALHEQ | |
| | | NQEQKRAAQRAAGPSVASRALQAARDRFPGLRRRRYHDPETAA | |
| | | ALLGEAETEF | |
| EBV, | KC207813 | MSGLLAAAYSQVYALAVELSVCARLDPRSLDVAAVVRNAGLLA | 47 |
| BALF3 | | ELEAILLPRLRRQNDRACSALSLELVHLLENSREASAALLAPGRK | |
| | | GTRVPPLRTPSVAYSVEFYGGHKVDVSLCLINDIEILMKRINSVFY | |
| | | CMSHTMGLESLERALDLLGRFRGVSPIPDPRLYITSVPCWRCVGE | |
| | | LMVLPNHGNPSTAEGTHVSCNHLAVPVNPEPVSGLFENEVRQAG | |
| | | LGHLLEAEEKARPGGPEEGAVPGPGRPEAEGATRALDTYNVFST | |
| | | VPPEVAELSELLYWNSGGHAIGATGQGEGGGHSRLSALFARERR | |
| | | LALVRRACEEALAGARLTHLFDAVAPGATERLFCGGVYSSSGDA | |
| | | VEALKADCAAAFTAHPQYRAILQKRNELYTRLNRAMQRLGRGE | |
| | | EEASRESPEVPRPAGAREPGPSGALSDALKRKEQYLRQVATEGL | |
| | | AKLQSCLAQQSETLTETLCLRVWGDVVYWELARMRNHFLYRR | |
| | | AFVSGPWEDRRAGEGAAFENSKYIKTHLFTQTLSSEHLHALTHSL | |
| | | YTFITGPLAEESGLFPPPSNVALARCCDAAGTLPHQKAFLTSLIWP | |
| | | GIEPSDWIETSFNSFYSVPGGSLASSQQILCRALREAVLTVSLYNK | |
| | | TWGRSLILRRADAVSPGQALPPDGLYLTYDSDRPLILLYKGRGW | |
| | | VFKDLYALLYLHLQMRDDSA | |
| EBV, | KC207813 | APGYAVEAVEGGLYPVARLDAWPYQGSQERLLVRQRTCGVTA | 48 |
| BARF0 | | ASQGHVAGWGKEPALLRQGPRDEGVQAVRQRVQVLRAQGLGK | |
| | | QVCFDVLGILKGGTLAGAPVLPGTRDEGPSVEEVVAHAGQLPVD | |
| | | HVPPDAQAQGLGQGLALLRQAGLQLGQTLGGHLAQVLLLALER | |
| | | VREGAGRAGLSCPSRPGHLRALPGRLLLASAQPLHGSVEPRVEL | |
| | | VPLLQDGPVLGVRREGGGAVRLQRLHRVARGAVDPAAEEPLCG | |
| | | PGSHGIKQVSQPCPRQRLLAGPPHQGQATLPGKQGREAGMSATL | |
| | | PLPRCTDSMAARVPIEELREFRHLRGHCREDVVGVQRSGRPLCL | |
| | | RPPRARDRALLWAARPRLLLSLQQVPEPSLPDFILKQSRDRLRIH | |
| | | RHRQVVTGDVGPLCRGRVAVVGQNHQLAHTAPAGHRGDVEAR | |
| | | VWDGTYAPKAAQQIQGPFQALQPHGVRHAIKHAIDSLH | |

For each epitope, the full-length amino acid sequence of the non-mutated protein epitope was derived. Any constituent 9mer or 1 Omer not found in the germline protein sequence was flagged and scored for binding potential on six common HLA alleles (HLA-A01:01, HLA-A02:01. HLA-A03:01, HLA-A24:02, HLA-B07:02, and HLA-B08:01) using available algorithms. Any peptide scoring better than 1000 nM was nominated.

TABLE 6

| | SEQ | | |
|---|---|---|---|
| | ID | | |
| Peptide | NO: | Virus, Gene | Affinity |
| CMSCCRSSR | 49 | HPV-16, E6 | A03.01 = 610 |
| CPEEKQRHL | 50 | HPV-16, E6 | B07.02 = 720 |
| CVYCKQQLL | 51 | HPV-16, E6 | B08.01 = 880 |
| CVYCKQQLLR | 52 | HPV-16, E6 | A03.01 = 250 |
| CYSLYGTTL | 53 | HPV-16, E6 | A24.02 = 290 |
| DKKQRFHNI | 54 | HPV-16, E6 | B08.01 = 200 |
| EYRHYCYSL | 55 | HPV-16, E6 | A24.02 = 350; B08.01 = 360 |
| FAFRDLCIV | 56 | HPV-16, E6 | A02.01 = 150 |
| IILECVYCK | 57 | HPV-16, E6 | A03.01 = 150 |
| ISEYRHYCY | 58 | HPV-16, E6 | A01.01 = 81 |
| IVYRDGNPY | 59 | HPV-16, E6 | A03.01 = 700 |
| IVYRDGNPYA | 60 | HPV-16, E6 | A02.01 = 760 |
| KFYSKISEY | 61 | HPV-16, E6 | A03.01 = 670 |
| KISEYRHYCY | 62 | HPV-16, E6 | A03.01 = 570 |
| KLPQLCTEL | 63 | HPV-16, E6 | A02.01 = 130 |
| LIRCINCQK | 64 | HPV-16, E6 | A03.01 = 230 |
| LLIRCINCQK | 65 | HPV-16, E6 | A03.01 = 130 |
| MHQKRTAMF | 66 | HPV-16, E6 | A24.02 = 980; B08.01 = 580 |
| NPYAVCDKCL | 67 | HPV-16, E6 | B07.02 = 550 |
| QYNKPLCDLL | 68 | HPV-16, E6 | A24.02 = 520 |
| RFHNIRGRW | 69 | HPV-16, E6 | A24.02 = 620 |
| RGRWTGRCM | 70 | HPV-16, E6 | B07.02 = 720 |
| RPRKLPQLC | 71 | HPV-16, E6 | B07.02 = 310 |
| RPRKLPQLCT | 72 | HPV-16, E6 | B07.02 = 79 |
| SEYRHYCYSL | 73 | HPV-16, E6 | B08.01 = 390 |
| SSRTRRETQL | 74 | HPV-16, E6 | B08.01 = 230 |
| TIHDIILECV | 75 | HPV-16, E6 | A02.01 = 140 |
| TTLEQQYNK | 76 | HPV-16, E6 | A03.01 = 520 |
| VYDFAFRDL | 77 | HPV-16, E6 | A24.02 = 600 |
| GIVCPICSQK | 78 | HPV-16, E7 | A03.01 = 200 |
| GTLGIVCPI | 79 | HPV-16, E7 | A02.01 = 120 |
| HGDTPTLHEY | 80 | HPV-16, E7 | A01.01 = 270 |
| IVCPICSQK | 81 | HPV-16, E7 | A03.01 = 200 |
| LLMGTLGIV | 82 | HPV-16, E7 | A02.01 = 20 |
| RAHYNIVTF | 83 | HPV-16, E7 | A24.02 = 330 |
| RLCVQSTHV | 84 | HPV-16, E7 | A02.01 = 770 |
| TLEDLLMGTL | 85 | HPV-16, E7 | A02.01 = 480 |
| TLHEYMLDL | 86 | HPV-16, E7 | A02.01 = 95 |
| TPTLHEYML | 87 | HPV-16, E7 | B07.02 = 490 |
| YMLDLQPET | 88 | HPV-16, E7 | A02.01 = 7 |
| YMLDLQPETT | 89 | HPV-16, E7 | A02.01 = 25 |
| AALASRRSSF | 90 | EBV, LF2 | B07.02 = 160 |
| ALASRRSSF | 91 | EBV, LF2 | B07.02 = 290; B08.01 = 41 |
| ALASRRSSFR | 92 | EBV, LF2 | A03.01 = 160 |
| ALTLPGNIVV | 93 | EBV, LF2 | A02.01 = 470 |
| APLSSKITA | 94 | EBV, LF2 | B07.02 = 490 |
| APLSSKITAI | 95 | EBV, LF2 | B07.02 = 31 |
| AQHTPPFLK | 96 | EBV, LF2 | A03.01 = 140 |
| AYPGGAHAAL | 97 | EBV, LF2 | A24.02 = 470 |
| CPSVGQILSF | 98 | EBV, LF2 | B07.02 = 190 |
| EVFLKPFFV | 99 | EBV, LF2 | A02.01 = 210 |
| FFGAEDNEVF | 100 | EBV, LF2 | A24.02 = 910 |
| FIEKGKFTWY | 101 | EBV, LF2 | A01.01 = 450 |
| FIYAGDPSI | 102 | EBV, LF2 | A02.01 = 13 |
| FLSGSPVTS | 103 | EBV, LF2 | A02.01 = 630 |
| FMRGVWKYEI | 104 | EBV, LF2 | A02.01 = 140; B08.01 = 340 |
| FTVAFLFFGA | 105 | EBV, LF2 | A02.01 = 17 |
| FTWYIVPIRL | 106 | EBV, LF2 | A02.01 = 63 |
| FVMHSDQDIV | 107 | EBV, LF2 | A02.01 = 130 |
| GPLNVKVGM | 108 | EBV, LF2 | B07.02 = 610 |
| GQSMAQAFFI | 109 | EBV, LF2 | A02.01 = 510 |
| HSDQDIVLSV | 110 | EBV, LF2 | A01.01 = 860 |
| HSSNPVFYV | 111 | EBV, LF2 | A02.01 = 800 |
| HSSNPVFYVY | 112 | EBV, LF2 | A01.01 = 170 |
| HTFPGKVCPV | 113 | EBV, LF2 | A02.01 = 200 |
| IAFRYNPYPK | 114 | EBV, LF2 | A03.01 = 58 |
| IIPPNFITEL | 115 | EBV, LF2 | A02.01 = 350 |
| ILPGQSDIQL | 116 | EBV, LF2 | A02.01 = 350 |
| IPPNFITEL | 117 | EBV, LF2 | B07.02 = 200 |
| ITELEYNNTY | 118 | EBV, LF2 | A01.01 = 61 |
| IVVSHSSNPV | 119 | EBV, LF2 | A02.01 = 160 |
| KFTWYIVPI | 120 | EBV, LF2 | A24.02 = 200 |
| KLNTSEPQI | 121 | EBV, LF2 | A02.01 = 270 |
| KLPSFSFNT | 122 | EBV, LF2 | A02.01 = 63 |
| KLPSFSFNTT | 123 | EBV, LF2 | A02.01 = 520 |
| KNPYLYLQI | 124 | EBV, LF2 | A24.02 = 900 |
| KPDTSFMRGV | 125 | EBV, LF2 | B07.02 = 920 |
| KPGQTLKLTV | 126 | EBV, LF2 | B07.02 = 320 |
| KSYARIHTF | 127 | EBV, LF2 | A24.02 = 210 |
| KVCPVNAIRR | 128 | EBV, LF2 | A03.01 = 850 |
| KYEIFPSYV | 129 | EBV, LF2 | A24.02 = 740 |
| LAQHTPPFLK | 130 | EBV, LF2 | A03.01 = 440 |
| LDDVIIAFRY | 131 | EBV, LF2 | A01.01 = 860 |
| LFIEKGKFTW | 132 | EBV, LF2 | A24.02 = 230 |
| LLAQHTPPF | 133 | EBV, LF2 | A02.01 = 540; B07.02 = 750; B08.01 = 190 |
| LLAQHTPPFL | 134 | EBV, LF2 | A02.01 = 9.6; B08.01 = 480 |
| LLDDVIIAF | 135 | EBV, LF2 | A02.01 = 120 |
| LPGQSDIQL | 136 | EBV, LF2 | B07.02 = 470 |
| LPPCPSVGQI | 137 | EBV, LF2 | B07.02 = 930 |
| LPSFSFNTT | 138 | EBV, LF2 | B07.02 = 490 |
| LPSFSFNTTT | 139 | EBV, LF2 | B07.02 = 560 |
| LQLDAQCQEL | 140 | EBV, LF2 | A02.01 = 220 |
| LTLPGNIVV | 141 | EBV, LF2 | A02.01 = 880 |
| MAQAFFIYA | 142 | EBV, LF2 | A02.01 = 450 |
| NPYLYLQIL | 143 | EBV, LF2 | B07.02 = 170; B08.01 = 150 |
| NSLRRLRPT | 144 | EBV, LF2 | B08.01 = 370 |
| NTTTYGSRY | 145 | EBV, LF2 | A01.01 = 210 |
| NTYEAPLSSK | 146 | EBV, LF2 | A03.01 = 120 |
| PFLKSYARI | 147 | EBV, LF2 | A24.02 = 390 |
| PSYVRVTNK | 148 | EBV, LF2 | A03.01 = 620 |
| PYPKSHWRW | 149 | EBV, LF2 | A24.02 = 100 |
| QIFLSGSPV | 150 | EBV, LF2 | A02.01 = 380 |
| QLDAQCQEL | 151 | EBV, LF2 | A02.01 = 370 |
| QSMAQAFFI | 152 | EBV, LF2 | A02.01 = 890 |
| QSMAQAFFIY | 153 | EBV, LF2 | A01.01 = 190 |
| RLVKNPYLY | 154 | EBV, LF2 | A03.01 = 410 |
| RLVKNPYLYL | 155 | EBV, LF2 | A02.01 = 120 |
| RPTEKPDTSF | 156 | EBV, LF2 | B07.02 = 39 |
| RSLFIEKGK | 157 | EBV, LF2 | A03.01 = 430 |
| RSSFRNSLR | 158 | EBV, LF2 | A03.01 = 230 |
| RSSFRNSLRR | 159 | EBV, LF2 | A03.01 = 490 |
| RVSVDTPDLK | 160 | EBV, LF2 | A03.01 = 600 |
| RYFGSPVII | 161 | EBV, LF2 | A24.02 = 25 |
| RYFTVAFLF | 162 | EBV, LF2 | A24.02 = 3.7 |
| RYFTVAFLFF | 163 | EBV, LF2 | A24.02 = 5.2 |
| RYIQRQGCAL | 164 | EBV, LF2 | A24.02 = 450 |
| RYNPYPKSHW | 165 | EBV, LF2 | A24.02 = 530 |
| SFKLPSFSF | 166 | EBV, LF2 | A24.02 = 98 |
| SFMRGVWKY | 167 | EBV, LF2 | A24.02 = 460 |
| SLPTFERINK | 168 | EBV, LF2 | A03.01 = 420 |
| SLRRLRPTEK | 169 | EBV, LF2 | A03.01 = 120 |
| SMAQAFFIY | 170 | EBV, LF2 | A0l.01 = 870; A03.01 = 340 |
| SMAQAFFIYA | 171 | EBV, LF2 | A02.01 = 18 |
| SPVTSQDECL | 172 | EBV, LF2 | B07.02 = 540 |
| SQDECLPYL | 173 | EBV, LF2 | A02.01 = 18 |
| SQDECLPYLL | 174 | EBV, LF2 | A02.01 = 45 |
| SRRSSFRNSL | 175 | EBV, LF2 | B07.02 = 620; B08.01 = 150 |
| SSFRNSLRR | 176 | EBV, LF2 | A03.01 = 350 |
| SSNPVFYVY | 177 | EBV, LF2 | A01.01 = 110 |
| SVGQILSFK | 178 | EBV, LF2 | A03.01 = 73 |
| TFERINKTF | 179 | EBV, LF2 | A24.02 = 540 |
| TIMRRYIQR | 180 | EBV, LF2 | A03.01 = 280 |
| TLLDDVIIA | 181 | EBV, LF2 | A02.01 = 51 |
| TLLDDVIIAF | 182 | EBV, LF2 | A02.01 = 650 |
| TPDLKREGPL | 183 | EBV, LF2 | B07.02 = 100 |
| TSFMRGVWK | 184 | EBV, LF2 | A03.01 = 83 |
| TSFMRGVWKY | 185 | EBV, LF2 | A01.01 = 730 |
| TSQDECLPY | 186 | EBV, LF2 | A01.01 = 74 |
| TTYGSRYFTV | 187 | EBV, LF2 | A02.01 = 130 |
| TVAFLFFGA | 188 | EBV, LF2 | A02.01 = 290 |
| TVRNISNNPI | 189 | EBV, LF2 | B07.02 = 450 |
| TWYIVPIRL | 190 | EBV, LF2 | A24.02 = 680 |
| TYEAPLSSKI | 191 | EBV, LF2 | A24.02 = 420 |
| TYGSRYFTV | 192 | EBV, LF2 | A24.02 = 19 |
| VFLKPFFVM | 193 | EBV, LF2 | A24.02 = 600 |
| VFYVYPQEW | 194 | EBV, LF2 | A24.02 = 110 |
| VFYVYPQEWK | 195 | EBV, LF2 | A03.01 = 650 |
| VLNPRSLFI | 196 | EBV, LF2 | A02.01 = 110 |
| VLSVLNPRSL | 197 | EBV, LF2 | A02.01 = 680 |
| VMHSDQDIV | 198 | EBV, LF2 | A02.01 = 440 |
| VMHSDQDIVL | 199 | EBV, LF2 | A02.01 = 910 |
| VSHSSNPVFY | 200 | EBV, LF2 | A01.01 = 330 |
| VTSQDECLPY1 | 201 | EBV, LF2 | A01.01 = 26 |
| VVSHSSNPV | 202 | EBV, LF2 | A02.01 = 690 |
| WYIVPIRLV | 203 | EBV, LF2 | A24.02 = 700 |
| YARIHTFPG | 204 | EBV, LF2 | B08.01 = 430 |
| YARIHTFPGK | 205 | EBV, LF2 | A03.01 = 660 |
| YFTVAFLFF | 206 | EBV, LF2 | A24.02 = 62 |
| YIQRQGCAL | 207 | EBV, LF2 | B07.02 = 500; B08.01 = 890 |
| YIVPIRLVK | 208 | EBV, LF2 | A03.01 = 300 |
| YLLAQHTPPF | 209 | EBV, LF2 | A02.01 = 69; A24.02 = 550; B08.01 = 310 |
| YLYLQILPG | 210 | EBV, LF2 | A02.01 = 320 |
| YLYLQILPGQ | 211 | EBV, LF2 | A02.01 = 800 |
| YPGGAHAAL | 212 | EBV, LF2 | B07.02 = 7; B08.01 = 700 |
| YPGGAHAALA | 213 | EBV, LF2 | B07.02 = 410 |
| YVRVTNKQVL | 214 | EBV, LF2 | B07.02 = 70; B08.01 = 470 |
| AGRYRRILL | 215 | EBV, BALF5 | B08.01 = 810 |
| AIKCTCNAV | 216 | EBV, BALF5 | B08.01 = 760 |
| ALAAHTTRSL | 217 | EBV, BALF5 | A02.01 = 93; B07.02 = 200; B08.01 = 340 |
| ALAFDIECL | 218 | EBV, BALF5 | A02.01 = 70 |
| ALAPSPDAWA | 219 | EBV, BALF5 | A02.01 = 280 |
| ALKASTFDR | 220 | EBV, BALF5 | A03.01 = 1000 |
| ALSPANLQA | 221 | EBV, BALF5 | A02.01 = 530 |
| ALSPANLQAL | 222 | EBV, BALF5 | A02.01 = 65 |
| ALSVLQNFTA | 223 | EBV, BALF5 | A02.01 = 260 |
| APLNPEGQL | 224 | EBV, BALF5 | B07.02 = 85 |
| APQGLDVEF | 225 | EBV, BALF5 | B07.02 = 280 |
| APSPDAWAPL | 226 | EBV, BALF5 | B07.02 = 18 |
| ASLLTSWLAK | 227 | EBV, BALF5 | A03.01 = 140 |
| AVYGFTGVA | 228 | EBV, BALF5 | A02.01 = 830 |
| AVYQKFVER | 229 | EBV, BALF5 | A03.01 = 340 |
| AYKSTQMPHL | 230 | EBV, BALF5 | A24.02 = 770 |
| CLFDNNSGA | 231 | EBV, BALF5 | A02.01 = 130 |
| CLFDNNSGAA | 232 | EBV, BALF5 | A02.01 = 390 |
| CLGEEGFPT | 233 | EBV, BALF5 | A02.01 = 680 |
| CLGEEGFPTA | 234 | EBV, BALF5 | A02.01 = 120 |
| CLSIAETVTL | 235 | EBV, BALF5 | A02.01 = 580 |
| CVNVFGQQAY | 236 | EBV, BALF5 | A01.01 = 750 |
| DARVKEAASL | 237 | EBV, BALF5 | B08.01 = 330 |
| DLLNHFVIHV | 238 | EBV, BALF5 | A02.01 = 290 |
| DLREGRVPM | 239 | EBV, BALF5 | B07.02 = 690; B08.01 = 600 |
| DMLYAFFQL | 240 | EBV, BALF5 | A02.01 = 340 |
| DNDFVTFGWY | 241 | EBV, BALF5 | A01.01 = 820 |
| DRARHIYSI | 242 | EBV, BALF5 | B08.01 = 350 |
| DVRGPQPPL | 243 | EBV, BALF5 | B07.02 = 870 |
| EAGRYRRIL | 244 | EBV, BALF5 | B08.01 = 770 |
| ELSRKLSAYK | 245 | EBV, BALF5 | A03.01 = 450 |
| EMAEDPAYA | 246 | EBV, BALF5 | A02.01 = 480 |
| EYLRLIPKCF | 247 | EBV, BALF5 | A24.02 = 310 |
| FLASLLTSW | 248 | EBV, BALF5 | A02.01 = 150 |
| FLASLLTSWL | 249 | EBV, BALF5 | A02.01 = 4.1 |
| FLRANTKVRI | 250 | EBV, BALF5 | B08.01 = 260 |
| FLRPNKGLL | 251 | EBV, BALF5 | B08.01 = 250 |
| FLRPNKGLLK | 252 | EBV, BALF5 | A03.01 = 96 |
| FPTATNEADL | 253 | EBV, BALF5 | B07.02 = 180 |
| FQESFTQGL | 254 | EBV, BALF5 | A02.01 = 920 |
| FQLIRDLSV | 255 | EBV, BALF5 | A02.01 = 210; B08.01 = 390 |
| FQTPGAAGV | 256 | EBV, BALF5 | A02.01 = 48 |
| FQTPGAAGVV | 257 | EBV, BALF5 | A02.01 = 640 |
| FSESETLRF | 258 | EBV, BALF5 | A01.01 = 180 |
| FTQGLPVGFL | 259 | EBV, BALF5 | A02.01 = 960 |
| FVAPISLEA | 260 | EBV, BALF5 | A02.01 = 26 |
| FVEALSPANL | 261 | EBV, BALF5 | A02.01 = 860 |
| FVIHVEVAEI | 262 | EBV, BALF5 | A02.01 = 64 |
| FVKKHVHESF | 263 | EBV, BALF5 | B08.01 = 130 |
| FVLDNDFVT | 264 | EBV, BALF5 | A02.01 = 650 |
| FVLDNDFVTF | 265 | EBV, BALF5 | A24.02 = 750 |
| FVQTRCRRV | 266 | EBV, BALF5 | B08.01 = 340 |
| FVQTRCRRVL | 267 | EBV, BALF5 | B07.02 = 330; B08.01 = 100 |
| FYASAPQGL | 268 | EBV, BALF5 | A24.02 = 100 |
| GLDVEFAVL | 269 | EBV, BALF5 | A02.01 = 640 |
| GLFPCLSIA | 270 | EBV, BALF5 | A02.01 = 33 |
| GLFYNPFLR | 271 | EBV, BALF5 | A03.01 = 100 |
| GLIPIDMYA | 272 | EBV, BALF5 | A02.01 = 28 |
| GLIPIDMYAV | 273 | EBV, BALF5 | A02.01 = 5.9 |
| GLPVGFLPV | 274 | EBV, BALF5 | A02.01 = 68 |
| GLPVGFLPVI | 275 | EBV, BALF5 | A02.01 = 330 |
| GMYCVQDSA | 276 | EBV, BALF5 | A02.01 = 100 |
| GMYCVQDSAL | 277 | EBV, BALF5 | A02.01 = 64 |
| GPEGRRRLGM | 278 | EBV, BALF5 | B07.02 = 47 |
| GQQAYFYASA | 279 | EBV, BALF5 | A02.01 = 800 |
| GVANGLFPCL | 280 | EBV, BALF5 | A02.01 = 340 |
| GVYHFVKKH | 281 | EBV, BALF5 | A03.01 = 990 |
| GYNVANFDW | 282 | EBV, BALF5 | A24.02 = 270 |
| HIYSINPASL | 283 | EBV, BALF5 | A02.01 = 360 |
| HLAVYQKFV | 284 | EBV, BALF5 | A02.01 = 140 |
| HPNSVCHVA | 285 | EBV, BALF5 | B07.02 = 530 |
| HPNSVCHVAT | 286 | EBV, BALF5 | B07.02 = 380 |
| HVATWLQDK | 287 | EBV, BALF5 | A03.01 = 380 |
| HVHESFLASL | 288 | EBV, BALF5 | A02.01 = 670; B07.02 = 260; B08.01 = 990 |
| HVYDILETV | 289 | EBV, BALF5 | A02.01 = 12 |
| HVYDILETVY | 290 | EBV, BALF5 | A03.01 = 990 |
| HYKEIPRLF | 291 | EBV, BALF5 | A24.02 = 71 |
| IAHIPCRRVL | 292 | EBV, BALF5 | B07.02 = 620 |
| IIQAHNLCY | 293 | EBV, BALF5 | A0l.01 = 600; A03.01 = 790 |
| ILDKQQLAI | 294 | EBV, BALF5 | A02.01 = 180 |
| ILDKQQLAIK | 295 | EBV, BALF5 | A03.01 = 310 |
| ILDRARHIY | 296 | EBV, BALF5 | A0l.01 = 160 |
| ILETVYTHEK | 297 | EBV, BALF5 | A03.01 = 290 |
| ILNQAYTDL | 298 | EBV, BALF5 | A02.01 = 750 |
| ILQISCVLW | 299 | EBV, BALF5 | A24.02 = 990 |
| IPRLFAAGPE | 300 | EBV, BALF5 | B07.02 = 640 |
| IPRLQHRDSY | 301 | EBV, BALF5 | B07.02 = 360 |
| IPSDKQGYV | 302 | EBV, BALF5 | B07.02 = 610 |
| IPSDKQGYVV | 303 | EBV, BALF5 | B07.02 = 150 |
| IQYVFVEPK | 304 | EBV, BALF5 | A03.01 = 78 |
| ITGLIPIDMY | 305 | EBV, BALF5 | A0l.01 = 340 |
| ITKKRYVGV | 306 | EBV, BALF5 | B08.01 = 520 |
| ITKKRYVGVL | 307 | EBV, BALF5 | B08.01 = 240 |
| ITLSHPNSV | 308 | EBV, BALF5 | A02.01 = 610 |
| IVIKLLGRRK | 309 | EBV, BALF5 | A03.01 = 350 |
| IYGDTDSLF | 310 | EBV, BALF5 | A24.02 = 37 |
| IYGDTDSLFI | 311 | EBV, BALF5 | A24.02 = 95 |
| IYSINPASL | 312 | EBV, BALF5 | A24.02 = 110 |
| KAIKKLLAA | 313 | EBV, BALF5 | B08.01 = 790 |
| KEYLRLIPK | 314 | EBV, BALF5 | A03.01 = 810 |
| KGFLRANTK | 315 | EBV, BALF5 | A03.01 = 170 |
| KIAHIPCRR | 316 | EBV, BALF5 | A03.01 = 340 |
| KITLSHPNSV | 317 | EBV, BALF5 | A02.01 = 920 |
| KLDTVARHL | 318 | EBV, BALF5 | A02.01 = 900 |
| KLDTVARHLL | 319 | EBV, BALF5 | A02.01 = 170 |
| KLLQGAANI | 320 | EBV, BALF5 | A02.01 = 25 |
| KLLQGAANIL | 321 | EBV, BALF5 | A02.01 = 63 |
| KLSAYKSTQM | 322 | EBV, BALF5 | A02.01 = 620 |
| KLSLSDYKL | 323 | EBV, BALF5 | A02.01 = 160 |
| KPDKEYLRL | 324 | EBV, BALF5 | B07.02 = 810 |
| KTACKFVQTR | 325 | EBV, BALF5 | A03.01 = 740 |
| KTEMAEDPAY | 326 | EBV, BALF5 | A01.01 = 200 |
| KTFSCLMLI | 327 | EBV, BALF5 | A02.01 = 46; A24.02 = 400 |
| KTLMKGVELV | 328 | EBV, BALF5 | A02.01 = 730 |
| KVRITGLIPI | 329 | EBV, BALF5 | B07.02 = 260 |
| KVTRRSIMGY | 330 | EBV, BALF5 | A03.01 = 140 |
| LAAHTTRSL | 331 | EBV, BALF5 | B07.02 = 76 |
| LAKRKAIKKL | 332 | EBV, BALF5 | B08.01 = 180 |
| LDRARHIYSI | 333 | EBV, BALF5 | B08.01 = 410 |
| LILQISCVL | 334 | EBV, BALF5 | A02.01 = 810 |
| LIPIDMYAV | 335 | EBV, BALF5 | A02.01 = 150 |
| LITKKRYVGV | 336 | EBV, BALF5 | B08.01 = 860 |
| LLNHFVIHV | 337 | EBV, BALF5 | A02.01 = 7.3 |
| LLQGAANIL | 338 | EBV, BALF5 | A02.01 = 360 |
| LLTSWLAKRK | 339 | EBV, BALF5 | A03.01 = 240 |
| LMKGVELVRK | 340 | EBV, BALF5 | A03.01 = 130 |
| LMLITKKRYV | 341 | EBV, BALF5 | A02.01 = 910 |
| LPVGFLPVI | 342 | EBV, BALF5 | B07.02 = 530 |
| LQISCVLWST | 343 | EBV, BALF5 | A02.01 = 220 |
| LSRKLSAYK | 344 | EBV, BALF5 | A03.01 = 94 |
| LTDGKTLMK | 345 | EBV, BALF5 | A0l.01 = 410; A03.01 = 610 |
| LTGGVYHFV | 346 | EBV, BALF5 | A02.01 = 200 |
| LTGGVYHFVK | 347 | EBV, BALF5 | A03.01 = 330 |
| LTSWLAKRK | 348 | EBV, BALF5 | A03.01 = 560 |
| LVMDLLNHFV | 349 | EBV, BALF5 | A02.01 = 5.3 |
| LVVDFASLY | 350 | EBV, BALF5 | A0l.01 = 460 |
| MLERAKAFV | 351 | EBV, BALF5 | A02.01 = 400 |
| MLITKKRYV | 352 | EBV, BALF5 | A02.01 = 190; B08.01 = 130 |
| MLITKKRYVG | 353 | EBV, BALF5 | B08.01 = 560 |
| MLYAFFQLI | 354 | EBV, BALF5 | A02.01 = 6.6; A24.02 = 800; B08.01 = 640 |
| MLYAFFQLIR | 355 | EBV, BALF5 | A03.01 = 40 |
| MPHLAVYQKF | 356 | EBV, BALF5 | B07.02 = 150 |
| MSGGLFYNPF | 357 | EBV, BALF5 | A24.02 = 490 |
| MYAVCRDKL | 358 | EBV, BALF5 | A24.02 = 210 |
| NPEGQLRVI | 359 | EBV, BALF5 | B07.02 = 650 |
| NPFLRPNKGL | 360 | EBV, BALF5 | B07.02 = 140; B08.01 = 630 |
| NTKVRITGL | 361 | EBV, BALF5 | B08.01 = 120 |
| NTKVRITGLI | 362 | EBV, BALF5 | B08.01 = 940 |
| NVANFDWPY | 363 | EBV, BALF5 | A0l.01 = 300 |
| NVANFDWPYI | 364 | EBV, BALF5 | A02.01 = 320 |
| PLSGFYNSPV | 365 | EBV, BALF5 | A02.01 = 520 |
| QIHDRIQYV | 366 | EBV, BALF5 | A02.01 = 180 |
| QIRVFSCLL | 367 | EBV, BALF5 | B08.01 = 960 |
| QLIRDLSVEI | 368 | EBV, BALF5 | A02.01 = 94 |
| QMPHLAVYQK | 369 | EBV, BALF5 | A03.01 = 900 |
| QQIRVFSCLL | 370 | EBV, BALF5 | A02.01 = 980 |
| QTRCRRVLDL | 371 | EBV, BALF5 | B08.01 = 200 |
| RAKAFVEAL | 372 | EBV, BALF5 | B07.02 = 270; B08.01 = 990 |
| RIFEANVDA | 373 | EBV, BALF5 | A02.01 = 870 |
| RIQYVFVEPK | 374 | EBV, BALF5 | A03.01 = 85 |
| RLFAAGPEGR | 375 | EBV, BALF5 | A03.01 = 260 |
| RLIPKCFQT | 376 | EBV, BALF5 | A02.01 = 130 |
| RLTGGVYHF | 377 | EBV, BALF5 | A24.02 = 340 |
| RLTGGVYHFV | 378 | EBV, BALF5 | A02.01 = 8.1 |
| RPGEDYESF | 379 | EBV, BALF5 | B07.02 = 630 |
| RPHDAGKGF | 380 | EBV, BALF5 | B07.02 = 21 |
| RPHDAGKGFL | 381 | EBV, BALF5 | B07.02 = 9.1 |
| RTMLERAKAF | 382 | EBV, BALF5 | B07.02 = 920 |
| RTPCRVSVEK | 383 | EBV, BALF5 | A03.01 = 430 |
| RVFSCLLAA | 384 | EBV, BALF5 | A02.01 = 69; A03.01 = 810 |
| RVFSCLLAAA | 385 | EBV, BALF5 | A02.01 = 58 |
| RVIYGDTDSL | 386 | EBV, BALF5 | B07.02 = 560 |
| RVKEAASLL | 387 | EBV, BALF5 | B07.02 = 380 |
| RVLDLVLADA | 388 | EBV, BALF5 | A02.01 = 670 |
| RYRRILLTL | 389 | EBV, BALF5 | A24.02 = 23 |
| SFLASLLTSW | 390 | EBV, BALF5 | A24.02 = 56 |
| SFTQGLPVGF | 391 | EBV, BALF5 | A24.02 = 350 |
| SIMGYGNHA | 392 | EBV, BALF5 | A02.01 = 760 |
| SINPASLGK | 393 | EBV, BALF5 | A03.01 = 63 |
| SLFVAPISL | 394 | EBV, BALF5 | A02.01 = 30 |
| SLLTSWLAK | 395 | EBV, BALF5 | A03.01 = 23 |
| SLLTSWLAKR | 396 | EBV, BALF5 | A03.01 = 490 |
| SLSDYKLDTV | 397 | EBV, BALF5 | A02.01 = 22 |
| SLYPSIIQA | 398 | EBV, BALF5 | A02.01 = 23 |
| SLYPSIIQAH | 399 | EBV, BALF5 | A03.01 = 240 |
| SPANLQALA | 400 | EBV, BALF5 | B07.02 = 500 |
| SPLDFHVYDI | 401 | EBV, BALF5 | B07.02 = 570 |
| STFDRRTPCR | 402 | EBV, BALF5 | A03.01 = 600 |
| STGEEAGRY | 403 | EBV, BALF5 | A01.01 = 130 |
| STQMPHLAVY | 404 | EBV, BALF5 | A01.01 = 130 |
| SWLAKRKAI | 405 | EBV, BALF5 | B08.01 = 340 |
| SWPSYQALAF | 406 | EBV, BALF5 | A24.02 = 49 |
| SYQALAFDI | 407 | EBV, BALF5 | A24.02 = 13 |
| TKKRYVGVL | 408 | EBV, BALF5 | B08.01 = 460 |
| TLMKGVELV | 409 | EBV, BALF5 | A02.01 = 7.9 |
| TMLERAKAF | 410 | EBV, BALF5 | B08.01 = 100 |
| TMLERAKAFV | 411 | EBV, BALF5 | A02.01 = 33; B08.01 = 840 |
| TVARHLLGAK | 412 | EBV, BALF5 | A03.01 = 90 |
| VARHLLGAK | 413 | EBV, BALF5 | A03.01 = 600 |
| VARHLLGAKK | 414 | EBV, BALF5 | A03.01 = 820 |
| VIDILNQAY | 415 | EBV, BALF5 | A01.01 = 34 |
| VIKLLGRRK | 416 | EBV, BALF5 | A03.01 = 720 |
| VIQPLSGFY | 417 | EBV, BALF5 | A0l.01 = 990; A03.01 = 920 |
| VIYGDTDSL | 418 | EBV, BALF5 | A02.01 = 680 |
| VLADARVKEA | 419 | EBV, BALF5 | A02.01 = 450 |
| VLDDGQQIRV | 420 | EBV, BALF5 | A02.01 = 17 |
| VLDLVLADA | 421 | EBV, BALF5 | A02.01 = 540 |
| VLTDGKTLMK | 422 | EBV, BALF5 | A03.01 = 200 |
| VLVVDFASL | 423 | EBV, BALF5 | A02.01 = 190 |
| VLWSTGEEA | 424 | EBV, BALF5 | A02.01 = 210 |
| VMDLLNHFV | 425 | EBV, BALF5 | A01.01 = 780; A02.01 = 9.7 |
| VMDLLNHFVI | 426 | EBV, BALF5 | A02.01 = 79 |
| VTFGWYSCR | 427 | EBV, BALF5 | A03.01 = 290 |
| VTFGWYSCRR | 428 | EBV, BALF5 | A03.01 = 230 |
| VTRRSIMGY | 429 | EBV, BALF5 | A03.01 = 250 |
| VYTHEKCAVI | 430 | EBV, BALF5 | A24.02 = 490 |
| WLAKRKAIK | 431 | EBV, BALF5 | A03.01 = 470 |
| WLAKRKAIKK | 432 | EBV, BALF5 | A03.01 = 190 |
| WLQDKHGCRI | 433 | EBV, BALF5 | A02.01 = 790; B08.01 = 810 |
| WPSYQALAF | 434 | EBV, BALF5 | B07.02 = 31 |
| WSTGEEAGRY | 435 | EBV, BALF5 | A0l.01 = 370 |
| YAERHGVPV | 436 | EBV, BALF5 | B08.01 = 700 |
| YAVCRDKLSL | 437 | EBV, BALF5 | B08.01 = 330 |
| YFYASAPQGL | 438 | EBV, BALF5 | A24.02 = 650 |
| YILDRARHI | 439 | EBV, BALF5 | A02.01 = 740; B08.01 = 770 |
| YLRLIPKCF | 440 | EBV, BALF5 | B08.01 = 330 |
| YPSIIQAHNL | 441 | EBV, BALF5 | B07.02 = 76 |
| YQGATVIQPL | 442 | EBV, BALF5 | A02.01 = 62 |
| YSINPASLGK | 443 | EBV, BALF5 | A03.01 = 230 |
| YTDLREGRV | 444 | EBV, BALF5 | A0l.01 = 510 |
| YVFVEPKGGV | 445 | EBV, BALF5 | A02.01 = 650 |
| AYSARPPPL | 446 | EBV, RPMS1 | A24.02 = 550 |
| CAYSARPPPL | 447 | EBV, RPMS1 | B07.02 = 420; B08.01 = 770 |
| GARRRARCPA | 448 | EBV, RPMS1 | B08.01 = 240 |
| GPGRRARSCL | 449 | EBV, RPMS1 | B07.02 = 52; B08.01 = 650 |
| MAGARRRARC | 450 | EBV, RPMS1 | B08.01 = 460 |
| RPGPGRRARS | 451 | EBV, RPMS1 | B07.02 = 520 |
| RPPHSRTRA | 452 | EBV, RPMS1 | B07.02 = 93 |
| RRRARCPASA | 453 | EBV, RPMS1 | B08.01 = 610 |
| SGQPRWWPW | 454 | EBV, RPMS1 | A24.02 = 350 |
| STRGRRISA | 455 | EBV, RPMS1 | B07.02 = 990; B08.01 = 240 |
| WPWGSPPPL | 456 | EBV, RPMS1 | B07.02 = 7.8 |
| WWPWGSPPPL | 457 | EBV, RPMS1 | A24.02 = 650 |
| AMFCAWKLY | 458 | EBV, A73 | A03.01 = 120 |
| AMFCAWKLYS | 459 | EBV, A73 | A02.01 = 700 |
| AVKAAAQSAF | 460 | EBV, A73 | B07.02 = 750 |
| CAWKLYSRK | 461 | EBV, A73 | A03.01 = 350 |
| FADAWRMARY | 462 | EBV, A73 | A01.01 = 11 |
| KLYSRKMPS | 463 | EBV, A73 | A03.01 = 400 |
| KLYSRKMPSR | 464 | EBV, A73 | A03.01 = 19 |
| KPPTVLTRPA | 465 | EBV, A73 | B07.02 = 540 |
| KTLEARCSSR | 466 | EBV, A73 | A03.01 = 770 |
| MARYWGCAV | 467 | EBV, A73 | B07.02 = 37; B08.01 = 51 |
| MARYWGCAVK | 468 | EBV, A73 | A03.01 = 420 |
| MPSRSKTLEA | 469 | EBV, A73 | B07.02 = 92; B08.01 = 910 |
| MSMPPKGFLK | 470 | EBV, A73 | A03.01 = 29 |
| PPTVLTRPAM | 471 | EBV, A73 | B07.02 = 350 |
| RGWRARRLGK | 472 | EBV, A73 | A03.01 = 290 |
| RKMPSRSKTL | 473 | EBV, A73 | B07.02 = 580 |
| RLGKESWFA | 474 | EBV, A73 | A02.01 = 56 |
| RLLNKPPTV | 475 | EBV, A73 | A02.01 = 21 |
| RLLNKPPTVL | 476 | EBV, A73 | A02.01 = 290 |
| RMARYWGCAV | 477 | EBV, A73 | A02.01 = 41; B08.01 = 740 |
| RPAMFCAWKL | 478 | EBV, A73 | B07.02 = 40 |
| SMPPKGFLK | 479 | EBV, A73 | A03.01 = 200 |
| SMPPKGFLKK | 480 | EBV, A73 | A03.01 = 230 |
| SPACQTRDT | 481 | EBV, A73 | B07.02 = 440 |
| SWFADAWRM | 482 | EBV, A73 | A24.02 = 530 |
| VLTRPAMFCA | 483 | EBV, A73 | A02.01 = 650 |
| WRMARYWGC | 484 | EBV, A73 | B08.01 = 880 |
| WRMARYWGCA | 485 | EBV, A73 | B08.01 = 190 |
| YSRKMPSRSK | 486 | EBV, A73 | A03.01 = 680 |
| AARDRFPGL | 487 | EBV, BALF4 | B07.02 = 720; B08.01 = 270 |
| AARGSTSAA | 488 | EBV, BALF4 | B07.02 = 110 |
| AARGSTSAAV | 489 | EBV, BALF4 | B07.02 = 61 |
| AFLDKGTYTL | 490 | EBV, BALF4 | A24.02 = 960 |
| ALHEQNQEQK | 491 | EBV, BALF4 | A03.01 = 730 |
| APGKSLGTL | 492 | EBV, BALF4 | B07.02 = 16 |
| APPAARGST | 493 | EBV, BALF4 | B07.02 = 170 |
| AQNIAGLRK | 494 | EBV, BALF4 | A03.01 = 570 |
| ATLQTFISL | 495 | EBV, BALF4 | A02.01 = 980 |
| ATRQQTSFPF | 496 | EBV, BALF4 | B07.02 = 720 |
| ATVQIQFAY | 497 | EBV, BALF4 | A0l.01 = 170 |
| CLEQKRQNM | 498 | EBV, BALF4 | B08.01 = 650 |
| CLITDMMAK | 499 | EBV, BALF4 | A03.01 = 74 |
| CPLQHWQTF | 500 | EBV, BALF4 | B07.02 = 88; B08.01 = 630 |
| CQATSQYYF | 501 | EBV, BALF4 | A24.02 = 620 |
| CYSRPLVSF | 502 | EBV, BALF4 | A24.02 = 12 |
| DMMAKSNSPF | 503 | EBV, BALF4 | B08.01 = 190 |
| DSFHVRTNYK | 504 | EBV, BALF4 | A03.01 = 850 |
| ELMDSLGSV | 505 | EBV, BALF4 | A02.01 = 21 |
| ELYDAPGWLI | 506 | EBV, BALF4 | A02.01 = 400 |
| ENRTAYCPL | 507 | EBV, BALF4 | B08.01 = 680 |
| EQKRQNMVL | 508 | EBV, BALF4 | B08.01 = 470 |
| ETDQMDTIY | 509 | EBV, BALF4 | A01.01 = 13 |
| ETMCYSRPL | 510 | EBV, BALF4 | B08.01 = 160 |
| FFKNPFGGML | 511 | EBV, BALF4 | B08.01 = 980 |
| FISLNTSLI | 512 | EBV, BALF4 | A02.01 = 140 |
| FITSGGLLL | 513 | EBV, BALF4 | A02.01 = 350 |
| FITSGGLLLA | 514 | EBV, BALF4 | A02.01 = 330 |
| FLDKGTYTL | 515 | EBV, BALF4 | A02.01 = 3 |
| FLDKGTYTLS | 516 | EBV, BALF4 | A02.01 = 200 |
| FLTKKMTEV | 517 | EBV, BALF4 | A02.01 = 10; B08.01 = 980 |
| FPGLRRRRY | 518 | EBV, BALF4 | B07.02 = 850 |
| FQAQNIAGL | 519 | EBV, BALF4 | A02.01 = 25 |
| FQSGNEIHV | 520 | EBV, BALF4 | A02.01 = 79 |
| FSFINDTKTY | 521 | EBV, BALF4 | A01.01 = 740 |
| FVDGLGELM | 522 | EBV, BALF4 | A01.01 = 760 |
| FVTNTTVGI | 523 | EBV, BALF4 | A02.01 = 340 |
| FYDGKNKETF | 524 | EBV, BALF4 | A24.02 = 190 |
| GFISFFKNPF | 525 | EBV, BALF4 | A24.02 = 160 |
| GIATLQTFI | 526 | EBV, BALF4 | A02.01 = 490 |
| GIFREYNFQA | 527 | EBV, BALF4 | A02.01 = 310 |
| GLFSSLVSG | 528 | EBV, BALF4 | A02.01 = 870 |
| GLFSSLVSGF | 529 | EBV, BALF4 | A02.01 = 850 |
| GLGELMDSL | 530 | EBV, BALF4 | A02.01 = 73 |
| GLLLAWLPL | 531 | EBV, BALF4 | A02.01 = 85 |
| GLLLAWLPLT | 532 | EBV, BALF4 | A02.01 = 280 |
| GLLMVFKDNI | 533 | EBV, BALF4 | A02.01 = 610 |
| GLRKDLDNAV | 534 | EBV, BALF4 | A02.01 = 680 |
| GMLILVLVA | 535 | EBV, BALF4 | A02.01 = 570 |
| GPSVASRAL | 536 | EBV, BALF4 | B07.02 = 7.9 |
| GQEAITYFI | 537 | EBV, BALF4 | A02.01 = 430 |
| GTDNEIFLTK | 538 | EBV, BALF4 | A03.01 = 690 |
| GTLNNPATV | 539 | EBV, BALF4 | A02.01 = 940 |
| GTYTLSWKL | 540 | EBV, BALF4 | A02.01 = 410 |
| GVNITVNLK | 541 | EBV, BALF4 | A03.01 = 120 |
| HTEGLLMVFK | 542 | EBV, BALF4 | A03.01 = 450 |
| HVYNDYHHFK | 543 | EBV, BALF4 | A03.01 = 22 |
| HWQTFDSTI | 544 | EBV, BALF4 | A24.02 = 490 |
| ILIYNGWYA | 545 | EBV, BALF4 | A02.01 = 15 |
| IQFAYDSLR | 546 | EBV, BALF4 | A03.01 = 920 |
| IQFAYDSLRR | 547 | EBV, BALF4 | A03.01 = 610 |
| IYNGWYADSV | 548 | EBV, BALF4 | A24.02 = 290 |
| IYQCYNAVKM | 549 | EBV, BALF4 | A24.02 = 140 |
| KGTYTLSWK | 550 | EBV, BALF4 | A03.01 = 570 |
| KIVTNILIY | 551 | EBV, BALF4 | A03.01 = 460 |
| KMTEVCQAT | 552 | EBV, BALF4 | A02.01 = 400 |
| KMTKDGLTRV | 553 | EBV, BALF4 | A02.01 = 96 |
| KPTGGLANGV | 554 | EBV, BALF4 | B07.02 = 580 |
| KSNSPFDFFV | 555 | EBV, BALF4 | A02.01 = 570 |
| KTMHEKYEAV | 556 | EBV, BALF4 | A02.01 = 250 |
| LARAWCLEQK | 557 | EBV, BALF4 | A03.01 = 920 |
| LFSSLVSGF | 558 | EBV, BALF4 | A24.02 = 490 |
| LILVLVAGV | 559 | EBV, BALF4 | A02.01 = 58 |
| LILVLVAGVV | 560 | EBV, BALF4 | A02.01 = 940 |
| LLAALACRL | 561 | EBV, BALF4 | A02.01 = 37 |
| LLAWLPLTPR | 562 | EBV, BALF4 | A03.01 = 440 |
| LLLAWLPLT | 563 | EBV, BALF4 | A02.01 = 21 |
| LLMVFKDNI | 564 | EBV, BALF4 | A02.01 = 130 |
| LLMVFKDNII | 565 | EBV, BALF4 | A02.01 = 320; B08.01 = 700 |
| LPLTPRSLA | 566 | EBV, BALF4 | B07.02 = 250 |
| LPLTPRSLAT | 567 | EBV, BALF4 | B07.02 = 110 |
| LRRQINRML | 568 | EBV, BALF4 | B08.01 = 770 |
| LTPRSLATV | 569 | EBV, BALF4 | A02.01 = 900 |
| LVAGVVILV | 570 | EBV, BALF4 | A02.01 = 46 |
| LVSGFISFFK | 571 | EBV, BALF4 | A03.01 = 27 |
| LYDAPGWLI | 572 | EBV, BALF4 | A24.02 = 110 |
| LYDAPGWLIW | 573 | EBV, BALF4 | A24.02 = 170 |
| MCYSRPLVSF | 574 | EBV, BALF4 | B08.01 = 760 |
| MLILVLVAGV | 575 | EBV, BALF4 | A02.01 = 7.6 |
| MLYPGIDEL | 576 | EBV, BALF4 | A02.01 = 4.2 |
| MLYPGIDELA | 577 | EBV, BALF4 | A02.01 = 16 |
| MMAKSNSPF | 578 | EBV, BALF4 | A02.01 = 920; A24.02 = 240; B07.02 = 290; B08.01 = 630 |
| MSQQPVQMLY | 579 | EBV, BALF4 | A01.01 = 67 |
| MTRRRVLSV | 580 | EBV, BALF4 | B07.02 = 200; B08.01 = 7.4 |
| MTRRRVLSVV | 581 | EBV, BALF4 | B07.02 = 490; B08.01 = 39 |
| MVFKDNIIPY | 582 | EBV, BALF4 | A03.01 = 360 |
| MVLRELTKI | 583 | EBV, BALF4 | A02.01 = 580 |
| NIDFASLELY | 584 | EBV, BALF4 | A01.01 = 24 |
| NIIPYSFKV | 585 | EBV, BALF4 | A02.01 = ll |
| NILIYNGWYA | 586 | EBV, BALF4 | A02.01 = 470 |
| NLTELTTPT | 587 | EBV, BALF4 | A02.01 = 1000 |
| NMVLRELTK | 588 | EBV, BALF4 | A03.01 = 840 |
| NPATVQIQF | 589 | EBV, BALF4 | B07.02 = 810 |
| NPFGGMLIL | 590 | EBV, BALF4 | B07.02 = 110 |
| NPQGERRAF | 591 | EBV, BALF4 | B07.02 = 48 |
| NPQGERRAFL | 592 | EBV, BALF4 | B07.02 = 73 |
| NPTTVMSSI | 593 | EBV, BALF4 | B07.02 = 240 |
| PPAAPGKSL | 594 | EBV, BALF4 | B07.02 = 84 |
| PPAARGSTSA | 595 | EBV, BALF4 | B07.02 = 800 |
| QMDTIYQCY | 596 | EBV, BALF4 | A01.01 = 75 |
| QMLYPGIDEL | 597 | EBV, BALF4 | A02.01 = 210 |
| QPAPPATTV | 598 | EBV, BALF4 | B07.02 = 95 |
| QQTSFPFRV | 599 | EBV, BALF4 | A02.01 = 400 |
| QTVEMSPFY | 600 | EBV, BALF4 | A01.01 = 320 |
| QVNKTMHEK | 601 | EBV, BALF4 | A03.01 = 520 |
| QYYFQSGNEI | 602 | EBV, BALF4 | A24.02 = 320 |
| RMLGDLARA | 603 | EBV, BALF4 | A02.01 = 47 |
| RMLGDLARAW | 604 | EBV, BALF4 | A24.02 = 1000 |
| RPLVSFSFI | 605 | EBV, BALF4 | B07.02 = 87 |
| RQQTSFPFR | 606 | EBV, BALF4 | A03.01 = 790 |
| RQQTSFPFRV | 607 | EBV, BALF4 | A02.01 = 250 |
| RTAYCPLQH | 608 | EBV, BALF4 | A03.01 = 420 |
| RTNYKIVDY | 609 | EBV, BALF4 | A03.01 = 890 |
| RTRQMSQQPV | 610 | EBV, BALF4 | B07.02 = 160 |
| RTRTTVNCL | 611 | EBV, BALF4 | B07.02 = 220 |
| RVLSVVVLL | 612 | EBV, BALF4 | A02.01 = 150 |
| RVLSVVVLLA | 613 | EBV, BALF4 | A02.01 = 500 |
| RYASQTELY | 614 | EBV, BALF4 | A24.02 = 870 |
| RYTKGQEAI | 615 | EBV, BALF4 | A24.02 = 350 |
| SFHVRTNYK | 616 | EBV, BALF4 | A03.01 = 1000 |
| SFHVRTNYKI | 617 | EBV, BALF4 | A24.02 = 580 |
| SFKVRSYTKI | 618 | EBV, BALF4 | B08.01 = 410 |
| SFPFRVCEL | 619 | EBV, BALF4 | A24.02 = 670; B08.01 = 970 |
| SITNLVSTV | 620 | EBV, BALF4 | A02.01 = 370 |
| SIYGKAVAA | 621 | EBV, BALF4 | A02.01 = 490 |
| SIYGKAVAAK | 622 | EBV, BALF4 | A03.01 = 17 |
| SLGSVGQSI | 623 | EBV, BALF4 | A02.01 = 790 |
| SLIENIDFA | 624 | EBV, BALF4 | A02.01 = 7.7 |
| SLIENIDFAS | 625 | EBV, BALF4 | A02.01 = 320 |
| SLNTSLIENI | 626 | EBV, BALF4 | A02.01 = 76 |
| SLRRQINRM | 627 | EBV, BALF4 | B08.01 = 77 |
| SLRRQINRML | 628 | EBV, BALF4 | B07.02 = 560; B08.01 = 80 |
| SLTRRTRQM | 629 | EBV, BALF4 | B08.01 = 55 |
| SLVSGFISF | 630 | EBV, BALF4 | A02.01 = 990 |
| SLVSGFISFF | 631 | EBV, BALF4 | A02.01 = 770 |
| SMRVPGSETM | 632 | EBV, BALF4 | B07.02 = 270; B08.01 = 780 |
| SPPSSPSPPA | 633 | EBV, BALF4 | B07.02 = 260 |
| SPSPPAPPA | 634 | EBV, BALF4 | B07.02 = 55 |
| SPSPPAPPAA | 635 | EBV, BALF4 | B07.02 = 58 |
| STIATETGK | 636 | EBV, BALF4 | A03.01 = 320 |
| STVGGLFSSL | 637 | EBV, BALF4 | A02.01 = 490 |
| SVGQSITNLV | 638 | EBV, BALF4 | A02.01 = 810 |
| SVVVLLAAL | 639 | EBV, BALF4 | A02.01 = 980 |
| SYTKIVTNI | 640 | EBV, BALF4 | A24.02 = 62 |
| SYTKIVTNIL | 641 | EBV, BALF4 | A24.02 = 120 |
| TFHERADSF | 642 | EBV, BALF4 | A24.02 = 860 |
| TFISLNTSLI | 643 | EBV, BALF4 | A24.02 = 200 |
| TIYQCYNAV | 644 | EBV, BALF4 | A02.01 = 160 |
| TIYQCYNAVK | 645 | EBV, BALF4 | A03.01 = 43 |
| TMCYSRPLV | 646 | EBV, BALF4 | A02.01 = 630 |
| TMHEKYEAV | 647 | EBV, BALF4 | A02.01 = 61; B08.01 = 300 |
| TTVMSSIYGK | 648 | EBV, BALF4 | A03.01 = 220 |
| TVGGLFSSLV | 649 | EBV, BALF4 | A02.01 = 690 |
| TVMSSIYGK | 650 | EBV, BALF4 | A03.01 = 44 |
| TYFITSGGLL | 651 | EBV, BALF4 | A24.02 = 480 |
| VILVISLTR | 652 | EBV, BALF4 | A03.01 = 750 |
| VILVISLTRR | 653 | EBV, BALF4 | A03.01 = 850 |
| VISVSQCVPV | 654 | EBV, BALF4 | A02.01 = 510 |
| VLLAALACR | 655 | EBV, BALF4 | A03.01 = 980 |
| VLLAALACRL | 656 | EBV, BALF4 | A02.01 = 31 |
| VLRRRRRDA | 657 | EBV, BALF4 | B08.01 = 190 |
| VLRRRRRDAG | 658 | EBV, BALF4 | B08.01 = 490 |
| VLSVVVLLA | 659 | EBV, BALF4 | A02.01 = 340 |
| VLSVVVLLAA | 660 | EBV, BALF4 | A02.01 = 250 |
| VLVAGVVIL | 661 | EBV, BALF4 | A02.01 = 150 |
| VLVAGVVILV | 662 | EBV, BALF4 | A02.01 = 21 |
| VMSSIYGKA | 663 | EBV, BALF4 | A02.01 = 910 |
| VMSSIYGKAV | 664 | EBV, BALF4 | A02.01 = 140 |
| VPPAAPGKSL | 665 | EBV, BALF4 | B07.02 = 82 |
| VPVNQATVTL | 666 | EBV, BALF4 | B07.02 = 42 |
| VSFSFINDTK | 667 | EBV, BALF4 | A03.01 = 220 |
| VSGFISFFK | 668 | EBV, BALF4 | A03.01 = 65 |
| VTDEGTSSF | 669 | EBV, BALF4 | A01.01 = 110 |
| VTDEGTSSFV | 670 | EBV, BALF4 | A01.01 = 220; A02.01 = 150 |
| VYVDRDGVNI | 671 | EBV, BALF4 | A24.02 = 690 |
| YADSVTNRH | 672 | EBV, BALF4 | A01.01 = 950 |
| YCPLQHWQTF | 673 | EBV, BALF4 | A24.02 = 690 |
| YFITSGGLLL | 674 | EBV, BALF4 | A24.02 = 630 |
| YSFKVRSYTK | 675 | EBV, BALF4 | A03.01 = 52 |
| YSRPLVSFSF | 676 | EBV, BALF4 | A24.02 = 900 |
| YTKGQEAITY | 677 | EBV, BALF4 | A01.01 = 940 |
| YYFQSGNEI | 678 | EBV, BALF4 | A24.02 = 38 |
| AAAYSQVYAL | 679 | EBV, BALF3 | B07.02 = 440 |
| AAFENSKYIK | 680 | EBV, BALF3 | A03.01 = 430 |
| AAFTAHPQYR | 681 | EBV, BALF3 | A03.01 = 790 |
| AAYSQVYAL | 682 | EBV, BALF3 | A02.01 = 610; B07.02 = 490; B08.01 = 980 |
| AGARLTHLF | 683 | EBV, BALF3 | A24.02 = 640 |
| AILLPRLRR | 684 | EBV, BALF3 | A03.01 = 490 |
| AILQKRNEL | 685 | EBV, BALF3 | B08.01 = 250 |
| ALAGARLTH | 686 | EBV, BALF3 | A03.01 = 800 |
| ALAGARLTHL | 687 | EBV, BALF3 | A02.01 = 120 |
| ALARCCDAA | 688 | EBV, BALF3 | A02.01 = 570 |
| ALAVELSVCA | 689 | EBV, BALF3 | A02.01 = 170 |
| ALDTYNVFST | 690 | EBV, BALF3 | A02.01 = 150 |
| ALFARERRL | 691 | EBV, BALF3 | B08.01 = 800 |
| ALKRKEQYL | 692 | EBV, BALF3 | B08.01 = 100 |
| ALLYLHLQM | 693 | EBV, BALF3 | A02.01 = 540 |
| ALPPDGLYL | 694 | EBV, BALF3 | A02.01 = 330 |
| ALPPDGLYLT | 695 | EBV, BALF3 | A02.01 = 940 |
| ALREAVLTV | 696 | EBV, BALF3 | A02.01 = 40 |
| ALSDALKRK | 697 | EBV, BALF3 | A03.01 = 320 |
| ALSLELVHL | 698 | EBV, BALF3 | A02.01 = 130 |
| ALSLELVHLL | 699 | EBV, BALF3 | A02.01 = 50 |
| ALTHSLYTF | 700 | EBV, BALF3 | A24.02 = 540 |
| ALTHSLYTFI | 701 | EBV, BALF3 | A02.01 = 35 |
| APGATERLF | 702 | EBV, BALF3 | B07.02 = 990 |
| APGRKGTRV | 703 | EBV, BALF3 | B07.02 = 210 |
| AVLTVSLYNK | 704 | EBV, BALF3 | A03.01 = 60 |
| AYSQVYALA | 705 | EBV, BALF3 | A24.02 = 540 |
| AYSQVYALAV | 706 | EBV, BALF3 | A24.02 = 330 |
| CARLDPRSL | 707 | EBV, BALF3 | B07.02 = 530; B08.01 = 940 |
| CLAQQSETL | 708 | EBV, BALF3 | A02.01 = 570 |
| CLINDIEIL | 709 | EBV, BALF3 | A02.01 = 220 |
| CLINDIEILM | 710 | EBV, BALF3 | A02.01 = 540 |
| DLLGRFRGV | 711 | EBV, BALF3 | B08.01 = 620 |
| DLYALLYLHL | 712 | EBV, BALF3 | A02.01 = 620 |
| DPRLYITSV | 713 | EBV, BALF3 | B07.02 = 430; B08.01 = 220 |
| DPRSLDVAAV | 714 | EBV, BALF3 | B07.02 = 580 |
| DSDRPLILLY | 715 | EBV, BALF3 | A01.01 = 10 |
| DWIETSFNSF | 716 | EBV, BALF3 | A24.02 = 810 |
| EILMKRINSV | 717 | EBV, BALF3 | B08.01 = 120 |
| ELARMRNHFL | 718 | EBV, BALF3 | B08.01 = 350 |
| ELYTRLNRA | 719 | EBV, BALF3 | B08.01 = 550 |
| ELYTRLNRAM | 720 | EBV, BALF3 | B08.01 = 130 |
| EVAELSELLY | 721 | EBV, BALF3 | A01.01 = 220 |
| FARERRLAL | 722 | EBV, BALF3 | B07.02 = 8.5; B08.01 = 3.6 |
| FARERRLALV | 723 | EBV, BALF3 | B07.02 = 910; B08.01 = 20 |
| FKDLYALLY | 724 | EBV, BALF3 | A01.01 = 480 |
| FLTSLIWPG | 725 | EBV, BALF3 | A02.01 = 30 |
| FLTSLIWPGI | 726 | EBV, BALF3 | A02.01 = 6.6 |
| FLYRRAFVS | 727 | EBV, BALF3 | A02.01 = 230; B08.01 = 600 |
| FLYRRAFVSG | 728 | EBV, BALF3 | A02.01 = 610; B08.01 = 380 |
| FPPPSNVAL | 729 | EBV, BALF3 | B07.02 = 44 |
| FYCMSHTMGL | 730 | EBV, BALF3 | A24.02 = 660 |
| GGHSRLSAL | 731 | EBV, BALF3 | B08.01 = 800 |
| GLAKLQSCL | 732 | EBV, BALF3 | A02.01 = 350 |
| GLAKLQSCLA | 733 | EBV, BALF3 | A02.01 = 390 |
| GLFENEVRQA | 734 | EBV, BALF3 | A02.01 = 140 |
| GLFPPPSNV | 735 | EBV, BALF3 | A02.01 = 25 |
| GLFPPPSNVA | 736 | EBV, BALF3 | A02.01 = 430 |
| GLLAAAYSQV | 737 | EBV, BALF3 | A02.01 = 10 |
| GLLAELEAI | 738 | EBV, BALF3 | A02.01 = 25 |
| GLLAELEAIL | 739 | EBV, BALF3 | A02.01 = 100 |
| GPSGALSDAL | 740 | EBV, BALF3 | B07.02 = 47 |
| HALTHSLYTF | 741 | EBV, BALF3 | A24.02 = 630 |
| HFLYRRAFV | 742 | EBV, BALF3 | B08.01 = 350 |
| HLFDAVAPG | 743 | EBV, BALF3 | A02.01 = 420 |
| HLFDAVAPGA | 744 | EBV, BALF3 | A02.01 = 12 |
| HLHALTHSL | 745 | EBV, BALF3 | A02.01 = 78; B07.02 = 450; B08.01 = 160 |
| HLHALTHSLY | 746 | EBV, BALF3 | A03.01 = 120 |
| HLLEAEEKA | 747 | EBV, BALF3 | A02.01 = 810 |
| HLLENSREA | 748 | EBV, BALF3 | A02.01 = 350 |
| HLQMRDDSA | 749 | EBV, BALF3 | B08.01 = 870 |
| HQKAFLTSL | 750 | EBV, BALF3 | B08.01 = 450 |
| HVSCNHLAV | 751 | EBV, BALF3 | A02.01 = 960; B07.02 = 950 |
| ILCRALREAV | 752 | EBV, BALF3 | A02.01 = 390 |
| ILLYKGRGWV | 753 | EBV, BALF3 | A02.01 = 930 |
| ILMKRINSV | 754 | EBV, BALF3 | A02.01 = 6.7; B08.01 = 9.4 |
| ILMKRINSVF | 755 | EBV, BALF3 | A24.02 = 660; B08.01 = 75 |
| IWPGIEPSDW | 756 | EBV, BALF3 | A24.02 = 350 |
| KTWGRSLIL | 757 | EBV, BALF3 | A02.01 = 650 |
| KTWGRSLILR | 758 | EBV, BALF3 | A03.01 = 120 |
| KVDVSLCLI | 759 | EBV, BALF3 | A02.01 = 900 |
| LAAAYSQVY | 760 | EBV, BALF3 | A01.01 = 800 |
| LAGARLTHL | 761 | EBV, BALF3 | B08.01 = 320 |
| LARMRNHFL | 762 | EBV, BALF3 | B07.02 = 350; B08.01 = 22 |
| LFARERRLAL | 763 | EBV, BALF3 | B08.01 = 280 |
| LGRFRGVSPI | 764 | EBV, BALF3 | B08.01 = 300 |
| LILRRADAV | 765 | EBV, BALF3 | B08.01 = 350 |
| LINDIEILM | 766 | EBV, BALF3 | A02.01 = 880 |
| LINDIEILMK | 767 | EBV, BALF3 | A03.01 = 190 |
| LLAAAYSQV | 768 | EBV, BALF3 | A02.01 = 12 |
| LLAAAYSQVY | 769 | EBV, BALF3 | A03.01 = 850 |
| LLAELEAIL | 770 | EBV, BALF3 | A02.01 = 28 |
| LLAELEAILL | 771 | EBV, BALF3 | A02.01 = 17 |
| LLYKGRGWV | 772 | EBV, BALF3 | A02.01 = 460 |
| LLYKGRGWVF | 773 | EBV, BALF3 | B08.01 = 720 |
| LLYLHLQMR | 774 | EBV, BALF3 | A03.01 = 410 |
| LLYWNSGGH | 775 | EBV, BALF3 | A03.01 = 610 |
| LLYWNSGGHA | 776 | EBV, BALF3 | A02.01 = 750 |
| LMKRINSVF | 777 | EBV, BALF3 | B08.01 = 130 |
| LMKRINSVFY | 778 | EBV, BALF3 | A03.01 = 450 |
| LPNHGNPST | 779 | EBV, BALF3 | B07.02 = 400 |
| LPNHGNPSTA | 780 | EBV, BALF3 | B07.02 = 240 |
| LSLELVHLL | 781 | EBV, BALF3 | A02.01 = 630 |
| LVRRACEEAL | 782 | EBV, BALF3 | B07.02 = 370 |
| LYALLYLHL | 783 | EBV, BALF3 | A24.02 = 64 |
| LYITSVPCW | 784 | EBV, BALF3 | A24.02 = 60 |
| LYKGRGWVF | 785 | EBV, BALF3 | A24.02 = 68 |
| LYNKTWGRSL | 786 | EBV, BALF3 | A24.02 = 630 |
| LYTFITGPL | 787 | EBV, BALF3 | A24.02 = 350 |
| LYWNSGGHAI | 788 | EBV, BALF3 | A24.02 = 150 |
| MSGLLAAAY | 789 | EBV, BALF3 | A01.01 = 91 |
| NSREASAAL | 790 | EBV, BALF3 | B07.02 = 230 |
| NVFSTVPPEV | 791 | EBV, BALF3 | A02.01 = 46 |
| PQYRAILQK | 792 | EBV, BALF3 | A03.01 = 850 |
| QVYALAVEL | 793 | EBV, BALF3 | A02.01 = 520 |
| RAILQKRNEL | 794 | EBV, BALF3 | B07.02 = 260 |
| RLDPRSLDV | 795 | EBV, BALF3 | A02.01 = 500 |
| RLFCGGVYS | 796 | EBV, BALF3 | A02.01 = 390 |
| RLFCGGVYSS | 797 | EBV, BALF3 | A02.01 = 150 |
| RLNRAMQRL | 798 | EBV, BALF3 | A02.01 = 810 |
| RLSALFARER | 799 | EBV, BALF3 | A03.01 = 400 |
| RLTHLFDAV | 800 | EBV, BALF3 | A02.01 = 90 |
| RMRNHFLYR | 801 | EBV, BALF3 | A03.01 = 17 |
| RMRNHFLYRR | 802 | EBV, BALF3 | A03.01 = 61 |
| RPAGAREPG | 803 | EBV, BALF3 | B07.02 = 200 |
| RPGGPEEGAV | 804 | EBV, BALF3 | B07.02 = 180 |
| RQAGLGHLL | 805 | EBV, BALF3 | A02.01 = 460 |
| RQVATEGLAK | 806 | EBV, BALF3 | A03.01 = 780 |
| RTPSVAYSV | 807 | EBV, BALF3 | A02.01 = 430 |
| SAALLAPGRK | 808 | EBV, BALF3 | A03.01 = 1000 |
| SLASSQQIL | 809 | EBV, BALF3 | A02.01 = 600 |
| SLCLINDIEI | 810 | EBV, BALF3 | A02.01 = 690 |
| SLILRRADA | 811 | EBV, BALF3 | B08.01 = 330 |
| SLILRRADAV | 812 | EBV, BALF3 | A02.01 = 490; B08.01 = 190 |
| SLYNKTWGR | 813 | EBV, BALF3 | A03.01 = 170 |
| SLYTFITGPL | 814 | EBV, BALF3 | A02.01 = 31; B08.01 = 790 |
| SPEVPRPAGA | 815 | EBV, BALF3 | B07.02 = 630 |
| SPIPDPRLYI | 816 | EBV, BALF3 | B07.02 = 150 |
| SQVYALAVEL | 817 | EBV, BALF3 | A02.01 = 340 |
| SVAYSVEFY | 818 | EBV, BALF3 | A01.01 = 690 |
| SVEFYGGHK | 819 | EBV, BALF3 | A03.01 = 900 |
| SVFYCMSHTM | 820 | EBV, BALF3 | A02.01 = 900 |
| TLSSEHLHAL | 821 | EBV, BALF3 | A02.01 = 99 |
| TLTETLCLRV | 822 | EBV, BALF3 | A02.01 = 45 |
| TPSVAYSVEF | 823 | EBV, BALF3 | B07.02 = 180 |
| TSFNSFYSV | 824 | EBV, BALF3 | A02.01 = 210 |
| TYDSDRPLI | 825 | EBV, BALF3 | A24.02 = 990 |
| VAELSELLY | 826 | EBV, BALF3 | A01.01 = 74 |
| VFKDLYALL | 827 | EBV, BALF3 | A24.02 = 390 |
| VFYCMSHTM | 828 | EBV, BALF3 | A24.02 = 350 |
| VLTVSLYNK | 829 | EBV, BALF3 | A03.01 = 88 |
| VPCWRCVGEL | 830 | EBV, BALF3 | B07.02 = 170 |
| VPGPGRPEA | 831 | EBV, BALF3 | B07.02 = 260 |
| VPPLRTPSV | 832 | EBV, BALF3 | B07.02 = 250; B08.01 = 470 |
| VPPLRTPSVA | 833 | EBV, BALF3 | B07.02 = 850 |
| VPRPAGARE | 834 | EBV, BALF3 | B07.02 = 720 |
| VPRPAGAREP | 835 | EBV, BALF3 | B07.02 = 620 |
| VVYWELARMR | 836 | EBV, BALF3 | A03.01 = 660 |
| VWGDVVYWEL | 837 | EBV, BALF3 | A24.02 = 97 |
| VYALAVELSV | 838 | EBV, BALF3 | A24.02 = 340 |
| WIETSFNSFY | 839 | EBV, BALF3 | A01.01 = 120 |
| WVFKDLYAL | 840 | EBV, BALF3 | A02.01 = 46 |
| WVFKDLYALL | 841 | EBV, BALF3 | A02.01 = 130 |
| YALAVELSV | 842 | EBV, BALF3 | A02.01 = 110 |
| YALLYLHLQM | 843 | EBV, BALF3 | B08.01 = 690 |
| YCMSHTMGL | 844 | EBV, BALF3 | A02.01 = 940; B08.01 = 420 |
| YLRQVATEGL | 845 | EBV, BALF3 | A02.01 = 180; B07.02 = 860; B08.01 = 570 |
| YLTYDSDRPL | 846 | EBV, BALF3 | A02.01 = 98 |
| YNKTWGRSL | 847 | EBV, BALF3 | B08.01 = 240 |
| YTFITGPLA | 848 | EBV, BALF3 | A02.01 = 960 |
| YWNSGGHAI | 849 | EBV, BALF3 | A24.02 = 770 |
| AARPRLLLSL | 850 | EBV, BARF0 | B07.02 = 58; B08.01 = 70 |
| AARVPIEEL | 851 | EBV, BARF0 | B07.02 = 950 |
| AGMSATLPL | 852 | EBV, BARF0 | B07.02 = 550 |
| ALLRQAGLQL | 853 | EBV, BARF0 | A02.01 = 450 |
| ALLWAARPR | 854 | EBV, BARF0 | A03.01 = 590 |
| ALLWAARPRL | 855 | EBV, BARF0 | A02.01 = 130 |
| ALPGRLLLA | 856 | EBV, BARF0 | A02.01 = 620 |
| APAGHRGDV | 857 | EBV, BARF0 | B07.02 = 23 |
| APAGHRGDVE | 858 | EBV, BARF0 | B07.02 = 840 |
| APGYAVEAV | 859 | EBV, BARF0 | B07.02 = 130 |
| AVEAVEGGLY | 860 | EBV, BARF0 | A01.01 = 540 |
| AVEGGLYPV | 861 | EBV, BARF0 | A02.01 = 63 |
| AVRLQRLHRV | 862 | EBV, BARF0 | B08.01 = 730 |
| AVRQRVQVL | 863 | EBV, BARF0 | B07.02 = 37; B08.01 = 47 |
| CPRQRLLAG | 864 | EBV, BARF0 | B07.02 = 190; B08.01 = 180 |
| CPSRPGHLRA | 865 | EBV, BARF0 | B07.02 = 680 |
| CTDSMAARV | 866 | EBV, BARF0 | A01.01 = 220 |
| EPRVELVPL | 867 | EBV, BARF0 | B07.02 = 64; B08.01 = 410 |
| EPRVELVPLL | 868 | EBV, BARF0 | B07.02 = 430 |
| FQALQPHGV | 869 | EBV, BARF0 | A02.01 = 44 |
| GLALLRQAGL | 870 | EBV, BARF0 | A02.01 = 910 |
| GLGKQVCFDV | 871 | EBV, BARF0 | A02.01 = 99 |
| GLGQGLALL | 872 | EBV, BARF0 | A02.01 = 96 |
| GMSATLPLPR | 873 | EBV, BARF0 | A03.01 = 410 |
| GPLCRGRVA | 874 | EBV, BARF0 | B07.02 = 150 |
| GPLCRGRVAV | 875 | EBV, BARF0 | B07.02 = 23; B08.01 = 360 |
| GPPHQGQATL | 876 | EBV, BARF0 | B07.02 = 180 |
| GPRDEGVQA | 877 | EBV, BARF0 | B07.02 = 670 |
| GPRDEGVQAV | 878 | EBV, BARF0 | B07.02 = 35 |
| GVQRSGRPL | 879 | EBV, BARF0 | B07.02 = 330 |
| GVRREGGGAV | 880 | EBV, BARF0 | B07.02 = 860 |
| HAIKHAIDSL | 881 | EBV, BARF0 | B07.02 = 700 |
| HLAQVLLLA | 882 | EBV, BARF0 | A02.01 = 99 |
| HLAQVLLLAL | 883 | EBV, BARF0 | A02.01 = 42 |
| HLRALPGRL | 884 | EBV, BARF0 | B07.02 = 970; B08.01 = 700 |
| HLRALPGRLL | 885 | EBV, BARF0 | B07.02 = 370; B08.01 = 700 |
| HLRGHCREDV | 886 | EBV, BARF0 | B08.01 = 680 |
| HQLAHTAPA | 887 | EBV, BARF0 | A02.01 = 360; B08.01 = 880 |
| ILKGGTLAGA | 888 | EBV, BARF0 | A02.01 = 860 |
| KQVCFDVLGI | 889 | EBV, BARF0 | A02.01 = 140 |
| LALLRQAGL | 890 | EBV, BARF0 | B08.01 = 170 |
| LCRGRVAVV | 891 | EBV, BARF0 | B08.01 = 400 |
| LLASAQPLH | 892 | EBV, BARF0 | A03.01 = 950 |
| LLLASAQPL | 893 | EBV, BARF0 | A02.01 = 20; B08.01 = 790 |
| LLQDGPVLGV | 894 | EBV, BARF0 | A02.01 = 18 |
| LLRQAGLQL | 895 | EBV, BARF0 | B07.02 = 110; B08.01 = 280 |
| LLVRQRTCGV | 896 | EBV, BARF0 | A02.01 = 300 |
| LLWAARPRL | 897 | EBV, BARF0 | A02.01 = 39 |
| LLWAARPRLL | 898 | EBV, BARF0 | A02.01 = 73; B08.01 = 860 |
| LPGKQGREA | 899 | EBV, BARF0 | B07.02 = 280 |
| LPGRLLLASA | 900 | EBV, BARF0 | B07.02 = 560 |
| LPLPRCTDSM | 901 | EBV, BARF0 | B07.02 = 40; B08.01 = 600 |
| LPRCTDSMA | 902 | EBV, BARF0 | B07.02 = 140 |
| LPRCTDSMAA | 903 | EBV, BARF0 | B07.02 = 44; B08.01 = 860 |
| LQDGPVLGV | 904 | EBV, BARF0 | A02.01 = 200 |
| LRIHRHRQV | 905 | EBV, BARF0 | B08.01 = 840 |
| LRIHRHRQVV | 906 | EBV, BARF0 | B08.01 = 490 |
| LVRQRTCGV | 907 | EBV, BARF0 | B07.02 = 690; B08.01 = 72 |
| LWAARPRLLL | 908 | EBV, BARF0 | A24.02 = 770 |
| LYPVARLDAW | 909 | EBV, BARF0 | A24.02 = 85 |
| MSATLPLPR | 910 | EBV, BARF0 | A03.01 = 760 |
| PPHQGQATL | 911 | EBV, BARF0 | B07.02 = 320 |
| PPRARDRAL | 912 | EBV, BARF0 | B07.02 = 44; B08.01 = 760 |
| PPRARDRALL | 913 | EBV, BARF0 | B07.02 = 230 |
| QPCPRQRLL | 914 | EBV, BARF0 | B07.02 = 540 |
| QPHGVRHAI | 915 | EBV, BARF0 | B07.02 = 22 |
| QTLGGHLAQV | 916 | EBV, BARF0 | A02.01 = 600 |
| QVLRAQGLGK | 917 | EBV, BARF0 | A03.01 = 430 |
| RALLWAARPR | 918 | EBV, BARF0 | A03.01 = 960 |
| RALPGRLLL | 919 | EBV, BARF0 | B07.02 = 230 |
| RARDRALLWA | 920 | EBV, BARF0 | B07.02 = 1000 |
| RIHRHRQVV | 921 | EBV, BARF0 | B07.02 = 340; B08.01 = 130 |
| RLLLASAQPL | 922 | EBV, BARF0 | A02.01 = 54 |
| RLLLSLQQV | 923 | EBV, BARF0 | A02.01 = 22 |
| RLRIHRHRQV | 924 | EBV, BARF0 | B07.02 = 530; B08.01 = 58 |
| RPGHLRALPG | 925 | EBV, BARF0 | B07.02 = 410 |
| RPLCLRPPRA | 926 | EBV, BARF0 | B07.02 = 320 |
| RPPRARDRA | 927 | EBV, BARF0 | B07.02 = 640 |
| RPPRARDRAL | 928 | EBV, BARF0 | B07.02 = 9.5 |
| RPRLLLSLQQ | 929 | EBV, BARF0 | B07.02 = 630 |
| RVQVLRAQGL | 930 | EBV, BARF0 | B07.02 = 700 |
| RVREGAGRA | 931 | EBV, BARF0 | B07.02 = 990 |
| RVREGAGRAG | 932 | EBV, BARF0 | B07.02 = 970 |
| RVWDGTYAPK | 933 | EBV, BARF0 | A03.01 = 52 |
| SLQQVPEPSL | 934 | EBV, BARF0 | A02.01 = 230 |
| SQGHVAGWGK | 935 | EBV, BARF0 | A03.01 = 950 |
| TLGGHLAQV | 936 | EBV, BARF0 | A02.01 = 18 |
| TLGGHLAQVL | 937 | EBV, BARF0 | A02.01 = 760 |
| TYAPKAAQQI | 938 | EBV, BARF0 | A24.02 = 61 |
| VLLLALERV | 939 | EBV, BARF0 | A02.01 = 31 |
| VLRAQGLGK | 940 | EBV, BARF0 | A03.01 = 33 |
| VPIEELREF | 941 | EBV, BARF0 | B07.02 = 790 |
| VPLLQDGPV | 942 | EBV, BARF0 | B07.02 = 170 |
| VPLLQDGPVL | 943 | EBV, BARF0 | B07.02 = 68 |
| VVAHAGQLPV | 944 | EBV, BARF0 | A02.01 = 530 |
| WAARPRLLL | 945 | EBV, BARF0 | B07.02 = 260 |
| WPYQGSQERL | 946 | EBV, BARF0 | B07.02 = 35 |
| YPVARLDAW | 947 | EBV, BARF0 | B07.02 = 250 |

### Example 2: HLA Class I and Class II Binding Assays

The following example of peptide binding to HLA molecules demonstrates quantification of binding affinities of HLA class I and class II peptides. Binding assays can be performed with peptides that are either motif-bearing or not motif-bearing.

Epstein-Barr virus (EBV)-transformed homozygous cell lines, fibroblasts, CIR, or 721.22 transfectants are used as sources of HLA class I molecules. Cell lysates are prepared and HLA molecules purified in accordance with disclosed protocols (Sidney et al., Current Protocols in Immunology 18.3.1 (1998); Sidney, et al., J. Immunol. 154:247 (1995); Sette, et al., Mol. Immunol. 31:813 (1994)). HLA molecules are purified from lysates by affinity chromatography. The lysates are passed over a column of Sepharose CL-4B beads coupled to an appropriate antibody. The anti-HLA column is then washed with 10 mM Tris-HCL, pH 8.0, in 1% NP-40, PBS, and PBS containing 0.4% n-octylglucoside and HLA molecules are eluted with 50 mM diethylamine in 0.15M NaCl containing 0.4% n-octylglucoside, pH 11.5. A 1/25 volume of 2.0M Tris, pH 6.8, is added to the eluate to reduce the pH to −8.0. Eluates are then concentrated by centrifugation in Centriprep 30 concentrators (Amicon, Beverly, MA). Protein content is evaluated by a BCA protein assay (Pierce Chemical Co., Rockford, IL) and confirmed by SDS-PAGE.

A detailed description of the protocol utilized to measure the binding of peptides to Class I and Class II MHC has been published (Sette et al., Mol. Immunol. 31:813, 1994; Sidney et al., in Current Protocols in Immunology, Margulies, Ed., John Wiley & Sons, New York, Section 18.3, 1998). Briefly, purified MHC molecules (5 to 500 nM) are incubated with various unlabeled peptide inhibitors and 1-10 nM 125I-radiolabeled probe peptides for 48 h in PBS containing 0.05% Nonidet P-40 (NP40) (or 20% w/v digitonin for H-2 IA assays) in the presence of a protease inhibitor cocktail. All assays are at pH 7.0 with the exception of DRB1*0301, which was performed at pH 4.5, and DRB1*1601 (DR2w21β1) and DRB4*0101 (DRw53), which were performed at pH 5.0.

Following incubation, MHC-peptide complexes are separated from free peptide by gel filtration on 7.8 mm×15 cm TSK200 columns (TosoHaas 16215, Montgomeryville, PA). Because the large size of the radiolabeled peptide used for the DRB1*1501 (DR2w2β1) assay makes separation of bound from unbound peaks more difficult under these conditions, all DRB1*1501 (DR2w2p1) assays were performed using a 7.8 mm×30 cm TSK2000 column eluted at 0.6 mLs/min. The eluate from the TSK columns is passed through a Beckman 170 radioisotope detector, and radioactivity is plotted and integrated using a Hewlett-Packard 3396A integrator, and the fraction of peptide bound is determined.

Radiolabeled peptides are iodinated using the chloramine-T method. Typically, in preliminary experiments, each MHC preparation is titered in the presence of fixed amounts of radiolabeled peptides to determine the concentration of HLA molecules necessary to bind 10-20% of the total radioactivity. All subsequent inhibition and direct binding assays are performed using these HLA concentrations.

Since under these conditions [label]<[HLA] and IC₅₀≥[HLA], the measured IC₅₀ values are reasonable approximations of the true K_{D} values. Peptide inhibitors are typically tested at concentrations ranging from 120 μg/ml to 1.2 ng/ml, and are tested in two to four completely independent experiments. To allow comparison of the data obtained in different experiments, a relative binding FIGURE is calculated for each peptide by dividing the IC₅₀ of a positive control for inhibition by the IC₅₀ for each tested peptide (typically unlabeled versions of the radiolabeled probe peptide). For database purposes, and inter-experiment comparisons, relative binding values are compiled. These values can subsequently be converted back into IC₅₀ nM values by dividing the IC₅₀ nM of the positive controls for inhibition by the relative binding of the peptide of interest. This method of data compilation has proven to be the most accurate and consistent for comparing peptides that have been tested on different days, or with different lots of purified MHC.

Because the antibody used for HLA-DR purification (LB3.1) is α-chain specific, β1 molecules are not separated from β3 (and/or β4 and β5) molecules. The β1 specificity of the binding assay is obvious in the cases of DRB1*0101 (DR1), DRB1*0802 (DR8w2), and DRB1*0803 (DR8w3), where no β3 is expressed. It has also been demonstrated for DRB1*0301 (DR3) and DRB3*0101 (DR52a), DRB1*0401 (DR4w4), DRB1*0404 (DR4w14), DRB1*0405 (DR4w15), DRB1*1101 (DR5), DRB1*1201 (DR5w12), DRB1*1302 (DR6w19) and DRB1*0701 (DR7). The problem of β chain specificity for DRB1*1501 (DR2w2β1), DRB5*0101 (DR2w2β2), DRB1*1601 (DR2w21β1), DRB5*0201 (DR51Dw21), and DRB4*0101 (DRw53) assays is circumvented by the use of fibroblasts. Development and validation of assays with regard to DRβ molecule specificity have been described previously (see, e.g., Southwood et al., J. Immunol. 160:3363-3373, 1998).

The live cell/flow cytometry-based assays can also be used. This is a well-established assay utilizing the TAP-deficient hybridoma cell line T2 (American Type Culture Collection (ATCC Accession No. CRL-1992), Manassas, Va.). The TAP deficiency in this cell line leads to inefficient loading of MHCI in the ER and an excess of empty MHCIs. Salter and Cresswell, EMBO J. 5:943-49 (1986); Salter, Immunogenetics 21:235-46 (1985). Empty MHCIs are highly unstable, and are therefore short-lived. When T2 cells are cultured at reduced temperatures, empty MHCIs appear transiently on the cell surface, where they can be stabilized by the exogenous addition of MHCI-binding peptides. To perform this binding assay, peptide-receptive MHCIs were induced by culturing aliquots of 10⁷ T2 cells overnight at 26° C. in serum free AIM-V medium alone, or in medium containing escalating concentrations (0.1 to 100 μM) of peptide. Cells were then washed twice with PBS, and subsequently incubated with a fluorescent tagged HLA-A0201-specific monoclonal antibody, BB7.2, to quantify cell surface expression. Samples were acquired on a FACS Calibur instrument (Becton Dickinson) and the mean fluorescence intensity (MFI) determined using the accompanying Cellquest software.

### Example 3: Confirmation of Immunogenicity

In vitro education (IVE) assays are used to test the ability of each test peptide to expand CD8+ T-cells. Mature professional APCs are prepared for these assays in the following way. 80-90×10⁶ PBMCs isolated from a healthy human donor are plated in 20 ml of RPMI media containing 2% human AB serum, and incubated at 37° C. for 2 hours to allow for plastic adherence by monocytes. Non-adherent cells are removed and the adherent cells are cultured in RPMI, 2% human AB serum, 800 IU/ml of GM-CSF and 500 IU/ml of IL-4. After 6 days, TNF-alpha is added to a final concentration of 10 ng/ml. On day 7, the dendritic cells (DC) are matured either by the addition of 12.5 μg/ml poly I:C or 0.3 μg/ml of CD40L. The mature dendritic cells (mDC) are harvested on day 8, washed, and either used directly or cryopreserved for future use.

For the IVE of CD8+ T-cells, aliquots of 2×10⁵ mDCs are pulsed with each peptide at a final concentration of 100 μM, incubated for 4 hours at 37° C., and then irradiated (2500 rads). The peptide-pulsed mDCs are washed twice in RPMI containing 2% human AB serum. 2×10⁵ mDCs and 2×10⁶ autologous CD8+ cells are plated per well of a 24-well plate in 2 ml of RPMI containing 2% human AB, 20 ng/ml IL-7 and 100 μg/ml of IL-12, and incubated for 12 days. The CD8+ T-cells are then re-stimulated with peptide-pulsed, irradiated mDCs. Two to three days later, 20 IU/ml IL-2 and 20 ng/IL7 are added. Expanding CD8+ T-cells are re-stimulated every 8-10 days, and are maintained in media containing IL-2 and IL-7. Cultures are monitored for peptide-specific T-cells using a combination of functional assays and/or tetramer staining. Parallel IVEs with the modified and parent peptides allowed for comparisons of the relative efficiency with which the peptides expanded peptide-specific T-cells.

### Quantitative and Functional Assessment of CD8+ T-Cells

### Tetramer Staining

MHC tetramers are purchased or manufactured on-site, and are used to measure peptide-specific T-cell expansion in the IVE assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T-cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that were CD8+/Tetramer+.

### ELISPOT

Peptide-specific T-cells are functionally enumerated using the ELISPOT assay (BD Biosciences), which measures the release of IFNgamma from T-cells on a single cell basis. Target cells (T2 or HLA-A0201 transfected CIRs) were pulsed with 10 μM peptide for 1 hour at 37° C., and washed three times. 1×10⁵ peptide-pulsed targets are co-cultured in the ELISPOT plate wells with varying concentrations of T-cells (5×10² to 2×10³) taken from the IVE culture. Plates are developed according to the manufacturer's protocol, and analyzed on an ELISPOT reader (Cellular Technology Ltd.) with accompanying software. Spots corresponding to the number of IFNgamma-producing T-cells are reported as the absolute number of spots per number of T-cells plated. T-cells expanded on modified peptides are tested not only for their ability to recognize targets pulsed with the modified peptide, but also for their ability to recognize targets pulsed with the parent peptide.

### CD107 Staining

CD107a and b are expressed on the cell surface of CD8+ T-cells following activation with cognate peptide. The lytic granules of T-cells have a lipid bilayer that contains lysosomal-associated membrane glycoproteins (“LAMPs”), which include the molecules CD107a and b. When cytotoxic T-cells are activated through the T-cell receptor, the membranes of these lytic granules mobilize and fuse with the plasma membrane of the T-cell. The granule contents are released, and this leads to the death of the target cell. As the granule membrane fuses with the plasma membrane, C107a and b are exposed on the cell surface, and therefore are markers of degranulation. Because degranulation as measured by CD107 a and b staining is reported on a single cell basis, the assay is used to functionally enumerate peptide-specific T-cells. To perform the assay, peptide is added to HLA-A0201-transfected cells CIR to a final concentration of 20 μM, the cells were incubated for 1 hour at 37° C., and washed three times. 1×10⁵ of the peptide-pulsed CIR cells were aliquoted into tubes, and antibodies specific for CD107 a and b are added to a final concentration suggested by the manufacturer (Becton Dickinson). Antibodies are added prior to the addition of T-cells in order to “capture” the CD107 molecules as they transiently appear on the surface during the course of the assay. 1×10⁵ T-cells from the IVE culture are added next, and the samples were incubated for 4 hours at 37° C. The T-cells are further stained for additional cell surface molecules such as CD8 and acquired on a FACS Calibur instrument (Becton Dickinson). Data is analyzed using the accompanying Cellquest software, and results were reported as the percentage of CD8+CD107 a and b+ cells.

### CTL Lysis

Cytotoxic activity is measured using a chromium release assay. Target T2 cells are labeled for 1 hour at 37° C. with Na⁵¹Cr and washed 5×10³ target T2 cells were then added to varying numbers of T-cells from the IVE culture. Chromium release is measured in supernatant harvested after 4 hours of incubation at 37° C. The percentage of specific lysis is calculated as: Experimental release-spontaneous release/Total release-spontaneous release×100

### Example 4: Selection of CTL and HTL Epitopes for Inclusion in an Tumor-Specific Vaccine

This example illustrates the procedure for the selection of peptide epitopes for vaccine compositions of the invention. The peptides in the composition can be in the form of a nucleic acid sequence, either single or one or more sequences (i.e., minigene) that encodes peptide(s), or may be single and/or polyepitopic peptides.

Epitopes are selected which, upon administration, mimic immune responses that have been observed to be correlated with tumor clearance. For example, vaccine can include 1-2 epitopes that come from at least one tumor antigen region. Epitopes from one region can be used in combination with epitopes from one or more additional tumor antigen regions.

Epitopes can be selected that have a binding affinity of an IC₅₀ of 500 nM or less for an HLA class I molecule, or for class II, an IC₅₀ of 1000 nM or less.

When creating a polyepitopic compositions, e.g. a minigene, it is typically desirable to generate the smallest peptide possible that encompasses the epitopes of interest. The principles employed are similar, if not the same, as those employed when selecting a peptide comprising nested epitopes. Additionally, however, upon determination of the nucleic acid sequence to be provided as a minigene, the peptide sequence encoded thereby is analyzed to determine whether any “junctional epitopes” have been created. A junctional epitope is a potential HLA binding epitope, as predicted, e.g., by motif analysis. Junctional epitopes are generally to be avoided because the recipient may bind to an HLA molecule and generate an immune response to that epitope, which is not present in a native protein sequence.

Peptide epitopes for inclusion in vaccine compositions are, for example, selected from those listed in the Tables. A vaccine composition comprised of selected peptides, when administered, is safe, efficacious, and elicits an immune response similar in magnitude of an immune response that inhibits tumor growth.

### Example 5: Peptide Composition for Prophylactic or Therapeutic Uses

Immunogenic or vaccine compositions of the invention are used to inhibit tumor growth. For example, a polyepitopic composition (or a nucleic acid comprising the same) containing multiple CTL and HTL epitopes is administered to individuals having tumors. The composition is provided as a single lipidated polypeptide that encompasses multiple epitopes. The composition is administered in an aqueous carrier comprised of alum. The dose of peptide for the initial immunization is from about 1 to about 50,000 μg, generally 100-5,000 μg, for a 70 kg patient. The initial administration is followed by booster dosages at 4 weeks followed by evaluation of the magnitude of the immune response in the patient, by techniques that determine the presence of epitope-specific CTL populations in a PBMC sample. Additional booster doses are administered as required. The composition is found to be both safe and efficacious to inhibit tumor growth.

Alternatively, the polyepitopic composition can be administered as a nucleic acid, for example as RNA, in accordance with methodologies known in the art and disclosed herein.

Non-mutated protein epitope binding agents, such as TCR or CARs can be can be administered in accordance with methodologies known in the art and disclosed herein. The binding agents can be administered as polypeptides or polynucleotides, for example RNA, encoding the binding agents, or as a cellular therapy, by administering cells expressing the binding agents.

Non-mutated protein epitope peptides, polynucleotides, binding agents, or cells expressing these molecules can be delivered to the same patient via multiple methodologies known in the art, and can further be combined with other cancer therapies (e.g., chemotherapy, surgery, radiation, checkpoint inhibitors, etc.).

### Example 6. Administration of Compositions Using Dendritic Cells

Vaccines comprising epitopes of the invention may be administered using dendritic cells. In this example, the peptide-pulsed dendritic cells can be administered to a patient to stimulate a CTL response in vivo. In this method dendritic cells are isolated, expanded, and pulsed with a vaccine comprising peptide CTL and HTL epitopes of the invention. The dendritic cells are infused back into the patient to elicit CTL and HTL responses in vivo. The induced CTL and HTL then destroy (CTL) or facilitate destruction (HTL) of the specific target tumor cells that bear the proteins from which the epitopes in the vaccine are derived.

Alternatively, ex vivo CTL or HTL responses to a particular tumor-associated antigen can be induced by incubating in tissue culture the patient's, or genetically compatible, CTL or HTL precursor cells together with a source of antigen-presenting cells, such as dendritic cells, and the appropriate immunogenic peptides.

After an appropriate incubation time (typically about 7-28 days), in which the precursor cells are activated and expanded into effector cells, the cells are infused back into the patient, where they will destroy (CTL) or facilitate destruction (HTL) of their specific target cells, i.e., tumor cells.

### EMBODIMENTS

Embodiment 1. An isolated antigenic peptide comprising an epitope from a sequence in Table 1 or 2.

Embodiment 2. An isolated antigenic peptide 100 amino acids or less in length which comprises an epitope from a sequence in Table 1 or 2.

Embodiment 3. An isolated antigenic peptide comprising an epitope from a sequence in Table 3 or 4.

Embodiment 4. An isolated antigenic peptide 100 amino acids or less in length which comprises an epitope from a sequence in Table 3 or 4.

Embodiment 5. An isolated antigenic peptide comprising an epitope from a sequence in Table 5 or 6.

Embodiment 6. An isolated antigenic peptide 100 amino acids or less in length which comprises an epitope from a sequence in Table 5 or 6.

Embodiment 7. The isolated antigenic peptide of embodiment 1 or 2, wherein the isolated antigenic peptide is a retroviral antigen.

Embodiment 8. The isolated antigenic peptide of embodiment 3 or 4, wherein the isolated antigenic peptide is a non-mutated overexpressed antigen.

Embodiment 9. The isolated antigenic peptide of embodiment 5 or 6, wherein the isolated antigenic peptide is a viral antigen.

Embodiment 10. The isolated antigenic peptide of any of embodiments 1-9, which is between about 5 to about 50 amino acids in length.

Embodiment 11. The isolated antigenic peptide of any of embodiments 1-10, which is between about 15 to about 35 amino acids in length.

Embodiment 12. The isolated antigenic peptide of embodiment 11, which is about 15 amino acids or less in length.

Embodiment 13. The isolated antigenic peptide of embodiment 12, which is between about 8 and about 11 amino acids in length.

Embodiment 14. The isolated antigenic peptide of embodiment 13, which is 9 or 10 amino acids in length.

Embodiment 15. The isolated antigenic peptide of any of embodiments 1-14, which binds major histocompatibility complex (MHC) class I.

Embodiment 16. The isolated antigenic peptide of embodiment 15, which binds MHC class I with a binding affinity of less than about 500 nM.

Embodiment 17. The isolated antigenic peptide of any of embodiments 1-6, which is about 30 amino acids or less in length.

Embodiment 18. The isolated antigenic peptide of embodiment 17, which is between about 6 and about 25 amino acids in length.

Embodiment 19. The isolated antigenic peptide of embodiment 18, which is between about 15 and about 24 amino acids in length.

Embodiment 20. The isolated antigenic peptide of embodiment 18, which is between about 9 and about 15 amino acids in length.

Embodiment 21. The isolated antigenic peptide of any of embodiments 1-6 and 17-20, which binds MHC class II.

Embodiment 22. The isolated antigenic peptide of embodiment 21, which binds MHC class II with a binding affinity of less than about 1000 nM.

Embodiment 23. The isolated antigenic peptide of any of embodiments 1-22, further comprising flanking amino acids.

Embodiment 24. The isolated antigenic peptide of embodiment 23, wherein the flanking amino acids are not native flanking amino acids.

Embodiment 25. The isolated antigenic peptide of any of embodiments 1-24, which is linked to at least a second antigenic peptide.

Embodiment 26. The isolated antigenic peptide of embodiment 25, wherein peptides are linked using a poly-glycine or poly-serine linker.

Embodiment 27. The isolated antigenic peptide of embodiment 25 or 26, wherein the second antigenic peptide binds MHC class I or class II with a binding affinity of less than about 1000 nM.

Embodiment 28. The isolated antigenic peptide of embodiment 27, wherein the second antigenic peptide binds MHC class I or class II with a binding affinity of less than about 500 nM.

Embodiment 29. The isolated antigenic peptide of embodiment 27 or 28, wherein both of the epitopes bind to human leukocyte antigen (HLA)-A, -B, -C, -DP, -DQ, or -DR.

Embodiment 30. The isolated antigenic peptide of any of embodiments 27-29, wherein the isolated antigenic peptide binds a class I HLA and the second antigenic peptide binds a class II HLA.

Embodiment 31. The isolated antigenic peptide of any of embodiments 27-29, wherein the isolated antigenic peptide binds a class II HLA and the second antigenic peptide binds a class I HLA.

Embodiment 32. The isolated antigenic peptide of any of embodiments 1-31, further comprising modifications which increase in vivo half-life, cellular targeting, antigen uptake, antigen processing, MHC affinity, MHC stability, or antigen presentation.

Embodiment 33. The isolated antigenic peptide of embodiment 32, wherein the modification is conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids.

Embodiment 34. The isolated antigenic peptide of embodiment 32, wherein the cells that are targeted are antigen presenting cells.

Embodiment 35. The isolated antigenic peptide of embodiment 34, wherein the antigen presenting cells are dendritic cells.

Embodiment 36. The isolated antigenic peptide of embodiment 35, wherein the dendritic cells are targeted using DEC205, XCR1, CD197, CD80, CD86, CD123, CD209, CD273, CD283, CD289, CD184, CD85h, CD85j, CD85k, CD85d, CD85g, CD85a, CD141, CD11c, CD83, TSLP receptor, or CD1a marker.

Embodiment 37. The isolated antigenic peptide of embodiment 36, wherein the dendritic cells are targeted using the CD141, DEC205, or XCR1 marker.

Embodiment 38. An in vivo delivery system comprising the isolated antigenic peptide of any of embodiments 1-37.

Embodiment 39. The delivery system of embodiment 38, wherein the delivery system includes cell-penetrating peptides, nanoparticulate encapsulation, virus like particles, or liposomes.

Embodiment 40. The delivery system of embodiment 38, wherein the cell-penetrating peptide is TAT peptide, herpes simplex virus VP22, transportan, or Antp.

Embodiment 41. A cell comprising the isolated antigenic peptide of any of embodiments 1-37.

Embodiment 42. The cell of embodiment 41, which is an antigen presenting cell.

Embodiment 43. The cell of embodiment 42, which is a dendritic cell.

Embodiment 44. A composition comprising the isolated antigenic peptide of any of embodiments 1-37.

Embodiment 45. The composition of embodiment 44, wherein the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the isolated antigenic peptides comprising a tumor-specific epitope defined in Table 1 or 2.

Embodiment 46. The composition of embodiment 44, wherein the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the isolated antigenic peptides comprising a tumor-specific epitope defined in Table 3 or 4.

Embodiment 47. The composition of embodiment 44, wherein the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the isolated antigenic peptides comprising a tumor-specific epitope defined in Table 5 or 6.

Embodiment 48. The composition of any of embodiments 45-47, wherein the composition comprises between 2 and 20 antigenic peptides.

Embodiment 49. The composition of any one of embodiments 45-48, wherein the composition further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, at least 26, at least 27, at least 28, at least 29, or at least 30 additional antigenic peptides.

Embodiment 50. The composition of embodiment 49, wherein the composition comprises between about 4 and about 20 additional antigenic peptides.

Embodiment 51. The composition of any of embodiments 44-50, wherein the additional antigenic peptide is specific for an individual patient's tumor.

Embodiment 52. The composition of embodiment 51, wherein the patient specific antigenic peptide is selected by identifying sequence differences between the genome, exome, and/or transcriptome of the patient's tumor sample and the genome, exome, and/or transcriptome of a non-tumor sample.

Embodiment 53. The composition of embodiment 47, wherein the samples are fresh or formalin-fixed paraffin embedded tumor tissues, freshly isolated cells, or circulating tumor cells.

Embodiment 54. The composition of embodiment 52 or 53, wherein the sequence differences are determined by Next Generation Sequencing.

Embodiment 55. An isolated polynucleotide encoding the isolated antigenic peptide of any of embodiments 1-10.

Embodiment 56. The isolated polynucleotide embodiment 55, which is RNA, optionally a self-amplifying RNA.

Embodiment 57. The isolated polynucleotide of embodiment 56, wherein the RNA is modified to increase stability, increase cellular targeting, increase translation efficiency, adjuvanticity, cytosol accessibility, and/or decrease cytotoxicity.

Embodiment 58. The isolated polynucleotide of embodiment 57, wherein the modification is conjugation to a carrier protein, conjugation to a ligand, conjugation to an antibody, codon optimization, increased GC-content, incorporation of modified nucleosides, incorporation of 5′-cap or cap analog, and/or incorporation of an unmasked poly-A sequence.

Embodiment 59. A cell comprising the polynucleotide of any of embodiments 55-58.

Embodiment 60. A vector comprising the polynucleotide of any one of embodiments 55-58.

Embodiment 61. The vector of embodiment 60, in which the polynucleotide is operably linked to a promoter.

Embodiment 62. The vector of embodiments 60 or 61, which is a self-amplifying RNA replicon, plasmid, phage, transposon, cosmid, virus, or virion.

Embodiment 63. The vector of embodiment 62, which is an adeno-associated virus, herpesvirus, lentivirus, or pseudotypes thereof.

Embodiment 64. An in vivo delivery system comprising the isolated polynucleotide of any of embodiments 55-58.

Embodiment 65. The delivery system of embodiment 60, wherein the delivery system includes spherical nucleic acids, viruses, virus-like particles, plasmids, bacterial plasmids, or nanoparticles.

Embodiment 66. A cell comprising the vector or delivery system of any of embodiments 60-65.

Embodiment 67. The cell of embodiment 66, which is an antigen presenting cell.

Embodiment 68. The cell of embodiment 67, which is a dendritic cell.

Embodiment 69. The cell of embodiment 68, which is an immature dendritic cell.

Embodiment 70. A composition comprising at least one polynucleotide of any of embodiments 55-58.

Embodiment 71. The composition of embodiment 70, wherein the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of the isolated polynucleotides.

Embodiment 72. The composition of embodiment 71, wherein the composition comprises between about 2 and about 20 polynucleotides.

Embodiment 73. The composition of any one of embodiments 70-72, wherein the composition further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 additional antigenic polynucleotides encoding for additional antigenic peptides.

Embodiment 74. The composition of embodiment 73, wherein the composition comprises between about 4 and about 20 additional antigenic polynucleotides.

Embodiment 75. The composition of embodiment 73, wherein the isolated polynucleotides and the additional antigenic polynucleotides are linked.

Embodiment 76. The composition of embodiment 75, wherein the polynucleotides are linked using nucleic acids that encode a poly-glycine or poly-serine linker.

Embodiment 77. The composition of any of embodiments 70-76, wherein at least one of the additional antigenic peptide is specific for an individual patient's tumor.

Embodiment 78. The composition of embodiment 77, wherein the patient specific antigenic peptide is selected by identifying sequence differences between the genome, exome, and/or transcriptome of the patient's tumor sample and the genome, exome, and/or transcriptome of a non-tumor sample.

Embodiment 79. The composition of embodiment 78, wherein the samples are fresh or formalin-fixed paraffin embedded tumor tissues, freshly isolated cells, or circulating tumor cells.

Embodiment 80. The composition of embodiments 78 or 79, wherein the sequence differences are determined by Next Generation Sequencing.

Embodiment 81. AT cell receptor (TCR) capable of binding at least one antigenic peptide listed in any of embodiments 1-34.

Embodiment 82. The TCR of embodiment 81, which is capable of binding the isolated antigenic peptide in the context of MHC class I or class II.

Embodiment 83. A chimeric antigen receptor comprising: (i) a T cell activation molecule; (ii) a transmembrane region; and (iii) an antigen recognition moiety capable of binding an isolated antigenic peptide of any one of embodiments 1-34.

Embodiment 84. The chimeric antigen receptor of embodiment 83, wherein CD3-zeta is the T cell activation molecule.

Embodiment 85. The chimeric antigen receptor of embodiment 83 or 84, further comprising at least one costimulatory signaling domain.

Embodiment 86. The chimeric antigen receptor of any of embodiments 83-85, wherein the signaling domain is CD28, 4-1BB, ICOS, OX40, ITAM, or Fc epsilon RI-gamma.

Embodiment 87. The chimeric antigen receptor of any of embodiments 83-86, wherein the antigen recognition moiety is capable of binding the isolated antigenic peptide in the context of MHC class I or class II.

Embodiment 88. The chimeric antigen receptor of any of embodiments 83-87, comprising the CD3-zeta, CD28, CTLA-4, ICOS, BTLA, KIR, LAG3, CD137, OX40, CD27, CD40L, Tim-3, A2aR, or PD-1 transmembrane region.

Embodiment 89. The chimeric antigen receptor of any of embodiments 83-88, wherein the tumor-specific epitope is located in the extracellular domain of a tumor associated polypeptide.

Embodiment 90. A T cell comprising the T cell receptor or chimeric antigen receptor of any of embodiments 83-88.

Embodiment 91. The T cell of embodiment 90, which is a helper or cytotoxic T cell.

Embodiment 92. A nucleic acid comprising a promoter operably linked to a polynucleotide encoding the T cell receptor of embodiment 81 or 82.

Embodiment 93. The nucleic acid of embodiment 92, wherein the TCR is capable of binding the at least one antigenic peptide in the context of major histocompatibility complex (MHC) class I or class II.

Embodiment 94. A nucleic acid comprising a promoter operably linked to a polynucleotide encoding the chimeric antigen receptor of any of embodiments 83-89.

Embodiment 95. The nucleic acid of embodiment 94, wherein the antigen recognition moiety is capable of binding the at least one antigenic peptide in the context of major histocompatibility complex (MHC) class I or class II.

Embodiment 96. The nucleic acid of embodiment 94 or 95, wherein the tumor-specific epitope is located in the extracellular domain of a tumor associated polypeptide.

Embodiment 97. The nucleic acid of any of embodiments 94-96, comprising the CD3-zeta, CD28, CTLA-4, ICOS, BTLA, KIR, LAG3, CD137, OX40, CD27, CD40L, Tim-3, A2aR, or PD-1 transmembrane region.

Embodiment 98. An antibody capable of binding at least one antigenic peptide listed in Table 1 or 2.

Embodiment 99. An antibody capable of binding at least one antigenic peptide listed in Table 3 or 4.

Embodiment 100. An antibody capable of binding at least one antigenic peptide listed in Table 5 or 6.

Embodiment 101. An antibody of embodiment 98, wherein the at least one antigenic peptide listed in Table 1 or 2 is a retroviral antigenic peptide.

Embodiment 102. An antibody of embodiment 99, wherein the at least one antigenic peptide listed in Table 3 or 4 is a non-mutated overexpressed antigenic peptide.

Embodiment 103. An antibody of embodiment 100, wherein the at least one antigenic peptide listed in Table 5 or 6 is a viral antigenic peptide.

Embodiment 104. A modified cell transfected or transduced with the nucleic acid of any one of embodiments 92-97.

Embodiment 105. The modified cell of embodiment 104, wherein the modified cell is a T cell, tumor infiltrating lymphocyte, NK-T cell, TCR-expressing cell, CD4+ T cell, CD8+ T cell, or NK cell.

Embodiment 106. A composition comprising the T cell receptor or chimeric antigen receptor of any of embodiments 81-89.

Embodiment 107. A composition comprising autologous patient T cells containing the T cell receptor or chimeric antigen receptor of any of embodiments 81-89.

Embodiment 108. The composition of embodiment 105 or 106, further comprising an immune checkpoint inhibitor.

Embodiment 109. The composition of embodiment 106 or 107, further comprising at least two immune checkpoint inhibitors.

Embodiment 110. The composition of embodiment 108 or 109, wherein each of the immune checkpoint inhibitors inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof.

Embodiment 111. The composition of embodiment 108 or 109, wherein each of the immune checkpoint inhibitors interacts with a ligand of a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof.

Embodiment 112. The composition of any of embodiments 44-54, 70-79, and 106-111, further comprising an immune modulator or adjuvant.

Embodiment 113. The composition of embodiment 112, wherein the immune modulator is a co-stimulatory ligand, a TNF ligand, an Ig superfamily ligand, CD28, CD80, CD86, ICOS, CD40L, OX40, CD27, GITR, CD30, DR3, CD69, or 4-1BB.

Embodiment 114. The composition of embodiment 112, wherein the immune modulator is at least one cancer cell or cancer cell extract.

Embodiment 115. The composition of embodiment 114, wherein the cancer cell is autologous to the subject in need of the composition.

Embodiment 116. The composition of embodiment 115, wherein the cancer cell has undergone lysis or been exposed to UV radiation.

Embodiment 117. The composition of embodiment 112, wherein the composition further comprises an adjuvant.

Embodiment 118. The composition of embodiment 117, wherein the adjuvant is selected from the group consisting of: Poly(I:C), Poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312 VG, Montanide ISA 206 VG, Montanide ISA 50 V2, Montanide ISA 51 VG, OK-432, OM-174, OM-197-MP-EC, ISA-TLR2 agonist, ONTAK, PepTel®. vector system, PLG microparticles, resiquimod, SRL172, virosomes and other virus-like particles, YF-17D, VEGF trap, R848, beta-glucan, Pam3Cys, Pam3CSK4, acrylic or methacrylic polymers, copolymers of maleic anhydride, and QS21 stimulon.

Embodiment 119. The composition of embodiment 118 or 118, wherein the adjuvant induces a humoral when administered to a subject.

Embodiment 120. The composition of embodiment 119, wherein the adjuvant induces a T helper cell type 1 when administered to a subject.

Embodiment 121. A method of inhibiting growth of a tumor cell expressing a tumor-specific epitope defined in Table 1 or 2, comprising contacting the tumor cell with the peptide, polynucleotide, delivery system, vector, composition, antibody, or cells of any of embodiments 1-120.

Embodiment 122. A method of inhibiting growth of a tumor cell expressing a tumor-specific epitope defined in Table 3 or 4, comprising contacting the tumor cell with the peptide, polynucleotide, delivery system, vector, composition, antibody, or cells of any of embodiments 1-120.

Embodiment 123. A method of inhibiting growth of a tumor cell expressing a tumor-specific epitope defined in Table 5 or 6, comprising contacting the tumor cell with the peptide, polynucleotide, delivery system, vector, composition, antibody, or cells of any of embodiments 1-120.

Embodiment 124. A method of treating cancer or initiating, enhancing, or prolonging an anti-tumor response in a subject in need thereof comprising administering to the subject the peptide, polynucleotide, vector, composition, antibody, or cells of any of embodiments 1-120.

Embodiment 125. The method of any of embodiments 121-124, wherein the subject is a human.

Embodiment 126. The method of embodiment 125, wherein the subject has cancer.

Embodiment 127. The method of embodiment 126, wherein the cancer is selected from the group consisting of urogenital, renal, gynecological, lung, gastrointestinal, head and neck cancer, malignant glioblastoma, malignant mesothelioma, non-metastatic or metastatic breast cancer, malignant melanoma, Merkel Cell Carcinoma or bone and soft tissue sarcomas, hematologic neoplasias, multiple myeloma, acute myelogenous leukemia, chronic myelogenous leukemia, myelodysplastic syndrome and acute lymphoblastic leukemia, non-small cell lung cancer (NSCLC), triple-negative breast cancer (TNBC), smoldering myeloma (SMM), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, esophageal cancers, hepatocellular cancers, cholangiocellular cancers, head and neck squamous cell cancer soft tissue sarcoma, and small cell lung cancer.

Embodiment 128. The method of any of embodiments 121-127, wherein the subject has undergone surgical removal of the tumor.

Embodiment 129. The method of any of embodiments 121-128, wherein the peptide, polynucleotide, vector, composition, or cells is administered via intravenous, intraperitoneal, intratumoral, intradermal, or subcutaneous administration.

Embodiment 130. The method of embodiment 129, wherein the peptide, polynucleotide, vector, composition, or cells is administered into an anatomic site that drains into a lymph node basin.

Embodiment 131. The method of embodiment 130, wherein administration is into multiple lymph node basins.

Embodiment 132. The method of any one of embodiments 121-131, wherein administration is by a subcutaneous or intradermal route.

Embodiment 133. The method of embodiment 129, wherein peptide is administered.

Embodiment 134. The method of embodiment 133, wherein administration is intratumorally.

Embodiment 135. The method of embodiment 129, wherein polynucleotide, optionally RNA, is administered.

Embodiment 136. The method of embodiment 129 or 135, wherein the polynucleotide is administered intravenously.

Embodiment 137. The method of embodiment 129, wherein the cell is a T cell or dendritic cell.

Embodiment 138. The method of embodiment 129 or 137, wherein the peptide or polynucleotide comprises an antigen presenting cell targeting moiety.

Embodiment 139. The method of any of embodiments 121-138, further comprising administering at least one immune checkpoint inhibitor to the subject.

Embodiment 140. The method of embodiment 139, wherein the checkpoint inhibitor is a biologic therapeutic or a small molecule.

Embodiment 141. The method of embodiment 139 or 140, wherein the checkpoint inhibitor is selected from the group consisting of a monoclonal antibody, a humanized antibody, a fully human antibody and a fusion protein or a combination thereof.

Embodiment 142. The method of any of embodiments 139-141, wherein the checkpoint inhibitor inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof.

Embodiment 143. The method of any of embodiments 139-142, wherein the checkpoint inhibitor interacts with a ligand of a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof.

Embodiment 144. The method of any of embodiments 139-143, wherein two or more checkpoint inhibitors are administered.

Embodiment 145. The method of embodiment 144, wherein the checkpoint inhibitors are: (i) ipilimumab or tremelimumab, and (ii) nivolumab.

Embodiment 146. The method of any of embodiments 139-145, wherein the checkpoint inhibitor and the composition are administered simultaneously or sequentially in any order.

Embodiment 147. The method of embodiment 146, wherein the peptide, polynucleotide, vector, composition, or cells is administered prior to the checkpoint inhibitor.

Embodiment 148. The method of embodiment 146, wherein the peptide, polynucleotide, vector, composition, or cells is administered after the checkpoint inhibitor.

Embodiment 149. The method of embodiment 146, wherein administration of the checkpoint inhibitor is continued throughout antigen peptide, polynucleotide, vector, composition, or cell therapy.

Embodiment 150. The method of any of embodiments 139-149, wherein the antigen peptide, polynucleotide, vector, composition, or cell therapy is administered to subjects that only partially respond or do not respond to checkpoint inhibitor therapy.

Embodiment 151. The method of any one of embodiments 121-138, wherein the composition is administered intravenously or subcutaneously.

Embodiment 152. The method of any one of embodiments 139-150, wherein the checkpoint inhibitor is administered intravenously or subcutaneously.

Embodiment 153. The method of any one of embodiments 139-151, wherein the checkpoint inhibitor is administered subcutaneously within about 2 cm of the site of administration of the composition.

Embodiment 154. The method of embodiment 153, wherein the composition is administered into the same draining lymph node as the checkpoint inhibitor.

Embodiment 155. The method of any of embodiments 121-154, further comprising administering an additional therapeutic agent to the subject either prior to, simultaneously with, or after treatment with the peptide, polynucleotide, vector, composition, or cells.

Embodiment 156. The method of embodiment 155, wherein the additional agent is a chemotherapeutic agent, an immunomodulatory drug, an immune metabolism modifying drug, a targeted therapy, radiation an anti-angiogenesis agent, or an agent that reduces immune-suppression.

Embodiment 157. The method of embodiment 156, wherein the chemotherapeutic agent is an alkylating agent, a topoisomerase inhibitor, an anti-metabolite, or an anti-mitotic agent.

Embodiment 158. The method of embodiment 155, wherein the additional agent is an anti-glucocorticoid induced tumor necrosis factor family receptor (GITR) agonistic antibody or antibody fragment, ibrutinib, docetaxeol, cisplatin, or cyclophosphamide.

Embodiment 159. The method of any of embodiments 121-158, which elicits a CD4+ T cell immune response.

Embodiment 160. The method of any of embodiments 121-159, which elicits a CD4+ T cell immune response and a CD8+ T cell immune response.

Embodiment 161. A method for stimulating an immune response in a subject, comprising administering an effective amount of modified cells or composition of any of embodiments 104-120.

Embodiment 162. The method of embodiment 161, wherein the immune response is cytotoxic and/or humoral immune response.

Embodiment 163. The method of embodiment 161, wherein the method stimulates a T cell-mediated immune response in a subject.

Embodiment 164. The method of embodiment 163, wherein the T cell-mediated immune response is directed against a target cell.

Embodiment 165. The method of embodiment 164, wherein the target cell is a tumor cell.

Embodiment 166. The method of any of embodiments 161-165, wherein the modified cells are transfected or transduced in vivo.

Embodiment 167. The method of any of embodiments 161-166, wherein the modified cells are transfected or transduced ex vivo.

Embodiment 168. The method of any of embodiments 161-167, wherein the modified cells are autologous patient T cells.

Embodiment 169. The method of embodiment 168, wherein the autologous patient T cells are obtained from a patient that has received an antigen peptide or nucleic acid vaccine.

Embodiment 170. The method of embodiment 169, wherein the antigen peptide or nucleic acid vaccine comprises at least one personalized antigen.

Embodiment 171. The method of embodiment 170, wherein the antigen peptide or nucleic acid vaccine comprises at least one additional antigenic peptide listed in Table 1 or 2.

Embodiment 172. The method of embodiment 170, wherein the antigen peptide or nucleic acid vaccine comprises at least one additional antigenic peptide listed in Table 3 or 4.

Embodiment 173. The method of embodiment 170, wherein the antigen peptide or nucleic acid vaccine comprises at least one additional antigenic peptide listed in Table 5 or 6.

Embodiment 174. The method of embodiment 171, wherein the at least one additional antigenic peptide listed in Table 1 or 2 is a retroviral antigenic peptide.

Embodiment 175. The method of embodiment 172, wherein the at least one additional antigenic peptide listed in Table 3 or 4 is a non-mutated overexpressed antigenic peptide.

Embodiment 176. The method of embodiment 173, wherein the at least one additional antigenic peptide listed in Table 5 or 6 is a viral antigenic peptide.

Embodiment 177. The method of any of embodiments 171-176, wherein the patient received a chemotherapeutic agent, an immunomodulatory drug, an immune metabolism modifying drug, targeted therapy or radiation prior to and/or during receipt of the antigen peptide or nucleic acid vaccine.

Embodiment 178. The method of any of embodiments 169-177, wherein the patient receives treatment with at least one checkpoint inhibitor.

Embodiment 179. The method of any of embodiments 169-178, wherein the autologous T cells are obtained from a patient that has already received at least one round of T cell therapy containing an antigen.

Embodiment 180. The method of any of embodiments 169-179, wherein the method further comprises adoptive T cell therapy.

Embodiment 181. The method of embodiment 180, wherein the adoptive T cell therapy comprises autologous T-cells.

Embodiment 182. The method of embodiment 181, wherein the autologous T-cells are targeted against tumor antigens.

Embodiment 183. The method of embodiment 180 or 181, wherein the adoptive T cell therapy further comprises allogenic T-cells.

Embodiment 184. The method of embodiment 183, wherein the allogenic T-cells are targeted against tumor antigens.

Embodiment 185. The method of any of embodiments 180-184, wherein the adoptive T cell therapy is administered before the checkpoint inhibitor.

Embodiment 186. A method for evaluating the efficacy of any of embodiments 121-185, comprising: (i) measuring the number or concentration of target cells in a first sample obtained from the subject before administering the modified cell, (ii) measuring the number concentration of target cells in a second sample obtained from the subject after administration of the modified cell, and (iii) determining an increase or decrease of the number or concentration of target cells in the second sample compared to the number or concentration of target cells in the first sample.

Embodiment 187. The method of embodiment 186, wherein treatment efficacy is determined by monitoring a clinical outcome; an increase, enhancement or prolongation of anti-tumor activity by T cells; an increase in the number of anti-tumor T cells or activated T cells as compared with the number prior to treatment; B cell activity; CD4 T cell activity; or a combination thereof.

Embodiment 188. The method of embodiment 187, wherein treatment efficacy is determined by monitoring a biomarker.

Embodiment 189. The method of embodiment 188, wherein the biomarker is selected from the group consisting of CEA, Her-2/neu, bladder tumor antigen, thyroglobulin, alpha-fetoprotein, PSA, CA 125, CA19.9, CA 15.3, leptin, prolactin, osteopontin, IGF-II, CD98, fascin, sPIgR, 14-3-3 eta, troponin I, and b-type natriuretic peptide.

Embodiment 190. The method of embodiment 187, wherein clinical outcome is selected from the group consisting of tumor regression; tumor shrinkage; tumor necrosis; anti-tumor response by the immune system; tumor expansion, recurrence or spread; or a combination thereof.

Embodiment 191. The method of embodiment 187, wherein the treatment effect is predicted by presence of T cells or by presence of a gene signature indicating T cell inflammation or a combination thereof.

Embodiment 192. A method of treating cancer or initiating, enhancing, or prolonging an anti-tumor response in a subject in need thereof comprising administering to the subject: the peptide, polynucleotide, vector, composition, antibody, or cells of any of embodiments 1-120; and at least one checkpoint inhibitor.

Embodiment 193. The method of embodiment 192, further comprising administration of an immunomodulator or adjuvant.

Embodiment 194. The method of embodiment 192, wherein the immunomodulator or adjuvant is selected from the group consisting of Poly(I:C), Poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312 VG, Montanide ISA 206 VG, Montanide ISA 50 V2, Montanide ISA 51 VG, OK-432, OM-174, OM-197-MP-EC, ISA-TLR2 agonist, ONTAK, PepTel® vector system, PLG microparticles, resiquimod, SRL172, virosomes and other virus-like particles, YF-17D, VEGF trap, R848, beta-glucan, Pam3Cys, Pam3CSK4, acrylic or methacrylic polymers, copolymers of maleic anhydride, and QS21 stimulon. a co-stimulatory ligand, a TNF ligand, an Ig superfamily ligand, CD28, CD80, CD86, ICOS, CD40L, OX40, CD27, GITR, CD30, DR3, CD69, or 4-1BB.

Embodiment 195. The method of embodiment 192, wherein the immunomodulator or adjuvant is Poly-ICLC.

Embodiment 196. The method of any one of embodiments 192-193, wherein the checkpoint inhibitor is an anti-PD1 antibody or antibody fragment.

Embodiment 197. The method of embodiment 194, wherein the inhibitor of the PD-1 pathway is nivolumab.

Embodiment 198. The method of any one of embodiments 192-193, wherein the checkpoint inhibitor is an anti-CTLA4 antibody or antibody fragment.

Embodiment 199. The method of embodiment 196, wherein the anti-CTLA4 antibody is ipilimumab or tremelimumab.

Embodiment 200. The method of any one of embodiments 192-197, wherein the method comprises administering both an anti-PD1 antibody and an anti-CTLA4 antibody.

Embodiment 201. The method of any one of embodiments 192-197, wherein administration of the checkpoint inhibitor is initiated before initiation of administration of the peptide, polynucleotide, vector, composition, antibody, or cell.

Embodiment 202. The method of any one of embodiments 192-197, wherein administration of the checkpoint inhibitor is initiated after initiation of administration of the peptide, polynucleotide, vector, composition, antibody, or cell.

Embodiment 203. The method of any one of embodiments 192-197, wherein administration of the checkpoint inhibitor is initiated simultaneously with the initiation of administration of the peptide, polynucleotide, vector, composition, antibody, or cell.

Embodiment 204. The method of any one of embodiments 192-201, wherein the peptide, polynucleotide, vector, composition, antibody, or cell is administered intravenously or subcutaneously.

Embodiment 205. The method of any one of embodiments 192-201, wherein the checkpoint inhibitor is administered intravenously or subcutaneously.

Embodiment 206. The method of any one of embodiments 192-203, wherein the checkpoint inhibitor is administered subcutaneously within about 2 cm of the site of administration of the peptide, polynucleotide, vector, composition, antibody, or cell.

Embodiment 207. The method of embodiment 204, wherein the peptide, polynucleotide, vector, composition, antibody, or cell is administered into the same draining lymph node as the checkpoint inhibitor.

Embodiment 208. A kit comprising an antigen therapeutic of any of embodiments 1-120.

Embodiment 209. The method of embodiment 124, wherein the cancer is selected from the group consisting of: CRC, head and neck, stomach, lung squamous, lung adeno, prostate, bladder, stomach, renal cell carcinoma, and uterine.

Embodiment 210. The method of embodiment 124, wherein the cancer is selected from the group consisting of: melanoma, lung squamous, DLBCL, uterine, head and neck, uterine, liver, and CRC.

Embodiment 211. The method of embodiment 124, wherein the cancer is selected from the group consisting of: cervical, head and neck, anal, stomach, Burkitt's lymphoma, and nasopharyngeal carcinoma.

Provided herein is an immunogenic vaccine composition comprising a peptide comprising at least 8 contiguous amino acids of a sequence in any one of Tables 1-6. In some embodiments, the peptide is a synthetic peptide. In some embodiments, the peptide is a recombinant peptide. In some embodiments, the peptide comprises a sequence from an endogenous retroviral protein. In some embodiments, the peptide comprises a sequence from an exogenous viral protein. In some embodiments, the peptide comprises a sequence of a protein expressed by a cancer cell of a subject with cancer, wherein the protein is expressed by the cancer cell at a level that is higher than a level expressed by a non-cancer cell of the subject. In some embodiments, the peptide is 100 amino acids or less in length. In some embodiments, the peptide is from about 5 to about 50 amino acids in length or from about 15 to about 35 amino acids in length. In some embodiments, the peptide is about 30 amino acids or less in length or about 15 amino acids or less in length. In some embodiments, the peptide comprises a sequence which binds a major histocompatibility complex (MHC) class I with a binding affinity of less than about 500 nM. In some embodiments, the peptide comprises a sequence which binds a major histocompatibility complex (MHC) class II with a binding affinity of less than about 1000 nM. In some embodiments, the peptide further comprises non-native amino acids flanking the at least 8 contiguous amino acids. In some embodiments, the composition further comprises a second peptide comprising at least 8 contiguous amino acids of a sequence in any one of Tables 1-6, wherein the second antigenic peptide binds MHC class I or class II with a binding affinity of less than about 1000 nM. In some embodiments, the peptides are linked using a poly-glycine or poly-serine linker. In some embodiments, the second antigenic peptide binds MHC class I or class II with a binding affinity of less than about 1000 nM or less than about 500 nM. In some embodiments, the peptide further comprises a modification which increases in vivo half-life, cellular targeting, antigen uptake, antigen processing, MHC affinity, MHC stability, or antigen presentation. In some embodiments, the modification is conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. In some embodiments, the peptide comprises a modification which increases targeting by antigen presenting cells. In some embodiments, the antigen presenting cells are dendritic cells. In some embodiments, the modification which increases targeting by the dendritic cells is a DEC205, XCR1, CD197, CD80, CD86, CD123, CD209, CD273, CD283, CD289, CD184, CD85h, CD85j, CD85k, CD85d, CD85g, CD85a, CD141, CD11c, CD83, TSLP receptor, or CD1a marker. In some embodiments, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 of peptides each comprising at least 8 contiguous amino acids of a sequence in any one of Tables 1-6. In some embodiments, the composition comprises from 2 to 20 peptides each comprising at least 8 contiguous amino acids of a sequence in any one of Tables 1-6. In some embodiments, the composition further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25, at least 26, at least 27, at least 28, at least 29, or at least 30 additional antigenic peptides. In some embodiments, the additional antigenic peptides are specific for an individual patient's tumor. In some embodiments, the additional antigenic peptides are selected by identifying sequence differences between the genome, exome, and/or transcriptome of the patient's tumor sample and the genome, exome, and/or transcriptome of a non-tumor sample. In some embodiments, identifying sequence differences comprises performing Next Generation Sequencing. Provided herein is a composition comprising an antigen presenting cell comprising a peptide comprising at least 8 contiguous amino acids of a sequence in any one of Tables 1-6. In some embodiments, the antigen presenting cell is a dendritic cell.

Provided herein is an in vivo delivery system comprising a composition described herein. In some embodiments, the delivery system includes a cell-penetrating peptide, nanoparticulate encapsulation, a virus like particle, or a liposome. In some embodiments, the cell-penetrating peptide is a TAT peptide, herpes simplex virus VP22, transportan, or Antp.

Provided herein is an immunogenic vaccine composition comprising a recombinant polynucleotide encoding a peptide comprising at least 8 contiguous amino acids of a sequence in any one of Tables 1-6. In some embodiments, the recombinant polynucleotide is RNA, optionally a self-amplifying RNA. In some embodiments, the RNA is modified to increase stability, increase cellular targeting, increase translation efficiency, adjuvanticity, cytosol accessibility, and/or decrease cytotoxicity. In some embodiments, the modification is conjugation to a carrier protein, conjugation to a ligand, conjugation to an antibody, codon optimization, increased GC-content, incorporation of modified nucleosides, incorporation of 5′-cap or cap analog, and/or incorporation of an unmasked poly-A sequence.

Provided herein is a composition comprising a cell comprising a recombinant polynucleotide encoding a peptide comprising at least 8 contiguous amino acids of a sequence in any one of Tables 1-6.

Provided herein is a composition comprising a vector comprising a polynucleotide comprising a sequence encoding a peptide comprising at least 8 contiguous amino acids of a sequence in any one of Tables 1-6. In some embodiments, the polynucleotide is operably linked to a promoter. In some embodiments, the polynucleotide is a self-amplifying RNA replicon, plasmid, phage, transposon, cosmid, virus, or virion. In some embodiments, the virus is an adeno-associated virus, herpesvirus, lentivirus, or pseudotypes thereof.

Provided herein is an in vivo delivery system comprising a composition described herein. In some embodiments, the delivery system includes spherical nucleic acids, viruses, virus-like particles, plasmids, bacterial plasmids, or nanoparticle.

Provided herein is a T cell receptor (TCR) that specifically binds to a peptide:MHC complex, wherein the peptide of the peptide of the peptide:MHC complex is a peptide comprising at least 8 contiguous amino acids of a sequence in any one of Tables 1-6.

Provided herein is a T cell comprising a T cell receptor (TCR) that specifically binds to a peptide:MHC complex, wherein the peptide of the peptide of the peptide:MHC complex is a peptide comprising at least 8 contiguous amino acids of a sequence in any one of Tables 1-6. In some embodiments, the T cell is a helper or cytotoxic T cell. In some embodiments, the T cell is an autologous patient T cell.

Provided herein is a method of treating cancer in a subject in need thereof comprising administering to the subject a composition described herein; wherein the subject comprises cancer cells expressing a protein comprising at least 8 contiguous amino acids of a sequence in any one of Tables 1-6. In some embodiments, the subject is a human. In some embodiments, the cancer is selected from the group consisting of urogenital, gynecological, lung, gastrointestinal, head and neck cancer, malignant glioblastoma, malignant mesothelioma, non-metastatic or metastatic breast cancer, triple-negative breast cancer (TNBC), malignant melanoma, Merkel Cell Carcinoma or bone and soft tissue sarcomas, hematologic neoplasias, multiple myeloma, smoldering myeloma (SMM), acute myelogenous leukemia, chronic myelogenous leukemia, myelodysplastic syndrome and acute lymphoblastic leukemia, non-small cell lung cancer (NSCLC), breast cancer, metastatic colorectal cancers, hormone sensitive or hormone refractory prostate cancer, colorectal cancer, ovarian cancer, hepatocellular cancer, renal cell cancer, pancreatic cancer, gastric cancer, esophageal cancers, hepatocellular cancers, cholangiocellular cancers, head and neck squamous cell cancer soft tissue sarcoma, and small cell lung cancer. In some embodiments, the method further comprises administering at least one immune checkpoint inhibitor to the subject. In some embodiments, the checkpoint inhibitor inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof.

What is claimed is:

1. A method of treating a disease or condition in a subject in need thereof, the method comprising administering to the subject a polypeptide or a polynucleotide encoding the polypeptide, wherein the polypeptide comprises an amino acid sequence comprising at least 8 contiguous amino acids of a viral protein epitope sequence encoded by (I) an E6 gene of Human papillomavirus type 16 (HPV-16) or (II) an E7 gene of HPV-16, wherein:
(a) the subject has been determined to express an MHC encoded by an HLA B07:02 allele and wherein the amino acid sequence is encoded by the E6 gene of HPV-16 and comprises a sequence of RGRWTGRCM (SEQ ID NO: 70);
(b) the subject has been determined to express an MHC encoded by an HLA B08:01 allele and wherein the amino acid sequence is encoded by the E6 gene of HPV-16; or
(c) the subject has been determined to express an MHC encoded by an HLA A24:02 allele and
(i) the amino acid sequence is encoded by the E7 gene of HPV-16 and comprises a sequence of RAHYNIVTF (SEQ ID NO: 83), or
(ii) the amino acid sequence is encoded by the E6 gene of HPV-16 and comprises a sequence of MHQKRTAMF (SEQ ID NO: 66).

2. The method of claim 1, wherein the amino acid sequence comprises a first amino acid sequence comprising at least 8 contiguous amino acids of the viral protein epitope sequence encoded by the E6 gene of HPV-16, and a second amino acid sequence comprising at least 8 contiguous amino acids of the viral protein epitope sequence encoded by the E7 gene of HPV-16.

3. The method of claim 2, wherein the polypeptide comprises a first polypeptide comprising the first amino acid sequence and a second polypeptide comprising the second amino acid sequence.

4. The method of claim 1, wherein the method comprises administering to the subject the polynucleotide encoding the polypeptide, and wherein the polynucleotide is a messenger-RNA (mRNA).

5. The method of claim 4, wherein the mRNA is a part of a lipid nanoparticle.

6. The method of claim 3, wherein the first polypeptide or the second polypeptide is less than or equal to 600 amino acids in length.

7. The method of claim 1, wherein the polypeptide further comprises a modification which increases in vivo half-life, cellular targeting, antigen uptake, antigen processing, MHC affinity, MHC stability, or antigen presentation.

8. The method of claim 1, wherein the polypeptide further comprises a poly-glycine or poly-serine linker.

9. The method of claim 1, wherein the polypeptide further comprises a peptide sequence of QYIKANSKFIGITE (SEQ ID NO: 1).

10. The method of claim 1, wherein the disease or condition is a cancer expressing an antigen encoded by the E6 gene of HPV-16 or the E7 gene of HPV-16.

11. The method of claim 1, wherein the disease or condition is a cancer selected from the group consisting of cervical cancer, head and neck cancer, anal cancer, urogenital cancer, and gynecological cancer.

12. The method of claim 1, further comprising administering an additional therapeutic agent to the subject, wherein the additional therapeutic agent comprises a chemotherapeutic agent, an immunotherapeutic agent, or an immune checkpoint inhibitor.

13. The method of claim 1, wherein the subject expresses the MHC encoded by the HLA B07:02 allele.

14. The method of claim 13, wherein the amino acid sequence is encoded by the E6 gene of HPV-16 and comprises the sequence of RGRWTGRCM (SEQ ID NO: 70).

15. The method of claim 1, wherein the subject expresses the MHC encoded by the HLA B08:01 allele and the amino acid sequence is encoded by the E6 gene of HPV-16.

16. The method of claim 15, wherein the amino acid sequence comprises a sequence selected from the group consisting of CVYCKQQLL (SEQ ID NO: 51), DKKQRFHNI (SEQ ID NO: 54), EYRHYCYSL (SEQ ID NO: 55), MHQKRTAMF (SEQ ID NO: 66), SEYRHYCYSL (SEQ ID NO: 73), and SSRTRRETQL (SEQ ID NO: 74).

17. The method of claim 1, wherein the subject expresses the MHC encoded by the HLA A24:02 allele, and wherein the amino acid sequence is encoded by the E7 gene of HPV-16 and comprises the sequence of RAHYNIVTF (SEQ ID NO: 83).

18. The method of claim 1, wherein the subject expresses the MHC encoded by the HLA A24:02 allele, and wherein the amino acid sequence is encoded by the E6 gene of HPV-16 and comprises the sequence of MHQKRTAMF (SEQ ID NO: 66).

19. The method of claim 15, wherein the amino acid sequence comprises a sequence of CVYCKQQLL (SEQ ID NO: 51).

20. The method of claim 15, wherein the amino acid sequence comprises a sequence selected from the group consisting of DKKQRFHNI (SEQ ID NO: 54), EYRHYCYSL (SEQ ID NO: 55), MHQKRTAMF (SEQ ID NO: 66), SEYRHYCYSL (SEQ ID NO: 73), and SSRTRRETQL (SEQ ID NO: 74).

21. The method of claim 1, wherein the subject is human.


SEQUENCE LISTING (947 sequences)

SEQ ID NO: 1 - protein, 14 aa, Clostridium tetani
QYIKANSKFIGITE

SEQ ID NO: 2 - protein, 21 aa, Plasmodium falciparum
DIEKKIAKMEKASSVFNVVNS

SEQ ID NO: 3 - protein, 16 aa, Streptococcus sp.
GAVDSILGGVATYGAA

SEQ ID NO: 4 - protein, 13 aa, unidentified
  VARIANT 1: note = /replace="D-Ala"
  VARIANT 3: note = /replace="Cyclohexylalanine" or "Phenylalanine"
  VARIANT 13: note = /replace="D-Ala"
  REGION 1..13: note = MISC_FEATURE - /note="Variant residues given in the sequence have nopreference with respect to those in the annotationsfor variant positions"
  REGION 1..13: note = source = /note="Description of Unknown:PADRE sequence"
AKYVWANTLKAAA

SEQ ID NO: 5 - protein, 93 aa, Homo sapiens
MGNLPPSIPPSPLACVLKNLKPLQLTPDLKPKCLIFFCNTAWPQYKLDNGSKWPENGTFDFSILQDLNNSCRKMGKWSEVPDVQAFFYTSVPS

SEQ ID NO: 6 - protein, 144 aa, Homo sapiens [sequence omitted]

SEQ ID NO: 7 - protein, 160 aa, Homo sapiens
MACIYPTTFYTSLPTKSLNMGISLTTILILSVAVLLSTAAPPSCRECYQSLHYRGEMQQYFTYHTHIERSCYGNLIEECVESGKSYYKVKNLGVCGSRNGAICPRGKQWLCFTKIGQWGVNTQVLEDIKREQIIAKAKASKPTTPPENRPRHFHSFIQKL

SEQ ID NO: 8 - protein, 38 aa, Homo sapiens [sequence omitted]

SEQ ID NO: 9 - protein, 242 aa, Homo sapiens
RLFLTKPGKEIGPALAQWWPKVCAEDNPPGLAVNQAPVLREVKPEAQPVRQNQYPVPREALEGIQVHLKHLRTFGIIVPCQSPWNTPLLPVPKPGTKDYRPVQDLRLVNQATVTFHPTVPNPYTLLGLLPAKDSWFTCLDLKDAFFSIRLAPESQKLFAFQWEDPGSGVTTHYTWTRLPQGFKNFPHHLWGGTGSRPPKVSCQRPRLRVVPVHRQPPAGTPHGSRVRQRNRRPASAPGGLWV

SEQ ID NO: 10 - protein, 100 aa, Homo sapiens [sequence omitted]

SEQ ID NO: 11 - protein, 254 aa, Homo sapiens
NADLLAAAIRGVPLKGQGNGGSRKNTQSDRPRLQRNQCAYCKETGHWKDKCPQLKEKQGGSEQKTPDKDEGALFNLAEGLLDRRGPGSRAPKEPMVRMTVGGKDIKFLVNTGAEHSVVTTPVAPLSKKAIDIIGATGVLTKQAFCLPRTCSVGGHEVIHQFLYIPDCPLPLLGRDLLSKLRAIFLYQARLFTTEVAWNRSYHGPDSSPRGRVATLPNQTRQRDRASSGPVVAKSMRRRQPSWIGSQSSSCTQGS

SEQ ID NO: 12 - protein, 31 aa, Homo sapiens
SLFLHKTSVREVLSATIPATFLGSLTWKRGD

SEQ ID NO: 13 - protein, 257 aa, Homo sapiens
MQNEAIEQVRAICLRAWGKIQDPGTAFPINSIRQGSKEPYPDFVARLQDAAQKSITDDNARKVIVELMAYENANPECQSAIKPLKGKVPAGVDVITEYVKACDGIGGAMHKAMLMAQAMRGLTLGGQVRTFGKKCYNCGQIGHLKRSCPVLNKQNIINQAITAKNKKPSGLCPKCGKGKHWANQCHSKFDKDGQPLSGNRKRGQPQAPQQTGAFPVQLFVPQGFQGQQPLQKIPPLQGVSQLQQSNSCPAPQQAAPQ

SEQ ID NO: 14 - protein, 95 aa, Homo sapiens
MEWIKYSICTLNKSNCYACAHGRPEAQIVPFPLRWSSSRPSMGCMVALFQDSTAWGNISCQALSLLYPEVQHPAGQPPRAIQLPSPNVSFISCLS

SEQ ID NO: 15 - protein, 529 aa, Homo sapiens [sequence omitted]

SEQ ID NO: 16 - protein, 1142 aa, Homo sapiens
MGDKDMPTAGMPSLLQSSSESPQSCPEGEDSQSPLQIPQSSPESDDTLYPLQSPQSRSEGEDSSDPLQRPPEGKDSQSPLQIPQSSPEGDDTQSPLQNSQSSPEGKDSLSPLEISQSPPEGEDVQSPLQNPASSFFSSALLSIFQSSPESTQSPFEGFPQSVLQIPVSAASSSTLVSIFQSSPESTQSPFEGFPQSPLQIPVSRSFSSTLLSIFQSSPERTQSTFEGFAQSPLQIPVSPSSSSTLLSLFQSFSERTQSTFEGFAQSSLQIPVSPSFSSTLVSLFQSSPERTQSTFEGFPQSPLQIPVSSSSSSTLLSLFQSSPERTHSTFEGFPQSLLQIPMTSSFSSTLLSIFQSSPESAQSTFEGFPQSPLQIPGSPSFSSTLLSLFQSSPERTHSTFEGFPQSPLQIPMTSSFSSTLLSILQSSPESAQSAFEGFPQSPLQIPVSSSFSYTLLSLFQSSPERTHSTFEGFPQSPLQIPVSSSSSSSTLLSLFQSSPECTQSTFEGFPQSPLQIPQSPPEGENTHSPLQIVPSLPEWEDSLSPHYFPQSPPQGEDSLSPHYFPQSPPQGEDSLSPHYFPQSPQGEDSLSPHYFPQSPPQGEDSMSPLYFPQSPLQGEEFQSSLQSPVSICSSSTPSSLPQSFPESSQSPPEGPVQSPLHSPQSPPEGMHSQSPLQSPESAPEGEDSLSPLQIPQSPLEGEDSLSSLHFPQSPPEWEDSLSPLHFPQFPPQGEDFQSSLQSPVSICSSSTSLSLPQSFPESPQSPPEGPAQSPLQRPVSSFFSYTLASLLQSSHESPQSPPEGPAQSPLQSPVSSFPSSTSSSLSQSSPVSSFPSSTSSSLSKSSPESPLQSPVISFSSSTSLSPFSEESSSPVDEYTSSSDTLLESDSLTDSESLIESEPLFTYTLDEKVDELARFLLLKYQVKQPITKAEMLTNVISRYTGYFPVIFRKAREFIEILFGISLREVDPDDSYVFVNTLDLTSEGCLSDEQGMSQNRLLILILSIIFIKGTYASEEVIWDVLSGIGVRAGREHFAFGEPRELLTKVWVQEHYLEYREVPNSSPPRYEFLWGPRAHSEVIKRKVVEFLAMLKNTVPITFPSSYKDALKDVEERAQAIIDTTDDSTATESASSSVMSPSFSSE

SEQ ID NO: 17 - protein, 369 aa, Homo sapiens
MPRAPKRQRCMPEEDLQSQSETQGLEGAQAPLAVEEDASSSTSTSSSFPSSFPSSSSSSSSSCYPLIPSTPEEVSADDETPNPPQSAQIACSSPSVVASLPLDQSDEGSSSQKEESPSTLQVLPDSESLPRSEIDEKVTDLVQFLLFKYQMKEPITKAEILESVIRNYEDHFPLLFSEASECMLLVFGIDVKEVDPTGHSFVLVTSLGLTYDGMLSDVQSMPKTGILILILSIVFIEGYCTPEEVIWEALNMMGLYDGMEHLIYGEPRKLLTQDWVQENYLEYRQVPGSDPARYEFLWGPRAHAEIRKMSLLKFLAKVNGSDPRSFPLWYEEALKDEEERAQDRIATTDDTTAMASASSSATGSFSYPE

SEQ ID NO: 18 - protein, 336 aa, Homo sapiens
MPRGQASKRRAREKRRQARGEDQCLGGAQATAAEKEKLPSSSSPACQSPPQSFPNAGIPQESQRASYPSSPASAVSLTSSDEGAKGQKGESPNSFHGPSSSESTGRDLLNTKTGELVQFLLNKYIRKEPITREAMLKVINRKYKQHFPEILRRSTENVEVVFGLYLKEMDPSRQSYVLVGKLDFPNQGSLSDGGGFPLSGLLMVLLSTIFMHGNRATEEEMWECLNALGMYKGRKHFIYGEPQELVTKDLVREGYLEYQQVPSSDPPRYEFLWGPRARAETSKMKVLEFVAKLNDTVASTYKSRYEEALREEEEQARARAVARDSARARASRSFQP

SEQ ID NO: 19 - protein, 345 aa, Homo sapiens [sequence omitted]

SEQ ID NO: 20 - protein, 324 aa, Homo sapiens
MSQDQESPRCTHDQHLQTFSETQSLEVAQVSKALEKTLLSSSHPLVPGKLKEAPAAKAESPLEVPQSFCSSSIAVTTTSSSESDEASSNQEEEDSPSSSEDTSDPRNVPADALDQKVAFLVNFMLHKCQMKKPITKADMLKIIIKDDESHFSEILLRASEHLEMIFGLDVVEVDPTTHCYGLFIKLGLTYDGMLSGEKGVPKTGLLIIVLGVIFMKGNRATEEEVWEVLNLTGVYSGKKHFIFGEPRMLITKDFVKEKYLEYQQVANSDPARYEFLWGPRAKAETSKMKVLEFVAKVHGSYPHSFPSQYAEALKEEEERARARI

SEQ ID NO: 21 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 22 - protein, 318 aa, Homo sapiens
MLLGQKSQRYKAEEGLQAQGEAPGLMDVQIPTAEEQKAASSSSTLIMGTLEEVTDSGSPSPPQSPEGASSSLTVTDSTLWSQSDEGSSSNEEEGPSTSPDPAHLESLFREALDEKVAELVRFLLRKYQIKEPVTKAEMLESVIKNYKNHFPDIFSKASECMQVIFGIDVKEVDPAGHSYILVTCLGLSYDGLLGDDQSTPKTGLLIIVLGMILMEGSRAPEEAIWEALSVMGLYDGREHSVYWKLRKLLTQEWVQENYLEYRQAPGSDPVRYEFLWGPRALAETSYVKVLEHVVRVNARVRISYPSLHEEALGEEKGV

SEQ ID NO: 23 - protein, 346 aa, Homo sapiens
MPRGQKSKLRAREKRQRTRGQTQDLKVGQPTAAEKEESPSSSSSVLRDTASSSLAFGIPQEPQREPPTTSAAAAMSCTGSDKGDESQDEENASSSQASTSTERSLKDSLTRKTKMLVQFLLYKYKMKEPTTKAEMLKIISKKYKEHFPEIFRKVSQRTELVFGLALKEVNPTTHSYILVSMLGPNDGNQSSAWTLPRNGLLMPLLSVIFLNGNCAREEEIWEFLNMLGIYDGKRHLIFGEPRKLITQDLVQEKYLEYQQVPNSDPPRYQFLWGPRAHAETSKMKVLEFLAKVNDTTPNNFPLLYEEALRDEEERAGARPRVAARRGTTAMTSAYSRATSSSSSQPM

SEQ ID NO: 24 - protein, 189 aa, Homo sapiens
MTDKTEKVAVDPETVFKRPRECDSPSYQKRQRMALLARKQGAGDSLIAGSAMSKEKKLMTGHAIPPSQLDSQIDDFTGFSKDGMMQKPGSNAPVGGNVTSNFSGDDLECRGIASSPKSQQEINADIKCQVVKEIRCLGRKYEKIFEMLEGVQGPTAVRKRFFESIIKEAARCMRRDFVKHLKKKLKRMI

SEQ ID NO: 25 - protein, 532 aa, Homo sapiens
MQGPWVLLLLGLRLQLSLGIIPVEEENPDFWNRQAAEALGAAKKLQPAQTAAKNLIIFLGDGMGVSTVTAARILKGQKKDKLGPETFLAMDRFPYVALSKTYSVDKHVPDSGATATAYLCGVKGNFQTIGLSAAARFNQCNTTRGNEVISVMNRAKKAGKSVGVVTTTRVQHASPAGAYAHTVNRNWYSDADVPASARQEGCQDIATQLISNMDIDVILGGGRKYMFPMGTPDPEYPDDYSQGGTRLDGKNLVQEWLAKHQGARYVWNRTELLQASLDPSVTHLMGLFEPGDMKYEIHRDSTLDPSLMEMTEAALLLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDGETHAGEDVAVFARGPQAHLVHGVQEQTFIAHVMAFAACLEPYTACDLAPRAGTTDAAHPGPSVVPALLPLLAGTLLLLGTATAP

SEQ ID NO: 26 - protein, 471 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 27 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 28 - protein, 552 aa, Homo sapiens
MSPLWWGFLLSCLGCKILPGAQGQFPRVCMTVDSLVNKECCPRLGAESANVCGSQQGRGQCTEVRADTRPWSGPYILRNQDDRELWPRKFFHRTCKCTGNFAGYNCGDCKFGWTGPNCERKKPPVIRQNIHSLSPQEREQFLGALDLAKKRVHPDYVITTQHWLGLLGPNGTQPQFANCSVYDFFVWLHYYSVRDTLLGPGRPYRAIDFSHQGPAFVTWHRYHLLCLERDLQRLIGNESFALPYWNFATGRNECDVCTDQLFGAARPDDPTLISRNSRFSSWETVCDSLDDYNHLVTLCNGTYEGLLRRNQMGRNSMKLPTLKDIRDCLSLQKFDNPPFFQNSTFSFRNALEGFDKADGTLDSQVMSLHNLVHSFLNGTNALPHSAANDPIFVVISNRLLYNATTNILEHVRKEKATKELPSLHVLVLHSFTDAIFDEWMKRFNPPADAWPQELAPIGHNRMYNMVPFFPPVTNEELFLTSDQLGYSYAIDLPVSVEETPGWPTTLLVVMGTLVALVGLFVLLAFLQYRRLRKGYTPLMETHLSSKRYTEEA

SEQ ID NO: 29 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 30 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 31 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 32 - protein, 224 aa, Homo sapiens
MVAAVLLGLSWLCSPLGALVLDFNNIRSSADLHGARKGSQCLSDTDCNTRKFCLQPRDEKPFCATCRGLRRRCQRDAMCCPGTLCVNDVCTTMEDATPILERQLDEQDGTHAEGTTGHPVQENQPKRKPSIKKSQGRKGQEGESCLRTFDCGPGLCCARHFWTKICKPVLLEGQVCSRRGHKDTAQAPEIFQRCDCGPGLLCRSQLTSNRQHARLRVCQKIEKL

SEQ ID NO: 33 - protein, 193 aa, Homo sapiens
MDGGTLPRSAPPAPPVPVGCAARRRPASPELLRCSRRRRPATAETGGGAAAVARRNERERNRVKLVNLGFQALRQHVPHGGASKKLSKVETLRSAVEYIRALQRLLAEHDAVRNALAGGLRPQAVRPSAPRGPPGTTPVAASPSRASSSPGRGGSSEPGSPRSAYSSDDSGCEGALSPAERELLDFSSWLGGY

SEQ ID NO: 34 - protein, 139 aa, Homo sapiens
MSWRGRSTYYWPRPRRYVQPPEMIGPMRPEQFSDEVEPATPEEGEPATQRQDPAAAQEGEDEGASAGQGPKPEADSQEQGHPQTGCECEDGPDGQEMDPPNPEEVKTPEEEMRSHYVAQTGILWLLMNNCFLNLSPRKP

SEQ ID NO: 35 - protein, 116 aa, Homo sapiens [sequence omitted]

SEQ ID NO: 36 - protein, 530 aa, Homo sapiens
MGSNSGQAGRHIYKSLADDGPFDSVEPPKRPTSRLIMHSMAMFGREFCYAVEAAYVTPVLLSVGLPSSLYSIVWFLSPILGFLLQPVVGSASDHCRSRWGRRRPYILTLGVMMLVGMALYLNGATVVAALIANPRRKLVWAISVTMIGVVLFDFAADFIDGPIKAYLFDVCSHQDKEKGLHYHALFTGFGGALGYLLGAIDWAHLELGRLLGTEFQVMFFFSALVLTLCFTVHLCSISEAPLTEVAKGIPPQQTPQDPPLSSDGMYEYGSIEKVKNGYVNPELAMQGAKNKNHAEQTRRAMTLKSLLRALVNMPPHYRYLCISHLIGWTAFLSNMLFFTDFMGQIVYRGDPYSAHNSTEFLIYERGVEVGCWGFCINSVFSSLYSYFQKVLVSYIGLKGLYFTGYLLFGLGTGFIGLFPNVYSTLVLCSLFGVMSSTLYTVPFNLITEYHREEEKERQQAPGGDPDNSVRGKGMDCATLTCMVQLAQILVGGGLGFLVNTAGTVVVVVITASAVALIGCCFVALFVRYVD

SEQ ID NO: 37 - protein, 130 aa, Homo sapiens
MQAPWAGNRGWAGTREEVRDMSEHVTRSQSSERGNDQESSQPVGPVIVQQPTEEKRQEEEPPTDNQGIAPSGEIKNEGAPAVQGTDVEAFQQELALLKIEDAPGDGPDVREGTLPTFDPTKVLEAGEGQL

SEQ ID NO: 38 - protein, 111 aa, Homo sapiens
MSELLRARSQSSERGNDQESSQPVGSVIVQEPTEEKRQEEEPPTDNQGIAPSGEIENQAVPAFQGPDMEAFQQELALLKIEDEPGDGPDVREGIMPTFDLTKVLEAGDAQP

SEQ ID NO: 39 - protein, 668 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPVPGILLTGQEAGLGQVPLIVGILLVLMAVVLASLIYRRRLMKQDFSVPQLPHSSSHWLRLPRIFCSCPIGENSPLLSGQQV

SEQ ID NO: 40 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 41 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 42 - protein, 429 aa, Epstein-Barr virus
MAEAYPGGAHAALASRRSSFRNSLRRLRPTEKPDTSFMRGVWKYEIFPSYVRVTNKQVLQLDAQCQELPPCPSVGQILSFKLPSFSFNTTTYGSRYFTVAFLFFGAEDNEVFLKPFFVMHSDQDIVLSVLNPRSLFIEKGKFTWYIVPIRLVKNPYLYLQILPGQSDIQLTRSCTQSGDKLNTSEPQIFLSGSPVTSQDECLPYLLAQHTPPFLKSYARIHTFPGKVCPVNAIRRGKGYVRVSVDTPDLKREGPLNVKVGMTLLDDVIIAFRYNPYPKSHWRWDGESTDIRYFGSPVIIPPNFITELEYNNTYEAPLSSKITAIVVSHSSNPVFYVYPQEWKPGQTLKLTVRNISNNPITIVTGQSMAQAFFIYAGDPSISTIMRRYIQRQGCALTLPGNIVVESSSLPTFERINKTFNGNIVASEGTL

SEQ ID NO: 43 - protein, 1015 aa, Epstein-Barr virus
MSGGLFYNPFLRPNKGLLKKPDKEYLRLIPKCFQTPGAAGVVDVRGPQPPLCFYQDSLTVVGGDEDGKGMWWRQRAQEGTARPEADTHGSPLDFHVYDILETVYTHEKCAVIPSDKQGYVVPCGIVIKLLGRRKADGASVCVNVFGQQAYFYASAPQGLDVEFAVLSALKASTFDRRTPCRVSVEKVTRRSIMGYGNHAGDYHKITLSHPNSVCHVATWLQDKHGCRIFEANVDATRRFVLDNDFVTFGWYSCRRAIPRLQHRDSYAELEYDCEVGDLSVRREDSSWPSYQALAFDIECLGEEGFPTATNEADLILQISCVLWSTGEEAGRYRRILLTLGTCEDIEGVEVYEFPSELDMLYAFFQLIRDLSVEIVTGYNVANFDWPYILDRARHIYSINPASLGKIRAGGVCEVRRPHDAGKGFLRANTKVRITGLIPIDMYAVCRDKLSLSDYKLDTVARHLLGAKKEDVHYKEIPRLFAAGPEGRRRLGMYCVQDSALVMDLLNHFVIHVEVAEIAKIAHIPCRRVLDDGQQIRVFSCLLAAAQKENFILPMPSASDRDGYQGATVIQPLSGFYNSPVLVVDFASLYPSIIQAHNLCYSTMITPGEEHRLAGLRPGEDYESFRLTGGVYHFVKKHVHESFLASLLTSWLAKRKAIKKLLAACEDPRQRTILDKQQLAIKCTCNAVYGFTGVANGLFPCLSIAETVTLQGRTMLERAKAFVEALSPANLQALAPSPDAWAPLNPEGQLRVIYGDTDSLFIECRGFSESETLRFAEALAAHTTRSLFVAPISLEAEKTFSCLMLITKKRYVGVLTDGKTLMKGVELVRKTACKFVQTRCRRVLDLVLADARVKEAASLLSHRPFQESFTQGLPVGFLPVIDILNQAYTDLREGRVPMGELCFSTELSRKLSAYKSTQMPHLAVYQKFVERNEELPQIHDRIQYVFVEPKGGVKGARKTEMAEDPAYAERHGVPVAVDHYFDKLLQGAANILQCLFDNNSGAALSVLQNFTARPPF

SEQ ID NO: 44 - protein, 103 aa, Epstein-Barr virus
MAGARRRARCPASAGCAYSARPPPLSTRGRRISAGSGQPRWWPWGSPPPLDTRYRRPGPGRRARSCLHAGPRGRPPHSRTRARRTSPGAGGGGWRGGSCTSQR

SEQ ID NO: 45 - protein, 126 aa, Epstein-Barr virus
MSMPPKGFLKKEMKPETRLLNKPPTVLTRPAMFCAWKLYSRKMPSRSKTLEARCSSRPPCDSPACQTRDTGCPRRSGTGRRGWRARRLGKESWFADAWRMARYWGCAVKAAAQSAFSASTASPEEL

SEQ ID NO: 46 - protein, 857 aa, Epstein-Barr virus
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPPAARGSTSAAVLRRRRRDAGNATTPVPPAAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 47 - protein, 685 aa, Epstein-Barr virus
MSGLLAAAYSQVYALAVELSVCARLDPRSLDVAAVVRNAGLLAELEAILLPRLRRQNDRACSALSLELVHLLENSREASAALLAPGRKGTRVPPLRTPSVAYSVEFYGGHKVDVSLCLINDIEILMKRINSVFYCMSHTMGLESLERALDLLGRFRGVSPIPDPRLYITSVPCWRCVGELMVLPNHGNPSTAEGTHVSCNHLAVPVNPEPVSGLFENEVRQAGLGHLLEAEEKARPGGPEEGAVPGPGRPEAEGATRALDTYNVFSTVPPEVAELSELLYWNSGGHAIGATGQGEGGGHSRLSALFARERRLALVRRACEEALAGARLTHLFDAVAPGATERLFCGGVYSSSGDAVEALKADCAAAFTAHPQYRAILQKRNELYTRLNRAMQRLGRGEEEASRESPEVPRPAGAREPGPSGALSDALKRKEQYLRQVATEGLAKLQSCLAQQSETLTETLCLRVWGDVVYWELARMRNHFLYRRAFVSGPWEDRRAGEGAAFENSKYIKTHLFTQTLSSEHLHALTHSLYTFITGPLAEESGLFPPPSNVALARCCDAAGTLPHQKAFLTSLIWPGIEPSDWIETSFNSFYSVPGGSLASSQQILCRALREAVLTVSLYNKTWGRSLILRRADAVSPGQALPPDGLYLTYDSDRPLILLYKGRGWVFKDLYALLYLHLQMRDDSA

SEQ ID NO: 48 - protein, 471 aa, Epstein-Barr virus
APGYAVEAVEGGLYPVARLDAWPYQGSQERLLVRQRTCGVTAASQGHVAGWGKEPALLRQGPRDEGVQAVRQRVQVLRAQGLGKQVCFDVLGILKGGTLAGAPVLPGTRDEGPSVEEVVAHAGQLPVDHVPPDAQAQGLGQGLALLRQAGLQLGQTLGGHLAQVLLLALERVREGAGRAGLSCPSRPGHLRALPGRLLLASAQPLHGSVEPRVELVPLLQDGPVLGVRREGGGAVRLQRLHRVARGAVDPAAEEPLCGPGSHGIKQVSQPCPRQRLLAGPPHQGQATLPGKQGREAGMSATLPLPRCTDSMAARVPIEELREFRHLRGHCREDVVGVQRSGRPLCLRPPRARDRALLWAARPRLLLSLQQVPEPSLPDFILKQSRDRLRIHRHRQVVTGDVGPLCRGRVAVVGQNHQLAHTAPAGHRGDVEARVWDGTYAPKAAQQIQGPFQALQPHGVRHAIKHAIDSLH

SEQ ID NO: 49 - protein, 9 aa, Human papillomavirus type 16
CMSCCRSSR

SEQ ID NO: 50 - protein, 9 aa, Human papillomavirus type 17
CPEEKQRHL

SEQ ID NO: 51 - protein, 9 aa, Human papillomavirus type 18
CVYCKQQLL

SEQ ID NO: 52 - protein, 10 aa, Human papillomavirus type 19
CVYCKQQLLR

SEQ ID NO: 53 - protein, 9 aa, Human papillomavirus type 20
CYSLYGTTL

SEQ ID NO: 54 - protein, 9 aa, Human papillomavirus type 21
DKKQRFHNI

SEQ ID NO: 55 - protein, 9 aa, Human papillomavirus type 22
EYRHYCYSL

SEQ ID NO: 56 - protein, 9 aa, Human papillomavirus type 23
FAFRDLCIV

SEQ ID NO: 57 - protein, 9 aa, Human papillomavirus type 24
IILECVYCK

SEQ ID NO: 58 - protein, 9 aa, Human papillomavirus type 25
ISEYRHYCY

SEQ ID NO: 59 - protein, 9 aa, Human papillomavirus type 26
IVYRDGNPY

SEQ ID NO: 60 - protein, 10 aa, Human papillomavirus type 27
IVYRDGNPYA

SEQ ID NO: 61 - protein, 9 aa, Human papillomavirus type 28
KFYSKISEY

SEQ ID NO: 62 - protein, 10 aa, Human papillomavirus type 29
KISEYRHYCY

SEQ ID NO: 63 - protein, 9 aa, Human papillomavirus type 30
KLPQLCTEL

SEQ ID NO: 64 - protein, 9 aa, Human papillomavirus type 31
LIRCINCQK

SEQ ID NO: 65 - protein, 10 aa, Human papillomavirus type 32
LLIRCINCQK

SEQ ID NO: 66 - protein, 9 aa, Human papillomavirus type 33
MHQKRTAMF

SEQ ID NO: 67 - protein, 10 aa, Human papillomavirus type 34
NPYAVCDKCL

SEQ ID NO: 68 - protein, 10 aa, Human papillomavirus type 35
QYNKPLCDLL

SEQ ID NO: 69 - protein, 9 aa, Human papillomavirus type 36
RFHNIRGRW

SEQ ID NO: 70 - protein, 9 aa, Human papillomavirus type 37
RGRWTGRCM

SEQ ID NO: 71 - protein, 9 aa, Human papillomavirus type 38
RPRKLPQLC

SEQ ID NO: 72 - protein, 10 aa, Human papillomavirus type 39
RPRKLPQLCT

SEQ ID NO: 73 - protein, 10 aa, Human papillomavirus type 40
SEYRHYCYSL

SEQ ID NO: 74 - protein, 10 aa, Human papillomavirus type 41
SSRTRRETQL

SEQ ID NO: 75 - protein, 10 aa, Human papillomavirus type 42
TIHDIILECV

SEQ ID NO: 76 - protein, 9 aa, Human papillomavirus type 43
TTLEQQYNK

SEQ ID NO: 77 - protein, 9 aa, Human papillomavirus type 44
VYDFAFRDL

SEQ ID NO: 78 - protein, 10 aa, Human papillomavirus type 45
GIVCPICSQK

SEQ ID NO: 79 - protein, 9 aa, Human papillomavirus type 46
GTLGIVCPI

SEQ ID NO: 80 - protein, 10 aa, Human papillomavirus type 47
HGDTPTLHEY

SEQ ID NO: 81 - protein, 9 aa, Human papillomavirus type 48
IVCPICSQK

SEQ ID NO: 82 - protein, 9 aa, Human papillomavirus type 49
LLMGTLGIV

SEQ ID NO: 83 - protein, 9 aa, Human papillomavirus type 50
RAHYNIVTF

SEQ ID NO: 84 - protein, 9 aa, Human papillomavirus type 51
RLCVQSTHV

SEQ ID NO: 85 - protein, 10 aa, Human papillomavirus type 52
TLEDLLMGTL

SEQ ID NO: 86 - protein, 9 aa, Human papillomavirus type 53
TLHEYMLDL

SEQ ID NO: 87 - protein, 9 aa, Human papillomavirus type 54
TPTLHEYML

SEQ ID NO: 88 - protein, 9 aa, Human papillomavirus type 55
YMLDLQPET

SEQ ID NO: 89 - protein, 10 aa, Human papillomavirus type 56
YMLDLQPETT

SEQ ID NO: 90 - protein, 10 aa, Epstein-Barr virus
AALASRRSSF

SEQ ID NO: 91 - protein, 9 aa, Epstein-Barr virus
ALASRRSSF

SEQ ID NO: 92 - protein, 10 aa, Epstein-Barr virus
ALASRRSSFR

SEQ ID NO: 93 - protein, 10 aa, Epstein-Barr virus
ALTLPGNIVV

SEQ ID NO: 94 - protein, 9 aa, Epstein-Barr virus
APLSSKITA

SEQ ID NO: 95 - protein, 10 aa, Epstein-Barr virus
APLSSKITAI

SEQ ID NO: 96 - protein, 9 aa, Epstein-Barr virus
AQHTPPFLK

SEQ ID NO: 97 - protein, 10 aa, Epstein-Barr virus
AYPGGAHAAL

SEQ ID NO: 98 - protein, 10 aa, Epstein-Barr virus
CPSVGQILSF

SEQ ID NO: 99 - protein, 9 aa, Epstein-Barr virus
EVFLKPFFV

SEQ ID NO: 100 - protein, 10 aa, Epstein-Barr virus
FFGAEDNEVF

SEQ ID NO: 101 - protein, 10 aa, Epstein-Barr virus
FIEKGKFTWY

SEQ ID NO: 102 - protein, 9 aa, Epstein-Barr virus
FIYAGDPSI

SEQ ID NO: 103 - protein, 9 aa, Epstein-Barr virus
FLSGSPVTS

SEQ ID NO: 104 - protein, 10 aa, Epstein-Barr virus
FMRGVWKYEI

SEQ ID NO: 105 - protein, 10 aa, Epstein-Barr virus
FTVAFLFFGA

SEQ ID NO: 106 - protein, 10 aa, Epstein-Barr virus
FTWYIVPIRL

SEQ ID NO: 107 - protein, 10 aa, Epstein-Barr virus
FVMHSDQDIV

SEQ ID NO: 108 - protein, 9 aa, Epstein-Barr virus
GPLNVKVGM

SEQ ID NO: 109 - protein, 10 aa, Epstein-Barr virus
GQSMAQAFFI

SEQ ID NO: 110 - protein, 10 aa, Epstein-Barr virus
HSDQDIVLSV

SEQ ID NO: 111 - protein, 9 aa, Epstein-Barr virus
HSSNPVFYV

SEQ ID NO: 112 - protein, 10 aa, Epstein-Barr virus
HSSNPVFYVY

SEQ ID NO: 113 - protein, 10 aa, Epstein-Barr virus
HTFPGKVCPV

SEQ ID NO: 114 - protein, 10 aa, Epstein-Barr virus
IAFRYNPYPK

SEQ ID NO: 115 - protein, 10 aa, Epstein-Barr virus
IIPPNFITEL

SEQ ID NO: 116 - protein, 10 aa, Epstein-Barr virus
ILPGQSDIQL

SEQ ID NO: 117 - protein, 9 aa, Epstein-Barr virus
IPPNFITEL

SEQ ID NO: 118 - protein, 10 aa, Epstein-Barr virus
ITELEYNNTY

SEQ ID NO: 119 - protein, 10 aa, Epstein-Barr virus
IVVSHSSNPV

SEQ ID NO: 120 - protein, 9 aa, Epstein-Barr virus
KFTWYIVPI

SEQ ID NO: 121 - protein, 9 aa, Epstein-Barr virus
KLNTSEPQI

SEQ ID NO: 122 - protein, 9 aa, Epstein-Barr virus
KLPSFSFNT

SEQ ID NO: 123 - protein, 10 aa, Epstein-Barr virus
KLPSFSFNTT

SEQ ID NO: 124 - protein, 9 aa, Epstein-Barr virus
KNPYLYLQI

SEQ ID NO: 125 - protein, 10 aa, Epstein-Barr virus
KPDTSFMRGV

SEQ ID NO: 126 - protein, 10 aa, Epstein-Barr virus
KPGQTLKLTV

SEQ ID NO: 127 - protein, 9 aa, Epstein-Barr virus
KSYARIHTF

SEQ ID NO: 128 - protein, 10 aa, Epstein-Barr virus
KVCPVNAIRR

SEQ ID NO: 129 - protein, 9 aa, Epstein-Barr virus
KYEIFPSYV

SEQ ID NO: 130 - protein, 10 aa, Epstein-Barr virus
LAQHTPPFLK

SEQ ID NO: 131 - protein, 10 aa, Epstein-Barr virus
LDDVIIAFRY

SEQ ID NO: 132 - protein, 10 aa, Epstein-Barr virus
LFIEKGKFTW

SEQ ID NO: 133 - protein, 9 aa, Epstein-Barr virus
LLAQHTPPF

SEQ ID NO: 134 - protein, 10 aa, Epstein-Barr virus
LLAQHTPPFL

SEQ ID NO: 135 - protein, 9 aa, Epstein-Barr virus
LLDDVIIAF

SEQ ID NO: 136 - protein, 9 aa, Epstein-Barr virus
LPGQSDIQL

SEQ ID NO: 137 - protein, 10 aa, Epstein-Barr virus
LPPCPSVGQI

SEQ ID NO: 138 - protein, 9 aa, Epstein-Barr virus
LPSFSFNTT

SEQ ID NO: 139 - protein, 10 aa, Epstein-Barr virus
LPSFSFNTTT

SEQ ID NO: 140 - protein, 10 aa, Epstein-Barr virus
LQLDAQCQEL

SEQ ID NO: 141 - protein, 9 aa, Epstein-Barr virus
LTLPGNIVV

SEQ ID NO: 142 - protein, 9 aa, Epstein-Barr virus
MAQAFFIYA

SEQ ID NO: 143 - protein, 9 aa, Epstein-Barr virus
NPYLYLQIL

SEQ ID NO: 144 - protein, 9 aa, Epstein-Barr virus
NSLRRLRPT

SEQ ID NO: 145 - protein, 9 aa, Epstein-Barr virus
NTTTYGSRY

SEQ ID NO: 146 - protein, 10 aa, Epstein-Barr virus
NTYEAPLSSK

SEQ ID NO: 147 - protein, 9 aa, Epstein-Barr virus
PFLKSYARI

SEQ ID NO: 148 - protein, 9 aa, Epstein-Barr virus
PSYVRVTNK

SEQ ID NO: 149 - protein, 9 aa, Epstein-Barr virus
PYPKSHWRW

SEQ ID NO: 150 - protein, 9 aa, Epstein-Barr virus
QIFLSGSPV

SEQ ID NO: 151 - protein, 9 aa, Epstein-Barr virus
QLDAQCQEL

SEQ ID NO: 152 - protein, 9 aa, Epstein-Barr virus
QSMAQAFFI

SEQ ID NO: 153 - protein, 10 aa, Epstein-Barr virus
QSMAQAFFIY

SEQ ID NO: 154 - protein, 9 aa, Epstein-Barr virus
RLVKNPYLY

SEQ ID NO: 155 - protein, 10 aa, Epstein-Barr virus
RLVKNPYLYL

SEQ ID NO: 156 - protein, 10 aa, Epstein-Barr virus
RPTEKPDTSF

SEQ ID NO: 157 - protein, 9 aa, Epstein-Barr virus
RSLFIEKGK

SEQ ID NO: 158 - protein, 9 aa, Epstein-Barr virus
RSSFRNSLR

SEQ ID NO: 159 - protein, 10 aa, Epstein-Barr virus
RSSFRNSLRR

SEQ ID NO: 160 - protein, 10 aa, Epstein-Barr virus
RVSVDTPDLK

SEQ ID NO: 161 - protein, 9 aa, Epstein-Barr virus
RYFGSPVII

SEQ ID NO: 162 - protein, 9 aa, Epstein-Barr virus
RYFTVAFLF

SEQ ID NO: 163 - protein, 10 aa, Epstein-Barr virus
RYFTVAFLFF

SEQ ID NO: 164 - protein, 10 aa, Epstein-Barr virus
RYIQRQGCAL

SEQ ID NO: 165 - protein, 10 aa, Epstein-Barr virus
RYNPYPKSHW

SEQ ID NO: 166 - protein, 9 aa, Epstein-Barr virus
SFKLPSFSF

SEQ ID NO: 167 - protein, 9 aa, Epstein-Barr virus
SFMRGVWKY

SEQ ID NO: 168 - protein, 10 aa, Epstein-Barr virus
SLPTFERINK

SEQ ID NO: 169 - protein, 10 aa, Epstein-Barr virus
SLRRLRPTEK

SEQ ID NO: 170 - protein, 9 aa, Epstein-Barr virus
SMAQAFFIY

SEQ ID NO: 171 - protein, 10 aa, Epstein-Barr virus
SMAQAFFIYA

SEQ ID NO: 172 - protein, 10 aa, Epstein-Barr virus
SPVTSQDECL

SEQ ID NO: 173 - protein, 9 aa, Epstein-Barr virus
SQDECLPYL

SEQ ID NO: 174 - protein, 10 aa, Epstein-Barr virus
SQDECLPYLL

SEQ ID NO: 175 - protein, 10 aa, Epstein-Barr virus
SRRSSFRNSL

SEQ ID NO: 176 - protein, 9 aa, Epstein-Barr virus
SSFRNSLRR

SEQ ID NO: 177 - protein, 9 aa, Epstein-Barr virus
SSNPVFYVY

SEQ ID NO: 178 - protein, 9 aa, Epstein-Barr virus
SVGQILSFK

SEQ ID NO: 179 - protein, 9 aa, Epstein-Barr virus
TFERINKTF

SEQ ID NO: 180 - protein, 9 aa, Epstein-Barr virus
TIMRRYIQR

SEQ ID NO: 181 - protein, 9 aa, Epstein-Barr virus
TLLDDVIIA

SEQ ID NO: 182 - protein, 10 aa, Epstein-Barr virus
TLLDDVIIAF

SEQ ID NO: 183 - protein, 10 aa, Epstein-Barr virus
TPDLKREGPL

SEQ ID NO: 184 - protein, 9 aa, Epstein-Barr virus
TSFMRGVWK

SEQ ID NO: 185 - protein, 10 aa, Epstein-Barr virus
TSFMRGVWKY

SEQ ID NO: 186 - protein, 9 aa, Epstein-Barr virus
TSQDECLPY

SEQ ID NO: 187 - protein, 10 aa, Epstein-Barr virus
TTYGSRYFTV

SEQ ID NO: 188 - protein, 9 aa, Epstein-Barr virus
TVAFLFFGA

SEQ ID NO: 189 - protein, 10 aa, Epstein-Barr virus
TVRNISNNPI

SEQ ID NO: 190 - protein, 9 aa, Epstein-Barr virus
TWYIVPIRL

SEQ ID NO: 191 - protein, 10 aa, Epstein-Barr virus
TYEAPLSSKI

SEQ ID NO: 192 - protein, 9 aa, Epstein-Barr virus
TYGSRYFTV

SEQ ID NO: 193 - protein, 9 aa, Epstein-Barr virus
VFLKPFFVM

SEQ ID NO: 194 - protein, 9 aa, Epstein-Barr virus
VFYVYPQEW

SEQ ID NO: 195 - protein, 10 aa, Epstein-Barr virus
VFYVYPQEWK

SEQ ID NO: 196 - protein, 9 aa, Epstein-Barr virus
VLNPRSLFI

SEQ ID NO: 197 - protein, 10 aa, Epstein-Barr virus
VLSVLNPRSL

SEQ ID NO: 198 - protein, 9 aa, Epstein-Barr virus
VMHSDQDIV

SEQ ID NO: 199 - protein, 10 aa, Epstein-Barr virus
VMHSDQDIVL

SEQ ID NO: 200 - protein, 10 aa, Epstein-Barr virus
VSHSSNPVFY

SEQ ID NO: 201 - protein, 10 aa, Epstein-Barr virus
VTSQDECLPY

SEQ ID NO: 202 - protein, 9 aa, Epstein-Barr virus
VVSHSSNPV

SEQ ID NO: 203 - protein, 9 aa, Epstein-Barr virus
WYIVPIRLV

SEQ ID NO: 204 - protein, 9 aa, Epstein-Barr virus
YARIHTFPG

SEQ ID NO: 205 - protein, 10 aa, Epstein-Barr virus
YARIHTFPGK

SEQ ID NO: 206 - protein, 9 aa, Epstein-Barr virus
YFTVAFLFF

SEQ ID NO: 207 - protein, 9 aa, Epstein-Barr virus
YIQRQGCAL

SEQ ID NO: 208 - protein, 9 aa, Epstein-Barr virus
YIVPIRLVK

SEQ ID NO: 209 - protein, 10 aa, Epstein-Barr virus
YLLAQHTPPF

SEQ ID NO: 210 - protein, 9 aa, Epstein-Barr virus
YLYLQILPG

SEQ ID NO: 211 - protein, 10 aa, Epstein-Barr virus
YLYLQILPGQ

SEQ ID NO: 212 - protein, 9 aa, Epstein-Barr virus
YPGGAHAAL

SEQ ID NO: 213 - protein, 10 aa, Epstein-Barr virus
YPGGAHAALA

SEQ ID NO: 214 - protein, 10 aa, Epstein-Barr virus
YVRVTNKQVL

SEQ ID NO: 215 - protein, 9 aa, Epstein-Barr virus
AGRYRRILL

SEQ ID NO: 216 - protein, 9 aa, Epstein-Barr virus
AIKCTCNAV

SEQ ID NO: 217 - protein, 10 aa, Epstein-Barr virus
ALAAHTTRSL

SEQ ID NO: 218 - protein, 9 aa, Epstein-Barr virus
ALAFDIECL

SEQ ID NO: 219 - protein, 10 aa, Epstein-Barr virus
ALAPSPDAWA

SEQ ID NO: 220 - protein, 9 aa, Epstein-Barr virus
ALKASTFDR

SEQ ID NO: 221 - protein, 9 aa, Epstein-Barr virus
ALSPANLQA

SEQ ID NO: 222 - protein, 10 aa, Epstein-Barr virus
ALSPANLQAL

SEQ ID NO: 223 - protein, 10 aa, Epstein-Barr virus
ALSVLQNFTA

SEQ ID NO: 224 - protein, 9 aa, Epstein-Barr virus
APLNPEGQL

SEQ ID NO: 225 - protein, 9 aa, Epstein-Barr virus
APQGLDVEF

SEQ ID NO: 226 - protein, 10 aa, Epstein-Barr virus
APSPDAWAPL

SEQ ID NO: 227 - protein, 10 aa, Epstein-Barr virus
ASLLTSWLAK

SEQ ID NO: 228 - protein, 9 aa, Epstein-Barr virus
AVYGFTGVA

SEQ ID NO: 229 - protein, 9 aa, Epstein-Barr virus
AVYQKFVER

SEQ ID NO: 230 - protein, 10 aa, Epstein-Barr virus
AYKSTQMPHL

SEQ ID NO: 231 - protein, 9 aa, Epstein-Barr virus
CLFDNNSGA

SEQ ID NO: 232 - protein, 10 aa, Epstein-Barr virus
CLFDNNSGAA

SEQ ID NO: 233 - protein, 9 aa, Epstein-Barr virus
CLGEEGFPT

SEQ ID NO: 234 - protein, 10 aa, Epstein-Barr virus
CLGEEGFPTA

SEQ ID NO: 235 - protein, 10 aa, Epstein-Barr virus
CLSIAETVTL

SEQ ID NO: 236 - protein, 10 aa, Epstein-Barr virus
CVNVFGQQAY

SEQ ID NO: 237 - protein, 10 aa, Epstein-Barr virus
DARVKEAASL

SEQ ID NO: 238 - protein, 10 aa, Epstein-Barr virus
DLLNHFVIHV

SEQ ID NO: 239 - protein, 9 aa, Epstein-Barr virus
DLREGRVPM

SEQ ID NO: 240 - protein, 9 aa, Epstein-Barr virus
DMLYAFFQL

SEQ ID NO: 241 - protein, 10 aa, Epstein-Barr virus
DNDFVTFGWY

SEQ ID NO: 242 - protein, 9 aa, Epstein-Barr virus
DRARHIYSI

SEQ ID NO: 243 - protein, 9 aa, Epstein-Barr virus
DVRGPQPPL

SEQ ID NO: 244 - protein, 9 aa, Epstein-Barr virus
EAGRYRRIL

SEQ ID NO: 245 - protein, 10 aa, Epstein-Barr virus
ELSRKLSAYK

SEQ ID NO: 246 - protein, 9 aa, Epstein-Barr virus
EMAEDPAYA

SEQ ID NO: 247 - protein, 10 aa, Epstein-Barr virus
EYLRLIPKCF

SEQ ID NO: 248 - protein, 9 aa, Epstein-Barr virus
FLASLLTSW

SEQ ID NO: 249 - protein, 10 aa, Epstein-Barr virus
FLASLLTSWL

SEQ ID NO: 250 - protein, 10 aa, Epstein-Barr virus
FLRANTKVRI

SEQ ID NO: 251 - protein, 9 aa, Epstein-Barr virus
FLRPNKGLL

SEQ ID NO: 252 - protein, 10 aa, Epstein-Barr virus
FLRPNKGLLK

SEQ ID NO: 253 - protein, 10 aa, Epstein-Barr virus
FPTATNEADL

SEQ ID NO: 254 - protein, 9 aa, Epstein-Barr virus
FQESFTQGL

SEQ ID NO: 255 - protein, 9 aa, Epstein-Barr virus
FQLIRDLSV

SEQ ID NO: 256 - protein, 9 aa, Epstein-Barr virus
FQTPGAAGV

SEQ ID NO: 257 - protein, 10 aa, Epstein-Barr virus
FQTPGAAGVV

SEQ ID NO: 258 - protein, 9 aa, Epstein-Barr virus
FSESETLRF

SEQ ID NO: 259 - protein, 10 aa, Epstein-Barr virus
FTQGLPVGFL

SEQ ID NO: 260 - protein, 9 aa, Epstein-Barr virus
FVAPISLEA

SEQ ID NO: 261 - protein, 10 aa, Epstein-Barr virus
FVEALSPANL

SEQ ID NO: 262 - protein, 10 aa, Epstein-Barr virus
FVIHVEVAEI

SEQ ID NO: 263 - protein, 10 aa, Epstein-Barr virus
FVKKHVHESF

SEQ ID NO: 264 - protein, 9 aa, Epstein-Barr virus
FVLDNDFVT

SEQ ID NO: 265 - protein, 10 aa, Epstein-Barr virus
FVLDNDFVTF

SEQ ID NO: 266 - protein, 9 aa, Epstein-Barr virus
FVQTRCRRV

SEQ ID NO: 267 - protein, 10 aa, Epstein-Barr virus
FVQTRCRRVL

SEQ ID NO: 268 - protein, 9 aa, Epstein-Barr virus
FYASAPQGL

SEQ ID NO: 269 - protein, 9 aa, Epstein-Barr virus
GLDVEFAVL

SEQ ID NO: 270 - protein, 9 aa, Epstein-Barr virus
GLFPCLSIA

SEQ ID NO: 271 - protein, 9 aa, Epstein-Barr virus
GLFYNPFLR

SEQ ID NO: 272 - protein, 9 aa, Epstein-Barr virus
GLIPIDMYA

SEQ ID NO: 273 - protein, 10 aa, Epstein-Barr virus
GLIPIDMYAV

SEQ ID NO: 274 - protein, 9 aa, Epstein-Barr virus
GLPVGFLPV

SEQ ID NO: 275 - protein, 10 aa, Epstein-Barr virus
GLPVGFLPVI

SEQ ID NO: 276 - protein, 9 aa, Epstein-Barr virus
GMYCVQDSA

SEQ ID NO: 277 - protein, 10 aa, Epstein-Barr virus
GMYCVQDSAL

SEQ ID NO: 278 - protein, 10 aa, Epstein-Barr virus
GPEGRRRLGM

SEQ ID NO: 279 - protein, 10 aa, Epstein-Barr virus
GQQAYFYASA

SEQ ID NO: 280 - protein, 10 aa, Epstein-Barr virus
GVANGLFPCL

SEQ ID NO: 281 - protein, 9 aa, Epstein-Barr virus
GVYHFVKKH

SEQ ID NO: 282 - protein, 9 aa, Epstein-Barr virus
GYNVANFDW

SEQ ID NO: 283 - protein, 10 aa, Epstein-Barr virus
HIYSINPASL

SEQ ID NO: 284 - protein, 9 aa, Epstein-Barr virus
HLAVYQKFV

SEQ ID NO: 285 - protein, 9 aa, Epstein-Barr virus
HPNSVCHVA

SEQ ID NO: 286 - protein, 10 aa, Epstein-Barr virus
HPNSVCHVAT

SEQ ID NO: 287 - protein, 9 aa, Epstein-Barr virus
HVATWLQDK

SEQ ID NO: 288 - protein, 10 aa, Epstein-Barr virus
HVHESFLASL

SEQ ID NO: 289 - protein, 9 aa, Epstein-Barr virus
HVYDILETV

SEQ ID NO: 290 - protein, 10 aa, Epstein-Barr virus
HVYDILETVY

SEQ ID NO: 291 - protein, 9 aa, Epstein-Barr virus
HYKEIPRLF

SEQ ID NO: 292 - protein, 10 aa, Epstein-Barr virus
IAHIPCRRVL

SEQ ID NO: 293 - protein, 9 aa, Epstein-Barr virus
IIQAHNLCY

SEQ ID NO: 294 - protein, 9 aa, Epstein-Barr virus
ILDKQQLAI

SEQ ID NO: 295 - protein, 10 aa, Epstein-Barr virus
ILDKQQLAIK

SEQ ID NO: 296 - protein, 9 aa, Epstein-Barr virus
ILDRARHIY

SEQ ID NO: 297 - protein, 10 aa, Epstein-Barr virus
ILETVYTHEK

SEQ ID NO: 298 - protein, 9 aa, Epstein-Barr virus
ILNQAYTDL

SEQ ID NO: 299 - protein, 9 aa, Epstein-Barr virus
ILQISCVLW

SEQ ID NO: 300 - protein, 10 aa, Epstein-Barr virus
IPRLFAAGPE

SEQ ID NO: 301 - protein, 10 aa, Epstein-Barr virus
IPRLQHRDSY

SEQ ID NO: 302 - protein, 9 aa, Epstein-Barr virus
IPSDKQGYV

SEQ ID NO: 303 - protein, 10 aa, Epstein-Barr virus
IPSDKQGYVV

SEQ ID NO: 304 - protein, 9 aa, Epstein-Barr virus
IQYVFVEPK

SEQ ID NO: 305 - protein, 10 aa, Epstein-Barr virus
ITGLIPIDMY

SEQ ID NO: 306 - protein, 9 aa, Epstein-Barr virus
ITKKRYVGV

SEQ ID NO: 307 - protein, 10 aa, Epstein-Barr virus
ITKKRYVGVL

SEQ ID NO: 308 - protein, 9 aa, Epstein-Barr virus
ITLSHPNSV

SEQ ID NO: 309 - protein, 10 aa, Epstein-Barr virus
IVIKLLGRRK

SEQ ID NO: 310 - protein, 9 aa, Epstein-Barr virus
IYGDTDSLF

SEQ ID NO: 311 - protein, 10 aa, Epstein-Barr virus
IYGDTDSLFI

SEQ ID NO: 312 - protein, 9 aa, Epstein-Barr virus
IYSINPASL

SEQ ID NO: 313 - protein, 9 aa, Epstein-Barr virus
KAIKKLLAA

SEQ ID NO: 314 - protein, 9 aa, Epstein-Barr virus
KEYLRLIPK

SEQ ID NO: 315 - protein, 9 aa, Epstein-Barr virus
KGFLRANTK

SEQ ID NO: 316 - protein, 9 aa, Epstein-Barr virus
KIAHIPCRR

SEQ ID NO: 317 - protein, 10 aa, Epstein-Barr virus
KITLSHPNSV

SEQ ID NO: 318 - protein, 9 aa, Epstein-Barr virus
KLDTVARHL

SEQ ID NO: 319 - protein, 10 aa, Epstein-Barr virus
KLDTVARHLL

SEQ ID NO: 320 - protein, 9 aa, Epstein-Barr virus
KLLQGAANI

SEQ ID NO: 321 - protein, 10 aa, Epstein-Barr virus
KLLQGAANIL

SEQ ID NO: 322 - protein, 10 aa, Epstein-Barr virus
KLSAYKSTQM

SEQ ID NO: 323 - protein, 9 aa, Epstein-Barr virus
KLSLSDYKL

SEQ ID NO: 324 - protein, 9 aa, Epstein-Barr virus
KPDKEYLRL

SEQ ID NO: 325 - protein, 10 aa, Epstein-Barr virus
KTACKFVQTR

SEQ ID NO: 326 - protein, 10 aa, Epstein-Barr virus
KTEMAEDPAY

SEQ ID NO: 327 - protein, 9 aa, Epstein-Barr virus
KTFSCLMLI

SEQ ID NO: 328 - protein, 10 aa, Epstein-Barr virus
KTLMKGVELV

SEQ ID NO: 329 - protein, 10 aa, Epstein-Barr virus
KVRITGLIPI

SEQ ID NO: 330 - protein, 10 aa, Epstein-Barr virus
KVTRRSIMGY

SEQ ID NO: 331 - protein, 9 aa, Epstein-Barr virus
LAAHTTRSL

SEQ ID NO: 332 - protein, 10 aa, Epstein-Barr virus
LAKRKAIKKL

SEQ ID NO: 333 - protein, 10 aa, Epstein-Barr virus
LDRARHIYSI

SEQ ID NO: 334 - protein, 9 aa, Epstein-Barr virus
LILQISCVL

SEQ ID NO: 335 - protein, 9 aa, Epstein-Barr virus
LIPIDMYAV

SEQ ID NO: 336 - protein, 10 aa, Epstein-Barr virus
LITKKRYVGV

SEQ ID NO: 337 - protein, 9 aa, Epstein-Barr virus
LLNHFVIHV

SEQ ID NO: 338 - protein, 9 aa, Epstein-Barr virus
LLQGAANIL

SEQ ID NO: 339 - protein, 10 aa, Epstein-Barr virus
LLTSWLAKRK

SEQ ID NO: 340 - protein, 10 aa, Epstein-Barr virus
LMKGVELVRK

SEQ ID NO: 341 - protein, 10 aa, Epstein-Barr virus
LMLITKKRYV

SEQ ID NO: 342 - protein, 9 aa, Epstein-Barr virus
LPVGFLPVI

SEQ ID NO: 343 - protein, 10 aa, Epstein-Barr virus
LQISCVLWST

SEQ ID NO: 344 - protein, 9 aa, Epstein-Barr virus
LSRKLSAYK

SEQ ID NO: 345 - protein, 9 aa, Epstein-Barr virus
LTDGKTLMK

SEQ ID NO: 346 - protein, 9 aa, Epstein-Barr virus
LTGGVYHFV

SEQ ID NO: 347 - protein, 10 aa, Epstein-Barr virus
LTGGVYHFVK

SEQ ID NO: 348 - protein, 9 aa, Epstein-Barr virus
LTSWLAKRK

SEQ ID NO: 349 - protein, 10 aa, Epstein-Barr virus
LVMDLLNHFV

SEQ ID NO: 350 - protein, 9 aa, Epstein-Barr virus
LVVDFASLY

SEQ ID NO: 351 - protein, 9 aa, Epstein-Barr virus
MLERAKAFV

SEQ ID NO: 352 - protein, 9 aa, Epstein-Barr virus
MLITKKRYV

SEQ ID NO: 353 - protein, 10 aa, Epstein-Barr virus
MLITKKRYVG

SEQ ID NO: 354 - protein, 9 aa, Epstein-Barr virus
MLYAFFQLI

SEQ ID NO: 355 - protein, 10 aa, Epstein-Barr virus
MLYAFFQLIR

SEQ ID NO: 356 - protein, 10 aa, Epstein-Barr virus
MPHLAVYQKF

SEQ ID NO: 357 - protein, 10 aa, Epstein-Barr virus
MSGGLFYNPF

SEQ ID NO: 358 - protein, 9 aa, Epstein-Barr virus
MYAVCRDKL

SEQ ID NO: 359 - protein, 9 aa, Epstein-Barr virus
NPEGQLRVI

SEQ ID NO: 360 - protein, 10 aa, Epstein-Barr virus
NPFLRPNKGL

SEQ ID NO: 361 - protein, 9 aa, Epstein-Barr virus
NTKVRITGL

SEQ ID NO: 362 - protein, 10 aa, Epstein-Barr virus
NTKVRITGLI

SEQ ID NO: 363 - protein, 9 aa, Epstein-Barr virus
NVANFDWPY

SEQ ID NO: 364 - protein, 10 aa, Epstein-Barr virus
NVANFDWPYI

SEQ ID NO: 365 - protein, 10 aa, Epstein-Barr virus
PLSGFYNSPV

SEQ ID NO: 366 - protein, 9 aa, Epstein-Barr virus
QIHDRIQYV

SEQ ID NO: 367 - protein, 9 aa, Epstein-Barr virus
QIRVFSCLL

SEQ ID NO: 368 - protein, 10 aa, Epstein-Barr virus
QLIRDLSVEI

SEQ ID NO: 369 - protein, 10 aa, Epstein-Barr virus
QMPHLAVYQK

SEQ ID NO: 370 - protein, 10 aa, Epstein-Barr virus
QQIRVFSCLL

SEQ ID NO: 371 - protein, 10 aa, Epstein-Barr virus
QTRCRRVLDL

SEQ ID NO: 372 - protein, 9 aa, Epstein-Barr virus
RAKAFVEAL

SEQ ID NO: 373 - protein, 9 aa, Epstein-Barr virus
RIFEANVDA

SEQ ID NO: 374 - protein, 10 aa, Epstein-Barr virus
RIQYVFVEPK

SEQ ID NO: 375 - protein, 10 aa, Epstein-Barr virus
RLFAAGPEGR

SEQ ID NO: 376 - protein, 9 aa, Epstein-Barr virus
RLIPKCFQT

SEQ ID NO: 377 - protein, 9 aa, Epstein-Barr virus
RLTGGVYHF

SEQ ID NO: 378 - protein, 10 aa, Epstein-Barr virus
RLTGGVYHFV

SEQ ID NO: 379 - protein, 9 aa, Epstein-Barr virus
RPGEDYESF

SEQ ID NO: 380 - protein, 9 aa, Epstein-Barr virus
RPHDAGKGF

SEQ ID NO: 381 - protein, 10 aa, Epstein-Barr virus
RPHDAGKGFL

SEQ ID NO: 382 - protein, 10 aa, Epstein-Barr virus
RTMLERAKAF

SEQ ID NO: 383 - protein, 10 aa, Epstein-Barr virus
RTPCRVSVEK

SEQ ID NO: 384 - protein, 9 aa, Epstein-Barr virus
RVFSCLLAA

SEQ ID NO: 385 - protein, 10 aa, Epstein-Barr virus
RVFSCLLAAA

SEQ ID NO: 386 - protein, 10 aa, Epstein-Barr virus
RVIYGDTDSL

SEQ ID NO: 387 - protein, 9 aa, Epstein-Barr virus
RVKEAASLL

SEQ ID NO: 388 - protein, 10 aa, Epstein-Barr virus
RVLDLVLADA

SEQ ID NO: 389 - protein, 9 aa, Epstein-Barr virus
RYRRILLTL

SEQ ID NO: 390 - protein, 10 aa, Epstein-Barr virus
SFLASLLTSW

SEQ ID NO: 391 - protein, 10 aa, Epstein-Barr virus
SFTQGLPVGF

SEQ ID NO: 392 - protein, 9 aa, Epstein-Barr virus
SIMGYGNHA

SEQ ID NO: 393 - protein, 9 aa, Epstein-Barr virus
SINPASLGK

SEQ ID NO: 394 - protein, 9 aa, Epstein-Barr virus
SLFVAPISL

SEQ ID NO: 395 - protein, 9 aa, Epstein-Barr virus
SLLTSWLAK

SEQ ID NO: 396 - protein, 10 aa, Epstein-Barr virus
SLLTSWLAKR

SEQ ID NO: 397 - protein, 10 aa, Epstein-Barr virus
SLSDYKLDTV

SEQ ID NO: 398 - protein, 9 aa, Epstein-Barr virus
SLYPSIIQA

SEQ ID NO: 399 - protein, 10 aa, Epstein-Barr virus
SLYPSIIQAH

SEQ ID NO: 400 - protein, 9 aa, Epstein-Barr virus
SPANLQALA

SEQ ID NO: 401 - protein, 10 aa, Epstein-Barr virus
SPLDFHVYDI

SEQ ID NO: 402 - protein, 10 aa, Epstein-Barr virus
STFDRRTPCR

SEQ ID NO: 403 - protein, 9 aa, Epstein-Barr virus
STGEEAGRY

SEQ ID NO: 404 - protein, 10 aa, Epstein-Barr virus
STQMPHLAVY

SEQ ID NO: 405 - protein, 9 aa, Epstein-Barr virus
SWLAKRKAI

SEQ ID NO: 406 - protein, 10 aa, Epstein-Barr virus
SWPSYQALAF

SEQ ID NO: 407 - protein, 9 aa, Epstein-Barr virus
SYQALAFDI

SEQ ID NO: 408 - protein, 9 aa, Epstein-Barr virus
TKKRYVGVL

SEQ ID NO: 409 - protein, 9 aa, Epstein-Barr virus
TLMKGVELV

SEQ ID NO: 410 - protein, 9 aa, Epstein-Barr virus
TMLERAKAF

SEQ ID NO: 411 - protein, 10 aa, Epstein-Barr virus
TMLERAKAFV

SEQ ID NO: 412 - protein, 10 aa, Epstein-Barr virus
TVARHLLGAK

SEQ ID NO: 413 - protein, 9 aa, Epstein-Barr virus
VARHLLGAK

SEQ ID NO: 414 - protein, 10 aa, Epstein-Barr virus
VARHLLGAKK

SEQ ID NO: 415 - protein, 9 aa, Epstein-Barr virus
VIDILNQAY

SEQ ID NO: 416 - protein, 9 aa, Epstein-Barr virus
VIKLLGRRK

SEQ ID NO: 417 - protein, 9 aa, Epstein-Barr virus
VIQPLSGFY

SEQ ID NO: 418 - protein, 9 aa, Epstein-Barr virus
VIYGDTDSL

SEQ ID NO: 419 - protein, 10 aa, Epstein-Barr virus
VLADARVKEA

SEQ ID NO: 420 - protein, 10 aa, Epstein-Barr virus
VLDDGQQIRV

SEQ ID NO: 421 - protein, 9 aa, Epstein-Barr virus
VLDLVLADA

SEQ ID NO: 422 - protein, 10 aa, Epstein-Barr virus
VLTDGKTLMK

SEQ ID NO: 423 - protein, 9 aa, Epstein-Barr virus
VLVVDFASL

SEQ ID NO: 424 - protein, 9 aa, Epstein-Barr virus
VLWSTGEEA

SEQ ID NO: 425 - protein, 9 aa, Epstein-Barr virus
VMDLLNHFV

SEQ ID NO: 426 - protein, 10 aa, Epstein-Barr virus
VMDLLNHFVI

SEQ ID NO: 427 - protein, 9 aa, Epstein-Barr virus
VTFGWYSCR

SEQ ID NO: 428 - protein, 10 aa, Epstein-Barr virus
VTFGWYSCRR

SEQ ID NO: 429 - protein, 9 aa, Epstein-Barr virus
VTRRSIMGY

SEQ ID NO: 430 - protein, 10 aa, Epstein-Barr virus
VYTHEKCAVI

SEQ ID NO: 431 - protein, 9 aa, Epstein-Barr virus
WLAKRKAIK

SEQ ID NO: 432 - protein, 10 aa, Epstein-Barr virus
WLAKRKAIKK

SEQ ID NO: 433 - protein, 10 aa, Epstein-Barr virus
WLQDKHGCRI

SEQ ID NO: 434 - protein, 9 aa, Epstein-Barr virus
WPSYQALAF

SEQ ID NO: 435 - protein, 10 aa, Epstein-Barr virus
WSTGEEAGRY

SEQ ID NO: 436 - protein, 9 aa, Epstein-Barr virus
YAERHGVPV

SEQ ID NO: 437 - protein, 10 aa, Epstein-Barr virus
YAVCRDKLSL

SEQ ID NO: 438 - protein, 10 aa, Epstein-Barr virus
YFYASAPQGL

SEQ ID NO: 439 - protein, 9 aa, Epstein-Barr virus
YILDRARHI

SEQ ID NO: 440 - protein, 9 aa, Epstein-Barr virus
YLRLIPKCF

SEQ ID NO: 441 - protein, 10 aa, Epstein-Barr virus
YPSIIQAHNL

SEQ ID NO: 442 - protein, 10 aa, Epstein-Barr virus
YQGATVIQPL

SEQ ID NO: 443 - protein, 10 aa, Epstein-Barr virus
YSINPASLGK

SEQ ID NO: 444 - protein, 9 aa, Epstein-Barr virus
YTDLREGRV

SEQ ID NO: 445 - protein, 10 aa, Epstein-Barr virus
YVFVEPKGGV

SEQ ID NO: 446 - protein, 9 aa, Epstein-Barr virus
AYSARPPPL

SEQ ID NO: 447 - protein, 10 aa, Epstein-Barr virus
CAYSARPPPL

SEQ ID NO: 448 - protein, 10 aa, Epstein-Barr virus
GARRRARCPA

SEQ ID NO: 449 - protein, 10 aa, Epstein-Barr virus
GPGRRARSCL

SEQ ID NO: 450 - protein, 10 aa, Epstein-Barr virus
MAGARRRARC

SEQ ID NO: 451 - protein, 10 aa, Epstein-Barr virus
RPGPGRRARS

SEQ ID NO: 452 - protein, 9 aa, Epstein-Barr virus
RPPHSRTRA

SEQ ID NO: 453 - protein, 10 aa, Epstein-Barr virus
RRRARCPASA

SEQ ID NO: 454 - protein, 9 aa, Epstein-Barr virus
SGQPRWWPW

SEQ ID NO: 455 - protein, 9 aa, Epstein-Barr virus
STRGRRISA

SEQ ID NO: 456 - protein, 9 aa, Epstein-Barr virus
WPWGSPPPL

SEQ ID NO: 457 - protein, 10 aa, Epstein-Barr virus
WWPWGSPPPL

SEQ ID NO: 458 - protein, 9 aa, Epstein-Barr virus
AMFCAWKLY

SEQ ID NO: 459 - protein, 10 aa, Epstein-Barr virus
AMFCAWKLYS

SEQ ID NO: 460 - protein, 10 aa, Epstein-Barr virus
AVKAAAQSAF

SEQ ID NO: 461 - protein, 9 aa, Epstein-Barr virus
CAWKLYSRK

SEQ ID NO: 462 - protein, 10 aa, Epstein-Barr virus
FADAWRMARY

SEQ ID NO: 463 - protein, 9 aa, Epstein-Barr virus
KLYSRKMPS

SEQ ID NO: 464 - protein, 10 aa, Epstein-Barr virus
KLYSRKMPSR

SEQ ID NO: 465 - protein, 10 aa, Epstein-Barr virus
KPPTVLTRPA

SEQ ID NO: 466 - protein, 10 aa, Epstein-Barr virus
KTLEARCSSR

SEQ ID NO: 467 - protein, 9 aa, Epstein-Barr virus
MARYWGCAV

SEQ ID NO: 468 - protein, 10 aa, Epstein-Barr virus
MARYWGCAVK

SEQ ID NO: 469 - protein, 10 aa, Epstein-Barr virus
MPSRSKTLEA

SEQ ID NO: 470 - protein, 10 aa, Epstein-Barr virus
MSMPPKGFLK

SEQ ID NO: 471 - protein, 10 aa, Epstein-Barr virus
PPTVLTRPAM

SEQ ID NO: 472 - protein, 10 aa, Epstein-Barr virus
RGWRARRLGK

SEQ ID NO: 473 - protein, 10 aa, Epstein-Barr virus
RKMPSRSKTL

SEQ ID NO: 474 - protein, 9 aa, Epstein-Barr virus
RLGKESWFA

SEQ ID NO: 475 - protein, 9 aa, Epstein-Barr virus
RLLNKPPTV

SEQ ID NO: 476 - protein, 10 aa, Epstein-Barr virus
RLLNKPPTVL

SEQ ID NO: 477 - protein, 10 aa, Epstein-Barr virus
RMARYWGCAV

SEQ ID NO: 478 - protein, 10 aa, Epstein-Barr virus
RPAMFCAWKL

SEQ ID NO: 479 - protein, 9 aa, Epstein-Barr virus
SMPPKGFLK

SEQ ID NO: 480 - protein, 10 aa, Epstein-Barr virus
SMPPKGFLKK

SEQ ID NO: 481 - protein, 9 aa, Epstein-Barr virus
SPACQTRDT

SEQ ID NO: 482 - protein, 9 aa, Epstein-Barr virus
SWFADAWRM

SEQ ID NO: 483 - protein, 10 aa, Epstein-Barr virus
VLTRPAMFCA

SEQ ID NO: 484 - protein, 9 aa, Epstein-Barr virus
WRMARYWGC

SEQ ID NO: 485 - protein, 10 aa, Epstein-Barr virus
WRMARYWGCA

SEQ ID NO: 486 - protein, 10 aa, Epstein-Barr virus
YSRKMPSRSK

SEQ ID NO: 487 - protein, 9 aa, Epstein-Barr virus
AARDRFPGL

SEQ ID NO: 488 - protein, 9 aa, Epstein-Barr virus
AARGSTSAA

SEQ ID NO: 489 - protein, 10 aa, Epstein-Barr virus
AARGSTSAAV

SEQ ID NO: 490 - protein, 10 aa, Epstein-Barr virus
AFLDKGTYTL

SEQ ID NO: 491 - protein, 10 aa, Epstein-Barr virus
ALHEQNQEQK

SEQ ID NO: 492 - protein, 9 aa, Epstein-Barr virus
APGKSLGTL

SEQ ID NO: 493 - protein, 9 aa, Epstein-Barr virus
APPAARGST

SEQ ID NO: 494 - protein, 9 aa, Epstein-Barr virus
AQNIAGLRK

SEQ ID NO: 495 - protein, 9 aa, Epstein-Barr virus
ATLQTFISL

SEQ ID NO: 496 - protein, 10 aa, Epstein-Barr virus
ATRQQTSFPF

SEQ ID NO: 497 - protein, 9 aa, Epstein-Barr virus
ATVQIQFAY

SEQ ID NO: 498 - protein, 9 aa, Epstein-Barr virus
CLEQKRQNM

SEQ ID NO: 499 - protein, 9 aa, Epstein-Barr virus
CLITDMMAK

SEQ ID NO: 500 - protein, 9 aa, Epstein-Barr virus
CPLQHWQTF

SEQ ID NO: 501 - protein, 9 aa, Epstein-Barr virus
CQATSQYYF

SEQ ID NO: 502 - protein, 9 aa, Epstein-Barr virus
CYSRPLVSF

SEQ ID NO: 503 - protein, 10 aa, Epstein-Barr virus
DMMAKSNSPF

SEQ ID NO: 504 - protein, 10 aa, Epstein-Barr virus
DSFHVRTNYK

SEQ ID NO: 505 - protein, 9 aa, Epstein-Barr virus
ELMDSLGSV

SEQ ID NO: 506 - protein, 10 aa, Epstein-Barr virus
ELYDAPGWLI

SEQ ID NO: 507 - protein, 9 aa, Epstein-Barr virus
ENRTAYCPL

SEQ ID NO: 508 - protein, 9 aa, Epstein-Barr virus
EQKRQNMVL

SEQ ID NO: 509 - protein, 9 aa, Epstein-Barr virus
ETDQMDTIY

SEQ ID NO: 510 - protein, 9 aa, Epstein-Barr virus
ETMCYSRPL

SEQ ID NO: 511 - protein, 10 aa, Epstein-Barr virus
FFKNPFGGML

SEQ ID NO: 512 - protein, 9 aa, Epstein-Barr virus
FISLNTSLI

SEQ ID NO: 513 - protein, 9 aa, Epstein-Barr virus
FITSGGLLL

SEQ ID NO: 514 - protein, 10 aa, Epstein-Barr virus
FITSGGLLLA

SEQ ID NO: 515 - protein, 9 aa, Epstein-Barr virus
FLDKGTYTL

SEQ ID NO: 516 - protein, 10 aa, Epstein-Barr virus
FLDKGTYTLS

SEQ ID NO: 517 - protein, 9 aa, Epstein-Barr virus
FLTKKMTEV

SEQ ID NO: 518 - protein, 9 aa, Epstein-Barr virus
FPGLRRRRY

SEQ ID NO: 519 - protein, 9 aa, Epstein-Barr virus
FQAQNIAGL

SEQ ID NO: 520 - protein, 9 aa, Epstein-Barr virus
FQSGNEIHV

SEQ ID NO: 521 - protein, 10 aa, Epstein-Barr virus
FSFINDTKTY

SEQ ID NO: 522 - protein, 9 aa, Epstein-Barr virus
FVDGLGELM

SEQ ID NO: 523 - protein, 9 aa, Epstein-Barr virus
FVTNTTVGI

SEQ ID NO: 524 - protein, 10 aa, Epstein-Barr virus
FYDGKNKETF

SEQ ID NO: 525 - protein, 10 aa, Epstein-Barr virus
GFISFFKNPF

SEQ ID NO: 526 - protein, 9 aa, Epstein-Barr virus
GIATLQTFI

SEQ ID NO: 527 - protein, 10 aa, Epstein-Barr virus
GIFREYNFQA

SEQ ID NO: 528 - protein, 9 aa, Epstein-Barr virus
GLFSSLVSG

SEQ ID NO: 529 - protein, 10 aa, Epstein-Barr virus
GLFSSLVSGF

SEQ ID NO: 530 - protein, 9 aa, Epstein-Barr virus
GLGELMDSL

SEQ ID NO: 531 - protein, 9 aa, Epstein-Barr virus
GLLLAWLPL

SEQ ID NO: 532 - protein, 10 aa, Epstein-Barr virus
GLLLAWLPLT

SEQ ID NO: 533 - protein, 10 aa, Epstein-Barr virus
GLLMVFKDNI

SEQ ID NO: 534 - protein, 10 aa, Epstein-Barr virus
GLRKDLDNAV

SEQ ID NO: 535 - protein, 9 aa, Epstein-Barr virus
GMLILVLVA

SEQ ID NO: 536 - protein, 9 aa, Epstein-Barr virus
GPSVASRAL

SEQ ID NO: 537 - protein, 9 aa, Epstein-Barr virus
GQEAITYFI

SEQ ID NO: 538 - protein, 10 aa, Epstein-Barr virus
GTDNEIFLTK

SEQ ID NO: 539 - protein, 9 aa, Epstein-Barr virus
GTLNNPATV

SEQ ID NO: 540 - protein, 9 aa, Epstein-Barr virus
GTYTLSWKL

SEQ ID NO: 541 - protein, 9 aa, Epstein-Barr virus
GVNITVNLK

SEQ ID NO: 542 - protein, 10 aa, Epstein-Barr virus
HTEGLLMVFK

SEQ ID NO: 543 - protein, 10 aa, Epstein-Barr virus
HVYNDYHHFK

SEQ ID NO: 544 - protein, 9 aa, Epstein-Barr virus
HWQTFDSTI

SEQ ID NO: 545 - protein, 9 aa, Epstein-Barr virus
ILIYNGWYA

SEQ ID NO: 546 - protein, 9 aa, Epstein-Barr virus
IQFAYDSLR

SEQ ID NO: 547 - protein, 10 aa, Epstein-Barr virus
IQFAYDSLRR

SEQ ID NO: 548 - protein, 10 aa, Epstein-Barr virus
IYNGWYADSV

SEQ ID NO: 549 - protein, 10 aa, Epstein-Barr virus
IYQCYNAVKM

SEQ ID NO: 550 - protein, 9 aa, Epstein-Barr virus
KGTYTLSWK

SEQ ID NO: 551 - protein, 9 aa, Epstein-Barr virus
KIVTNILIY

SEQ ID NO: 552 - protein, 9 aa, Epstein-Barr virus
KMTEVCQAT

SEQ ID NO: 553 - protein, 10 aa, Epstein-Barr virus
KMTKDGLTRV

SEQ ID NO: 554 - protein, 10 aa, Epstein-Barr virus
KPTGGLANGV

SEQ ID NO: 555 - protein, 10 aa, Epstein-Barr virus
KSNSPFDFFV

SEQ ID NO: 556 - protein, 10 aa, Epstein-Barr virus
KTMHEKYEAV

SEQ ID NO: 557 - protein, 10 aa, Epstein-Barr virus
LARAWCLEQK

SEQ ID NO: 558 - protein, 9 aa, Epstein-Barr virus
LFSSLVSGF

SEQ ID NO: 559 - protein, 9 aa, Epstein-Barr virus
LILVLVAGV

SEQ ID NO: 560 - protein, 10 aa, Epstein-Barr virus
LILVLVAGVV

SEQ ID NO: 561 - protein, 9 aa, Epstein-Barr virus
LLAALACRL

SEQ ID NO: 562 - protein, 10 aa, Epstein-Barr virus
LLAWLPLTPR

SEQ ID NO: 563 - protein, 9 aa, Epstein-Barr virus
LLLAWLPLT

SEQ ID NO: 564 - protein, 9 aa, Epstein-Barr virus
LLMVFKDNI

SEQ ID NO: 565 - protein, 10 aa, Epstein-Barr virus
LLMVFKDNII

SEQ ID NO: 566 - protein, 9 aa, Epstein-Barr virus
LPLTPRSLA

SEQ ID NO: 567 - protein, 10 aa, Epstein-Barr virus
LPLTPRSLAT

SEQ ID NO: 568 - protein, 9 aa, Epstein-Barr virus
LRRQINRML

SEQ ID NO: 569 - protein, 9 aa, Epstein-Barr virus
LTPRSLATV

SEQ ID NO: 570 - protein, 9 aa, Epstein-Barr virus
LVAGVVILV

SEQ ID NO: 571 - protein, 10 aa, Epstein-Barr virus
LVSGFISFFK

SEQ ID NO: 572 - protein, 9 aa, Epstein-Barr virus
LYDAPGWLI

SEQ ID NO: 573 - protein, 10 aa, Epstein-Barr virus
LYDAPGWLIW

SEQ ID NO: 574 - protein, 10 aa, Epstein-Barr virus
MCYSRPLVSF

SEQ ID NO: 575 - protein, 10 aa, Epstein-Barr virus
MLILVLVAGV

SEQ ID NO: 576 - protein, 9 aa, Epstein-Barr virus
MLYPGIDEL

SEQ ID NO: 577 - protein, 10 aa, Epstein-Barr virus
MLYPGIDELA

SEQ ID NO: 578 - protein, 9 aa, Epstein-Barr virus
MMAKSNSPF

SEQ ID NO: 579 - protein, 10 aa, Epstein-Barr virus
MSQQPVQMLY

SEQ ID NO: 580 - protein, 9 aa, Epstein-Barr virus
MTRRRVLSV

SEQ ID NO: 581 - protein, 10 aa, Epstein-Barr virus
MTRRRVLSVV

SEQ ID NO: 582 - protein, 10 aa, Epstein-Barr virus
MVFKDNIIPY

SEQ ID NO: 583 - protein, 9 aa, Epstein-Barr virus
MVLRELTKI

SEQ ID NO: 584 - protein, 10 aa, Epstein-Barr virus
NIDFASLELY

SEQ ID NO: 585 - protein, 9 aa, Epstein-Barr virus
NIIPYSFKV

SEQ ID NO: 586 - protein, 10 aa, Epstein-Barr virus
NILIYNGWYA

SEQ ID NO: 587 - protein, 9 aa, Epstein-Barr virus
NLTELTTPT

SEQ ID NO: 588 - protein, 9 aa, Epstein-Barr virus
NMVLRELTK

SEQ ID NO: 589 - protein, 9 aa, Epstein-Barr virus
NPATVQIQF

SEQ ID NO: 590 - protein, 9 aa, Epstein-Barr virus
NPFGGMLIL

SEQ ID NO: 591 - protein, 9 aa, Epstein-Barr virus
NPQGERRAF

SEQ ID NO: 592 - protein, 10 aa, Epstein-Barr virus
NPQGERRAFL

SEQ ID NO: 593 - protein, 9 aa, Epstein-Barr virus
NPTTVMSSI

SEQ ID NO: 594 - protein, 9 aa, Epstein-Barr virus
PPAAPGKSL

SEQ ID NO: 595 - protein, 10 aa, Epstein-Barr virus
PPAARGSTSA

SEQ ID NO: 596 - protein, 9 aa, Epstein-Barr virus
QMDTIYQCY

SEQ ID NO: 597 - protein, 10 aa, Epstein-Barr virus
QMLYPGIDEL

SEQ ID NO: 598 - protein, 9 aa, Epstein-Barr virus
QPAPPATTV

SEQ ID NO: 599 - protein, 9 aa, Epstein-Barr virus
QQTSFPFRV

SEQ ID NO: 600 - protein, 9 aa, Epstein-Barr virus
QTVEMSPFY

SEQ ID NO: 601 - protein, 9 aa, Epstein-Barr virus
QVNKTMHEK

SEQ ID NO: 602 - protein, 10 aa, Epstein-Barr virus
QYYFQSGNEI

SEQ ID NO: 603 - protein, 9 aa, Epstein-Barr virus
RMLGDLARA

SEQ ID NO: 604 - protein, 10 aa, Epstein-Barr virus
RMLGDLARAW

SEQ ID NO: 605 - protein, 9 aa, Epstein-Barr virus
RPLVSFSFI

SEQ ID NO: 606 - protein, 9 aa, Epstein-Barr virus
RQQTSFPFR

SEQ ID NO: 607 - protein, 10 aa, Epstein-Barr virus
RQQTSFPFRV

SEQ ID NO: 608 - protein, 9 aa, Epstein-Barr virus
RTAYCPLQH

SEQ ID NO: 609 - protein, 9 aa, Epstein-Barr virus
RTNYKIVDY

SEQ ID NO: 610 - protein, 10 aa, Epstein-Barr virus
RTRQMSQQPV

SEQ ID NO: 611 - protein, 9 aa, Epstein-Barr virus
RTRTTVNCL

SEQ ID NO: 612 - protein, 9 aa, Epstein-Barr virus
RVLSVVVLL

SEQ ID NO: 613 - protein, 10 aa, Epstein-Barr virus
RVLSVVVLLA

SEQ ID NO: 614 - protein, 9 aa, Epstein-Barr virus
RYASQTELY

SEQ ID NO: 615 - protein, 9 aa, Epstein-Barr virus
RYTKGQEAI

SEQ ID NO: 616 - protein, 9 aa, Epstein-Barr virus
SFHVRTNYK

SEQ ID NO: 617 - protein, 10 aa, Epstein-Barr virus
SFHVRTNYKI

SEQ ID NO: 618 - protein, 10 aa, Epstein-Barr virus
SFKVRSYTKI

SEQ ID NO: 619 - protein, 9 aa, Epstein-Barr virus
SFPFRVCEL

SEQ ID NO: 620 - protein, 9 aa, Epstein-Barr virus
SITNLVSTV

SEQ ID NO: 621 - protein, 9 aa, Epstein-Barr virus
SIYGKAVAA

SEQ ID NO: 622 - protein, 10 aa, Epstein-Barr virus
SIYGKAVAAK

SEQ ID NO: 623 - protein, 9 aa, Epstein-Barr virus
SLGSVGQSI

SEQ ID NO: 624 - protein, 9 aa, Epstein-Barr virus
SLIENIDFA

SEQ ID NO: 625 - protein, 10 aa, Epstein-Barr virus
SLIENIDFAS

SEQ ID NO: 626 - protein, 10 aa, Epstein-Barr virus
SLNTSLIENI

SEQ ID NO: 627 - protein, 9 aa, Epstein-Barr virus
SLRRQINRM

SEQ ID NO: 628 - protein, 10 aa, Epstein-Barr virus
SLRRQINRML

SEQ ID NO: 629 - protein, 9 aa, Epstein-Barr virus
SLTRRTRQM

SEQ ID NO: 630 - protein, 9 aa, Epstein-Barr virus
SLVSGFISF

SEQ ID NO: 631 - protein, 10 aa, Epstein-Barr virus
SLVSGFISFF

SEQ ID NO: 632 - protein, 10 aa, Epstein-Barr virus
SMRVPGSETM

SEQ ID NO: 633 - protein, 10 aa, Epstein-Barr virus
SPPSSPSPPA

SEQ ID NO: 634 - protein, 9 aa, Epstein-Barr virus
SPSPPAPPA

SEQ ID NO: 635 - protein, 10 aa, Epstein-Barr virus
SPSPPAPPAA

SEQ ID NO: 636 - protein, 9 aa, Epstein-Barr virus
STIATETGK

SEQ ID NO: 637 - protein, 10 aa, Epstein-Barr virus
STVGGLFSSL

SEQ ID NO: 638 - protein, 10 aa, Epstein-Barr virus
SVGQSITNLV

SEQ ID NO: 639 - protein, 9 aa, Epstein-Barr virus
SVVVLLAAL

SEQ ID NO: 640 - protein, 9 aa, Epstein-Barr virus
SYTKIVTNI

SEQ ID NO: 641 - protein, 10 aa, Epstein-Barr virus
SYTKIVTNIL

SEQ ID NO: 642 - protein, 9 aa, Epstein-Barr virus
TFHERADSF

SEQ ID NO: 643 - protein, 10 aa, Epstein-Barr virus
TFISLNTSLI

SEQ ID NO: 644 - protein, 9 aa, Epstein-Barr virus
TIYQCYNAV

SEQ ID NO: 645 - protein, 10 aa, Epstein-Barr virus
TIYQCYNAVK

SEQ ID NO: 646 - protein, 9 aa, Epstein-Barr virus
TMCYSRPLV

SEQ ID NO: 647 - protein, 9 aa, Epstein-Barr virus
TMHEKYEAV

SEQ ID NO: 648 - protein, 10 aa, Epstein-Barr virus
TTVMSSIYGK

SEQ ID NO: 649 - protein, 10 aa, Epstein-Barr virus
TVGGLFSSLV

SEQ ID NO: 650 - protein, 9 aa, Epstein-Barr virus
TVMSSIYGK

SEQ ID NO: 651 - protein, 10 aa, Epstein-Barr virus
TYFITSGGLL

SEQ ID NO: 652 - protein, 9 aa, Epstein-Barr virus
VILVISLTR

SEQ ID NO: 653 - protein, 10 aa, Epstein-Barr virus
VILVISLTRR

SEQ ID NO: 654 - protein, 10 aa, Epstein-Barr virus
VISVSQCVPV

SEQ ID NO: 655 - protein, 9 aa, Epstein-Barr virus
VLLAALACR

SEQ ID NO: 656 - protein, 10 aa, Epstein-Barr virus
VLLAALACRL

SEQ ID NO: 657 - protein, 9 aa, Epstein-Barr virus
VLRRRRRDA

SEQ ID NO: 658 - protein, 10 aa, Epstein-Barr virus
VLRRRRRDAG

SEQ ID NO: 659 - protein, 9 aa, Epstein-Barr virus
VLSVVVLLA

SEQ ID NO: 660 - protein, 10 aa, Epstein-Barr virus
VLSVVVLLAA

SEQ ID NO: 661 - protein, 9 aa, Epstein-Barr virus
VLVAGVVIL

SEQ ID NO: 662 - protein, 10 aa, Epstein-Barr virus
VLVAGVVILV

SEQ ID NO: 663 - protein, 9 aa, Epstein-Barr virus
VMSSIYGKA

SEQ ID NO: 664 - protein, 10 aa, Epstein-Barr virus
VMSSIYGKAV

SEQ ID NO: 665 - protein, 10 aa, Epstein-Barr virus
VPPAAPGKSL

SEQ ID NO: 666 - protein, 10 aa, Epstein-Barr virus
VPVNQATVTL

SEQ ID NO: 667 - protein, 10 aa, Epstein-Barr virus
VSFSFINDTK

SEQ ID NO: 668 - protein, 9 aa, Epstein-Barr virus
VSGFISFFK

SEQ ID NO: 669 - protein, 9 aa, Epstein-Barr virus
VTDEGTSSF

SEQ ID NO: 670 - protein, 10 aa, Epstein-Barr virus
VTDEGTSSFV

SEQ ID NO: 671 - protein, 10 aa, Epstein-Barr virus
VYVDRDGVNI

SEQ ID NO: 672 - protein, 9 aa, Epstein-Barr virus
YADSVTNRH

SEQ ID NO: 673 - protein, 10 aa, Epstein-Barr virus
YCPLQHWQTF

SEQ ID NO: 674 - protein, 10 aa, Epstein-Barr virus
YFITSGGLLL

SEQ ID NO: 675 - protein, 10 aa, Epstein-Barr virus
YSFKVRSYTK

SEQ ID NO: 676 - protein, 10 aa, Epstein-Barr virus
YSRPLVSFSF

SEQ ID NO: 677 - protein, 10 aa, Epstein-Barr virus
YTKGQEAITY

SEQ ID NO: 678 - protein, 9 aa, Epstein-Barr virus
YYFQSGNEI

SEQ ID NO: 679 - protein, 10 aa, Epstein-Barr virus
AAAYSQVYAL

SEQ ID NO: 680 - protein, 10 aa, Epstein-Barr virus
AAFENSKYIK

SEQ ID NO: 681 - protein, 10 aa, Epstein-Barr virus
AAFTAHPQYR

SEQ ID NO: 682 - protein, 9 aa, Epstein-Barr virus
AAYSQVYAL

SEQ ID NO: 683 - protein, 9 aa, Epstein-Barr virus
AGARLTHLF

SEQ ID NO: 684 - protein, 9 aa, Epstein-Barr virus
AILLPRLRR

SEQ ID NO: 685 - protein, 9 aa, Epstein-Barr virus
AILQKRNEL

SEQ ID NO: 686 - protein, 9 aa, Epstein-Barr virus
ALAGARLTH

SEQ ID NO: 687 - protein, 10 aa, Epstein-Barr virus
ALAGARLTHL

SEQ ID NO: 688 - protein, 9 aa, Epstein-Barr virus
ALARCCDAA

SEQ ID NO: 689 - protein, 10 aa, Epstein-Barr virus
ALAVELSVCA

SEQ ID NO: 690 - protein, 10 aa, Epstein-Barr virus
ALDTYNVFST

SEQ ID NO: 691 - protein, 9 aa, Epstein-Barr virus
ALFARERRL

SEQ ID NO: 692 - protein, 9 aa, Epstein-Barr virus
ALKRKEQYL

SEQ ID NO: 693 - protein, 9 aa, Epstein-Barr virus
ALLYLHLQM

SEQ ID NO: 694 - protein, 9 aa, Epstein-Barr virus
ALPPDGLYL

SEQ ID NO: 695 - protein, 10 aa, Epstein-Barr virus
ALPPDGLYLT

SEQ ID NO: 696 - protein, 9 aa, Epstein-Barr virus
ALREAVLTV

SEQ ID NO: 697 - protein, 9 aa, Epstein-Barr virus
ALSDALKRK

SEQ ID NO: 698 - protein, 9 aa, Epstein-Barr virus
ALSLELVHL

SEQ ID NO: 699 - protein, 10 aa, Epstein-Barr virus
ALSLELVHLL

SEQ ID NO: 700 - protein, 9 aa, Epstein-Barr virus
ALTHSLYTF

SEQ ID NO: 701 - protein, 10 aa, Epstein-Barr virus
ALTHSLYTFI

SEQ ID NO: 702 - protein, 9 aa, Epstein-Barr virus
APGATERLF

SEQ ID NO: 703 - protein, 9 aa, Epstein-Barr virus
APGRKGTRV

SEQ ID NO: 704 - protein, 10 aa, Epstein-Barr virus
AVLTVSLYNK

SEQ ID NO: 705 - protein, 9 aa, Epstein-Barr virus
AYSQVYALA

SEQ ID NO: 706 - protein, 10 aa, Epstein-Barr virus
AYSQVYALAV

SEQ ID NO: 707 - protein, 9 aa, Epstein-Barr virus
CARLDPRSL

SEQ ID NO: 708 - protein, 9 aa, Epstein-Barr virus
CLAQQSETL

SEQ ID NO: 709 - protein, 9 aa, Epstein-Barr virus
CLINDIEIL

SEQ ID NO: 710 - protein, 10 aa, Epstein-Barr virus
CLINDIEILM

SEQ ID NO: 711 - protein, 9 aa, Epstein-Barr virus
DLLGRFRGV

SEQ ID NO: 712 - protein, 10 aa, Epstein-Barr virus
DLYALLYLHL

SEQ ID NO: 713 - protein, 9 aa, Epstein-Barr virus
DPRLYITSV

SEQ ID NO: 714 - protein, 10 aa, Epstein-Barr virus
DPRSLDVAAV

SEQ ID NO: 715 - protein, 10 aa, Epstein-Barr virus
DSDRPLILLY

SEQ ID NO: 716 - protein, 10 aa, Epstein-Barr virus
DWIETSFNSF

SEQ ID NO: 717 - protein, 10 aa, Epstein-Barr virus
EILMKRINSV

SEQ ID NO: 718 - protein, 10 aa, Epstein-Barr virus
ELARMRNHFL

SEQ ID NO: 719 - protein, 9 aa, Epstein-Barr virus
ELYTRLNRA

SEQ ID NO: 720 - protein, 10 aa, Epstein-Barr virus
ELYTRLNRAM

SEQ ID NO: 721 - protein, 10 aa, Epstein-Barr virus
EVAELSELLY

SEQ ID NO: 722 - protein, 9 aa, Epstein-Barr virus
FARERRLAL

SEQ ID NO: 723 - protein, 10 aa, Epstein-Barr virus
FARERRLALV

SEQ ID NO: 724 - protein, 9 aa, Epstein-Barr virus
FKDLYALLY

SEQ ID NO: 725 - protein, 9 aa, Epstein-Barr virus
FLTSLIWPG

SEQ ID NO: 726 - protein, 10 aa, Epstein-Barr virus
FLTSLIWPGI

SEQ ID NO: 727 - protein, 9 aa, Epstein-Barr virus
FLYRRAFVS

SEQ ID NO: 728 - protein, 10 aa, Epstein-Barr virus
FLYRRAFVSG

SEQ ID NO: 729 - protein, 9 aa, Epstein-Barr virus
FPPPSNVAL

SEQ ID NO: 730 - protein, 10 aa, Epstein-Barr virus
FYCMSHTMGL

SEQ ID NO: 731 - protein, 9 aa, Epstein-Barr virus
GGHSRLSAL

SEQ ID NO: 732 - protein, 9 aa, Epstein-Barr virus
GLAKLQSCL

SEQ ID NO: 733 - protein, 10 aa, Epstein-Barr virus
GLAKLQSCLA

SEQ ID NO: 734 - protein, 10 aa, Epstein-Barr virus
GLFENEVRQA

SEQ ID NO: 735 - protein, 9 aa, Epstein-Barr virus
GLFPPPSNV

SEQ ID NO: 736 - protein, 10 aa, Epstein-Barr virus
GLFPPPSNVA

SEQ ID NO: 737 - protein, 10 aa, Epstein-Barr virus
GLLAAAYSQV

SEQ ID NO: 738 - protein, 9 aa, Epstein-Barr virus
GLLAELEAI

SEQ ID NO: 739 - protein, 10 aa, Epstein-Barr virus
GLLAELEAIL

SEQ ID NO: 740 - protein, 10 aa, Epstein-Barr virus
GPSGALSDAL

SEQ ID NO: 741 - protein, 10 aa, Epstein-Barr virus
HALTHSLYTF

SEQ ID NO: 742 - protein, 9 aa, Epstein-Barr virus
HFLYRRAFV

SEQ ID NO: 743 - protein, 9 aa, Epstein-Barr virus
HLFDAVAPG

SEQ ID NO: 744 - protein, 10 aa, Epstein-Barr virus
HLFDAVAPGA

SEQ ID NO: 745 - protein, 9 aa, Epstein-Barr virus
HLHALTHSL

SEQ ID NO: 746 - protein, 10 aa, Epstein-Barr virus
HLHALTHSLY

SEQ ID NO: 747 - protein, 9 aa, Epstein-Barr virus
HLLEAEEKA

SEQ ID NO: 748 - protein, 9 aa, Epstein-Barr virus
HLLENSREA

SEQ ID NO: 749 - protein, 9 aa, Epstein-Barr virus
HLQMRDDSA

SEQ ID NO: 750 - protein, 9 aa, Epstein-Barr virus
HQKAFLTSL

SEQ ID NO: 751 - protein, 9 aa, Epstein-Barr virus
HVSCNHLAV

SEQ ID NO: 752 - protein, 10 aa, Epstein-Barr virus
ILCRALREAV

SEQ ID NO: 753 - protein, 10 aa, Epstein-Barr virus
ILLYKGRGWV

SEQ ID NO: 754 - protein, 9 aa, Epstein-Barr virus
ILMKRINSV

SEQ ID NO: 755 - protein, 10 aa, Epstein-Barr virus
ILMKRINSVF

SEQ ID NO: 756 - protein, 10 aa, Epstein-Barr virus
IWPGIEPSDW

SEQ ID NO: 757 - protein, 9 aa, Epstein-Barr virus
KTWGRSLIL

SEQ ID NO: 758 - protein, 10 aa, Epstein-Barr virus
KTWGRSLILR

SEQ ID NO: 759 - protein, 9 aa, Epstein-Barr virus
KVDVSLCLI

SEQ ID NO: 760 - protein, 9 aa, Epstein-Barr virus
LAAAYSQVY

SEQ ID NO: 761 - protein, 9 aa, Epstein-Barr virus
LAGARLTHL

SEQ ID NO: 762 - protein, 9 aa, Epstein-Barr virus
LARMRNHFL

SEQ ID NO: 763 - protein, 10 aa, Epstein-Barr virus
LFARERRLAL

SEQ ID NO: 764 - protein, 10 aa, Epstein-Barr virus
LGRFRGVSPI

SEQ ID NO: 765 - protein, 9 aa, Epstein-Barr virus
LILRRADAV

SEQ ID NO: 766 - protein, 9 aa, Epstein-Barr virus
LINDIEILM

SEQ ID NO: 767 - protein, 10 aa, Epstein-Barr virus
LINDIEILMK

SEQ ID NO: 768 - protein, 9 aa, Epstein-Barr virus
LLAAAYSQV

SEQ ID NO: 769 - protein, 10 aa, Epstein-Barr virus
LLAAAYSQVY

SEQ ID NO: 770 - protein, 9 aa, Epstein-Barr virus
LLAELEAIL

SEQ ID NO: 771 - protein, 10 aa, Epstein-Barr virus
LLAELEAILL

SEQ ID NO: 772 - protein, 9 aa, Epstein-Barr virus
LLYKGRGWV

SEQ ID NO: 773 - protein, 10 aa, Epstein-Barr virus
LLYKGRGWVF

SEQ ID NO: 774 - protein, 9 aa, Epstein-Barr virus
LLYLHLQMR

SEQ ID NO: 775 - protein, 9 aa, Epstein-Barr virus
LLYWNSGGH

SEQ ID NO: 776 - protein, 10 aa, Epstein-Barr virus
LLYWNSGGHA

SEQ ID NO: 777 - protein, 9 aa, Epstein-Barr virus
LMKRINSVF

SEQ ID NO: 778 - protein, 10 aa, Epstein-Barr virus
LMKRINSVFY

SEQ ID NO: 779 - protein, 9 aa, Epstein-Barr virus
LPNHGNPST

SEQ ID NO: 780 - protein, 10 aa, Epstein-Barr virus
LPNHGNPSTA

SEQ ID NO: 781 - protein, 9 aa, Epstein-Barr virus
LSLELVHLL

SEQ ID NO: 782 - protein, 10 aa, Epstein-Barr virus
LVRRACEEAL

SEQ ID NO: 783 - protein, 9 aa, Epstein-Barr virus
LYALLYLHL

SEQ ID NO: 784 - protein, 9 aa, Epstein-Barr virus
LYITSVPCW

SEQ ID NO: 785 - protein, 9 aa, Epstein-Barr virus
LYKGRGWVF

SEQ ID NO: 786 - protein, 10 aa, Epstein-Barr virus
LYNKTWGRSL

SEQ ID NO: 787 - protein, 9 aa, Epstein-Barr virus
LYTFITGPL

SEQ ID NO: 788 - protein, 10 aa, Epstein-Barr virus
LYWNSGGHAI

SEQ ID NO: 789 - protein, 9 aa, Epstein-Barr virus
MSGLLAAAY

SEQ ID NO: 790 - protein, 9 aa, Epstein-Barr virus
NSREASAAL

SEQ ID NO: 791 - protein, 10 aa, Epstein-Barr virus
NVFSTVPPEV

SEQ ID NO: 792 - protein, 9 aa, Epstein-Barr virus
PQYRAILQK

SEQ ID NO: 793 - protein, 9 aa, Epstein-Barr virus
QVYALAVEL

SEQ ID NO: 794 - protein, 10 aa, Epstein-Barr virus
RAILQKRNEL

SEQ ID NO: 795 - protein, 9 aa, Epstein-Barr virus
RLDPRSLDV

SEQ ID NO: 796 - protein, 9 aa, Epstein-Barr virus
RLFCGGVYS

SEQ ID NO: 797 - protein, 10 aa, Epstein-Barr virus
RLFCGGVYSS

SEQ ID NO: 798 - protein, 9 aa, Epstein-Barr virus
RLNRAMQRL

SEQ ID NO: 799 - protein, 10 aa, Epstein-Barr virus
RLSALFARER

SEQ ID NO: 800 - protein, 9 aa, Epstein-Barr virus
RLTHLFDAV

SEQ ID NO: 801 - protein, 9 aa, Epstein-Barr virus
RMRNHFLYR

SEQ ID NO: 802 - protein, 10 aa, Epstein-Barr virus
RMRNHFLYRR

SEQ ID NO: 803 - protein, 9 aa, Epstein-Barr virus
RPAGAREPG

SEQ ID NO: 804 - protein, 10 aa, Epstein-Barr virus
RPGGPEEGAV

SEQ ID NO: 805 - protein, 9 aa, Epstein-Barr virus
RQAGLGHLL

SEQ ID NO: 806 - protein, 10 aa, Epstein-Barr virus
RQVATEGLAK

SEQ ID NO: 807 - protein, 9 aa, Epstein-Barr virus
RTPSVAYSV

SEQ ID NO: 808 - protein, 10 aa, Epstein-Barr virus
SAALLAPGRK

SEQ ID NO: 809 - protein, 9 aa, Epstein-Barr virus
SLASSQQIL

SEQ ID NO: 810 - protein, 10 aa, Epstein-Barr virus
SLCLINDIEI

SEQ ID NO: 811 - protein, 9 aa, Epstein-Barr virus
SLILRRADA

SEQ ID NO: 812 - protein, 10 aa, Epstein-Barr virus
SLILRRADAV

SEQ ID NO: 813 - protein, 9 aa, Epstein-Barr virus
SLYNKTWGR

SEQ ID NO: 814 - protein, 10 aa, Epstein-Barr virus
SLYTFITGPL

SEQ ID NO: 815 - protein, 10 aa, Epstein-Barr virus
SPEVPRPAGA

SEQ ID NO: 816 - protein, 10 aa, Epstein-Barr virus
SPIPDPRLYI

SEQ ID NO: 817 - protein, 10 aa, Epstein-Barr virus
SQVYALAVEL

SEQ ID NO: 818 - protein, 9 aa, Epstein-Barr virus
SVAYSVEFY

SEQ ID NO: 819 - protein, 9 aa, Epstein-Barr virus
SVEFYGGHK

SEQ ID NO: 820 - protein, 10 aa, Epstein-Barr virus
SVFYCMSHTM

SEQ ID NO: 821 - protein, 10 aa, Epstein-Barr virus
TLSSEHLHAL

SEQ ID NO: 822 - protein, 10 aa, Epstein-Barr virus
TLTETLCLRV

SEQ ID NO: 823 - protein, 10 aa, Epstein-Barr virus
TPSVAYSVEF

SEQ ID NO: 824 - protein, 9 aa, Epstein-Barr virus
TSFNSFYSV

SEQ ID NO: 825 - protein, 9 aa, Epstein-Barr virus
TYDSDRPLI

SEQ ID NO: 826 - protein, 9 aa, Epstein-Barr virus
VAELSELLY

SEQ ID NO: 827 - protein, 9 aa, Epstein-Barr virus
VFKDLYALL

SEQ ID NO: 828 - protein, 9 aa, Epstein-Barr virus
VFYCMSHTM

SEQ ID NO: 829 - protein, 9 aa, Epstein-Barr virus
VLTVSLYNK

SEQ ID NO: 830 - protein, 10 aa, Epstein-Barr virus
VPCWRCVGEL

SEQ ID NO: 831 - protein, 9 aa, Epstein-Barr virus
VPGPGRPEA

SEQ ID NO: 832 - protein, 9 aa, Epstein-Barr virus
VPPLRTPSV

SEQ ID NO: 833 - protein, 10 aa, Epstein-Barr virus
VPPLRTPSVA

SEQ ID NO: 834 - protein, 9 aa, Epstein-Barr virus
VPRPAGARE

SEQ ID NO: 835 - protein, 10 aa, Epstein-Barr virus
VPRPAGAREP

SEQ ID NO: 836 - protein, 10 aa, Epstein-Barr virus
VVYWELARMR

SEQ ID NO: 837 - protein, 10 aa, Epstein-Barr virus
VWGDVVYWEL

SEQ ID NO: 838 - protein, 10 aa, Epstein-Barr virus
VYALAVELSV

SEQ ID NO: 839 - protein, 10 aa, Epstein-Barr virus
WIETSFNSFY

SEQ ID NO: 840 - protein, 9 aa, Epstein-Barr virus
WVFKDLYAL

SEQ ID NO: 841 - protein, 10 aa, Epstein-Barr virus
WVFKDLYALL

SEQ ID NO: 842 - protein, 9 aa, Epstein-Barr virus
YALAVELSV

SEQ ID NO: 843 - protein, 10 aa, Epstein-Barr virus
YALLYLHLQM

SEQ ID NO: 844 - protein, 9 aa, Epstein-Barr virus
YCMSHTMGL

SEQ ID NO: 845 - protein, 10 aa, Epstein-Barr virus
YLRQVATEGL

SEQ ID NO: 846 - protein, 10 aa, Epstein-Barr virus
YLTYDSDRPL

SEQ ID NO: 847 - protein, 9 aa, Epstein-Barr virus
YNKTWGRSL

SEQ ID NO: 848 - protein, 9 aa, Epstein-Barr virus
YTFITGPLA

SEQ ID NO: 849 - protein, 9 aa, Epstein-Barr virus
YWNSGGHAI

SEQ ID NO: 850 - protein, 10 aa, Epstein-Barr virus
AARPRLLLSL

SEQ ID NO: 851 - protein, 9 aa, Epstein-Barr virus
AARVPIEEL

SEQ ID NO: 852 - protein, 9 aa, Epstein-Barr virus
AGMSATLPL

SEQ ID NO: 853 - protein, 10 aa, Epstein-Barr virus
ALLRQAGLQL

SEQ ID NO: 854 - protein, 9 aa, Epstein-Barr virus
ALLWAARPR

SEQ ID NO: 855 - protein, 10 aa, Epstein-Barr virus
ALLWAARPRL

SEQ ID NO: 856 - protein, 9 aa, Epstein-Barr virus
ALPGRLLLA

SEQ ID NO: 857 - protein, 9 aa, Epstein-Barr virus
APAGHRGDV

SEQ ID NO: 858 - protein, 10 aa, Epstein-Barr virus
APAGHRGDVE

SEQ ID NO: 859 - protein, 9 aa, Epstein-Barr virus
APGYAVEAV

SEQ ID NO: 860 - protein, 10 aa, Epstein-Barr virus
AVEAVEGGLY

SEQ ID NO: 861 - protein, 9 aa, Epstein-Barr virus
AVEGGLYPV

SEQ ID NO: 862 - protein, 10 aa, Epstein-Barr virus
AVRLQRLHRV

SEQ ID NO: 863 - protein, 9 aa, Epstein-Barr virus
AVRQRVQVL

SEQ ID NO: 864 - protein, 9 aa, Epstein-Barr virus
CPRQRLLAG

SEQ ID NO: 865 - protein, 10 aa, Epstein-Barr virus
CPSRPGHLRA

SEQ ID NO: 866 - protein, 9 aa, Epstein-Barr virus
CTDSMAARV

SEQ ID NO: 867 - protein, 9 aa, Epstein-Barr virus
EPRVELVPL

SEQ ID NO: 868 - protein, 10 aa, Epstein-Barr virus
EPRVELVPLL

SEQ ID NO: 869 - protein, 9 aa, Epstein-Barr virus
FQALQPHGV

SEQ ID NO: 870 - protein, 10 aa, Epstein-Barr virus
GLALLRQAGL

SEQ ID NO: 871 - protein, 10 aa, Epstein-Barr virus
GLGKQVCFDV

SEQ ID NO: 872 - protein, 9 aa, Epstein-Barr virus
GLGQGLALL

SEQ ID NO: 873 - protein, 10 aa, Epstein-Barr virus
GMSATLPLPR

SEQ ID NO: 874 - protein, 9 aa, Epstein-Barr virus
GPLCRGRVA

SEQ ID NO: 875 - protein, 10 aa, Epstein-Barr virus
GPLCRGRVAV

SEQ ID NO: 876 - protein, 10 aa, Epstein-Barr virus
GPPHQGQATL

SEQ ID NO: 877 - protein, 9 aa, Epstein-Barr virus
GPRDEGVQA

SEQ ID NO: 878 - protein, 10 aa, Epstein-Barr virus
GPRDEGVQAV

SEQ ID NO: 879 - protein, 9 aa, Epstein-Barr virus
GVQRSGRPL

SEQ ID NO: 880 - protein, 10 aa, Epstein-Barr virus
GVRREGGGAV

SEQ ID NO: 881 - protein, 10 aa, Epstein-Barr virus
HAIKHAIDSL

SEQ ID NO: 882 - protein, 9 aa, Epstein-Barr virus
HLAQVLLLA

SEQ ID NO: 883 - protein, 10 aa, Epstein-Barr virus
HLAQVLLLAL

SEQ ID NO: 884 - protein, 9 aa, Epstein-Barr virus
HLRALPGRL

SEQ ID NO: 885 - protein, 10 aa, Epstein-Barr virus
HLRALPGRLL

SEQ ID NO: 886 - protein, 10 aa, Epstein-Barr virus
HLRGHCREDV

SEQ ID NO: 887 - protein, 9 aa, Epstein-Barr virus
HQLAHTAPA

SEQ ID NO: 888 - protein, 10 aa, Epstein-Barr virus
ILKGGTLAGA

SEQ ID NO: 889 - protein, 10 aa, Epstein-Barr virus
KQVCFDVLGI

SEQ ID NO: 890 - protein, 9 aa, Epstein-Barr virus
LALLRQAGL

SEQ ID NO: 891 - protein, 9 aa, Epstein-Barr virus
LCRGRVAVV

SEQ ID NO: 892 - protein, 9 aa, Epstein-Barr virus
LLASAQPLH

SEQ ID NO: 893 - protein, 9 aa, Epstein-Barr virus
LLLASAQPL

SEQ ID NO: 894 - protein, 10 aa, Epstein-Barr virus
LLQDGPVLGV

SEQ ID NO: 895 - protein, 9 aa, Epstein-Barr virus
LLRQAGLQL

SEQ ID NO: 896 - protein, 10 aa, Epstein-Barr virus
LLVRQRTCGV

SEQ ID NO: 897 - protein, 9 aa, Epstein-Barr virus
LLWAARPRL

SEQ ID NO: 898 - protein, 10 aa, Epstein-Barr virus
LLWAARPRLL

SEQ ID NO: 899 - protein, 9 aa, Epstein-Barr virus
LPGKQGREA

SEQ ID NO: 900 - protein, 10 aa, Epstein-Barr virus
LPGRLLLASA

SEQ ID NO: 901 - protein, 10 aa, Epstein-Barr virus
LPLPRCTDSM

SEQ ID NO: 902 - protein, 9 aa, Epstein-Barr virus
LPRCTDSMA

SEQ ID NO: 903 - protein, 10 aa, Epstein-Barr virus
LPRCTDSMAA

SEQ ID NO: 904 - protein, 9 aa, Epstein-Barr virus
LQDGPVLGV

SEQ ID NO: 905 - protein, 9 aa, Epstein-Barr virus
LRIHRHRQV

SEQ ID NO: 906 - protein, 10 aa, Epstein-Barr virus
LRIHRHRQVV

SEQ ID NO: 907 - protein, 9 aa, Epstein-Barr virus
LVRQRTCGV

SEQ ID NO: 908 - protein, 10 aa, Epstein-Barr virus
LWAARPRLLL

SEQ ID NO: 909 - protein, 10 aa, Epstein-Barr virus
LYPVARLDAW

SEQ ID NO: 910 - protein, 9 aa, Epstein-Barr virus
MSATLPLPR

SEQ ID NO: 911 - protein, 9 aa, Epstein-Barr virus
PPHQGQATL

SEQ ID NO: 912 - protein, 9 aa, Epstein-Barr virus
PPRARDRAL

SEQ ID NO: 913 - protein, 10 aa, Epstein-Barr virus
PPRARDRALL

SEQ ID NO: 914 - protein, 9 aa, Epstein-Barr virus
QPCPRQRLL

SEQ ID NO: 915 - protein, 9 aa, Epstein-Barr virus
QPHGVRHAI

SEQ ID NO: 916 - protein, 10 aa, Epstein-Barr virus
QTLGGHLAQV

SEQ ID NO: 917 - protein, 10 aa, Epstein-Barr virus
QVLRAQGLGK

SEQ ID NO: 918 - protein, 10 aa, Epstein-Barr virus
RALLWAARPR

SEQ ID NO: 919 - protein, 9 aa, Epstein-Barr virus
RALPGRLLL

SEQ ID NO: 920 - protein, 10 aa, Epstein-Barr virus
RARDRALLWA

SEQ ID NO: 921 - protein, 9 aa, Epstein-Barr virus
RIHRHRQVV

SEQ ID NO: 922 - protein, 10 aa, Epstein-Barr virus
RLLLASAQPL

SEQ ID NO: 923 - protein, 9 aa, Epstein-Barr virus
RLLLSLQQV

SEQ ID NO: 924 - protein, 10 aa, Epstein-Barr virus
RLRIHRHRQV

SEQ ID NO: 925 - protein, 10 aa, Epstein-Barr virus
RPGHLRALPG

SEQ ID NO: 926 - protein, 10 aa, Epstein-Barr virus
RPLCLRPPRA

SEQ ID NO: 927 - protein, 9 aa, Epstein-Barr virus
RPPRARDRA

SEQ ID NO: 928 - protein, 10 aa, Epstein-Barr virus
RPPRARDRAL

SEQ ID NO: 929 - protein, 10 aa, Epstein-Barr virus
RPRLLLSLQQ

SEQ ID NO: 930 - protein, 10 aa, Epstein-Barr virus
RVQVLRAQGL

SEQ ID NO: 931 - protein, 9 aa, Epstein-Barr virus
RVREGAGRA

SEQ ID NO: 932 - protein, 10 aa, Epstein-Barr virus
RVREGAGRAG

SEQ ID NO: 933 - protein, 10 aa, Epstein-Barr virus
RVWDGTYAPK

SEQ ID NO: 934 - protein, 10 aa, Epstein-Barr virus
SLQQVPEPSL

SEQ ID NO: 935 - protein, 10 aa, Epstein-Barr virus
SQGHVAGWGK

SEQ ID NO: 936 - protein, 9 aa, Epstein-Barr virus
TLGGHLAQV

SEQ ID NO: 937 - protein, 10 aa, Epstein-Barr virus
TLGGHLAQVL

SEQ ID NO: 938 - protein, 10 aa, Epstein-Barr virus
TYAPKAAQQI

SEQ ID NO: 939 - protein, 9 aa, Epstein-Barr virus
VLLLALERV

SEQ ID NO: 940 - protein, 9 aa, Epstein-Barr virus
VLRAQGLGK

SEQ ID NO: 941 - protein, 9 aa, Epstein-Barr virus
VPIEELREF

SEQ ID NO: 942 - protein, 9 aa, Epstein-Barr virus
VPLLQDGPV

SEQ ID NO: 943 - protein, 10 aa, Epstein-Barr virus
VPLLQDGPVL

SEQ ID NO: 944 - protein, 10 aa, Epstein-Barr virus
VVAHAGQLPV

SEQ ID NO: 945 - protein, 9 aa, Epstein-Barr virus
WAARPRLLL

SEQ ID NO: 946 - protein, 10 aa, Epstein-Barr virus
WPYQGSQERL

SEQ ID NO: 947 - protein, 9 aa, Epstein-Barr virus
YPVARLDAW